(19) United States
(12) Patent Application Publication
(10) Pub. No.: US 2015/0272934 A1
(43) Pub. Date: Oct. 1, 2015

(54) USE OF CSE INHIBITORS FOR THE TREATMENT OF CUTANEOUS INJURIES OR CONDITIONS AND SLEEP-RELATED BREATHING DISORDERS

(71) Applicant: SOVA PHARMACEUTICALS, INC., La Jolla, CA (US)

(72) Inventors: Gregory Stein, San Diego, CA (US); Srinivas G. Rao, Encinitas, CA (US); Sergio G. Durón, San Diego, CA (US); Justin Chapman, San Diego, CA (US); Simon G. Sydserff, San Diego, CA (US)

(21) Appl. No.: 14/416,603

(22) PCT Filed: Jul. 23, 2013
(86) PCT No.: PCT/US2013/051746

§ 371 Date: Jan. 22, 2015

Related U.S. Application Data
(60) Provisional application No. 61/675,757, filed on Jul. 25, 2012.
(60) Provisional application No. 61/675,755, filed on Jul. 25, 2012.

(51) Int. Cl. A61K 31/4245; A61K 45/06; A61K 31/4196; A61K 31/198; A61K 31/41

(52) U.S. Cl. CPC A61K 31/4245; A61K 31/198; A61K 31/41; A61K 31/4196; A61K 45/06

(57) ABSTRACT

Described are methods of treatment of cutaneous injuries or conditions comprising administration of CSE inhibitors to individuals in need thereof. Also described herein are methods of treatment of sleep-related breathing disorders comprising administration of CSE inhibitors to individuals in need thereof.

### CROSS-REFERENCE

This application claims the benefit of U.S. Application No. 61/675,755, filed Jul. 25, 2012, and U.S. Application No. 61/675,757, filed Jul. 25, 2012, both of which are incorporated by reference in their entirery.

### BACKGROUND OF THE INVENTION

Treatment of cutaneous injuries or conditions is an ongoing medical problem. In some cases, despite treatment, individuals have to cope with the sequelae of scarring and/or contractures.

Sleep-related breathing disorders (SRBDs) include a continuum of conditions ranging from primary snoring to upper airway resistance syndrome (UARS) to obstructive sleep apnea (OSA) or, in some cases, obesity hypoventilation syndrome (OHS). They are omnipresent in our society and are gaining recognition for their effects on quality of life as well as their direct relationship with well-accepted diseases such as hypertension, stroke, diabetes, and congestive heart failure.

### SUMMARY OF THE INVENTION

Provided herein, in some embodiments, are methods for the treatment of cutaneous injuries or conditions comprising administration of therapeutically effective amounts of cystathionine γ-lyase (CSE) inhibitors to individuals in need thereof. In some embodiments, a cutaneous injury or condition is a skin injury such as a skin burn or a contracture (permanent tightening of the skin subsequent to an injury). In some embodiments, a cutaneous injury or condition is a skin deformity such as a boil, a pustule, a pimple, a blister, a skin ulcer (e.g., diabetic foot ulcer) and the like. In some embodiments, a cutaneous injury or condition is an allergic skin inflammation—such as a rash, or hives—, is associated with an infection—e.g., staphylococcal scalded skin syndrome, toxic shock syndrome—, or both—e.g., toxic epidermal necrolysis, Steven-Johnson syndrome and the like. In some embodiments, a cutaneous injury or condition is an inherited condition; e.g., epidermolysis bullosa. In some embodiments, the methods of treatment described herein improve wound healing (e.g., burn wound healing). Further provided herein are methods for promoting wound healing comprising administration of any CSE inhibitor described herein (e.g., compounds of Formula 1-I, Formula 1-II, Formula 1-IIa, Formula 1-III, Formula 1-IV, Formula 1-IVa, Formula 2-1, Formula 241, Formula 2-III, Formula 24V, Formula 2-V, or Formula 2-VI) to an individual in need thereof.

Provided herein is a method for treating a cutaneous injury or condition selected from a cutaneous burn, a cutaneous contracture, cutaneous scarring, cutaneous skin ulcers, pustules, blisters, staphylococcal scalded skin syndrome, toxic epidermal necrolysis, Stevens-Johnson Syndrome, epidermolysis bullosa and toxic shock syndrome in an individual in need thereof comprising administering a therapeutically effective amount of a cystathionine gamma lyase (CSE) inhibitor to the individual in need thereof.

In some embodiments of the method, the cutaneous injury or condition is a cutaneous burn

In some embodiments of the method, the burn is a thermal, chemical, or electrical burn.

In some embodiments of the method, the cutaneous burn is a burn injury due to fire, a scald, chemical burn, road rash, radiation burn, prolonged transfer of heat from an object to the skin, electric burn, sun burn, burn injury due to lightning strike, or inflamed skin due to contact with an allergen. In some embodiments, the cutaneous burn is a severe partial thickness or full thickness burn. In some embodiments of the method, the cutaneous injury or condition is a cutaneous contracture. In some embodiments of the method, the cutaneous contracture is associated with a deep tissue burn injury, a burn, or skin graft surgery. In some embodiments of the method, the blisters are associated with epidermolysis, chafing, scalds, insect bites or a cutaneous burn. In some embodiments of the method, the cutaneous scarring is further associated with fibrosis.

In some embodiments of the method, the cutaneous injury or condition is staphylococcal scalded skin syndrome, toxic epidermal necrolysis, Stevens-Johnson Syndrome, epidermolysis bullosa or toxic shock syndrome.

In some embodiments of the method, the CSE inhibitor is administered orally. In some embodiments of the method, the CSE inhibitor is administered topically on the skin. In some embodiments of the method, the CSE inhibitor is administered as a wash for the affected area. In some embodiments of the method, the CSE inhibitor is administered intravenously.

In some embodiments of the method, the CSE inhibitor is administered in combination with an anti-inflammatory agent, a pain medication, an antiseptic agent or a local anesthetic. In some embodiments of the method, the CSE inhibitor is administered in combination with a wound dressing.

In some embodiments of the method, the CSE inhibitor is L-propargylglycine. In some embodiments of the method, the CSE inhibitor is beta-cyanoalanine. In some embodiments of the method, the CSE inhibitor is 2-aminopent-4-ynoic acid, (S)-2-aminopent-4-ynoic acid, 2-amino-3-cyanopropanoic acid, (S)-2-amino-3-cyanopropanoic acid, 2-hydrazinylacetic acid hydrochloride, 2-(2-(propan-2-ylidene)hydrazinyl)acetic acid, 4-((2-(1H-tetrazol-5-yl)hydrazinyl)methyl)-N,N-dimethylaniline, (E)-4-((2-(1H-tetrazol-5-yl)hydrazono)methyl)-N,N-diethylaniline, (E)-1-((2-(1H-tetrazol-5-yl)hydrazono)methyl)naphthalen-2-ol, (E)-5-(2-(benzo[d][1,3]dioxol-5-ylmethylene)hydrazinyl)-1H-tetrazole, (E)-4-((2-(1H-tetrazol-5-yl)hydrazono)methyl)phenol, (E)-5-(2-(4-nitrobenzylidene)hydrazinyl)-1H-tetrazole, (E)-5-(2-(furan-2-ylmethylene)hydrazinyl)-1H-tetrazole, 5-hydrazinyl-1H-tetrazole, 5-(1-methylhydrazinyl)-1H-tetrazole, 5-(1-methylhydrazinyl)-1H-1,2,4-triazol-3(2H)-one, 5-(1-ethylhydrazinyl)-1H-1,2,4-triazol-3(2H)-one, or 5-(hydrazinylmethyl)-1H-tetrazole.

Also described herein are inhibitors of cystathionine-γ-lyase (CSE) having the structure of Formula (1-I), (1-II), (1-IIa), (1-III), (1-IV), (1-IVa), (2-I), (2-II), (2-III), (2-IV), (2-V), or (2-VI). Also disclosed herein are methods for synthesizing such CSE inhibitors and methods for using such CSE inhibitors in the treatment of diseases wherein CSE inhibition provides therapeutic benefit to the patient having the disease. Further described are pharmaceutical formulations that include a CSE inhibitor.

Provided herein, in some embodiments, is a method for treating or preventing or reducing incidence or severity of a sleep-related breathing disorder (SRBD) or its sequelae in an individual in need thereof comprising administering a therapeutically effective amount of a cystathionine gamma lyase (CSE) inhibitor to the individual in need thereof. Also provided herein, in some embodiments, are methods for treating or preventing or reducing the incidence or severity of a sleep-related breathing disorder (SRBD) or its sequelae in individuals in need thereof comprising administration of a compound of Formula (1-I), (1-II), (1-IIa), (1-III), (1-IV), (1-IVa), (2-I), (2-II), (2-III), (2-IV), (2-V), or (2-VI) to the individual in need thereof. In some of such embodiments, the individual is suffering from or suspected to be suffering from an SRBD selected from central sleep apnea (CSA), Cheyne-Stokes breathing (CSB), obesity hypoventilation syndrome (OHS), congenital central hypoventilation syndrome (CCHS), obstructive sleep apnea (OSA), obstructive sleep apnea syndrome (OSAS), upper airway resistance syndrome (UARS), idiopathic central sleep apnea (ICSA), opioid-induced CSA, apnea of prematurity, primary snoring, high altitude periodic breathing, chronic mountain sickness, impaired respiratory motor control associated with stroke, or impaired respiratory motor control associated with a neurologic disorder.

Also provided herein, in some embodiments, are methods for treating or preventing or reducing the incidence or severity of an SRBD or its sequelae in individuals in need thereof comprising administration of 2-aminopent-4-ynoic acid, (S)-2-aminopent-4-ynoic acid, 2-amino-3-cyanopropanoic acid, (S)-2-amino-3-cyanopropanoic acid, 2-hydrazinylacetic acid hydrochloride, 2-(2-(propan-2-ylidene)hydrazinyl)acetic acid, 4-((2-(1H-tetrazol-5-yl)hydrazinyl)methyl)-N,N-dimethylaniline, (E)-4-((2-(1H-tetrazol-5-yl)hydrazono)methyl)-N,N-diethylaniline, (E)-1-((2-(1H-tetrazol-5-yl)hydrazono)methyl)naphthalen-2-ol, (E)-5-(2-(benzo[d][1,3]dioxol-5-ylmethylene)hydrazinyl)-1H-tetrazole, (E)-4-((2-(1H-tetrazol-5-yl)hydrazono)methyl)phenol, (E)-5-(2-(4-nitrobenzylidene)hydrazinyl)-1H-tetrazole, (E)-5-(2-(furan-2-ylmethylene)hydrazinyl)-1H-tetrazole, 5-hydrazinyl-1H-tetrazole, 5-(1-methylhydrazinyl)-1H-tetrazole, 5-(1-methylhydrazinyl)-1H-1,2,4-triazol-3(2H)-one, 5-(1-ethylhydrazinyl)-1H-1,2,4-triazol-3(2H)-one, or 5-(hydrazinylmethyl)-1H-tetrazole to the individual in need thereof. In some of such embodiments, the individual is suffering from or suspected to be suffering from an SRBD selected from central sleep apnea (CSA), Cheyne-Stokes breathing-central sleep apnea (CSB-CSA), obesity hypoventilation syndrome (OHS), congenital central hypoventilation syndrome (CCHS), obstructive sleep apnea (OSA), obstructive sleep apnea syndrome (OSAS), upper airway resistance syndrome (UARS), idiopathic central sleep apnea (ICSA), opioid-induced CSA, apnea of prematurity, primary snoring, high altitude periodic breathing, chronic mountain sickness, impaired respiratory motor control associated with stroke, or impaired respiratory motor control associated with a neurologic disorder.

In some specific embodiments of the method, the individual is suffering from or suspected to be suffering from central sleep apnea (CSA). In some specific embodiments, the individual is suffering from or suspected to be suffering from Cheyne-Stokes breathing-central sleep apnea (CSB-CSA). In some specific embodiments, the individual is suffering from or suspected to be suffering from obesity hypoventilation syndrome (OHS). In some specific embodiments, the individual is suffering from or suspected to be suffering from congenital central hypoventilation syndrome (CCHS). In some specific embodiments, the individual is suffering from or suspected to be suffering from obstructive sleep apnea (OSA). In some specific embodiments, the individual is suffering from or suspected to be suffering from obstructive sleep apnea syndrome (OSAS). In some specific embodiments, the individual is suffering from or suspected to be suffering from upper airway resistance syndrome (UARS). In some specific embodiments, the individual is suffering from or suspected to be suffering from idiopathic central sleep apnea (ICSA). In some specific embodiments, the individual is suffering from or suspected to be suffering from opioid-induced CSA. In some specific embodiments, the individual is suffering from or suspected to be suffering from apnea of prematurity. In some specific embodiments, the individual is suffering from or suspected to be suffering from primary snoring. In some specific embodiments, the individual is suffering from or suspected to be suffering from high altitude periodic breathing. In some specific embodiments, the individual is suffering from or suspected to be suffering from chronic mountain sickness. In some specific embodiments, the individual is suffering from or suspected to be suffering from impaired respiratory motor control associated with stroke. In some specific embodiments, the individual is suffering from or suspected to be suffering from impaired respiratory motor control associated with a neurologic disorder.

In some embodiments the SRBD is a symptom of myasthenia gravis, amyotrophic lateral sclerosis, post-polio syndrome, myopathies, congenital myopathies, neuropathies, myotonic dystrophy, Duchenne's dystrophy, mitochondrial encephalomyopathy, stroke, epilepsy, Parkinsonism, Alzheimer's disease, Huntington's disease, congenital muscular dystrophy, cerebral palsy, spinal muscular atrophy, transverse myelitis, or poliomyelitis. In some specific embodiments, the SRBD is a symptom of myasthenia gravis. In some specific embodiments, the SRBD is a symptom of amyotrophic lateral sclerosis. In some specific embodiments, the SRBD is a symptom of post-polio syndrome. In some specific embodiments, the SRBD is a symptom of myopathies. In some specific embodiments, the SRBD is a symptom of congenital myopathies. In some specific embodiments, the SRBD is a symptom of neuropathies. In some specific embodiments, the SRBD is a symptom of myotonic dystrophy. In some specific embodiments, the SRBD is a symptom of Duchenne's dystrophy. In some specific embodiments, the SRBD is a symptom of myasthenia gravis. In some specific embodiments, the SRBD is a symptom of mitochondrial encephalomyopathy. In some specific embodiments, the SRBD is a symptom of stroke. In some specific embodiments, the SRBD is a symptom of epilepsy. In some specific embodiments, the SRBD is a symptom of Parkinsonism. In some specific embodiments, the SRBD is a symptom of Alzheimer's disease. In some specific embodiments, the SRBD is a symptom of Huntington's disease. In some specific embodiments, the SRBD is a symptom of congenital muscular dystrophy. In some specific embodiments, the SRBD is a symptom of cerebral palsy. In some specific embodiments, the SRBD is a symptom of spinal muscular atrophy. In some specific embodiments, the SRBD is a symptom of transverse myelitis. In some specific embodiments, the SRBD is a symptom of poliomyelitis.

Disclosed herein, in certain embodiments, are methods of treating an individual with an SRBD wherein the individual with the SRBD is using a continuous positive airway pressure (CPAP) device, an adaptive servo-ventilation (ASV) device, or any other device that provides positve pressure support to the airway, either actively or passively, for treatment of their SRBD.

In some embodiments, the method further comprises administrating a second agent selected from carbonic anhydrase inhibitors, cholinesterase inhibitors, adenosine inhibitors, progestational agents, opiod antagonists, central nervous system stimulants, selective serotonin reuptake inhibitors (SSRIs), antidepressants, antihypertensives, calcium channel antagonists, ACE inhibitors, respiratory stimulants, alpha-2 adrenergic agonists, gamma aminobutyric acid agonists, and glutamate antagonists. In some embodiments, the method further comprises administering a second agent selected from acetazolamide, theophylline, progesterone, donepezil, naloxone, nicotine, paroxetine, protriptyline, metoprolol, cilazapril, propranolol, atenolol, hydrochlorothiazide, isradipine, spirapril, doxapram, clonidine, baclofen, and sabeluzole.

In some embodiments of the method, the compound of Formula (1-I), (1-II), (1-IIa), (1-III), (1-IV), (1-IVa), (2-I), (2-II), (2-III), (2-IV), (2-V), or (2-VI) that inhibits or partially inhibits the activity of cystathionine-γ-lyase (CSE) is administered orally, subcutaneously, topically, intramuscularly, or intravenously. In some embodiments of the method, 2-aminopent-4-ynoic acid, (S)-2-aminopent-4-ynoic acid, 2-amino-3-cyanopropanoic acid, (S)-2-amino-3-cyanopropanoic acid, 2-hydrazinylacetic acid hydrochloride, 2-(2-(propan-2-ylidene)hydrazinyl)acetic acid, 4-((2-(1H-tetrazol-5-yl)hydrazinyl)methyl)-N,N-dimethylaniline, (E)-4-((2-(1H-tetrazol-5-yl)hydrazono)methyl)-N,N-diethylaniline, (E)-1-((2-(1H-tetrazol-5-yl)hydrazono)methyl)naphthalen-2-ol, (E)-5-(2-(benzo[d][1,3]dioxol-5-ylmethylene)hydrazinyl)-1H-tetrazole, (E)-4-((2-(1H-tetrazol-5-yl)hydrazono)methyl)phenol, (E)-5-(2-(4-nitrobenzylidene)hydrazinyl)-1H-tetrazole, (E)-5-(2-(furan-2-ylmethylene)hydrazinyl)-1H-tetrazole, 5-hydrazinyl-1H-tetrazole, 5-(1-methylhydrazinyl)-1H-tetrazole, 5-(1-methylhydrazinyl)-1H-1,2,4-triazol-3(2H)-one, 5-(1-ethylhydrazinyl)-1H-1,2,4-triazol-3(2H)-one, or 5-(hydrazinylmethyl)-1H-tetrazole that inhibits or partially inhibits the activity of cystathionine-γ-lyase (CSE) is administered orally, subcutaneously, topically, intramuscularly, or intravenously.

In some of the aforementioned embodiments of the method, a compound of Formula (1-I), (1-II), (1-IIa), (1-III), (1-IV), (1-IVa), (2-V), (2-IV), (2-III), (2-IV), (2-V), or (2-VI) inhibits or partially inhibits the activity of cystathionine-gamma-lyase (CSE). In some embodiments, the compound of Formula (1-I), (1-II), (1-IIa), (1-III), (1-IV), (1-IVa), (2-I), (2-II), (2-III), (2-IV), (2-V), or (2-VI) that inhibits or partially inhibits the activity of CSE reduces the chemosensitivity of the carotid body to the partial pressure of oxygen in arterial blood, reduces the chemosensitivity of the carotid body to the partial pressure of carbon dioxide in arterial blood, reduces the loop gain of the ventilatory drive control system, lowers blood pressure, or dampens carotid sinus nerve activity in an individual in need thereof, or a combination thereof.

In some specific embodiments of the method, the compound of Formula (1-I), (1-II), (1-IIa), (1-III), (1-IV), (1-IVa), (2-I), (2-II), (2-III), (2-IV), (2-V), or (2-VI) that inhibits or partially inhibits the activity of CSE reduces the chemosensitivity of the carotid body in an individual in need thereof. In some embodiments, the compound of Formula (1-I), (1-II), (1-IIa), (1-III), (1-IV), (1-IVa), (2-I), (2-II), (2-III), (2-IV), (2-V), or (2-VI) that inhibits or partially inhibits the activity of CSE reduces the chemosensitivity of the carotid body to the partial pressure of oxygen in arterial blood. In some embodiments, the compound of Formula (1-I), (MI), (1-IIa), (1-III), (1-IV), (1-IVa), (2-I), (2-II), (2-III), (2-IV), (2-V), or (2-VI) that inhibits or partially inhibits the activity of CSE reduces the loop gain of the ventilatory drive control system in an individual in need thereof. In some embodiments, the compound of Formula (1-1), (1-II), (1-IIa), (1-III), (1-IV), (1-IVa), (2-I), (2-II), (2-III), (2-IV), (2-V), or (2-VI) that inhibits or partially inhibits the activity of CSE reduces blood pressure in an individual in need thereof. In some embodiments, the compound of Formula (1-I), (1-II), (1-IIa), (1-III), (1-IV), (1-IVa), (2-1), (2-II), (2-III), (2-IV), (2-V), or (2-VI) that inhibits or partially inhibits the activity of CSE dampens carotid sinus nerve activity.

In some of the aforementioned embodiments of the method, 2-aminopent-4-ynoic acid, (S)-2-aminopent-4-ynoic acid, 2-amino-3-cyanopropanoic acid, (S)-2-amino-3-cyanopropanoic acid, 2-hydrazinylacetic acid hydrochloride, 2-(2-(propan-2-ylidene)hydrazinyl)acetic acid, 4-((2-(1H-tetrazol-5-yl)hydrazinyl)methyl)-N,N-dimethylaniline, (E)-4-((2-(1H-tetrazol-5-yl)hydrazono)methyl)-N,N-diethylaniline, (E)-1-((2-(1H-tetrazol-5-yl)hydrazono)methyl)naphthalen-2-ol, (E)-5-(2-(benzo[d][1,3]dioxo 1-5-ylmethylene)hydrazinyl)-1H-tetrazole, (E)-4-((2-(1H-tetrazol-5-yl)hydrazono)methyl)phenol, (E)-5-(2-(4-nitrobenzylidene)hydrazinyl)-1H-tetrazole, (E)-5-(2-(furan-2-ylmethylene)hydrazinyl)-1H-tetrazole, 5-hydrazinyl-1H-tetrazole, 5-(1-methylhydrazinyl)-1H-tetrazole, 5-(1-methylhydrazinyl)-1H-1,2,4-triazol-3(2H)-one, 5-(1-ethylhydrazinyl)-1H-1,2,4-triazol-3(2H)-one, or 5-(hydrazinylmethyl)-1H-tetrazole inhibits or partially inhibits the activity of cystathionine-gamma-lyase (CSE). In some embodiments, 2-aminopent-4-ynoic acid, (S)-2-aminopent-4-ynoic acid, 2-amino-3-cyanopropanoic acid, (S)-2-amino-3-cyanopropanoic acid, 2-hydrazinylacetic acid hydrochloride, 2-(2-(propan-2-ylidene)hydrazinyl)acetic acid, 4-((2-(1H-tetrazol-5-yl)hydrazinyl)methyl)-N,N-dimethylaniline, (E)-4-((2-(1H-tetrazol-5-yl)hydrazono)methyl)-N,N-diethylaniline, (E)-1-((2-(1H-tetrazol-5-yl)hydrazono)methyl)naphthalen-2-ol, (E)-5-(2-(benzo[d][1,3]dioxo 1-5-ylmethylene)hydrazinyl)-1H-tetrazole, (E)-4-((2-(1H-tetrazol-5-yl)hydrazono)methyl)phenol, (E)-5-(2-(4-nitrobenzylidene)hydrazinyl)-1H-tetrazole, (E)-5-(2-(furan-2-ylmethylene)hydrazinyl)-1H-tetrazole, 5-hydrazinyl-1H-tetrazole, 5-(1-methylhydrazinyl)-1H-tetrazole, 5-(1-methylhydrazinyl)-1H-1,2,4-triazol-3(2H)-one, 5-(1-ethylhydrazinyl)-1H-1,2,4-triazol-3(2H)-one, or 5-(hydrazinylmethyl)-1H-tetrazole reduces the chemosensitivity of the carotid body to the partial pressure of oxygen in arterial blood, reduces the chemosensitivity of the carotid body to the partial pressure of carbon dioxide in arterial blood, reduces the loop gain of the ventilatory drive control system, lowers blood pressure, or dampens carotid sinus nerve activity in an individual in need thereof or a combination thereof.

In some specific embodiments of the method, 2-aminopent-4-ynoic acid, (S)-2-aminopent-4-ynoic acid, 2-amino-3-cyanopropanoic acid, (S)-2-amino-3-cyanopropanoic acid, 2-hydrazinylacetic acid hydrochloride, 2-(2-(propan-2-ylidene)hydrazinyl)acetic acid, 4-((2-(1H-tetrazol-5-yl)hydrazinyl)methyl)-N,N-dimethylaniline, (E)-4-((2-(1H-tetrazol-5-yl)hydrazono)methyl)-N,N-diethylaniline, (E)-1-((2-(1H-tetrazol-5-yl)hydrazono)methyl)naphthalen-2-ol, (E)-5-(2-(benzo[d][1,3]dioxo 1-5-ylmethylene)hydrazinyl)-1H-tetrazole, (E)-4-((2-(1H-tetrazol-5-yl)hydrazono)methyl)phenol, (E)-5-(2-(4-nitrobenzylidene)hydrazinyl)-1H-tetrazole, (E)-5-(2-(furan-2-ylmethylene)hydrazinyl)-1H-tetrazole, 5-hydrazinyl-1H-tetrazole, 5-(1-methylhydrazinyl)-1H-tetrazole, 5-(1-methylhydrazinyl)-1H-1,2,4-triazol-3(2H)-one, 5-(1-ethylhydrazinyl)-1H-1,2,4-triazol-3(2H)-one, or 5-(hydrazinylmethyl)-1H-tetrazole reduces the chemosensitivity of the carotid body in an individual in need thereof. In some embodiments, 2-aminopent-4-ynoic acid, (S)-2-aminopent-4-ynoic acid, 2-amino-3-cyanopropanoic acid, (S)-2-amino-3-cyanopropanoic acid, 2-hydrazinylacetic acid hydrochloride, 2-(2-(propan-2-ylidene)hydrazinyl)acetic acid, 4-((2-(1H-tetrazol-5-yl)hydrazinyl)methyl)-N,N-dimethylaniline, (E)-4-((2-(1H-tetrazol-5-yl)hydrazono)methyl)-N,N-diethylaniline, (E)-1-((2-(1H-tetrazol-5-yl)hydrazono)methyl)naphthalen-2-ol, (E)-5-(2-(benzo[d][1,3]dioxo 1-5-ylmethylene)hydrazinyl)-1H-tetrazole, (E)-4-((2-(1H-tetrazol-5-yl)hydrazono)methyl)phenol, (E)-5-(2-(4-nitrobenzylidene)hydrazinyl)-1H-tetrazole, (E)-5-(2-(furan-2-ylmethylene)hydrazinyl)-1H-tetrazole, 5-hydrazinyl-1H-tetrazole, 5-(1-methylhydrazinyl)-1H-tetrazole, 5-(1-methylhydrazinyl)-1H-1,2,4-triazol-3(2H)-one, 5-(1-ethylhydrazinyl)-1H-1,2,4-triazol-3(2H)-one, or 5-(hydrazinylmethyl)-1H-tetrazole reduces the chemosensitivity of the carotid body to the partial pressure of oxygen in arterial blood. In some embodiments, 2-aminopent-4-ynoic acid, (S)-2-aminopent-4-ynoic acid, 2-amino-3-cyanopropanoic acid, (S)-2-amino-3-cyanopropanoic acid, 2-hydrazinylacetic acid hydrochloride, 2-(2-(propan-2-ylidene)hydrazinyl)acetic acid, 4-((2-(1H-tetrazol-5-yl)hydrazinyl)methyl)-N,N-dimethylaniline, (E)-4-((2-(1H-tetrazol-5-yl)hydrazono)methyl)-N,N-diethylaniline, (E)-1-((2-(1H-tetrazol-5-yl)hydrazono)methyl)naphthalen-2-ol, (E)-5-(2-(benzo[d][1,3]dioxo 1-5-ylmethylene)hydrazinyl)-1H-tetrazole, (E)-4-((2-(1H-tetrazol-5-yl)hydrazono)methyl)phenol, (E)-5-(2-(4-nitrobenzylidene)hydrazinyl)-1H-tetrazole, (E)-5-(2-(furan-2-ylmethylene)hydrazinyl)-1H-tetrazole, 5-hydrazinyl-1H-tetrazole, 5-(1-methylhydrazinyl)-1H-tetrazole, 5-(1-methylhydrazinyl)-1H-1,2,4-triazol-3(2H)-one, 5-(1-ethylhydrazinyl)-1H-1,2,4-triazol-3(2H)-one, or 5-(hydrazinylmethyl)-1H-tetrazole reduces the loop gain of the ventilatory drive control system in an individual in need thereof. In some embodiments, 2-aminopent-4-ynoic acid, (S)-2-aminopent-4-ynoic acid, 2-amino-3-cyanopropanoic acid, (S)-2-amino-3-cyanopropanoic acid, 2-hydrazinylacetic acid hydrochloride, 2-(2-(propan-2-ylidene)hydrazinyl)acetic acid, 4-((2-(1H-tetrazol-5-yl)hydrazinyl)methyl)-N,N-dimethylaniline, (E)-4-((2-(1H-tetrazol-5-yl)hydrazono)methyl)-N,N-diethylaniline, (E)-1-((2-(1H-tetrazol-5-yl)hydrazono)methyl)naphthalen-2-ol, (E)-5-(2-(benzo[d][1,3]dioxo 1-5-ylmethylene)hydrazinyl)-1H-tetrazole, (E)-4-((2-(1H-tetrazol-5-yl)hydrazono)methyl)phenol, (E)-5-(2-(4-nitrobenzylidene)hydrazinyl)-1H-tetrazole, (E)-5-(2-(furan-2-ylmethylene)hydrazinyl)-1H-tetrazole, 5-hydrazinyl-1H-tetrazole, 5-(1-methylhydrazinyl)-1H-tetrazole, 5-(1-methylhydrazinyl)-1H-1,2,4-triazol-3(2H)-one, 5-(1-ethylhydrazinyl)-1H-1,2,4-triazol-3(2H)-one, or 5-(hydrazinylmethyl)-1H-tetrazole reduces blood pressure in an individual in need thereof. In some embodiments, 2-aminopent-4-ynoic acid, (S)-2-aminopent-4-ynoic acid, 2-amino-3-cyanopropanoic acid, (S)-2-amino-3-cyanopropanoic acid, 2-hydrazinylacetic acid hydrochloride, 2-(2-(propan-2-ylidene)hydrazinyl)acetic acid, 4-((2-(1H-tetrazol-5-yl)hydrazinyl)methyl)-N,N-dimethylaniline, (E)-4-((2-(1H-tetrazol-5-yl)hydrazono)methyl)-N,N-diethylaniline, (E)-1-((2-(1H-tetrazol-5-yl)hydrazono)methyl)naphthalen-2-ol, (E)-5-(2-(benzo[d][1,3]dioxol-5-ylmethylene)hydrazinyl)-1H-tetrazole, (E)-4-((2-(1H-tetrazol-5-yl)hydrazono)methyl)phenol, (E)-5-(2-(4-nitrobenzylidene)hydrazinyl)-1H-tetrazole, (E)-5-(2-(furan-2-ylmethylene)hydrazinyl)-1H-tetrazole, 5-hydrazinyl-1H-tetrazole, 5-(1-methylhydrazinyl)-1H-tetrazole, 5-(1-methylhydrazinyl)-1H-1,2,4-triazol-3(2H)-one, 5-(1-ethylhydrazinyl)-1H-1,2,4-triazol-3(2H)-one, or 5-(hydrazinylmethyl)-1H-tetrazole dampens carotid sinus nerve activity.

In some embodiments of the method, the CSE inhibitor is a compound of Formula (14) having the structure:

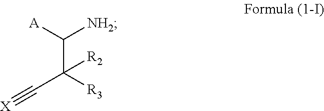

wherein:
A is a carboxylic acid isostere;
X is CR₁, or N;
R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl;
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;

or a pharmaceutically acceptable salt, solvate, or prodrug thereof.

In some embodiments of the method, the CSE inhibitor is a compound of Formula (1-II) having the structure:

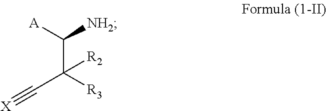

wherein:
A is a carboxylic acid isostere;
X is CR₁, or N;
R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl;
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;

or a pharmaceutically acceptable salt, solvate, or prodrug thereof.

In some embodiments of compounds of Formula (1-I) and Formula (1-II), A is a carboxylic acid isostere selected from

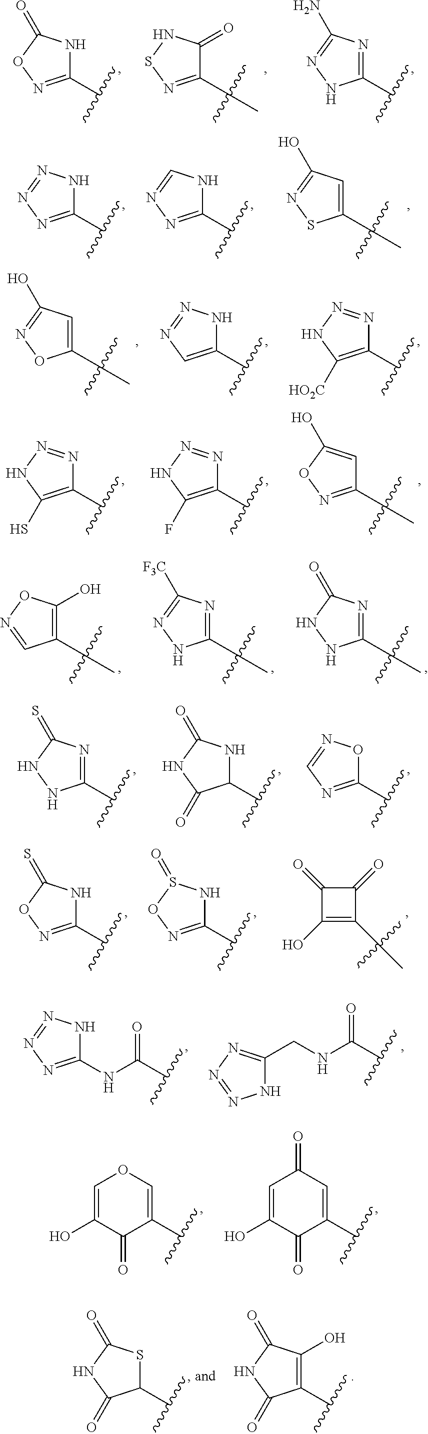

In some embodiments of compounds of Formula (1-I) and Formula (1-II), A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —B(OR₅)₂, —C(R₄)₂B(OR₅)₂, and —CON(R₄)C(R₄)₂B(OR₅)₂; wherein each R₄ is independently H, OH, substituted or unsubstituted alkyl, or substituted or unsubstituted aryl; and R₅ is H or C₁-C₆alkyl.

In some embodiments of compounds of Formula (1-I) and Formula (1-II), A is a carboxylic acid isostere selected from —SO₃H, —SO₂NHR₄, —P(O)(OR₄)₂, —P(O)(R₄)(OR₄), —C(O)R₄, —CON(R₄)₂, —CONHNHSO₂R₄, —CONHSO₂R₄, —C(R₄)₂B(OR₅)₂, and —CON(R₄)C(R₄)₂B(OR₅)₂; wherein each R₄ is independently H, OH, substituted or unsubstituted alkyl, substituted or unsubstituted heteroaryl, or substituted or unsubstituted aryl; and R₅ is H or C₁-C₆alkyl.

In some embodiments, for any of the preceding embodiments of compounds of Formula (1-I) and Formula (1-II), X is N. In some embodiments, for any of the preceding embodiments of compounds of Formula (1-I) and Formula (1-II), X is CR₁.

In some embodiments, for any of the preceding embodiments of compounds of Formula (1-I) and Formula (1-II), R₁ is H, substituted or unsubstituted alkyl, or substituted or unsubstituted heteroalkyl. In some embodiments, for any of the preceding embodiments of compounds of Formula (1-I) and Formula (1-II), R₁ is H. In some embodiments, for any of the preceding embodiments of compounds of Formula (1-I) and Formula (1-II), R₁ is CH₃. In some embodiments, for any of the preceding embodiments of compounds of Formula (1-I) and Formula (1-II), A is tetrazole, and R₂ and R₃ are each H.

In some embodiments of the method, the CSE inhibitor is a compound of Formula (1-III) having the structure:

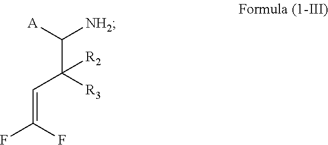

wherein:
A is a carboxylic acid isostere;
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;

or a pharmaceutically acceptable salt, solvate, or prodrug thereof.

In some embodiments of the method, the CSE inhibitor is a compound of Formula (1-IV) having the structure:

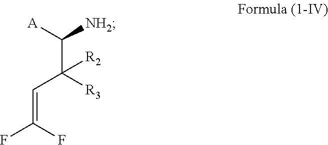

wherein:
A is a carboxylic acid isostere;
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;

or a pharmaceutically acceptable salt, solvate, or prodrug thereof.

In some embodiments of compounds of Formula (1-III) and Formula (1-Iv), A is a carboxylic acid isostere selected from

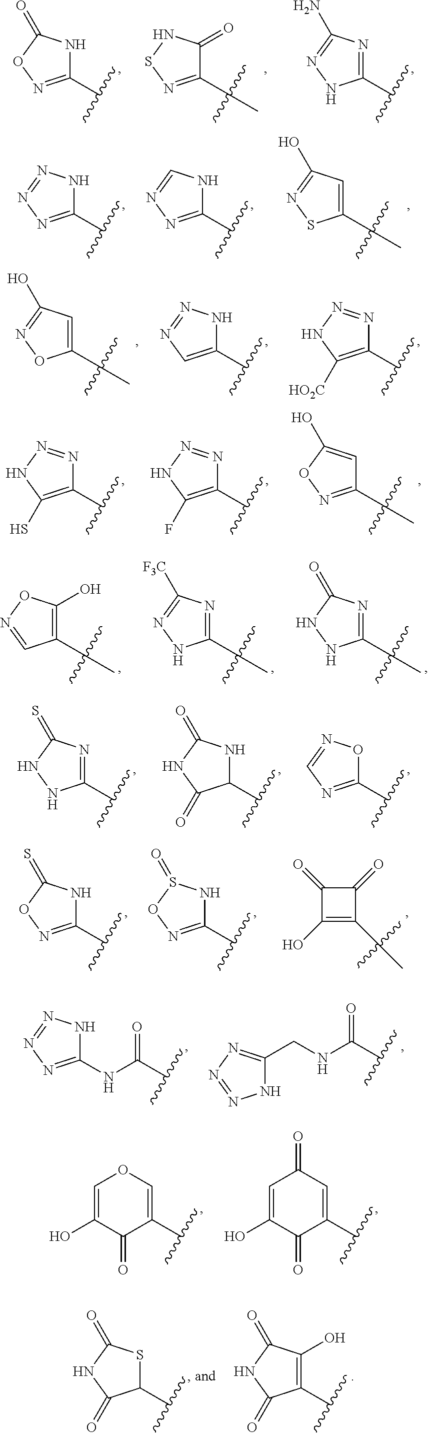

In some embodiments of compounds of Formula (1-III) and Formula (1-Iv), A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —B(OR₅)₂, —C(R₄)₂B(OR₅)₂, and —CON(R₄)C(R₄)₂B(OR₅)₂; wherein each R₄ is independently H, OH, substituted or unsubstituted alkyl, or substituted or unsubstituted aryl; and R₅ is H or C₁-C₆alkyl.

In some embodiments of compounds of Formula (1-III) and Formula (1-Iv), A is a carboxylic acid isostere selected from —SO₃H, —SO₂NHR₄, —P(O)(OR₄)₂, —P(O)(R₄)(OR₄), —C(O)R₄, —CON(R₄)₂, —CONHNHSO₂R₄, —CONHSO₂R₄, —C(R₄)₂B(OR₅)₂, and —CON(R₄)C(R₄)₂B(OR₅)₂; wherein each R₄ is independently H, OH, substituted or unsubstituted alkyl, substituted or unsubstituted heteroaryl, or substituted or unsubstituted aryl; and R₅ is H or C₁-C₆alkyl.

In some embodiments, for any of the preceding embodiments of compounds of Formula (1-III) and Formula (1-IV), R₂ and R₃ are each H.

In some embodiments, for any of the preceding embodiments of compounds of Formula (1-III) and Formula (1-IV), A is

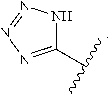 [SMILES: *c1nnn[nH]1 ; "."]

Provided herein is a pharmaceutical composition comprising a pharmaceutically acceptable excipient and a compound of any one of Formula (1-I), Formula (1-II), Formula (1-III) or Formula (1-IV), or a pharmaceutically acceptable salt, a pharmaceutically acceptable solvate, or a pharmaceutically acceptable prodrug thereof.

In some embodiments of the method, the CSE inhibitor is a compound of Formula (2-I) having the structure:

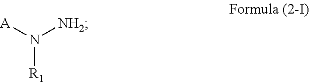

wherein:
A is a carboxylic acid isostere;
R₁ is substituted or unsubstituted C₃-C₆alkyl, substituted or unsubstituted heteroalkyl, substituted or unsubstituted heterocycloalkyl, substituted or unsubstituted aryl, or substituted or unsubstituted heteroaryl; or a pharmaceutically acceptable salt, solvate, or prodrug thereof.

In some embodiments of the method, the CSE inhibitor is a compound of Formula (2-II) having the structure:

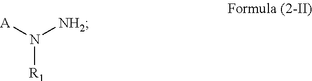

wherein:
R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl;
A is selected from

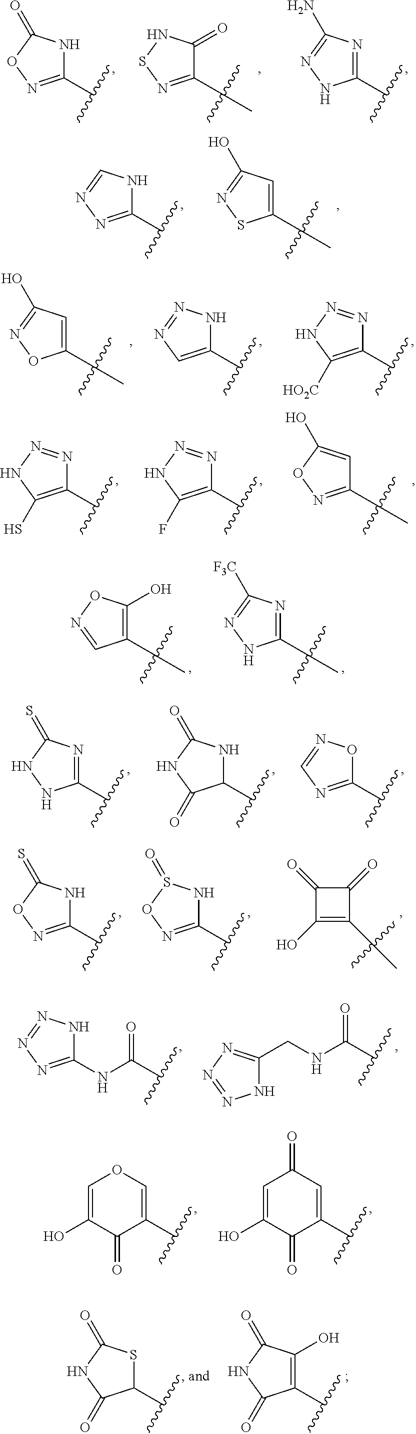

or

a pharmaceutically acceptable salt, solvate, or prodrug thereof.

In some embodiments of compounds of Formula (2-II), A is selected from

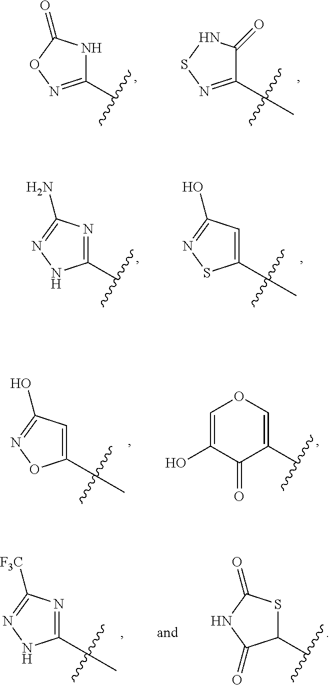

In some embodiments of the method, the CSE inhibitor is a compound of Formula (2-III) having the structure:

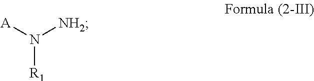

wherein:
R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl;
A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂; wherein each R₄ is independently H, OH, substituted or unsubstituted alkyl, or substituted or unsubstituted aryl; and R₅ is H or C₁-C₆alkyl; or

a pharmaceutically acceptable salt, solvate, or prodrug thereof.

In some of the preceding embodiments of compounds of Formula (2-II), or Formula (2-III), R₁ is H, substituted or unsubstituted alkyl, or substituted or unsubstituted heteroalkyl.

In some of the preceding embodiments of compounds of Formula (2-II), or Formula (2-III), R₁ is H. In some of the preceding embodiments of compounds of Formula (2-II), or Formula (2-III), R₁ substituted or unsubstituted C₁-C₄alkyl. In some of the preceding embodiments of compounds of Formula (2-II), or Formula (2-III), R₁ is —CH₃. In some of the preceding embodiments of compounds of Formula (2-II), or Formula (2-III), R₁ is —CH₂CH₃.

In some embodiments of the method, the CSE inhibitor is a compound of Formula (2-IV) having the structure:

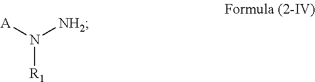

wherein:
A is

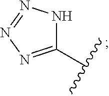 [SMILES: *c1nnn[nH]1]

R₁ is substituted or unsubstituted C₂-C₆alkyl, substituted or unsubstituted heteroalkyl, substituted or unsubstituted heterocycloalkyl, substituted or unsubstituted aryl, or substituted or unsubstituted heteroaryl; or a pharmaceutically acceptable salt, solvate, or prodrug thereof.

In some embodiment of compounds of Formula (2-IV), R₁ is H, substituted or unsubstituted C₂-C₆alkyl, or substituted or unsubstituted heteroalkyl. In some embodiment of compounds of Formula (2-IV), R₁ is H. In some embodiment of compounds of Formula (2-IV), R₁ substituted or unsubstituted C₂-C₆alkyl. In some embodiment of compounds of Formula (2-IV), R₁ is —CH₂CH₃.

In some embodiments of the method, the CSE inhibitor is a compound of Formula (2-V) having the structure:

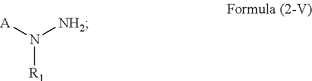

wherein:
A is

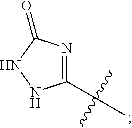 [SMILES: *c1nc(=O)[nH][nH]1]

R₁ is H, substituted or unsubstituted C₃-C₆alkyl, substituted or unsubstituted heteroalkyl, substituted or unsubstituted heterocycloalkyl, substituted or unsubstituted aryl, or substituted or unsubstituted heteroaryl; or a pharmaceutically acceptable salt, solvate, or prodrug thereof.

In some embodiments of compounds of Formula (2-V), R₁ is H, substituted or unsubstituted C₃-C₆alkyl, or substituted or unsubstituted heteroalkyl. In some embodiments of compounds of Formula (2-V), R₁ is H. In some embodiments of compounds of Formula (2-V), R₁ substituted or unsubstituted C₃-C₆alkyl.

Provided herein is a pharmaceutical composition comprising a pharmaceutically acceptable excipient and a compound of any one of Formula (24), Formula (2-II), Formula (2-III), Formula (2-IV), or Formula (2-V) or a pharmaceutically acceptable salt, a pharmaceutically acceptable solvate, or a pharmaceutically acceptable prodrug thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a brass comb used in the experiment of Example 3-2.

FIG. 2A shows gross differential cutaneous burn progression in treated animals over controls as described in Example 3-2. In animals receiving treatment with L-propargylglycine (FIG. 2A), the zones of stasis/interspaces appeared to maintain viability over the time course, while the same areas in control animals began to convert and become more necrotic.

FIG. 2B shows gross differential cutaneous burn progression in treated animals over controls as described in Example 3-2. In animals receiving treatment with 5-(1-methylhydrazinyl)-1H-tetrazole (Compound 1) (FIG. 2B), the zones of stasis/interspaces appeared to maintain viability over the time course, while the same areas in control animals began to convert and become more necrotic around Day 2.

FIG. 3 shows a graph resulting from use of a grading system to compare treated interspaces versus interspaces in control animals. Assigning a “less injured” versus “worse/the same” scale demonstrated that within 48-72 hours, most interspaces in treated animals appeared less injured than controls as described in Example 3-2 and the data were significantly distributed (L-propargylglycine p=0.0007 and 5-(1-methylhydrazinyl)-1H-tetrazole (Compound 1) p<0.001).

FIG. 4 shows results from LDI analysis. The experiment (Example 3-2) revealed a decrease in perfusion over time in the interspace areas in control animals. This corresponds with the conversion of these areas to more damaged, less viable tissue. Conversely, perfusion is maintained in the zones of stasis in animals treated with L-propargylglycine. A similar trend of maintenance of perfusion is indicated in animals treated with 5-(1-methylhydrazinyl)-1H-tetrazole (Compound 1).

FIG. 5 illustrates the hypoxic ventilatory response (HVR), which is a measure of peripheral chemosensitivity, in rats to an acute hypoxic challenge (FIO2=10%) given varying IP doses of propargylglycine (PAG). The doses ranged from none (NS=normal saline vehicle) to 100 mg/kg. At the 30 mg/kg and the 100 mg/kg doses, the HVR was significantly reduced compared to the vehicle-treated rats.

FIG. 6 shows a summary of respiratory and metabolic measurements (Example 3-3).

FIG. 7 shows respiratory measurements in the HVR assay (Example 3-3).

FIG. 8 shows metabolic measurements in the HVR assay (Example 3-3).

FIG. 9 shows changes in minute ventilation from normoxia to hypoxia (Example 3-3).

FIG. 10 shows average ΔV_{E} for all treatment groups (Example 3-3).

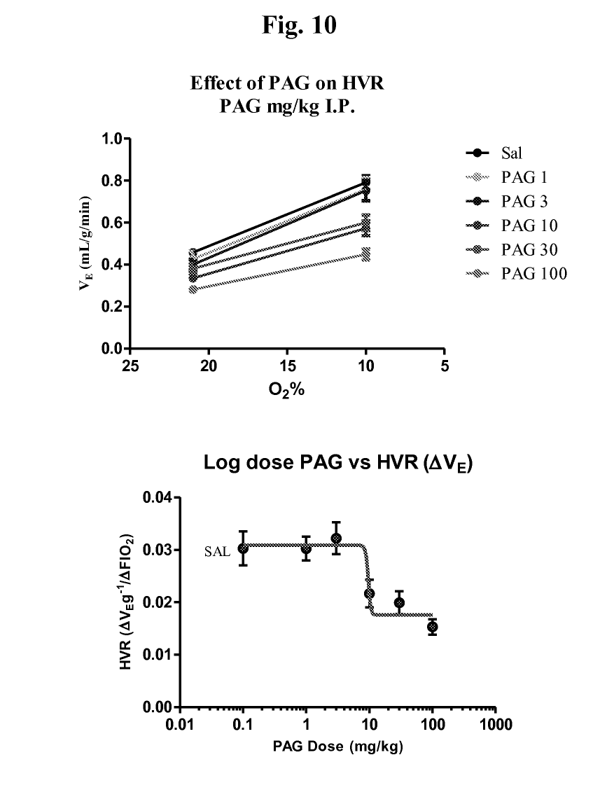

FIG. 11 shows a summary of respiratory and metabolic measurements (Example 3-4).

FIG. 12 shows respiratory measurements in the HVR assay (Example 3-4).

FIG. 13 shows minute ventilation in the HVR assay (Example 3-4).

FIG. 14 shows effect of carotid sinus nerve (CSN) transection on the HVR (Example 3-4).

### DETAILED DESCRIPTION OF THE INVENTION

Endogenous hydrogen sulfide is synthesized through degradation of L-cysteine by cystathionine-gamma-lyase (CSE) or cystathionine-beta synthase (CBS). The enzyme cystathionine-γ-lyase (CSE) converts cystathionine to L-cysteine, yielding pyruvate, ammonia and hydrogen sulfide. Hydrogen sulfide is a gaseous transmitter which plays a role in many physiological processes including vasodilation (e.g., smooth muscle relaxation and/or opening of vascular smooth muscle K channels), and neuromodulation (e.g., induction of hippocampal long-term potentiation). Studies have shown that hydrogen sulfide is also associated with inflammation (e.g., hindpaw edema), acute pancreatitis, endotoxemia and sepsis.

The pro-inflammatory activity of hydrogen sulfide plays a role in various cutaneous injuries or conditions described herein. Provided herein are methods for regulating hydrogen sulfide-associated inflammation comprising administering, systemically or locally, or a combination thereof, a CSE inhibitor to an individual in need thereof, thereby improving treatment outcomes for patients suffering from cutaneous injuries or conditions. Provided herein are methods for regulating wound healing associated with hydrogen sulfide-mediated inflammation comprising administering, systemically or locally, or a combination thereof, a CSE inhibitor to an individual in need thereof.

Cutaneous injuries or conditions include epidermal, dermal and/or subcutaneous injuries or conditions ranging from boils, pimples, blisters, hives, epidermolysis and/or necrolysis, to burn injuries (including burn injuries) and deep tissue burn injuries, and sequelae such as skin contractures and scarring of the skin. Provided herein are novel methods which modify treatment outcomes for patients suffering from cutaneous injuries or conditions. In some embodiments, the methods provided herein modify the cutaneous and/or subcutaneous wound healing process and improve treatment outcomes for individuals suffering from cutaneous injuries or conditions. Contemplated within the scope of embodiments presented herein are certain cutaneous injuries or conditions, and methods for treatment of such conditions, which are described below.

### Burn Injuries

A cutaneous injury or condition involving a skin burn (e.g., a contact burn) is typically an evolving injury. The histological description of a cutaneous burn injury is categorized in terms of specific areas of pathologic change. The initial surface injury (zone of coagulation) is caused by the heat or chemical insult and is an irreversible injury. In addition to the zone of coagulation, there is a deeper and broader area of progressive tissue injury (zone of stasis) where cells are viable but are vulnerable to further damage. The progressive injury in the tissue is typically due to capillary thrombosis from injured endothelium, often leading to ischemia-induced cell death. Early epithelial cell death in this area leads to slowing of healing. Epithelial cells in the zone of stasis may be subject to desiccation and/or inflammation-induced injury. Provided herein are methods for improving treatment outcomes for cutaneous burn injuries comprising administration of a CSE inhibitor to an individual in need thereof. In some of such embodiments, the methods allow for early interception and reduction of epithelial cell death, thus improving treatment outcomes for burn injuries.

The extent of cutaneous burn injury impacts patient morbidity and mortality. The zone of stasis and its potential for conversion into burn wounds, or alternatively, maintenance of its viability, is therefore of critical importance. Regulating inflammation systemically and/or locally (e.g., in the zone of stasist) improves patient outcome. Provided herein are methods for treatment of cutaneous injuries or conditions, including cutaneous burn injuries (e.g., contact burns), comprising administration of CSE inhibitors to individuals in need thereof.

The methods described herein are designed to impact overall patient outcome by reducing the production of hydrogen sulfide in areas of burn injuries and thereby improving patient outcome. In some cases, administration of CSE inhibitors described herein intercepts and/or reduces the impact of an extended inflammatory response, decreased blood flow, and cell death in the zone of stasis. The methods described herein allow for preservation of the viability of vulnerable tissue adjacent to burn injuries and thus prevent burn wound progression. Accordingly, provided herein are methods for prevention of cutaneous burn wound progression comprising administration of a CSE inhibitor to an individual in need thereof. Provided herein are methods for promotion of cutaneous burn wound healing comprising administration of a CSE inhibitor to an individual in need thereof. In any of the preceding embodiments, a cutaneous burn injury is an acute burn injury. In any of the preceding embodiments, a cutaneous burn injury is a contact burn injury. In any of the preceding embodiments, a cutaneous burn injury is a severe partial-thickness burn injury or a full-thickness burn injury.

As used herein, in one embodiment, a cutaneous burn injury is a contact burn, e.g., a chemical burn, or a burn from touching a hot object, a burn from contact with hot water or hot oil (a scald), or a burn from a fire. In additional embodiments, a contact burn is a skin abrasion from a fall (e.g., road rash due to a fall from a bicycle, or while skateboarding or roller blading). In yet other embodiments, a cutaneous burn is caused by an electric shock (e.g., lightning strike, contact with electrical objects). In yet other embodiments, a cutaneous burn is a radiation burn (e.g., a burn associated with radiotherapy for treatment of cancer), or a sun burn. In yet other embodiments, a cutaneous burn is due to inflamed skin caused by contact with an allergen (e.g., skin rash due to contact with poison oak, or a bee sting). In some embodiments, a cutaneous burn is caused by friction or chafing (e.g., blisters on feet due to new shoes).

In any of the preceding embodiments, a cutaneous burn is treated with local administration of a CSE inhibitor, or with systemic administration of a CSE inhibitor, or with a combination of both systemic and local administration of a CSE inhibitor.

In any of the preceding embodiments, a cutaneous burn is a first degree burn. In any of the preceding embodiments, a cutaneous burn is a second degree burn. In any of the preceding embodiments, a cutaneous burn is a third degree burn. In any of the preceding embodiments, a cutaneous burn is a fourth degree burn.

### Epidermolysis

Epidermolysis bullosa (EB) is a group of inherited bullous disorders characterized by blister formation in the skin and mucosal membranes in response to minor injury, heat, or friction from rubbing, scratching or adhesive tape. As a result, the skin is extremely fragile. Minor mechanical friction or trauma will separate the layers of the skin and form blisters. At present, there is no cure for epidermolysis bullosa. Current therapeutic approaches focus on addressing the symptoms, including pain prevention, wound prevention, infection and severe itching that occurs with continuous wound healing.

Accordingly, provided herein are methods for treating epidermolysis bullosa comprising administration of a CSE inhibitor to an individual in need thereof.

### Stevens-Johnson Syndrome, Toxic Epidermal Necrolysis

Stevens-Johnson syndrome (also known as erythema multiforme) is a rare, serious disorder in which skin and mucous membranes react severely to a medication or infection. Often, Stevens-Johnson syndrome is associated with a painful red or purplish rash that spreads and blisters, eventually causing the top layer of the skin to die and shed. The disease also affects mucosal membranes.

Toxic epidermal necrolysis is a more severe form of Stevens-Johnson syndrome. Stevens-Johnson syndrome presents a medical emergency that usually requires hospitalization.

Contemplated within the scope of embodiments presented herein are methods for treatment of cutaneous injuries or conditions associated with necrolysis of the skin comprising administration of a CSE inhibitor to an individual in need thereof. In some of such embodiments, the cutaneous injury or condition associated with necrolysis of the skin is Steven-Johnson syndrome. In some of such embodiments, the cutaneous injury or condition associated with necrolysis of the skin is toxic epidermal necrolysis.

### Staphylococcal Scalded Skin Syndrome

In some embodiments, a cutaneous injury or condition is associated with an infection. Staphylococcal scalded skin syndrome (SSSS), also known as Ritter von Ritterschein disease (in newborns), Ritter disease, and staphylococcal epidermal necrolysis, encompasses a spectrum of superficial blistering skin disorders caused by the exfoliative toxins of some strains of Staphylococcus aureus.

SSSS is a cutaneous injury or condition of acute exfoliation of the skin typically following an erythematous cellulitis. The severity of staphylococcal scalded skin syndrome varies from a few blisters localized to the site of infection to a severe exfoliation affecting almost the entire body.

Contemplated within the scope of embodiments presented herein are methods for treatment of cutaneous injuries or conditions associated with skin infections such as erythematous cellulitis comprising administration of a CSE inhibitor to an individual in need thereof. In some of such embodiments, the cutaneous injury or condition is staphylococcal scalded skin syndrome. Provided herein is a method for reducing wound progression associated with staphylococcal scalded skin syndrome comprising administration of a CSE inhibitor to an individual in need thereof.

### Contractures

A contracture is a permanent tightening of the skin that prevents normal movement of the associated body part and causes permanent deformity. In some cases, despite treatment of a cutaneous burn injury, an individual suffers from cutaneous contractures. In some embodiments, a cutaneous contracture is associated with a deep tissue burn injury (e.g., a contact burn) which develops when the normally elastic connective tissues are replaced by inelastic fibrous tissue. This makes the affected area resistant to stretching and prevents normal movement. In some embodiments, a cutaneous contracture is associated with skin graft surgery.

The methods of treatment described herein reduce or prevent the occurrence of cutaneous contractures (e.g., by preventing burn wound progression). Accordingly, provided herein are methods for reducing the occurrence of cutaneous contractures associated with cutaneous burn injuries and/or cutaneous conditions or surgery comprising administration of a CSE inhibitor to an individual in need thereof.

Contemplated within the scope of embodiments presented herein are methods for treatment of cutaneous contractures associated with skin graft surgery comprising administration of a CSE inhibitor to an individual in need thereof. Provided herein is a method for reducing contractures associated with cutaneous injuries or conditions (e.g., deep tissue burn injuries) comprising administration of a CSE inhibitor to an individual in need thereof. Provided herein is a method for reducing cutaneous contractures associated with cutaneous burns comprising administration of a CSE inhibitor to an individual in need thereof. Provided herein is a method for reducing occurrence of cutaneous contractures associated with skin graft surgery comprising administration of a CSE inhibitor to an individual in need thereof.

### Scarring and Fibrosis

In some cases, despite treatment of cutaneous injuries or conditions, an individual suffers from cutaneous scarring and/or fibrosis in the affected area. Provided herein is a method for reducing scarring associated with cutaneous injuries or conditions (e.g., contact burns or any other cutaneous condition described herein) and/or skin graft surgery comprising administration of a CSE inhibitor to an individual in need thereof. Provided herein is a method for reducing occurrence of fibrosis or treating fibrosis associated with skin graft surgery and/or cutaneous injuries or conditions (e.g., contact burns or any other condition described herein) comprising administration of a CSE inhibitor to an individual in need thereof.

### Allergic Conditions

In some embodiments, a cutaneous injury or condition is associated with contact with an allergen (e.g., a chemical, poison oak, or the like) or an insect bit (e.g., a bee sting). In some of such embodiments, the cutaneous injury or condition is manifested as a rash, blisters, hives and/or pustules. In some of such embodiments, the cutaneous injury or condition is associated with inflammatory edema.

Contemplated within the scope of embodiments presented herein are methods for the treatment of cutaneous injuries or conditions associated with contact with an allergen or an insect bite comprising administration of a CSE inhibitor to an individual in need thereof. Provided herein is a method for treating allergic hives comprising administration of a CSE inhibitor to an individual in need thereof. Provided herein is a method for treating skin conditions associated with insect bites comprising administration of a CSE inhibitor to an individual in need thereof. Provided herein is a method for treating blisters, pustules or rash associated with contact with an allergen comprising administration of a CSE inhibitor to an individual in need thereof. Provided herein is a method for treating any inflammatory edema comprising administration of a CSE inhibitor to an individual in need thereof.

### Ulcers

In some embodiments, a cutaneous injury or condition is ulcers (e.g., diabetic ulcers). Diabetic foot lesions are a common complication of diabetes. Diabetes is a leading cause of nontraumatic lower extremity amputations in the United States. Diabetic neuropathy tends to occur about 10 years after the onset of diabetes, and diabetic foot deformity and ulceration occur sometime thereafter.

Contemplated within the scope of embodiments presented herein are methods for the treatment of diabetic ulcers comprising administration of a CSE inhibitor to an individual in need thereof.

### Toxic Shock Syndrome

In some embodiments, a cutaneous injury or condition is associated with an infection. Toxic shock syndrome (TSS) (also known as toxic shock-like syndrome (TSLS) or streptococcal toxic shock syndrome (STSS)) is a potentially fatal illness caused by a bacterial toxin. The causative bacteria include Staphylococcus aureus and Streptococcus pyogenes. The symptoms of toxic shock syndrome include high fever, accompanied by low blood pressure, malaise and confusion, which can rapidly progress to stupor, coma, and multiple organ failure. A characteristic rash is often seen early in the course of illness and resembles a sunburn; the rash can involve any region of the body, including the lips, mouth, eyes, palms and soles. In patients who survive the initial onslaught of the infection, the rash desquamates, or peels off, after 10-14 days.

Provided herein are methods for treatment of toxic shock syndrome comprising administration of a CSE inhibitor to an individual in need thereof. In some of such embodiments, the method allows for treatment of the cutaneous rash associated with toxic shock syndrome.

### Sleep Related Breathing Disorders

Also described herein are methods for modulating the activity of the carotid body. In some embodiments, the methods described herein allow for modulation of chemosensitivity of the carotid body in response to hypoxia. In some embodiments, alteration of the response of the carotid body to hydrogen sulfide (H₂S) allows for treatment of SRBDs. In some embodiments, modulation of the sensory response of the carotid body reduces the activity of the carotid body in individuals in need thereof.

Provided herein are methods for modulating the activity of the carotid body in an individual. Also provided herein are methods for modulating gasotransmitter pathways associated with regulation of breathing. Gaseous messengers such as hydrogen sulfide, and carbon monoxide, play a role in oxygen sensing by the carotid body. Reflexes arising from the carotid body have been implicated in pathological situations including and not limited to SRBDs with recurrent apnea (i.e., periodic cessations of breathing) and/or hypoapnea (i.e., reduced breath amplitude). Patients with recurrent apnea experience periodic hypoxemia and/or intermittent hypoxia and are prone to autonomic morbidities including, for example, hypertension. SRBD includes a range of conditions that manifest pathologically as central apnea, obstructive apnea or mixed apnea.

Current therapy for SRBDs utilizes mechanical devices to aid breathing. Such assisted breathing and/or alleviation of apnea includes application of positive airway pressure to an individual in need thereof. The mode of application of positive airway pressure depends on whether the apneas are caused by hyperventilation or hypoventilation. Continuous positive airway pressure (CPAP) is suitable for patients whose central apneas are due to hyperventilation. CPAP reduces the frequency of apneas by preventing pharyngeal airway narrowing and occlusion during sleep. Another therapeutic approach involves the use of noninvasive positive pressure ventilation (NIPPV), such as pressure support ventilation (PSV) or bilevel positive airway pressure (BiPAP), with a set backup respiratory rate. However, in some instances, BiPAP without a backup respiratory rate exacerbates hyperventilation, hypocapnia, and central apnea by augmenting tidal volume. NIPPV potentially worsens alveolar ventilation. Adaptive servo-ventilation (ASV) provides a small but varying amount of inspiratory pressure superimposed on a low level of CPAP. The magnitude of the inspiratory pressure is reciprocal to the amount of respiratory effort. Supplemental oxygen and/or supplemental carbon dioxide are also used in current therapy under tightly controlled delivery. Current pharmacologic therapy includes the use of respiratory stimulants. However, none of these therapeutic approaches address the underlying pathology of SRBDs.

Described herein are therapeutic approaches for the treatment of SRBDs. In some embodiments, such methods allow for modulation of the activity of the carotid body, an organ involved in hypoxic sensing and control of breathing.

### Carotid Body

In adult mammals, carotid bodies are peripheral sensory organs responsible for monitoring arterial blood CO₂ and/or O₂ concentrations and relaying sensory information to the brainstem neurons associated with regulation of breathing and the cardiovascular system. The carotid body (carotid glomus or glomus caroticum) is a highly vascularized region located near the bifurcation of the carotid artery and comprises a cluster of peripheral chemoreceptors and supporting cells. The carotid body is linked to the central chemoreceptors in the brainstem and relays sensory information to brainstem neurons that are associated with regulation of breathing and/or the cardiovascular system. Carotid bodies are the primary mediators of ventilatory stimulation induced under conditions of acute hypoxia. Accordingly, further provided herein are methods of treatment of diseases or conditions that are associated with carotid body activity and/or control of ventilation in individuals in need thereof.

### Cystathionine γ-Lyase Enzyme (CSE)

CSE catalyzes the formation of cysteine from cystathionine, and also generates H₂S from cysteine. In some embodiments, CSE-derived-H₂S, a redox active gasotransmitter, plays a role in hypoxic sensing by the carotid body. Genetic or pharmacologic deletion of CSE impairs hypoxic sensing by the carotid body as well as in neonatal adrenal medullary chromaffin cells (AMC).

CSE is expressed in rat and mouse glomus cells, the main site of O₂ sensing in the carotid body. Described herein is a physiological role for H₂S generated by CSE in mediating hypoxic sensing by the carotid body. Chemoreceptor responses to acute hypoxia were markedly impaired in CSE knockout mice and following pharmacologic inhibition of CSE. Although hypoxic sensitivity was lost, sensory response to CO₂ was intact in mutant mice and CSE inhibitor treated rats. CSE^{-/-} mice exhibited selective loss of ventilatory response to hypoxia but not to CO₂, suggesting that CSE disruption impacts systemic responses to acute hypoxia by affecting the carotid body. CSE is also expressed in neonatal adrenal medullary chromaffin cells (AMC) of rats and mice whose hypoxia-evoked catecholamine secretion is greatly attenuated by CSE inhibitors and in CSE^{-/-} mice.

Described herein is the carotid body response to hypoxia in wild type (CSE^{+/+}) and CSE^{-/-} mice as well as in rats treated with a CSE inhibitor. The following observations indicate that H₂S generated by CSE mediates carotid body hypoxic sensing. First, hypoxia increased H₂S generation in the carotid body in a stimulus-dependent manner, an effect that was lost in CSE^{-/-} mice as well as in rats treated with a CSE inhibitor. Second, loss of hypoxia-evoked H₂S generation paralleled impaired hypoxic sensing by the carotid body. Third, an H₂S donor, but not L-cysteine, stimulated the carotid body with a time-course and magnitude comparable to that evoked by low O₂. An H₂S donor stimulated carotid body activity in CSE knockout mice, indicating that the loss of hypoxic sensitivity is due to absence of H₂S generation rather than impaired H₂S signaling. These findings demonstrate that during hypoxia, CSE is a source of H₂S generation in the carotid body, and suggest that CSE contributes to hypoxic sensing by catalyzing H₂S generation.

### Carotid Body Activity in Neonates

Carotid bodies are the main organs for sensing acute hypoxia in adults but in neonates they are relatively insensitive to low O₂. On the other hand, adrenal medullary chromaffin cells (AMC) are extremely sensitive to hypoxia in neonates, and low O₂ stimulates catecholamine secretion, which plays a role in maintaining homeostasis in neonates under hypoxic stress. Like glomus cells, neonatal AMC expressed CSE, and hypoxia-evoked catecholamine secretion was severely impaired in CSE^{-/-} mice and in rats treated with a CSE inhibitor. Since hypoxia also increased H₂S generation in adrenal glands, CSE-H₂S system mediates acute hypoxic sensing by neonatal AMC. Hypoxic sensitivity of AMC, however, declines with age. In some instances, AMC is associated with developmental decline in CSE expression.

Gasotransmitters

Physiologically, the carotid body is sensitive to changes in arterial blood flowing through it including changes in partial pressure of oxygen in arterial blood (Pa0₂) (e.g., hypoxia), and/or changes in partial pressure of carbon dioxide in arterial blood (PaCO₂) (e.g., hypocapnia, hypercapnia). Certain gasotransmitters are involved in hypoxic sensing by the carotid body including, and not limited to carbon monoxide, and hydrogen sulfide (H₂S ).

Described herein are studies that show that hydrogen sulfide (H₂S) is a physiologic gasotransmitter of the carotid body, enhancing its sensory response to hypoxia. Glomus cells, the site of O₂ sensing in the carotid body, express cystathionine gamma lyase (CSE), an H₂S generating enzyme, with hypoxia increasing H₂S generation in a stimulus-dependent manner. Mice with genetic deletion of CSE display severely impaired carotid body response and ventilatory stimulation to hypoxia as well as a loss of hypoxia-evoked H₂S generation. Pharmacologic inhibition of CSE elicits a similar phenotype in mice and rats. Hypoxia-evoked H₂S generation in the carotid body is regulated by interaction of CSE with hemeoxygenase-2, which generates carbon monoxide.

In some instances, inhibition of HO-2 reduces production of CO, thereby increasing the production of H₂S with subsequent augmentation of carotid body activity. In other embodiments, inhibition of CSE reduces production of H₂S thereby blunting the activity of the carotid body.

### Chemical Control of Ventilation

In normal individuals, balanced activity of two enzymes, cystathionine γ-lyase enzyme (CSE) and heme oxygenase-2 (HO-2), maintains adequate oxygenation during both waking and sleeping states. The enzyme CSE generates H₂S which in turn stimulates the activity of the carotid body. The enzyme HO-2 generates CO which serves as a gasotransmitter signal that suppresses H₂S generation by CSE, thereby reducing the activity of the carotid body.

Thus, where an individual suffers from an SRBD that involves hyperventilation, inhibition of CSE in glomus cells reduces activity of the carotid body, with concomitant dampening of carotid sinus nerve activity. Accordingly, provided herein are methods of treatment of disordered breathing comprising modulation (e.g., down-regulation) of gasotransmitter pathways implicated in the chemical control of breathing. In some embodiments, provided herein are methods of treatment of sleep disordered breathing comprising down-regulation of gasotransmitter pathways implicated in the chemical control of breathing (e.g., by reducing the production of H₂S in the carotid body).

### Central Sleep Apnea (CSA)

Disclosed herein, in certain embodiments, are methods of treating Central Sleep Apnea (CSA). Central sleep apnea is a disorder in which breathing repeatedly stops and starts during sleep. Central sleep apnea often occurs because the brain doesn't send proper signals to the muscles that control breathing, unlike obstructive sleep apnea, in which the inability to breathe normally is due to upper airway obstruction. Central sleep apnea is less common, accounting for fewer than 5 percent of sleep apnea cases.

Central sleep apnea may occur as a result of other conditions, such as heart failure and stroke. Sleeping at a high altitude also may cause central sleep apnea. Treatments for central sleep apnea may involve addressing predisposing conditions, using a device to assist breathing or using supplemental oxygen.

Common signs and symptoms of central sleep apnea include: (a) observed episodes of stopped breathing or abnormal breathing patterns during sleep; (b) abrupt awakenings accompanied by shortness of breath; (c) shortness of breath that's relieved by sitting up; (d) difficulty staying asleep (insomnia); (e) excessive daytime sleepiness (hypersomnia); (f) difficulty concentrating; (g) morning headaches; and (h) snoring.

Although snoring indicates some degree of increased obstruction to airflow, snoring may also be heard in the presence of central sleep apnea. However, snoring may not be as prominent with central sleep apnea as it is with obstructive sleep apnea.

Central sleep apnea often occurs when the brain fails to transmit signals to the breathing muscles. Central sleep apnea can be caused by a number of conditions that affect the ability of the brainstem, which links the brain to the spinal cord and controls many functions such as heart rate and breathing, to control breathing. The cause varies with the type of central sleep apnea. Types include idiopathic central sleep apnea, Cheyne-Stokes breating, medical condition-induced central sleep apnea, drug-induced sleep apnea, high-altitude periodic breathing, and complex sleep apnea. The cause of idiopathic central sleep apnea isn't known. It results in repeated pauses in breathing effort and airflow. Cheyne-Stokes breathing is most commonly associated with congestive heart failure, atrial fibrillation, or stroke and is characterized by a periodic, rhythmic, gradual increase and then decrease in breathing effort and airflow. During the weakest breathing effort, a total lack of airflow (central sleep apnea) can occur. In addition to congestive heart failure, atrial fibrillation, and stroke, several medical conditions may give rise to central sleep apnea. Any damage to the brainstem, which controls breathing, may impair the normal breathing process. Taking certain medications such as opioids, for example, morphine, oxycodone or codeine, may cause breathing to become irregular, to increase and decrease in a regular pattern, or to stop completely. A Cheyne-Stokes breathing pattern may occur upon exposure to a high-enough altitude, such as an altitude greater than 15,000 feet (about 4,500 meters). The change in oxygen at this altitude is the reason for the alternating rapid breathing (hyperventilation) and underbreathing. Some people with obstructive sleep apnea develop central sleep apnea while on treatment with continuous positive airway pressure (CPAP). This is known as complex sleep apnea because it is a combination of obstructive and central sleep apneas.

Treatments for central sleep apnea may comprise addressing associated medical problems, reduction of opioid medications, continuous positive airway pressure, bilevel positive airway pressure, adaptive servo-ventilation, supplemental oxygen, and medications. In some instances, treatment for central sleep apnea comprises addressing associated medical problems. Possible causes of central sleep apnea include other disorders, and treating those conditions may help the central sleep apnea. For example, appropriate therapy for heart failure may eliminate central sleep apnea.

In some embodiments, treatment for central sleep apnea comprises reduction of opioid medications. If opioid medications are causing the central sleep apnea, the dose of those medications may gradually be reduced.

In some embodiments, treatment for central sleep apnea comprises continuous positive airway pressure (CPAP). This method, also used to treat obstructive sleep apnea, involves wearing a mask over the nose during sleep. The mask is attached to a small pump that supplies pressurized air to hold open the upper airway. CPAP may prevent the airway closure that can trigger central sleep apnea. As with obstructive sleep apnea, it's important to use the device only as directed. If the mask is uncomfortable or the pressure feels too strong, adjustments can be made.

Cheyne-Stokes Breathing is currently treated by both pharmacological intervention (e.g., theophylline or acetazolamide) and the use of mechanical devices to aid breathing, e.g., devices that provide positive airway pressure. Continuous positive airway pressure (CPAP) and adaptive servo-ventilation (ASV) are suitable for CSB-CSA patients whose apneas are due to hyperventilation. CPAP and ASV reduce the frequency of apneas by preventing pharyngeal airway narrowing and occlusion during sleep. Successful treatment with CPAP or ASV has been shown to improve left ventricular function, quality of life, and ventilator efficiency during exercise. Successful use of CPAP or ASV is also associated with greater transplant-free survival. CPAP and ASV are only effective or well tolerated in about 50% of individuals with CSB-CSA. Thus, there is a need for a therapeutic approach that increases the efficacy of CPAP or ASV in individuals with CSB-CSA.

One of the causes of CSB-CSA is a hypersensitive chemoreflex feedback loop, or an “elevated loop gain”. Loop-gain can be calculated using two equations. The first is LG=G((PaCO₂—PICO₂)N_{L})T; wherein G is chemosensitivity, V_{L} is lung volume (i.e., functional residual capacity), and T is a “timing factor” that increases with a greater cycle duration of CSB-CSA. The loop gain may also be calculated with the equation: LG=(ΔV_{Drive})/(ΔV_{E})=(2Π)/(2ΠDR-sin(2ΠDR)); where DR is the “duty ratio”.

An LG greater than 1 predicts that small ventilatory disturbances will result in CSB-CSA; alternatively, a LG less than 1 predicts that small ventilatory disturbances will become damped and ventilation will be stable. Further, an LG greater than 1.2 predicts that a heart patient with CSB-CSA will not respond to the use of CPAP; an LG less than 1.2 predicts that a heart patient with CSB-CSA will respond to the use of CPAP.

The first equation demonstrates that chemosensitivity (G) affects loop gain. For example, increasing chemosensitivity increases loop gain; conversely, decreasing chemosensitivity decreases loop gain. In some embodiments, decreasing chemosensitivity in an individual with CSB-CSA and a loop gain greater than 1.2 increases the likelihood that the individual will respond positively to CPAP.

In some embodiments, a CSE inhibitor is administered as an adjuvant therapy to CPAP or ASV in individuals with CSB-CSA and a loop gain greater than 1.2. In some embodiments, administering a CSE inhibitor to an individual with CSB-CSA and a loop gain greater than 1.2 increases the likelihood that the individual will respond positively to CPAP or ASV therapy.

In some embodiments, treatment for central sleep apnea comprises bilevel positive airway pressure (BPAP). Unlike CPAP, which supplies steady, constant pressure to the upper airway as an individual breathes in and out, BPAP builds to a higher pressure when the individual inhales and decreases to a lower pressure when the individual exhales. The goal of this treatment is to boost the weak breathing pattern of central sleep apnea. Some BPAP devices can be set to automatically deliver a breath if the device detects a breath hasn't been taken after a certain number of seconds.

In some embodiments, treatment for central sleep apnea comprises adaptive servo-ventilation (ASV). Some studies have shown this airflow device to be more effective than the CPAP or BPAP for treating central sleep apnea. ASV is designed to treat central sleep apnea and complex sleep apnea by monitoring the normal breathing pattern and storing the information in a built-in computer. After the individual falls asleep, the machine uses pressure to regulate the breathing pattern and prevent pauses in breathing.

In some embodiments, treatment for central sleep apnea comprises supplemental oxygen. Using supplemental oxygen during sleep may help if individual's suffereing from central sleep apnea. Various forms of oxygen are available as well as different devices to deliver oxygen to the lungs.

In some embodiments, treatment for central sleep apnea comprises medications. Certain medications have been used to stimulate breathing in people with central sleep apnea. For example, some doctors prescribe acetazolamide to prevent central sleep apnea in high altitude.

Disclosed herein, in certain embodiments, are methods of treating Central Sleep Apnea in an individual in need thereof. In some embodiments, the methods comprise administering a CSE inhibitor. In some embodiments, the methods comprise administering a CSE inhibitor in combination with a second treatment regimen. In some embodiments, the methods comprise administering a CSE inhibitor before, simultaneously with, or after a second treatment regimen. In some embodiments, the methods comprise administering a CSE inhibitor in combination with a acetazolamide. In some embodiments, the methods comprise administering a CSE inhibitor in combination with CPAP therapy. In some embodiments, the methods comprise administering a CSE inhibitor in combination with a reduction of opioid medications. In some embodiments, the methods comprise administering a CSE inhibitor in combination with an adaptive servo-ventilation therapy. In some embodiments, the methods comprise administering a CSE inhibitor in combination with a supplemental oxygen.

### Apnea of Prematurity (AOP)

Disclosed herein, in certain embodiments, are methods of treating Apnea of Prematurity (AOP). Apnea of prematurity is defined as cessation of breathing by a premature infant that lasts for between 10 and 30 seconds. Apnea of prematurity is most commonly defined as cessation of breathing for more than 15 seconds. It may be accompanied by desaturation and bradycardia, the latter possibly resulting from hypoxic stimulation of the carotid body. The incidence of AOP is inversely related to gestational age. About 10% of infants born at or after 34 weeks develop AOP. About 60% of infants born at or before 28 weeks will develop AOP. Apnea of prematurity has been associated with intraventricular hemorrhage, hydrocephalus, prolonged medical ventilation, and poor developmental outcome in school age children. It may also result in ischemic brain injury. In some embodiments, a method disclosed herein comprises administering a CSE inhibitor to a premature infant diagnosed with AOP.

There appear to be multiple causes of AOP appear, many of which may be related to or exacerbated by immaturity in the PNS and CNS systems that regulate breathing and responses to hypoxia and hypercapnia. Premature infants respond to hyperoxia by depressing the activity of the carotid body; this in turn may induce AOP. Further, premature infants respond to hypoxia by a late depression in ventilation; the depression in ventilation does not contribute to the initiation of apneas (as most infants are not hypoxic prior to apnea) but may prolong apnea, or delay the recovery from apnea. Excessive chemoreceptor sensitivity in the carotid body, for example in response to repeated hypoxia, may destabilize breathing patterns and increase the activity of the carotid body in response to subsequent hypoxias. Furthermore, premature infants exhibit a pronounced decline in minute ventilation in response to hyperoxia that is associated with increased frequency of apnea; this may signify increased carotid body activity. Additionally, hypoxia-induced increases in ventilation correlate with a higher number of apneic episodes. Finally, it is thought that AOP may result from excessive activation of the carotid body in combination with small oscillations in CO₂. In some embodiments, administering a CSE inhibitor to a premature infant with AOP reduces the incidence of apnea, stabilizes breathing patterns, reduces depressions in ventilation, or a combination thereof.

Apnea of prematurity may be obstructive, central, or mixed. Obstructive apnea is responsible for about 10% of the incidence of apnea of prematurity. Central apnea is responsible for about 40% of the incidence of apnea of prematurity. Finally, mixed apnea appears to be responsible for the remaining 50% of apnea of prematurity. In some embodiments, a CSE inhibitor is administered to a premature infant with AOP caused by obstructive apnea. In some embodiments, a CSE inhibitor is administered to a premature infant with AOP caused by mixed apnea. In some embodiments, a CSE inhibitor is administered to a premature infant with AOP caused by central apnea.

Current treatment for Apnea of Prematurity (AOP) includes pharmacological intervention, CPAP, mechanical ventilation, and kinesthetic stimulation. Pharmacological interventions include methylxanthines (e.g., caffeine, theosphylline, and aminophylline), and doxapram. In some embodiments, AOP is treated by administering a CSE inhibitor in combination with a second treatment regimen.

Methylxanthines increases minute ventilation, improves CO₂ sensitivity decreases hypoxic depression of breathing, enhances diaphragmatic contractility, and decreases periodic breathing. Adverse events associated with methylxanthines include tachycardia, cardiac dysrhythmias, jitteriness, irritability, feed intolerance, vomiting, dieresis, and hyperglycemia. Further, methylxanthines are known to interact with multiple drugs. In some embodiments, AOP is treated by administering a CSE inhibitor in combination with a methylxanthine. In some embodiments, AOP is treated by administering a CSE inhibitor in combination with a methylxanthine selected from: caffeine, theosphylline, aminophylline, or a combination thereof. In some embodiments, administering a CSE inhibitor in combination with a methylxanthine enables a medical professional to use a lower dose of the methylxanthine. In some embodiments, the CSE inhibitor works synergistically with the methylxanthine.

Doxapram has also been used to treat AOP; however, it appears that the effects are not sustained longer than 48 hours after commencement of treatment. Further, administration of doxapram is associated with seizures, hypertension, hyperactivity hyperglycemia, and abdominal distension. In some embodiments, AOP is treated by administering a CSE inhibitor in combination with doxapram. In some embodiments, administering a CSE inhibitor in combination with doxapram enables a medical professional to use a lower dose of doxapram. In some embodiments, the CSE inhibitor works synergistically with doxapram.

CPAP is most often used as an adjuvant to pharmacological treatment, especially where significant episodes persist despite pharmacological treatment. The success of CPAP is likely attributable to the reduction in obstructive and mixed apneas. If the apnea is central apnea, nasal intermittent positive pressure ventilation (NIPPV) may be used. Adverse events associated with the use of CPAP in premature infants include barotraumas, abdominal distension, feeding intolerance, and local nasal irritation. In some embodiments, AOP is treated by CPAP therapy and administering a CSE inhibitor.

Disclosed herein, in certain embodiments, are methods of treating Apnea of Prematurity in an individual in need thereof. In some embodiments, the methods comprise administering a CSE inhibitor. In some embodiments, the methods comprise administering a CSE inhibitor in combination with a second treatment regimen. In some embodiments, the methods comprise administering a CSE inhibitor before, simultaneously with, or after a second treatment regimen. In some embodiments, the methods comprise administering a CSE inhibitor in combination with a methylxanthine. In some embodiments, the methods comprise administering a CSE inhibitor in combination with a methylxanthine selected from: caffeine, theosphylline, aminophylline, or a combination thereof. In some embodiments, the methods comprise administering a CSE inhibitor in combination with doxapram. In some embodiments, the methods comprise administering a CSE inhibitor in combination with CPAP therapy.

### Cheyne-Stokes Breathing

Disclosed herein, in certain embodiments, are methods of treating Cheyne-Stokes Breathing. Congestive heart failure patients suffer from a form of non-hypercapnic central sleep apnea characterized by a waxing (crescendo) and waning (decrescendo) pattern of ventilation in which breathing is rapid for a period and then absent for a period (Cheyne-Stokes breathing). Cheyne-Stoke breathing is triggered by hyperventilation. Hyperventilation causes a ventilatory overshoot and hypocapnia. Hypocapnia results in a decrease in ventilatory drive, resulting in a cessation of breathing (apnea). Apnea is then followed by hypercapnia and hypoxia. The carotid body responds to hypoxia and triggers hyperventilation. The result is a Cheyne-Stokes breathing pattern associated with central sleep apnea (CSB-CSA). CSB-CSA is thought to contribute to a poor prognosis by exposing patients to intermittent hypoxia, excessive sympathetic activation and ventricular irritability.

Disclosed herein, in certain embodiments, are methods of treating Cheyne-Stokes Breathing in an individual in need thereof which comprise administering a CSE inhibitor and CPAP or ASV therapy. In some embodiments, the methods comprise administering a CSE inhibitor to an individual that does not or did not respond to CPAP or ASV therapy. In some embodiments, the CSE inhibitor is administered before commencing CPAP or ASV therapy (e.g., a CPAP or ASV therapy regimen or a CPAP or ASV therapy session). In some embodiments, the CSE inhibitor is administered simultaneously with CPAP or ASV therapy.

### Obstructive Sleep Apnea

Disclosed herein, in certain embodiments, are methods of treating obstructive sleep apnea. Obstructive sleep apnea (OSA) or obstructive sleep apnea syndrome is the most common type of sleep apnea and is caused by obstruction of the upper airway. It is characterized by repetitive pauses in breathing during sleep, despite the effort to breathe, and is usually associated with a reduction in blood oxygen saturation. These pauses in breathing, called apneas (literally, “without breath”), typically last 20 to 40 seconds.

The individual with OSA is rarely aware of having difficulty breathing, even upon awakening. It is recognized as a problem by others witnessing the individual during episodes or is suspected because of its effects on the body (sequelae). OSA is commonly accompanied with snoring.

Symptoms may be present for years or even decades without identification, during which time the sufferer may become conditioned to the daytime sleepiness and fatigue associated with significant levels of sleep disturbance. Sufferers who generally sleep alone are often unaware of the condition, without a regular bed-partner to notice and make them aware of their symptoms.

As the muscle tone of the body ordinarily relaxes during sleep, and the airway at the throat is composed of walls of soft tissue, which can collapse, it is not surprising that breathing can be obstructed during sleep. Although a very minor degree of OSA is considered to be within the bounds of normal sleep, and many individuals experience episodes of OSA at some point in life, a small percentage of people are afflicted with chronic, severe OSA.

Many people experience episodes of OSA for only a short period of time. This can be the result of an upper respiratory infection that causes nasal congestion, along with swelling of the throat, or tonsillitis that temporarily produces very enlarged tonsils. The Epstein-Barr virus, for example, is known to be able to dramatically increase the size of lymphoid tissue during acute infection, and OSA is fairly common in acute cases of severe infectious mononucleosis. Temporary spells of OSA syndrome may also occur in individuals who are under the influence of a drug (such as alcohol) that may relax their body tone excessively and interfere with normal arousal from sleep mechanisms.

There are a variety of treatments for OSA; use is determined by an individual patient's medical history, the severity of the disorder and, most importantly, the specific cause of the obstruction. In acute infectious mononucleosis, for example, although the airway may be severely obstructed in the first 2 weeks of the illness, the presence of lymphoid tissue (suddenly enlarged tonsils and adenoids) blocking the throat is usually only temporary. A course of anti-inflammatory steroids such as prednisone (or another kind of glucocorticoid drug) is often given to reduce this lymphoid tissue. Although the effects of the steroids are short term, in most affected individuals, the tonsillar and adenoidal enlargement are also short term, and will be reduced on its own by the time a brief course of steroids is completed. In unusual cases where the enlarged lymphoid tissue persists after resolution of the acute stage of the Epstein-Barr infection, or in which medical treatment with anti-inflammatory steroids does not adequately relieve breathing, tonsillectomy and adenoidectomy may be urgently required.

OSA in children is sometimes due to chronically enlarged tonsils and adenoids. Tonsillectomy and adenoidectomy is curative. The operation may be far from trivial, especially in the worst apnea cases, in which growth is retarded and abnormalities of the right heart may have developed. Even in these extreme cases, the surgery tends to cure not only the apnea and upper airway obstruction, but allows normal subsequent growth and development. Once the high end-expiratory pressures are relieved, the cardiovascular complications reverse themselves. The postoperative period in these children requires special precautions (see “Surgery and obstructive sleep apnea syndrome” below).

The treatment of OSA in adults with poor oropharyngeal airways secondary to heavy upper body type is varied. Unfortunately, in this most common type of OSA, unlike some of the cases discussed above, reliable cures are not the rule.

Some treatments involve lifestyle changes, such as avoiding alcohol and medications that relax the central nervous system (for example, sedatives and muscle relaxants), losing weight, and quitting smoking. Some people are helped by special pillows or devices that keep them from sleeping on their backs, or oral appliances to keep the airway open during sleep. For those cases where these conservative methods are inadequate, doctors can recommend continuous positive airway pressure (CPAP), in which a face mask is attached to a tube and a machine that blows pressurized air into the mask and through the airway to keep it open. There are also surgical procedures intended to remove and tighten tissue and widen the airway, but none has been reproducibly successful. Some individuals may need a combination of therapies to successfully treat their condition. Home polysomnogram equipment can assist patients in reviewing treatment effectiveness. Though some equipment is marketed as sleep hygiene devices not intended for medical use, such as the Zeo, the equipment can prove invaluable under medical supervision to evaluate overall treatment effectiveness in conjunction with physician administered sleep studies.

Some patients may reduce apnea events through the use of nocturnal oxygen, as the use of nocturnal oxygen lowers respiration rate, which minimizes airway collapse.

The most widely used current therapeutic intervention is positive airway pressure whereby a breathing machine pumps a controlled stream of air through a mask worn over the nose, mouth, or both. The additional pressure splints or holds open the relaxed muscles, just as air in a balloon inflates it. There are several variants CPAP, VPAP, or APAP.

(CPAP), or continuous positive airway pressure, in which a computer controlled air flow generator, generates an airstream at a constant pressure. This pressure is prescribed by the patient's physician, based on an overnight test or titration. Newer CPAP models are available which slightly reduce pressure upon exhalation to increase patient comfort and compliance. CPAP is the most common treatment for obstructive sleep apnea.

(VPAP), or variable positive airway pressure, also known as bilevel or BiPAP, uses an electronic circuit to monitor the patient's breathing, and provides two different pressures, a higher one during inhalation and a lower pressure during exhalation. This system is more expensive, and is sometimes used with patients who have other coexisting respiratory problems and/or who find breathing out against an increased pressure to be uncomfortable or disruptive to their sleep.

(APAP), or automatic positive airway pressure, also known as “Auto CPAP”, is the newest form of such treatment. An APAP machine incorporates pressure sensors and a computer which continuously monitors the patient's breathing performance.[17][18] It adjusts pressure continuously, increasing it when the user is attempting to breathe but cannot, and decreasing it when the pressure is higher than necessary. Although FDA-approved, these devices are still considered experimental by many, and are not covered by most United States insurance plans under an APAP-specific code, but only at the rate of a standard CPAP machine.

A second type of physical intervention, a mandibular advancement splint (MAS), is sometimes prescribed for mild or moderate sleep apnea sufferers. The device is a mouthguard similar to those used in sports to protect the teeth. For apnea patients, it is designed to hold the lower jaw slightly down and forward relative to the natural, relaxed position. This position holds the tongue farther away from the back of the airway, and may be enough to relieve apnea or improve breathing for some patients. The FDA has approved only 16 types of oral appliances for the treatment of sleep apnea. A listing is available at its website.

Oral appliance therapy is less effective than CPAP, but is more ‘user friendly’. Side effects are common, but rarely is the patient aware of them.

There are no effective drug-based treatments for obstructive sleep apnea that have FDA approval. However, a clinical trial of mirtazapine, has shown early promise at the University of Illinois at Chicago. This small, early study found a 50% decrease in occurrence of apnea episodes and 28% decrease in sleep disruptions in 100% of patients (twelve patients) taking them. Nonetheless, due to the risk of weight gain and sedation (two risk factors and consequences of sleep apnea) it is not recommended. An effort to improve the effects of mirtazapine by combining it with another existing medication was cancelled during Phase IIa trials in 2006. Dr. David Carley and Dr. Miodrag Radulovacki, the sleep researchers who were behind the initial clinical trial of mirtazapine are now working on a new treatment that consists of two other existing medications taken off-label together for treatment of sleep apnea.

Other serotonin effecting agents that have been explored unsuccessfully as a treatment for apnea include fluoxetine, tryptophan and protriptyline.

Oral administration of the methylxanthine theophylline (chemically similar to caffeine) can reduce the number of episodes of apnea, but can also produce side effects such as heart palpitations and insomnia. Theophylline is generally ineffective in adults with OSA, but is sometimes used to treat central sleep apnea (see below), and infants and children with apnea.

When other treatments do not completely treat the OSA, drugs are sometimes prescribed to treat a patient's daytime sleepiness or somnolence. These range from stimulants such as amphetamines to modern anti-narcoleptic medicines. The anti-narcoleptic medicine modafinil is seeing increased use in this role as of 2004.

In most cases, weight loss will reduce the number and severity of apnea episodes. In the morbidly obese, a major loss of weight (such as what occurs after bariatric surgery) can sometimes cure the condition.

Some researchers believe that OSA is at root a neurological condition, in which nerves that control the tongue and soft palate fail to sufficiently stimulate those muscles, leading to over-relaxation and airway blockage. A few experiments and trial studies have explored the use of pacemakers and similar devices, programmed to detect breathing effort and deliver gentle electrical stimulation to the muscles of the tongue. This is not a common mode of treatment for OSA patients as of 2004, but it is an active field of research.

A small randomized controlled trial reported that compression stockings reduced the number of apneas and hypopnea, perhaps by “prevention of fluid accumulation in the legs during the day, and its nocturnal displacement into the neck at night.”

One study showed that playing the didgeridoo may reduce snoring and daytime sleepiness due to OSA. Since OSA is sometimes caused by hypotonicity (low tone) in the muscles of the throat, playing the didgeridoo may improve symptoms of sleep apnea by exercising muscles of the throat and increasing tone.

A study published in 2009 tested the effect of a set of oropharyngeal exercises developed from exercises used by speech-language pathologists to improve swallowing function. Participants with moderate OSA who performed the exercises every day showed a significant decrease in snoring frequency, snoring intensity, daytime sleepiness, sleep quality score, neck circumference, and AHI score when compared with a control group who performed sham exercises. The improvement in OSA shown by this group was comparable to the improvement shown in patients who use oral appliances to treat this condition.

Although this study was not designed to determine which specific exercises were beneficial, an editorial response to this study in the same journal argues that only two of the set of exercises were likely capable of effecting the improvements they reported. These two exercises included sucking the tongue upward against the palate for a total of three minutes throughout the day, and inflating a balloon by blowing forcefully and then breathing in deeply through the nose, repeated five times without removing the balloon from the mouth. The tongue exercise is intended to increase the strength of tongue protrusion, and the balloon exercise is intended to increase the strength of the pharyngeal wall. Although more research is needed to clarify the effects of oropharyngeal exercise on OSA, this recent study suggests a promising new approach to treating the condition.

Many people benefit from sleeping at a 30 degree elevation of the upper body or higher, as if in a recliner. Doing so helps prevent the gravitational collapse of the airway. Lateral positions (sleeping on a side), as opposed to supine positions (sleeping on the back), are also recommended as a treatment for sleep apnea, largely because the gravitational component is smaller than in the lateral position. A 30 degree elevation of the upper body can be achieved by sleeping in a recliner, an adjustable bed, or a bed wedge placed under the mattress. This approach can easily be used in combination with other treatments and may be particularly effective in very obese people.

Disclosed herein, in certain embodiments, are methods of treating obstructive sleep apnea. In some embodiments, the methods comprise administering a CSE inhibitor. In some embodiments, the methods comprise administering a CSE inhibitor in combination with a second treatment regimen. In some embodiments, the methods comprise administering a CSE inhibitor before, simultaneously with, or after a second treatment regimen. In some embodiments, the methods comprise administering a CSE inhibitor in combination with a CPAP, VPAP, or APAP therapy. In some embodiments, the methods comprise administering a CSE inhibitor in combination with mirtazapine. In some embodiments, the methods comprise administering a CSE inhibitor in combination with a methylxanthine theophylline.

Surgery and Anesthesia in Patients with Sleep Apnea

Many drugs and agents used during surgery to relieve pain and to depress consciousness remain in the body at low amounts for hours or even days afterwards. In an individual with either central, obstructive or mixed sleep apnea, these low doses may be enough to cause life-threatening irregularities in breathing.

Use of analgesics and sedatives in these patients postoperatively should therefore be minimized or avoided.

Surgery on the mouth and throat, as well as dental surgery and procedures, can result in postoperative swelling of the lining of the mouth and other areas that affect the airway. Even when the surgical procedure is designed to improve the airway, such as tonsillectomy and adenoidectomy or tongue reduction—swelling may negate some of the effects in the immediate postoperative period.

Individuals with sleep apnea generally require more intensive monitoring after surgery for these reasons.

A number of different surgeries are available to improve the size or tone of a patient's airway. For decades, tracheostomy was the only effective treatment for sleep apnea. It is used today only in rare, intractable cases that have withstood other attempts at treatment. Modern operations employ one or more of several options, tailored to each patient's needs. Success rates are directly proportional to the accuracy in the initial diagnosis of the site of obstruction.

Nasal surgery, including turbinectomy (removal or reduction of a nasal turbinate), or straightening of the nasal septum, in patients with nasal obstruction or congestion which reduces airway pressure and complicates OSA.

Tonsillectomy and/or adenoidectomy in an attempt to increase the size of the airway.

Removal or reduction of parts of the soft palate and some or all of the uvula, such as uvulopalatopharyngoplasty (UPPP) or laser-assisted uvulopalatoplasty (LAUP). Modern variants of this procedure sometimes use radiofrequency waves to heat and remove tissue.

Reduction of the tongue base, either with laser excision or radiofrequency ablation.

Genioglossus advancement, in which a small portion of the lower jaw that attaches to the tongue is moved forward, to pull the tongue away from the back of the airway.

Hyoid suspension, in which the hyoid bone in the neck, another attachment point for tongue muscles, is pulled forward in front of the larynx.

Maxillomandibular advancement (MMA) is the most effective sleep apnea surgical procedure currently available, with reduction of the AHI to less than 15 in over 90% of patients, and reduction of AHI to <5 in ˜45% of patients. MMA was once thought to be fairly invasive, but has shown to be less painful, in general, than a UPPP soft palate procedure. The associated surgical risks are low, including bleeding, infection, malocclusion, and permanent numbness of the chin and lip. In general, patient perceptions of surgical outcome have been very favorable.

The role of surgery in the treatment of sleep apnea has been questioned repeatedly, as the long term success rate of the procedures has come into question. Patient selection in the past was oftentimes quite poor, resulting in poor overall results. Potential surgical candidates should now be extensively examined to assure the site of obstruction is clearly evident prior to any surgical intervention. The patients' age, weight and other factors may make them a bad candidate for surgery. When a patient can tolerate it, positive air pressure treatment is the gold standard. However, surgical intervention is a viable option for those patients who cannot, or refuse, to use CPAP.

### Upper Airway Resistance Syndrome

Disclosed herein, in certain embodiments, are methods of treating upper airway resistance syndrome (UARS). Upper Airway Resistance Syndrome or UARS is a sleep disorder characterized by airway resistance to breathing during sleep. The primary symptoms include daytime sleepiness and excessive fatigue.

The following lifestyle changes may relieve symptoms of sleep apnea in some people: (a) avoiding alcohol or sedatives at bedtime, which can make symptoms worse; (b) avoiding sleeping on the back may help with mild sleep apnea; and (c) losing weight may decrease the number of apnea spells during the night

Continuous positive airway pressure (CPAP) is now the first treatment for obstructive sleep apnea in most people. CPAP is delivered by a machine with a tight-fitting face mask. Many patients have a hard time sleeping with CPAP therapy. Good follow-up and support from a sleep center can often help overcome any problems in using CPAP.

Some patients may need dental devices inserted into the mouth at night to keep the jaw forward.

Surgery may be an option in some cases. This may involve: (a) uvulopalatopharyngoplasty (UPPP), to remove excess tissue at the back of the throat. This surgery has not been proven to completely clear up sleep apnea. long-term side effects are also possible. More invasive surgeries, to correct problems with the face structures in rare cases when patients have severe sleep apnea and treatment has not helped

Tracheostomy, to create an opening in the windpipe to bypass the blocked airway if there are physical problems (rarely done). Surgery to remove the tonsils and adenoids often cures the condition in children. It does not seem to help most adults.

Disclosed herein, in certain embodiments, are methods of treating upper airway resistance syndrome (UARS). In some embodiments, the methods comprise administering a CSE inhibitor. In some embodiments, the methods comprise administering a CSE inhibitor in combination with a second treatment regimen. In some embodiments, the methods comprise administering a CSE inhibitor before, simultaneously with, or after a second treatment regimen. In some embodiments, the methods comprise administering a CSE inhibitor in combination with an oral systemic balance (OSB) orthotic.

### Idiopathic Central Sleep Apnea

Disclosed herein, in certain embodiments, are methods of treating idiopathic central sleep apnea (ICSA). Idiopathic Central Sleep Apnea (ICSA) is a relatively uncommon disorder and may constitute <5% of patients referred to a sleep clinic.

Patients with ICSA are commonly older men, and may present with complaints of restless sleep, insomnia, and/or daytime symptoms such as sleepiness and fatigue related to insomnia, sleep fragmentation, and arousals. Typically, these patients are thinner and snore less than patents with obstructive sleep apnea.

When patients are evaluated in a sleep medicine laboratory, ICSA is characterized by repetitive episodes of central apnea. However, the cycles of periodic breathing are shorter than those seen in patients that have congestive heart failure.

The diagnosis ICSA is often made after other potential causes of central sleep apnea have been excluded. As the name implies, the underlying mechanisms for this disorder are not fully understood. The cause of ICSA apnea isn't known. It results in repeated pauses in breathing effort and airflow.

Disclosed herein, in certain embodiments, are methods of treating idiopathic central sleep apnea (ICSA). In some embodiments, the methods comprise administering a CSE inhibitor. In some embodiments, the methods comprise administering a CSE inhibitor in combination with a second treatment regimen. In some embodiments, the methods comprise administering a CSE inhibitor before, simultaneously with, or after a second treatment regimen.

### Opioid-Induced CSA

Disclosed herein, in certain embodiments, are methods of treating opioid-induced CSA. In some embodiments, the methods comprise administering a CSE inhibitor. In some embodiments, the methods comprise administering a CSE inhibitor in combination with a second treatment regimen. In some embodiments, the methods comprise administering a CSE inhibitor before, simultaneously with, or after a second treatment regimen. In some embodiments, the methods comprise administering a CSE inhibitor in combination with a reduction of opioid medication.

### Obesity Hypoventilation Syndrome

Disclosed herein, in certain embodiments, are methods of treating Obesity hypoventilation syndrome (OHS). Obesity hypoventilation syndrome (also known as Pickwickian syndrome) is a condition in which severely overweight people fail to breathe rapidly enough or deeply enough, resulting in low blood oxygen levels and high blood carbon dioxide (CO2) levels. Many people with this condition also frequently stop breathing altogether for short periods of time during sleep (obstructive sleep apnea), resulting in many partial awakenings during the night, which leads to continual sleepiness during the day. The disease puts strain on the heart, which eventually may lead to the symptoms of heart failure, such as leg swelling and various other related symptoms. The most effective treatment is weight loss, but it is often possible to relieve the symptoms by nocturnal ventilation with positive airway pressure (CPAP) or related methods.

Obesity hypoventilation syndrome is defined as the combination of obesity (body mass index above 30 kg/m2), hypoxia (falling oxygen levels in blood) during sleep, and hypercapnia (increased blood carbon dioxide levels) during the day, resulting from hypoventilation (excessively slow or shallow breathing).

In people with stable OHS, the most important treatment is weight loss, by diet, through exercise, with medication, or sometimes weight loss surgery (bariatric surgery). This has been shown to improve the symptoms of OHS and resolution of the high carbon dioxide levels. Weight loss may take a long time and is not always successful. Bariatric surgery is avoided if possible, given the high rate of complications, but may be considered if other treatment modalities are ineffective in improving oxygen levels and symptoms. If the symptoms are significant, nighttime positive airway pressure (PAP) treatment is tried; this involves the use of a machine to assist with breathing. PAP exists in various forms, and the ideal strategy is uncertain. Some medications have been tried to stimulate breathing or correct underlying abnormalities; their benefit is again uncertain.

While many people with obesity hypoventilation syndrome are cared for on an outpatient basis, some deteriorate suddenly and when admitted to hospital may show severe abnormalities such as markedly deranged blood acidity (pH<7.25) or depressed level of consciousness due to very high carbon dioxide levels. On occasions, admission to an intensive care unit with intubation and mechanical ventilation is necessary. Otherwise, “bi-level” positive airway pressure (see the next section) is commonly used to stabilize the patient, followed by conventional treatment.

Positive airway pressure, initially in the form of continuous positive airway pressure (CPAP), is a useful treatment for obesity hypoventilation syndrome, particularly when obstructive sleep apnea co-exists. CPAP requires the nighttime use of a machine that delivers a continuous positive pressure to the airways and preventing the collapse of soft tissues in the throat during breathing; it is administered through a mask on either the mouth and nose together, or if that is not tolerated on the nose only (nasal CPAP). This relieves the features of obstructive sleep apnea, and is often sufficient to remove the resultant accumulation of carbon dioxide. The pressure is increased until the obstructive symptoms (snoring and periods of apnea) have disappeared. CPAP alone is effective in more than 50% of people with OHS.

In some occasions, the oxygen levels are persistently too low (oxygen saturations below 90%). In that case, the hypoventilation itself may be improved by switching from CPAP treatment to an alternate device that delivers “bi-level” positive pressure: higher pressure during inspiration (breathing in) and a lower pressure during expiration (breathing out). If this too is ineffective in increasing oxygen levels, addition of oxygen therapy may be necessary. As a last resort, tracheostomy may necessary; this involves making a surgical opening in the trachea to bypass obesity-related airway obstruction in the neck. This may be combined with mechanical ventilation with an assisted breathing device through the opening.

Other treatments for OHS include medroxyprogesterone, a form of the hormone progesterone, has been shown to improve the ventilatory response, but this has been poorly studied and is associated with an increased risk of thrombosis. Similarly, the drug acetazolamide can reduce bicarbonate levels, and thereby augment to normal ventilatory response, but this has been researched insufficiently to recommend wide application.

Disclosed herein, in certain embodiments, are methods of treating Obesity Hypoventilation Syndrome in an individual in need thereof. In some embodiments, the methods comprise administering a CSE inhibitor. In some embodiments, the methods comprise administering a CSE inhibitor in combination with a second treatment regimen. In some embodiments, the methods comprise administering a CSE inhibitor before, simultaneously with, or after a second treatment regimen. In some embodiments, the methods comprise administering a CSE inhibitor in combination with a medroxyprogesterone. In some embodiments, the methods comprise administering a CSE inhibitor in combination with acetazolamide. In some embodiments, the methods comprise administering a CSE inhibitor in combination with CPAP therapy.

### Congenital Central Hypoventilation Syndrome

Congenital central hypoventilation syndrome (CCHS) (also called primary alveolar hypoventilation Ondine's curse) is a respiratory disorder that is fatal if untreated. Persons afflicted with CCHS classically suffer from respiratory arrest during sleep. CCHS is congenital or developed due to severe neurological trauma to the brainstem. The diagnosis may be delayed because of variations in the severity of the manifestations or lack of awareness in the medical community, particularly in milder cases. There are also cases when the diagnosis is made in later life and middle age, although the symptoms are usually obvious in retrospect. Again, lack of awareness in the medical community may cause such a delay.

This is a very rare and serious form of central nervous system failure, involving an inborn failure of autonomic control of breathing. About 1 in 200,000 live born children have the condition. In 2006, there were only about 200 known cases worldwide. In all cases, episodes of apnea occur in sleep, but in a few patients, at the most severe end of the spectrum, apnea also occurs while awake

CCHS is associated with respiratory arrests during sleep and, with incomplete penetrance, to: neuroblastoma (tumors of the sympathetic ganglia), Hirschsprung disease (partial agenesis of the enteric nervous system), dysphagia (difficulty swallowing) and anomalies of the pupilla. Other symptoms include darkening of skin color from inadequate amounts of oxygen, drowsiness, fatigue, headaches, and an inability to sleep at night. Victims of Ondine's curse also suffer from a sensitivity to sedatives and opioids which make respiration even more difficult for the patient. A low concentration of oxygen in the red blood cells also may cause high blood pressure culminating in cor pulmonale or a failure of the right side of the heart.

CCHS is exhibited typically as a congenital disorder, but in rare circumstances, can also result from severe brain or spinal trauma (such as after an automobile accident, stroke, or as a complication of neurosurgery).

Medical investigation of patients with this syndrome has led to a deeper understanding of how the body and brain regulate breathing on a molecular level. PHOX2B can be associated with this condition. This homeobox gene is important for the normal development of the autonomic nervous system.

The disease used to be classified as a “neurocristopathy”, or disease of the neural crest because part of the autonomic nervous system (such as sympathetic ganglia) derives from the neural crest. However, this denomination is no longer favored because essential neurons of the autonomic nervous system, including those that underlie the defining symptom of the disease (respiratory arrests), are derived from the neural tube (the medulla), not from the neural crest, although such mixed embryological origins are also true for most other neurocristopathies.

Patients generally require tracheotomies and lifetime mechanical ventilation on a ventilator in order to survive. However, it has now been shown that Biphasic Cuirass Ventilation can effectively be used without the need for a tracheotomy. Other potential treatments for CCHS include oxygen therapy and medicine for stimulating the respiratory system. Currently problems arise with the extended use of ventilators including fatal infections and pneumonia.

Most people with CCHS do not survive infancy, unless they receive ventilatory assistance during sleep. An alternative to a mechanical ventilator is Phrenic Nerve Pacing/diaphragm pacing. Although rare, cases of long-term untreated CCHS have been reported.

Disclosed herein, in certain embodiments, are methods of treating Congenital Central Hypoventilation Syndrome (CCHS) in an individual in need thereof. In some embodiments, the methods comprise administering a CSE inhibitor. In some embodiments, the methods comprise administering a CSE inhibitor in combination with a second treatment regimen. In some embodiments, the methods comprise administering a CSE inhibitor before, simultaneously with, or after a second treatment regimen. In some embodiments, the methods comprise administering a CSE inhibitor in combination with an oxygen therapy. In some embodiments, the methods comprise administering a CSE inhibitor in combination with a tracheotomy. In some embodiments, the methods comprise administering a CSE inhibitor in combination with a ventilator.

### Primary Snoring

Disclosed herein, in certain embodiments, are methods of treating primary snoring in an individual in need thereof. Primary Snoring, also known as simple snoring, snoring without sleep apnea, noisy breathing during sleep, benign snoring, rhythmical snoring and continous snoring is characterized by loud upper airway breathing sounds in sleep without episodes of apnea (cessation of breath). Primary snoring can be treated by the use of oral/dental devices or surgery. There are mouth/oral devices (that help keep the airway open) on the market that may help to reduce snoring in three different ways. Some devices bring the jaw forward, elevate the soft palate or retain the tongue (from falling back in the airway and thus decreasing snoring). Surgery, such as uvulopalatopharyngoplasty (UPPP) or Laser-Assisted Uvulopalatoplasty (LAUP), that involves removing excess tissue from the throat, can also be used to treat snoring.

Disclosed herein, in certain embodiments, are methods of treating primary snoring in an individual in need thereof. In some embodiments, the methods comprise administering a CSE inhibitor. In some embodiments, the methods comprise administering a CSE inhibitor in combination with a second treatment regimen. In some embodiments, the methods comprise administering a CSE inhibitor before, simultaneously with, or after a second treatment regimen.

### High Altitude Periodic Breathing

Disclosed herein, in certain embodiments, are methods of treating high altitude periodic breathing in an individual in need thereof. High-altitude periodic breathing affects about a quarter of people who ascend to 2500 meters and almost 100% of those who ascend to 4000 meters or higher. It is characterized by central apneas, periodic breathing, insomnia, and sleep fragmentation. There are a variety of medications that may be beneficial, including sedative hypnotics, acetazolamide, steroids, and nonsteroidal anti-inflammatory drugs (NSAIDs). Pregnant women at high altitudes tend to have increased neonatal complications and high risk of low birthweight in newborns.

Disclosed herein, in certain embodiments, are methods of treating high altitude periodic breathing in an individual in need thereof. In some embodiments, the methods comprise administering a CSE inhibitor. In some embodiments, the methods comprise administering a CSE inhibitor in combination with a second treatment regimen. In some embodiments, the methods comprise administering a CSE inhibitor before, simultaneously with, or after a second treatment regimen.

### Chronic Mountain Sickness

Disclosed herein, in certain embodiments, are methods of treating chronic mountain sickness in an individual in need thereof. Chronic mountain sickness (CMS) is a disease that can develop during extended time living at altitude. It is also known as ‘Monge's disease’, after its first description in 1925 by Carlos Monge. While acute mountain sickness is experienced shortly after ascent to high altitude, chronic mountain sickness may develop after many years of living at high altitude. In medicine, high altitude is defined as over 2500 metres (8200 ft), but most cases of CMS occur at over 3000 m (10000 ft).

CMS is characterised by polycythemia (with subsequent increased hematocrit) and hypoxemia which both improve on descent from altitude. CMS is believed to arise because of an excessive production of red blood cells, which increases the oxygen carrying capacity of the blood [2] but may cause increased blood viscosity and uneven blood flow through the lungs (V/Q mismatch). However, CMS is also considered an adaptation of pulmonary and heart disease to life under chronic hypoxia at altitude.

The most frequent symptoms and signs of CMS are headache, dizziness, tinnitus, breathlessness, palpitations, sleep disturbance, fatigue, anorexia, mental confusion, cyanosis, and dilation of veins.

Clinical diagnosis by laboratory indicators have ranges of: Hb>200 g/L, Hct>65%, and arterial oxygen saturation (SaO2)<85% in both genders.

Treatment involves descent from altitude, where the symptoms will diminish and the hematocrit return to normal slowly. Acute treatment at altitude involves bleeding (phlebotomy), removal of circulating blood, to reduce the hematocrit; however this is not ideal for extended periods.

Disclosed herein, in certain embodiments, are methods of treating chronic mountain sickness in an individual in need thereof. In some embodiments, the methods comprise administering a CSE inhibitor. In some embodiments, the methods comprise administering a CSE inhibitor in combination with a second treatment regimen. In some embodiments, the methods comprise administering a CSE inhibitor before, simultaneously with, or after a second treatment regimen.

Impaired Respiratory Motor Control Associated with Stroke

Disclosed herein, in certain embodiments, are methods of treating impaired respiratory motor control associated with stroke in an individual in need thereof. In some embodiments, the methods comprise administering a CSE inhibitor. In some embodiments, the methods comprise administering a CSE inhibitor in combination with a second treatment regimen. In some embodiments, the methods comprise administering a CSE inhibitor before, simultaneously with, or after a second treatment regimen.

Disclosed herein, in certain embodiments, are methods of treating impaired respiratory motor control associated with a neurologic disorder in an individual in need thereof. In some embodiments, the methods comprise administering a CSE inhibitor. In some embodiments, the methods comprise administering a CSE inhibitor in combination with a second treatment regimen. In some embodiments, the methods comprise administering a CSE inhibitor before, simultaneously with, or after a second treatment regimen.

### Certain Terminology

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. In this application, the use of “or” or “and” means “and/or” unless stated otherwise. Furthermore, use of the term “including” as well as other forms, such as “include”, “includes,” and “included,” is not limiting. The section headings used herein are for organizational purposes only and are not to be construed as limiting the subject matter described.

As used herein, “administer” means to provide a treatment, for example to prescribe a treatment, apply a treatment, or distribute a treatment. In some instances, to administer means a medical professional prescribes a treatment which a patient applies (e.g., the patient applies a CPAP device, consumes a medication, or injects a medication). Administration of a medical treatment does not require the immediate or constant supervision of a medical professional.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one embodiment, the mammal is a human.

A “tissue” comprises two or more cells. The two or more cells may have a similar function and/or function. The tissue may be a connective tissue, epithelial tissue, muscular tissue, or nervous tissue. Alternatively, the tissue is a bone, tendon (both referred to as musculoskeletal grafts), cornea, skin, heart valve, or vein.

An “organ” comprises two or more tissues. The two or more tissues may perform a specific function or group of functions. In some instances, the organ is a lung, mouth, nose, parathyroid gland, pineal gland, pituitary gland, carotid body, salivary gland, skin, gall bladder, pancreas, small intestine, stomach, spleen, spinal cord, thymus, thyroid gland, trachea, uterus, or vermiform appendix. Alternatively, the organ is an adrenal gland, appendix, brain, bladder, kidney, intestine, large intestine, small intestine, liver, heart, or muscle.

The term “CSE inhibitor” encompasses a full or partial inhibitor of CSE enzymatic activity in the synthesis of hydrogen sulfide.

The terms “treat,” “treating” or “treatment,” as used herein, include alleviating, abating or ameliorating at least one symptom of a disease or condition, preventing additional symptoms, preventing progression of the condition, inhibiting the disease or condition, e.g., arresting the development of the disease or condition, relieving the disease or condition, causing regression of the disease or condition, relieving a condition caused by the disease or condition, or stopping the symptoms of the disease or condition. In one embodiment, treatment is prophylactic treatment. In another embodiment, treatment refers to therapeutic treatment.

The term “cutaneous” means of the skin or related to the skin, i.e., the external barrier covering the body including the epidermis, the dermis and subcutaneous layers. “Cutaneous” does not cover the lining of internal organs.

The term “cutaneous burn” refers to a cutaneous injury or wound which is not caused by cutting of the skin or tissue. In some embodiments, a cutaneous burn is caused by contact with a chemical, radiation, a hot object or liquid, fire, an allergen, an object carrying electric current, or any other cutaneous burn described herein. The contact with the agent causing a cutaneous burn may be of short duration, or may be prolonged contact. In some cases, a cutaneous burn is caused by exposure to radiation (e.g., a sun burn or a radiation burn associated with chemotherapy).

As used herein a severe partial-thickness refers to a burn that is a second degree burn and extends to the deep reticular dermis. As used herein, a full-thickness burn refers to a third degree burn that extends through the entire dermis. In some embodiments, a severe partial-thickness burn can cover from 1% to 100% of the total body surface area. In some embodiments, a severe partial thickness burn to full thickness burn exceeds 1% of the total body surface area. In some embodiments, a severe partial thickness burn to full thickness burn exceeds 5% of the total body surface area. In some embodiments, a severe partial thickness burn to full thickness burn exceeds 10% of the total body surface area. In some embodiments, a severe partial thickness burn to full thickness burn exceeds 15% of the total body surface area. In some embodiments, a severe partial thickness burn to full thickness burn exceeds 20% of the total body surface area. In some embodiments, a severe partial thickness burn to full thickness burn exceeds 25% of the total body surface area. In some embodiments, a severe partial thickness burn to full thickness burn exceeds 30% of the total body surface area. In some embodiments, a severe partial thickness burn to full thickness burn exceeds 35% of the total body surface area. In some embodiments, a severe partial thickness burn to full thickness burn exceeds 40% of the total body surface area. In some embodiments, a severe partial thickness burn to full thickness burn exceeds 45% of the total body surface area. In some embodiments, a severe partial thickness burn to full thickness burn exceeds 50% of the total body surface area. In some embodiments, a severe partial thickness burn to full thickness burn exceeds 55% of the total body surface area. In some embodiments, a severe partial thickness burn to full thickness burn exceeds 60% of the total body surface area. In some embodiments, a severe partial thickness burn to full thickness burn exceeds 65% of the total body surface area. In some embodiments, a severe partial thickness burn to full thickness burn exceeds 70% of the total body surface area. In some embodiments, a severe partial thickness burn to full thickness burn exceeds 75% of the total body surface area. In some embodiments, a severe partial thickness burn to full thickness burn exceeds 80% of the total body surface area. In some embodiments, a severe partial thickness burn to full thickness burn exceeds 85% of the total body surface area. In some embodiments, a severe partial thickness burn to full thickness burn exceeds 90% of the total body surface area. In some embodiments, a severe partial thickness burn to full thickness burn exceeds 95% of the total body surface area. In some embodiments, a severe partial thickness burn to full thickness burn covers 100% of the total body surface area.

“Activity of the carotid body” refers to the response of the carotid body to various signals. In some embodiments, such signals include pCO₂ or pO₂ in arterial blood. In some embodiments, such signals include presence or absence of certain gasotransmitters such as CO or H₂S in the carotid body or in the vicinity of the carotid body. In some embodiments, such signals include presence or absence of certain ions such as Ca²⁺ or K⁺ ions in the carotid body or in the vicinity of the carotid body. In some embodiments, such signals include action potentials of the nerves that innervate the carotid body.

“Chemosensitivity” of the carotid body refers to the magnitude of the response of the carotid body to a known level of stimulation by chemical messengers including and not limited to O₂, CO₂, CO, and H₂S. Increased chemosensitivity is defined as an increased and disproportionate response to one that is observed under normal physiologic conditions to a similar stimulus.

“Apnea” is the cessation, or near cessation, of airflow. It exists when airflow is less than 20 percent of baseline for at least 10 seconds in adults. These criteria may vary among sleep laboratories and in children. Apnea is most commonly detected using sensors placed at the nose and mouth of the sleeping patient. Inspiratory airflow is typically used to identify an apnea, although both inspiratory and expiratory airflow are usually abnormal. Some laboratories use surrogate measures instead, such as inspiratory chest wall expansion. Three types of apnea are observed during sleep:

“Eupnea” is normal, unlabored ventilation, i.e., resting respiration.

“Hypercapnia” or “hypercarbia” is the presence of excess CO₂ in the blood.

“Hypocapnia” is a state of reduced CO₂ in the blood.

Respiratory effort related arousals (RERAs) exist when there is a sequence of breaths that lasts at least 10 seconds, is characterized by increasing respiratory effort or flattening of the nasal pressure waveform, and leads to an arousal from sleep, but does not meet criteria of an apnea or hypopnea. The inspiratory airflow or tidal volume is maintained during these episodes, but requires increased respiratory effort. RERAs are often accompanied by a terminal snort or an abrupt change in respiratory measures. Daytime sleepiness, fatigue, or inattention can result from microarousals (i.e., electroencephalographic activation lasting three seconds or less), despite the absence of apneas or hypopneas. Snoring may or may not be a prominent complaint. These symptoms are reduced by treatment that alleviates RERAs. RERAs (>5 events per hour) that are associated with daytime sleepiness are a subtype of obstructive sleep apnea (OSA), also called Upper Airway Resistance Syndrome (UARS).

The Apnea-hypopnea index (AHI) is the average total number of apneas and hypopneas per hour of sleep.

The respiratory disturbance index (RDI) is the average total number of events (e.g., apneas, hypopneas, and RERAs) per hour of sleep.

The oxygen desaturation index (ODI) is the average number of times that the oxygen saturation falls by more than 3 or 4 percent per hour of sleep.

The arousal index (ArI) is the average total number of arousals or awakenings per hour of sleep. It is generally lower than the AHI or RDI because approximately 20 percent of apneas or hypopneas are not accompanied by arousals that are evident on polysomnography. However, the ArI can be greater than the AHI or RDI if arousals occur due to causes other than apneas or hypopneas. As examples, arousals can be caused by periodic limb movements, noise, and sleep state transitions.

Cheyne-Stokes breathing refers to a cyclic pattern of crescendo-decrescendo tidal volumes and central apneas, hypopneas, or both. It is commonly associated with heart failure or stroke.

Patients with a “hypoventilation syndromes” generally have mild hypercarbia or elevated serum bicarbonate levels when awake, which worsen during sleep. Hypoventilation syndromes include, and are not limited to, congenital central hypoventilation syndrome (CCHS) and obesity hypoventilation syndrome (OHS).

“Hypoventilation” during sleep is defined as an increase in the arterial carbon dioxide (PaCO₂) of 10 min Hg during sleep (compared with an awake supine value) that lasts at least 25 percent of the sleep time. Directly measuring the pCO₂ in an arterial blood gas during a sleep study is optimal, but impractical. Transcutaneous CO₂ measurements and expired end-tidal CO₂ are alternatives, but are not sufficiently accurate for routine studies. Sleep hypoventilation is usually presumed when persistent oxyhemoglobin desaturation is detected without an alternative explanation, such as apnea or hypopnea.

The term “optionally substituted” or “substituted” means that the referenced group substituted with one or more additional group(s). In certain embodiments, the one or more additional group(s) are individually and independently selected from amide, ester, alkyl, cycloalkyl, heteroalkyl, aryl, heteroaryl, heteroalicyclic, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, amido. In one embodiment, the referenced group is substituted with one or more halogen. In another embodiment, the referenced group is substituted with one or more alkyl.

An “alkyl” group refers to an aliphatic hydrocarbon group. Reference to an alkyl group includes “saturated alkyl” and/or “unsaturated alkyl”. The alkyl group, whether saturated or unsaturated, includes branched, straight chain, or cyclic groups. By way of example only, alkyl includes methyl, ethyl, propyl, iso-propyl, n-butyl, iso-butyl, sec-butyl, t-butyl, pentyl, iso-pentyl, neo-pentyl, and hexyl. In some embodiments, alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. A “lower alkyl” is a C₁-C₆ alkyl. A “heteroalkyl” group substitutes any one of the carbons of the alkyl group with a heteroatom having the appropriate number of hydrogen atoms attached (e.g., a CH₂ group to an NH group or an O group).

An “alkoxy” group refers to a (alkyl)O— group, where alkyl is as defined herein.

The term “alkylamine” refers to the —N(alkyl)ₓH_{y} group, wherein alkyl is as defined herein and x and y are selected from the group x=1, y=1 and x=2, y=0. When x=2, the alkyl groups, taken together with the nitrogen to which they are attached, optionally form a cyclic ring system.

An “amide” is a chemical moiety with formula C(O)NHR or NHC(O)R, where R is selected from alkyl, cycloalkyl, aryl, heteroaryl (bonded through a ring carbon) and heteroalicyclic (bonded through a ring carbon).

The term “ester” refers to a chemical moiety with formula —C(═O)OR, where R is selected from the group consisting of alkyl, cycloalkyl, aryl, heteroaryl and heteroalicyclic.

As used herein, the term “aryl” refers to an aromatic ring wherein each of the atoms forming the ring is a carbon atom. Aryl rings described herein include rings having five, six, seven, eight, nine, or more than nine carbon atoms. Aryl groups are optionally substituted. Examples of aryl groups include, but are not limited to phenyl, and naphthalenyl.

The term “cycloalkyl” refers to a monocyclic or polycyclic non-aromatic radical, wherein each of the atoms forming the ring (i.e. skeletal atoms) is a carbon atom. In various embodiments, cycloalkyls are saturated, or partially unsaturated. In some embodiments, cycloalkyls are fused with an aromatic ring. Cycloalkyl groups include groups having from 3 to 10 ring atoms. Illustrative examples of cycloalkyl groups include, but are not limited to, the following moieties:

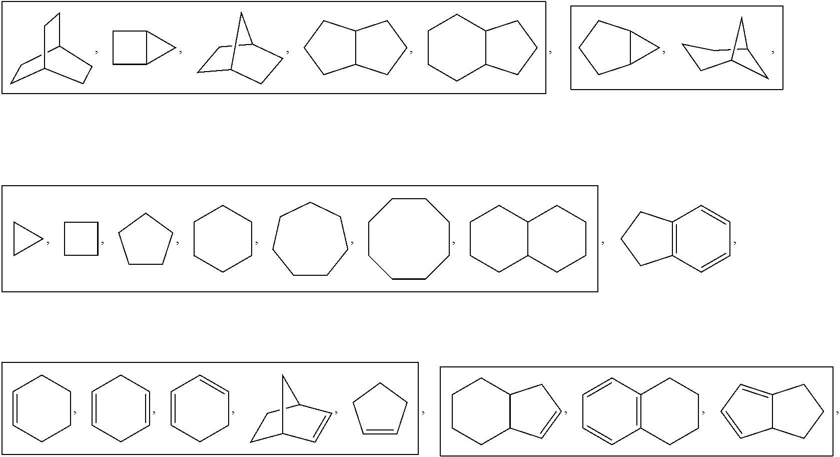

and the like. Monocyclic cycloalkyls include, but are not limited to, cyclopropyl, cyclobutyl, cyclopentyl, cyclohexyl, cycloheptyl, and cyclooctyl. Dicylclic cycloalkyls include, but are not limited to tetrahydronaphthyl, indanyl, tetrahydropentalene or the like. Polycyclic cycloalkyls include adamantane, norbornane or the like. The term cycloalkyl includes “unsaturated nonaromatic carbocyclyl” or “nonaromatic unsaturated carbocyclyl” groups both of which refer to a nonaromatic carbocycle, as defined herein, that contains at least one carbon carbon double bond or one carbon carbon triple bond.

The term “heterocyclo” refers to heteroaromatic and heteroalicyclic groups containing one to four ring heteroatoms each selected from O, S and N. In certain instances, each heterocyclic group has from 4 to 10 atoms in its ring system, and with the proviso that the ring of said group does not contain two adjacent O or S atoms. Non-aromatic heterocyclic groups include groups having 3 atoms in their ring system, but aromatic heterocyclic groups must have at least 5 atoms in their ring system. The heterocyclic groups include benzo-fused ring systems. An example of a 3-membered heterocyclic group is aziridinyl (derived from aziridine). An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. In certain embodiments, heteroaryl groups are monocyclic or polycyclic. Examples of monocyclic heteroaryl groups include and are not limited to:

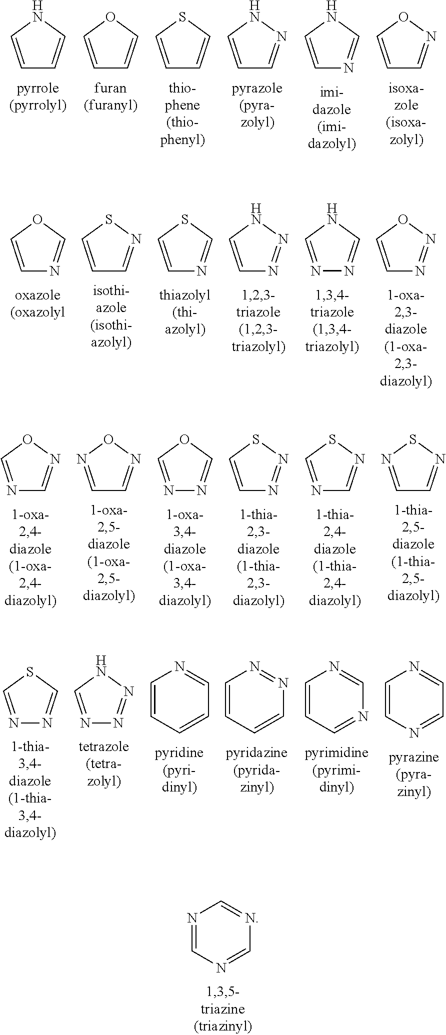

Examples of bicyclic heteroaryl groups include and are not limited to:

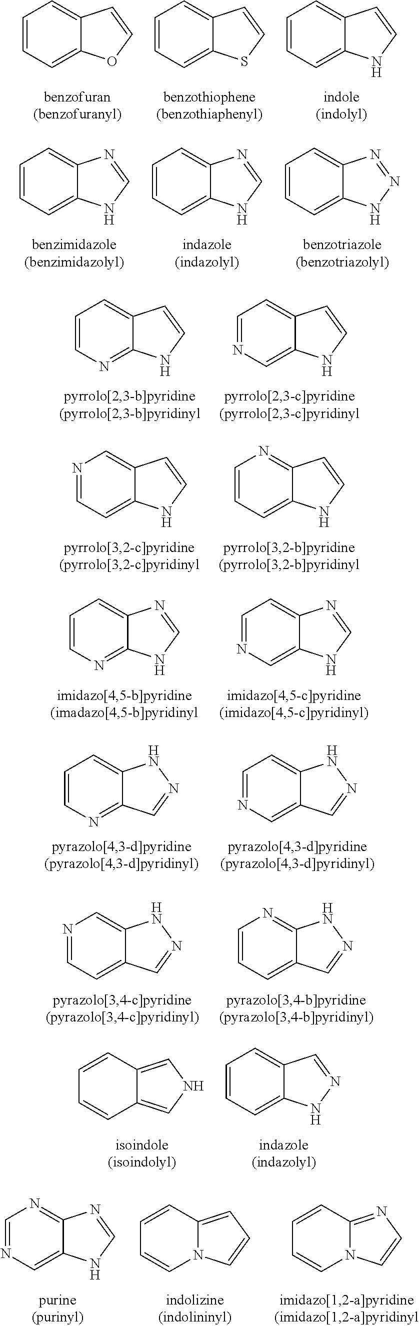

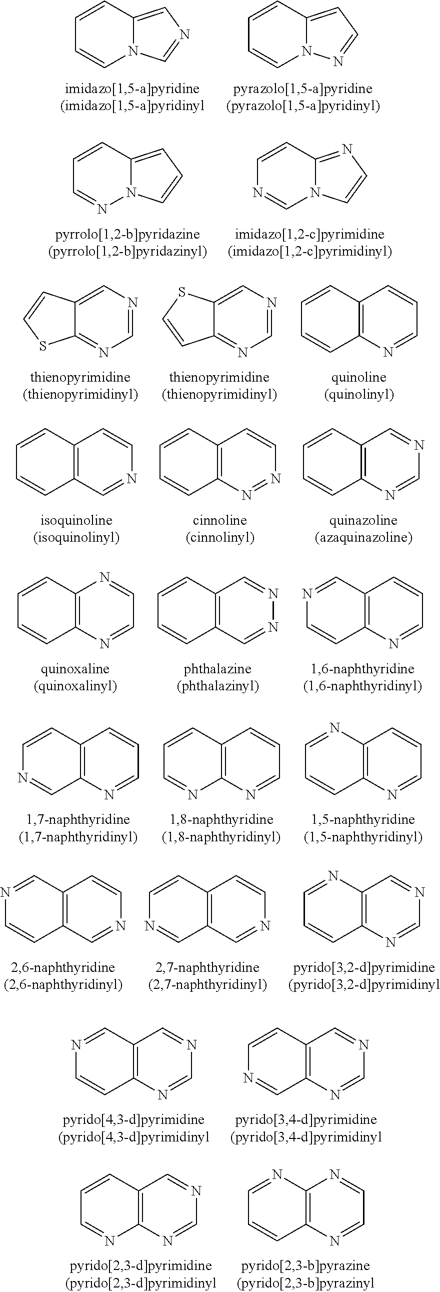

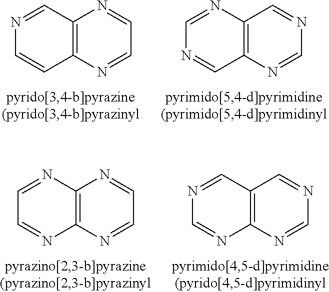 [SMILES: c1cc2nccnc2cn1 ; c1ncc2ncncc2n1 ; c1cnc2nccnc2n1 ; c1ncc2cncnc2n1]

or the like.

A “heteroalicyclic” group or “heterocyclo” group or “heterocycloalkyl” group or “heterocyclyl” group refers to a cycloalkyl group, wherein at least one skeletal ring atom is a heteroatom selected from nitrogen, oxygen and sulfur. In various embodiments, heterocycloalkyls are saturated, or partially unsaturated. In some embodiments, the radicals are fused with an aryl or heteroaryl. Example of saturated heterocyloalkyl groups include

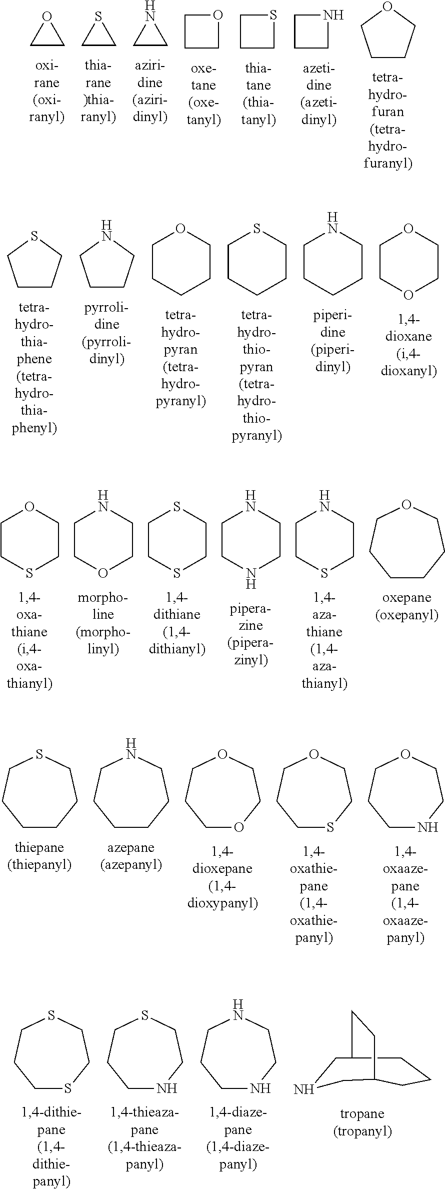

Examples of partially unsaturated heterocyclyl or heterocycloalkyl groups include

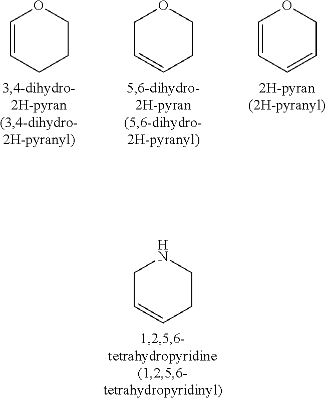 [SMILES: C1=COCCC1 ; C1=CCOCC1 ; C1=CCOC=C1 ; C1=CCNCC1]

Other illustrative examples of heterocyclo or heterocycloalkyl groups, also referred to as non-aromatic heterocycles, include:

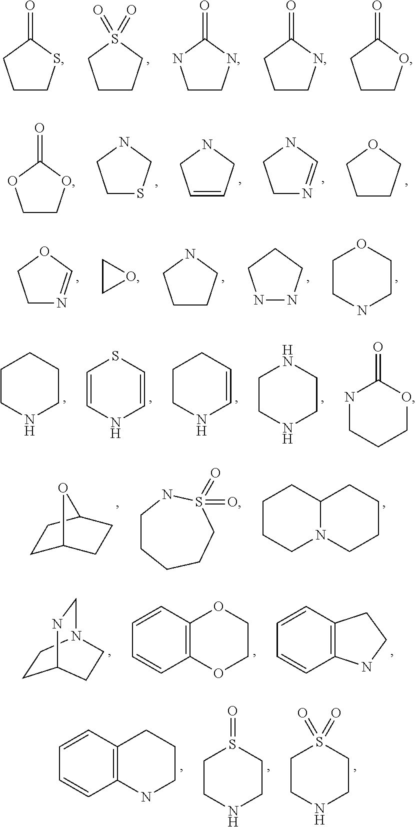

or the like.

The term heteroalicyclic also includes all ring forms of the carbohydrates, including but not limited to the monosaccharides, the disaccharides and the oligosaccharides.

The term “halo” or, alternatively, “halogen” means fluoro, chloro, bromo and iodo.

The terms “haloalkyl,” and “haloalkoxy” include alkyl and alkoxy structures that are substituted with one or more halogens. In embodiments, where more than one halogen is included in the group, the halogens are the same or they are different. The terms “fluoroalkyl” and “fluoroalkoxy” include haloalkyl and haloalkoxy groups, respectively, in which the halo is fluorine.

The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃.

A “cyano” group refers to a CN group.

An “isocyanato” group refers to a NCO group.

A “thiocyanato” group refers to a CNS group.

An “isothiocyanato” group refers to a NCS group.

“Alkoyloxy” refers to a RC(═O)O— group.

“Alkoyl” refers to a RC(═O)— group.

“Isosteres” of a chemical group are chemical groups that have different molecular formulae but exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated include SO₃H, —SO₂NHR₄, —P(O)(OR₄)₂, —P(O)(R₄)(OR₄), —CON(R₄)₂, —CONHNHSO₂R₄, —CONHSO₂R₄, —B(OR₅)₂, —C(R₄)₂B(OR₅)₂, and —CON(R₄)C(R₄)₂B(OR₅)₂; wherein each R₄ is independently H, OH, substituted or unsubstituted alkyl, or substituted or unsubstituted aryl; and R₅ is H or C₁-C₆alkyl. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of preferred carbocyclic and heterocyclic isosteres contemplated.

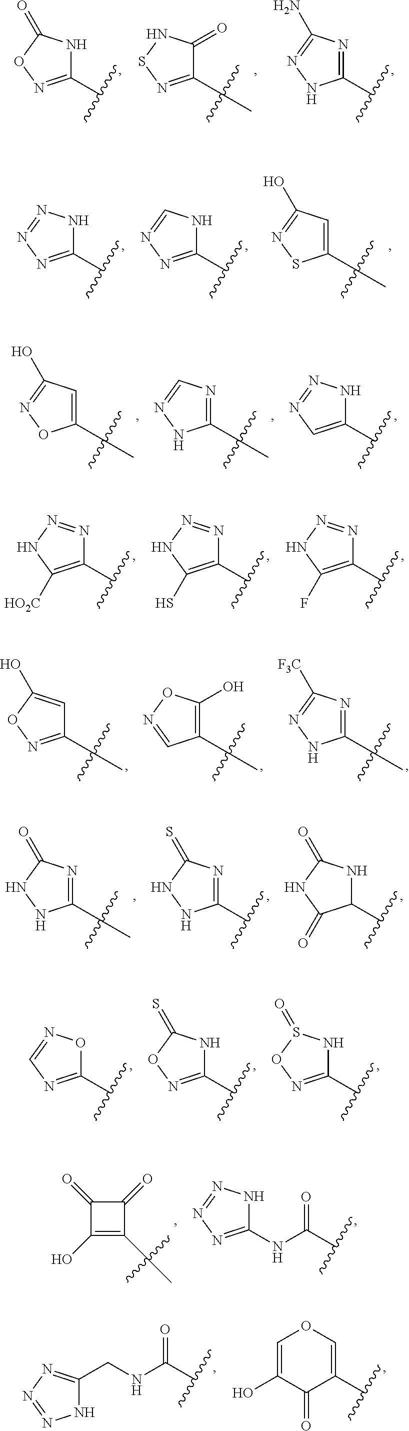

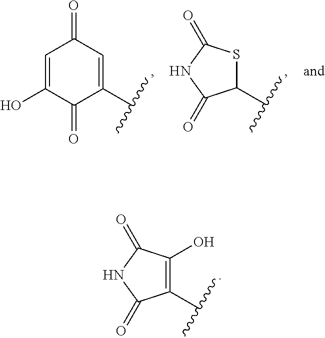 [SMILES: *C1=CC(=O)C=C(O)C1=O ; *C1SC(=O)NC1=O ; *C1=C(O)C(=O)NC1=O]

It is also contemplated that when chemical substituents are added to a carboxylic acid isostere then the inventive compound retains the properties of a carboxylic acid isostere. The present invention contemplates that when a carboxylic acid isostere is optionally substituted, then the substitution cannot eliminate the carboxylic acid isosteric properties of the inventive compound. It is contemplated that the placement of one or more substituents upon a carbocyclic or heterocyclic carboxylic acid isostere is not a substitution at one or more atom(s) which maintain(s) or is/are integral to the carboxylic acid isosteric properties of the compound, if such substituent(s) would destroy the carboxylic acid isosteric properties of the compound.

Other carboxylic acid isosteres not specifically exemplified or described in this specification are also contemplated by the present invention.

### CSE Inhibitor Compounds

In the following description of CSE inhibitory compounds suitable for use in the methods described herein, definitions of referred-to standard chemistry terms may be found in reference works (if not otherwise defined herein), including Carey and Sundberg “Advanced Organic Chemistry 4th Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the ordinary skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients.

Described herein are compounds of any of Formula (1-I), (1-II), (1-IIa), (1-III), (1-IV) or (1-IVa). Also described herein are pharmaceutically acceptable salts, pharmaceutically acceptable solvates, and pharmaceutically acceptable prodrugs of such compounds. Pharmaceutical compositions that include at least one such compound or a pharmaceutically acceptable salt, pharmaceutically acceptable solvate, or pharmaceutically acceptable prodrug of such compound, are provided. In certain embodiments, isomers and chemically protected forms of compounds having a structure represented by any of Formula (1-I), (1-II), (1-IIa), (1-III), (1-IV) or (1-IVa) are also provided.

In one aspect are compounds having the structure of Formula (1-I):

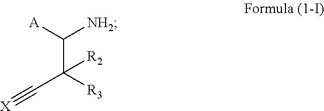

wherein:
A is a carboxylic acid isostere;

### X is CR₁, or N;

R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl;
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;
or a pharmaceutically acceptable salt, solvate, or prodrug thereof

In another aspect are compounds having the structure of Formula (1-II):

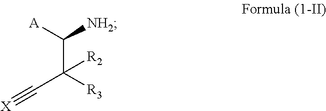

wherein:
A is a carboxylic acid isostere;

### X is CR₁, or N;

R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl;
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;
or a pharmaceutically acceptable salt, solvate, or prodrug thereof.

In another aspect are compounds having the structure of Formula (1-IIa):

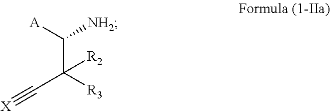

wherein:
A is a carboxylic acid isostere;

### X is CR₁, or N;

R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl;
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;
or a pharmaceutically acceptable salt, solvate, or prodrug thereof

In some embodiments is a compound of Formula (1-I), (1-II), or (1-IIa) wherein A is a carboxylic acid isostere selected from:

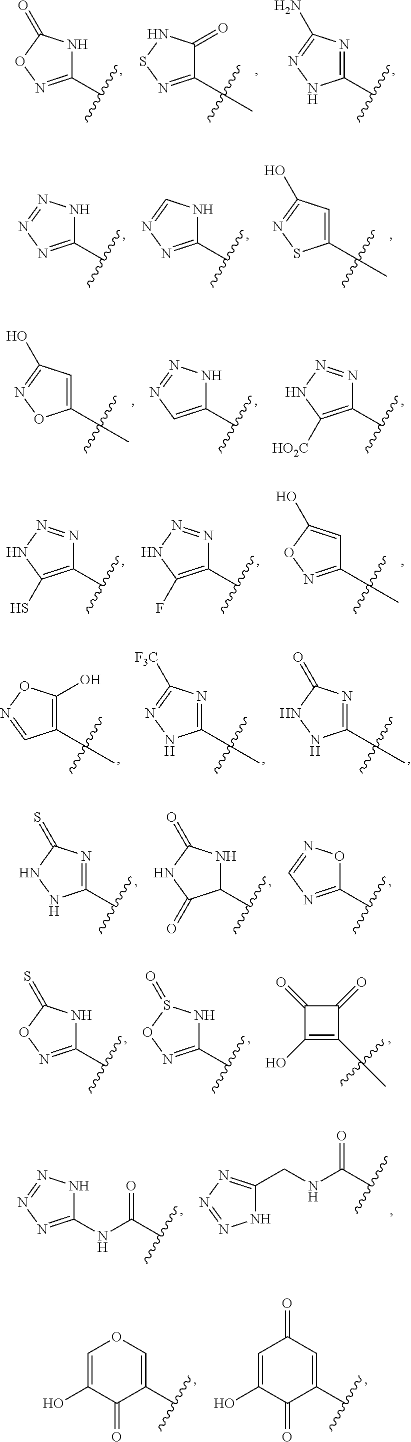

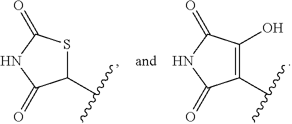 [SMILES: *C1SC(=O)NC1=O ; *C1=C(O)C(=O)NC1=O]

In some embodiments is a compound of Formula (1-I), (1-II), or (1-IIa) wherein A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —B(OR₅)₂, —C(R₄)₂B(OR₅)₂, and —CON(R₄)C(R₄)₂B(OR₅)₂; wherein each R₄ is independently H, OH, substituted or unsubstituted alkyl, or substituted or unsubstituted aryl; and R₅ is H or C₁-C₆alkyl.

In some embodiments is a compound of Formula (1-I), (1-II), or (1-IIa) wherein A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —B(OR₅)₂, —C(R₄)₂B(OR₅)₂, and —CON(R₄)C(R₄)₂B(OR₅)₂; wherein each R₄ is independently H, OH, substituted or unsubstituted alkyl, substituted or unsubstituted heteroaryl, or substituted or unsubstituted aryl; and R₅ is H or C₁-C₆alkyl.

In further embodiments is a compound of Formula (1-I), (1-II), or (1-IIa) wherein X is CR₁. In yet further embodiments is a compound of Formula (1-I), (1-II), or (1-IIa) wherein X is CR₁; and R₁ is H, substituted or unsubstituted alkyl, or substituted or unsubstituted heteroalkyl. In some embodiments is a compound of Formula (1-I), (1-II), or (1-IIa) wherein X is CR₁; and R₁ is H. In some embodiments is a compound of Formula (1-I), (1-II) or (1-IIa) wherein X is CR₁; and R₁ is substituted or unsubstituted alkyl. In some embodiments is a compound of Formula (1-I), (MI), or (1-IIa) wherein X is CR₁; and R₁ is CH₃. In yet further embodiments is a compound of Formula (1-I), (1-II), or (1-IIa) wherein X is CR₁; and R₁ is substituted or unsubstituted heteroalkyl. In other embodiments is a compound of Formula (1-I), (1-II), or (1-Ha) wherein X is CR₁; and R₁ is substituted or unsubstituted heterocycloalkyl. In some embodiments is a compound of Formula (1-I), (1-II), or (1-IIa) wherein X is CR₁; and R₁ is substituted or unsubstituted aryl. In other embodiments is a compound of Formula (1-I), (1-II), or (1-IIa) wherein X is CR₁; and R₁ is substituted or unsubstituted heteroaryl.

In some embodiments is a compound of Formula (1-I), (1-II), or (1-IIa) wherein X is N.

In another aspect are compounds having the structure of Formula (1-III):

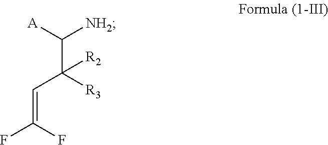

wherein:
A is a carboxylic acid isostere;
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;
or a pharmaceutically acceptable salt, solvate, or prodrug thereof

In another aspect are compounds having the structure of Formula (1-IV):

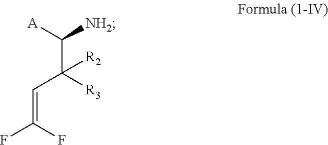

wherein:
A is a carboxylic acid isostere;
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;
or a pharmaceutically acceptable salt, solvate, or prodrug thereof.

In another aspect are compounds having the structure of Formula (1-IVa):

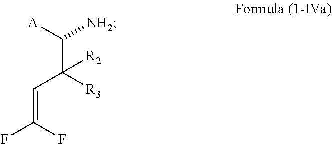

wherein:
A is a carboxylic acid isostere;
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;
or a pharmaceutically acceptable salt, solvate, or prodrug thereof.

In some embodiments is a compound of Formula (1-III), (1-IV), or (1-IVa) wherein A is a carboxylic acid isostere selected from:

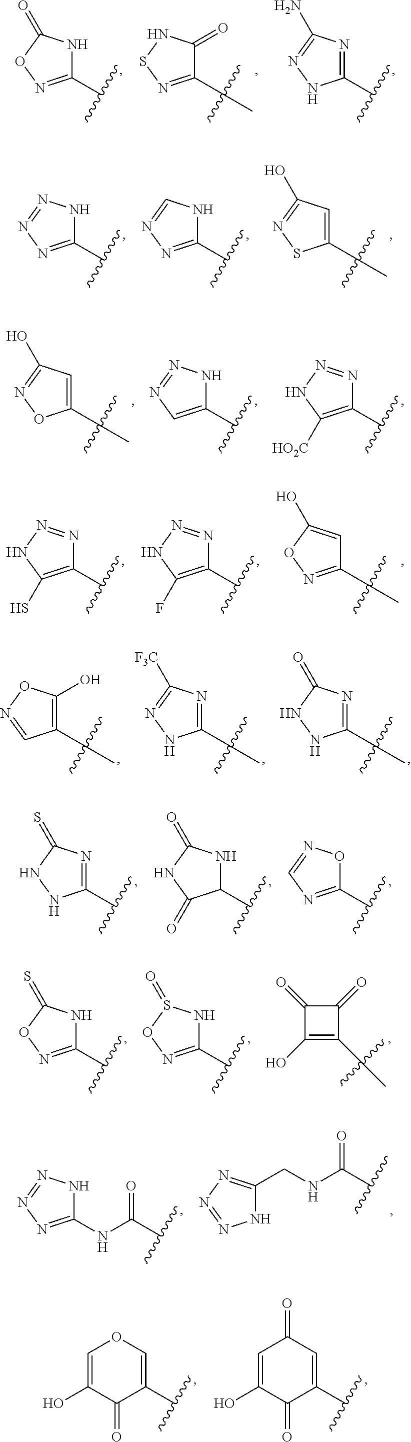

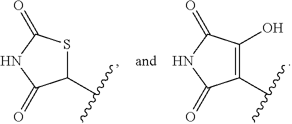 [SMILES: *C1SC(=O)NC1=O ; *C1=C(O)C(=O)NC1=O]

In some embodiments is a compound of Formula (1-III), (1-IV), or (1-IVa) wherein A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —B(OR₅)₂, —C(R₄)₂B(OR₅)₂, and —CON(R₄)C(R₄)₂B(OR₅)₂; wherein each R₄ is independently H, OH, substituted or unsubstituted alkyl, or substituted or unsubstituted aryl; and R₅ is H or C₁-C₆alkyl.

In some embodiments is a compound of Formula (1-III), (1-IV), or (1-IVa) wherein A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —B(OR₅)₂, —C(R₄)₂B(OR₅)₂, and —CON(R₄)C(R₄)₂B(OR₅)₂; wherein each R₄ is independently H, OH, substituted or unsubstituted alkyl, substituted or unsubstituted heteroaryl, or substituted or unsubstituted aryl; and R₅ is H or C₁-C₆alkyl.

In any of the aforementioned embodiments is a compound of Formula (1-I), (1-II), (1-Ha), (1-III), (1-IV) or (1-IVa) wherein R₂ and R₃ are each independently H, substituted or unsubstituted alkyl, or substituted or unsubstituted heteroalkyl. In any of the aforementioned embodiments is a compound of Formula (1-I), (1-II), (1-IIa), (1-III), (1-IV) or (1-IVa) wherein R₂ and R₃ are each H. In any of the aforementioned embodiments is a compound of Formula (1-I), (1-II), (1-IIa), (1-III), (1-IV) or (1-IVa) wherein R₂ and R₃ are each independently substituted or unsubstituted alkyl. In any of the aforementioned embodiments is a compound of Formula (1-I), (1-II), (1-IIa), (1-III), (1-IV) or (1-IVa) wherein R₂ and R₃ are each independently substituted or unsubstituted heteroalkyl.

In any of the aforementioned embodiments is a compound of Formula (1-I), (1-II), (1-IIa), (1-III), (1-IV) or (1-IVa) wherein A is

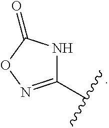 [SMILES: *c1noc(=O)[nH]1]

In any of the aforementioned embodiments is a compound of Formula (1-I), (1-II), (1-IIa), (1-III), (1-IV) or (1-IVa) wherein A is

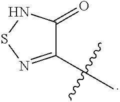 [SMILES: *c1ns[nH]c1=O]

In any of the aforementioned embodiments is a compound of Formula (1-I), (1-II), (1-IIa), (1-III), (1-IV) or (1-IVa) wherein A is

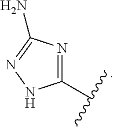 [SMILES: *c1nc(N)n[nH]1]

In any of the aforementioned embodiments is a compound of Formula (1-I), (1-II), (1-IIa), (1-III), (1-IV) or (1-IVa) wherein A is

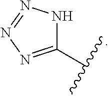 [SMILES: *c1nnn[nH]1]

In any of the aforementioned embodiments is a compound of Formula (1-I), (1-II), (1-IIa), (1-III), (1-IV) or (1-IVa) wherein A is

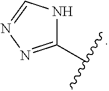 [SMILES: *c1nnc[nH]1]

In any of the aforementioned embodiments is a compound of Formula (1-I), (1-II), (1-IIa), (1-III), (1-IV) or (1-IVa) wherein A is

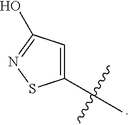 [SMILES: *c1cc(O)ns1]

In any of the aforementioned embodiments is a compound of Formula (1-I), (1-II), (1-IIa), (1-III), (1-IV) or (1-IVa) wherein A is

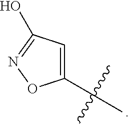 [SMILES: *c1cc(O)no1]

In any of the aforementioned embodiments is a compound of Formula (1-I), (1-II), (1-IIa), (1-III), (1-IV) or (1-IVa) wherein A is

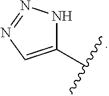 [SMILES: *c1cnn[nH]1]

In any of the aforementioned embodiments is a compound of Formula (1-I), (1-II), (1-IIa), (1-III), (1-IV) or (1-IVa) wherein A is

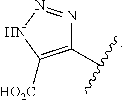 [SMILES: *c1nn[nH]c1C(=O)O]

In any of the aforementioned embodiments is a compound of Formula (1-I), (1-II), (1-IIa), (1-III), (1-IV) or (1-IVa) wherein A is

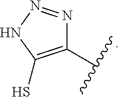 [SMILES: *c1nn[nH]c1S]

In any of the aforementioned embodiments is a compound of Formula (1-I), (1-II), (1-IIa), (1-III), (1-IV) or (1-IVa) wherein A is

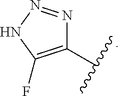 [SMILES: *c1nn[nH]c1F]

In any of the aforementioned embodiments is a compound of Formula (1-I), (1-II), (1-IIa), (1-III), (1-IV) or (1-IVa) wherein A is

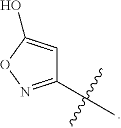 [SMILES: *c1cc(O)on1]

In any of the aforementioned embodiments is a compound of Formula (1-I), (1-II), (1-IIa), (1-III), (1-IV) or (1-IVa) wherein A is

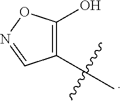 [SMILES: *c1cnoc1O]

In any of the aforementioned embodiments is a compound of Formula (1-I), (1-II), (1-IIa), (1-III), (1-IV) or (1-IVa) wherein A is

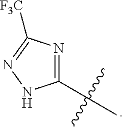 [SMILES: *c1nc(C(F)(F)F)n[nH]1]

In any of the aforementioned embodiments is a compound of Formula (1-I), (1-II), (1-IIa), (1-III), (1-IV) or (1-IVa) wherein A is

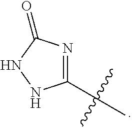 [SMILES: *c1nc(=O)[nH][nH]1]

In any of the aforementioned embodiments is a compound of Formula (1-I), (1-II), (1-IIa), (1-III), (1-IV) or (1-IVa) wherein A is

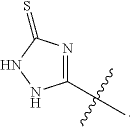 [SMILES: *c1nc(=S)[nH][nH]1]

In any of the aforementioned embodiments is a compound of Formula (1-I), (1-II), (1-IIa), (1-III), (1-IV) or (1-IVa) wherein A is

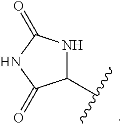 [SMILES: *C1NC(=O)NC1=O]

In any of the aforementioned embodiments is a compound of Formula (1-I), (1-II), (1-IIa), (1-III), (1-IV) or (1-IVa) wherein A is

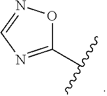 [SMILES: *c1ncno1]

In any of the aforementioned embodiments is a compound of Formula (1-I), (1-II), (1-IIa), (1-III), (1-IV) or (1-IVa) wherein A is

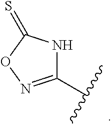 [SMILES: *c1noc(=S)[nH]1]

In any of the aforementioned embodiments is a compound of Formula (1-I), (1-II), (1-IIa), (1-III), (1-IV) or (1-IVa) wherein A is

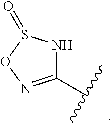 [SMILES: *C1=NOS(=O)N1]

In any of the aforementioned embodiments is a compound of Formula (1-I), (1-II), (1-IIa), (1-III), (1-IV) or (1-IVa) wherein A is

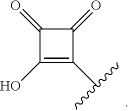 [SMILES: *c1c(O)c(=O)c1=O]

In any of the aforementioned embodiments is a compound of Formula (1-I), (1-II), (1-IIa), (1-III), (1-IV) or (1-IVa) wherein A is

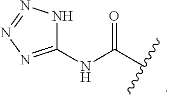 [SMILES: *C(=O)Nc1nnn[nH]1]

In any of the aforementioned embodiments is a compound of Formula (1-I), (1-II), (1-IIa), (1-III), (1-IV) or (1-IVa) wherein A is

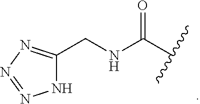 [SMILES: *C(=O)NCc1nnn[nH]1]

In any of the aforementioned embodiments is a compound of Formula (1-I), (1-II), (1-IIa), (1-III), (1-IV) or (1-IVa) wherein A is

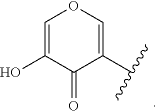 [SMILES: *c1cocc(O)c1=O]

In any of the aforementioned embodiments is a compound of Formula (1-I), (1-II), (1-IIa), (1-III), (1-IV) or (1-IVa) wherein A is

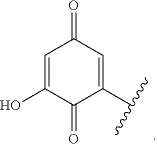 [SMILES: *C1=CC(=O)C=C(O)C1=O]

In any of the aforementioned embodiments is a compound of Formula (1-I), (1-II), (1-11a), (1-III), (1-IV) or (1-IVa) wherein A is

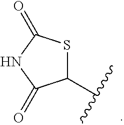 [SMILES: *C1SC(=O)NC1=O]

In any of the aforementioned embodiments is a compound of Formula (1-I), (1-II), (1-IIa), (1-III), (1-IV) or (1-IVa) wherein A is

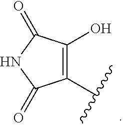 [SMILES: *C1=C(O)C(=O)NC1=O]

In some embodiments is a compound selected from:

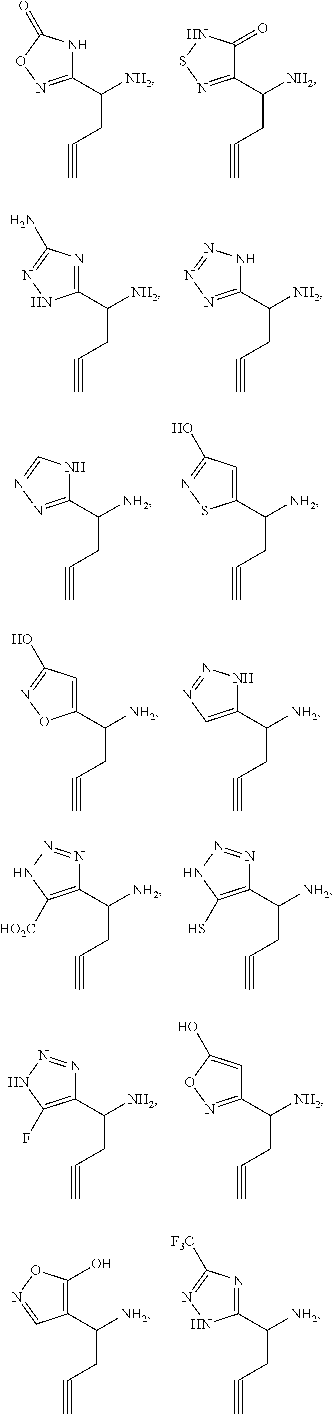

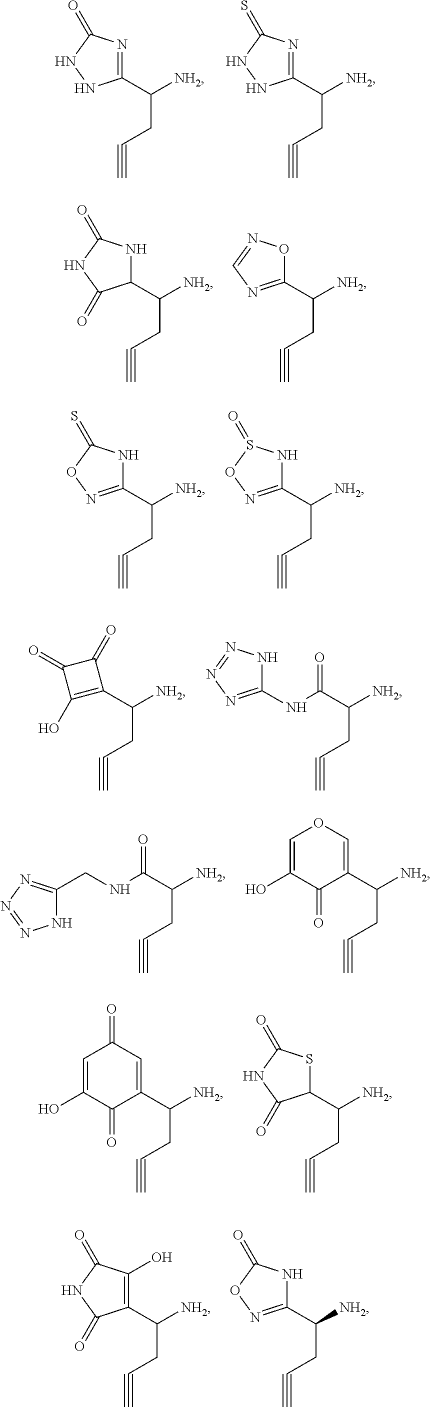

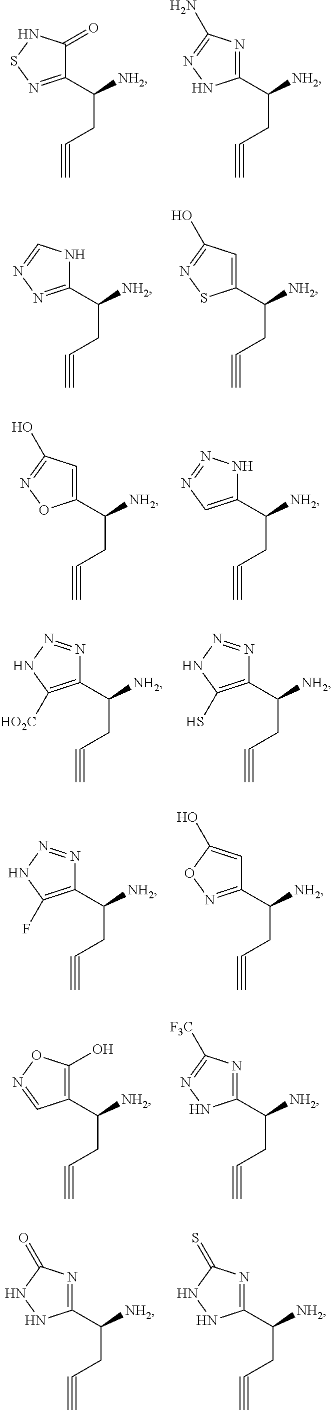

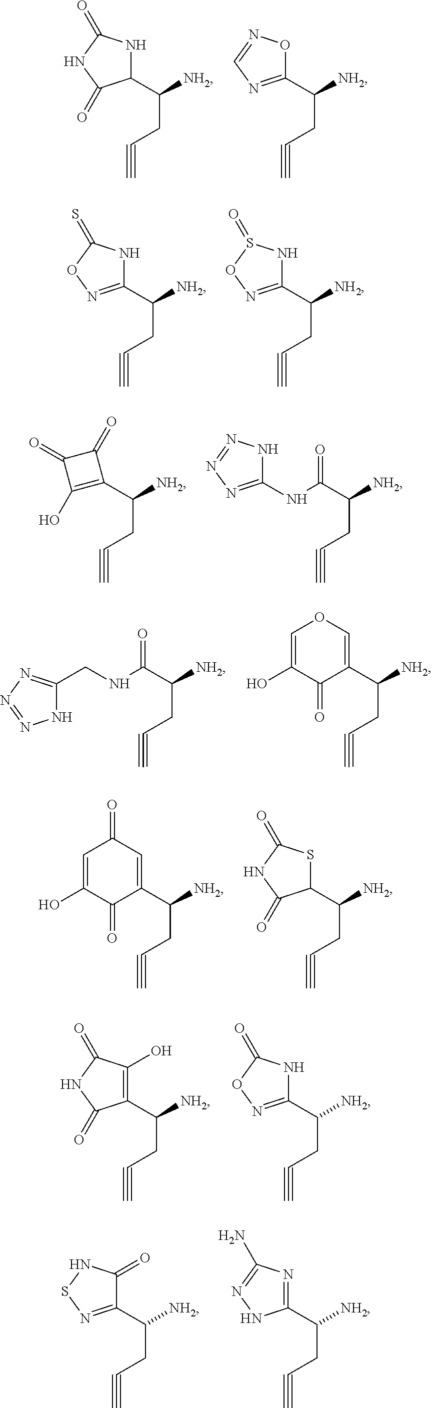

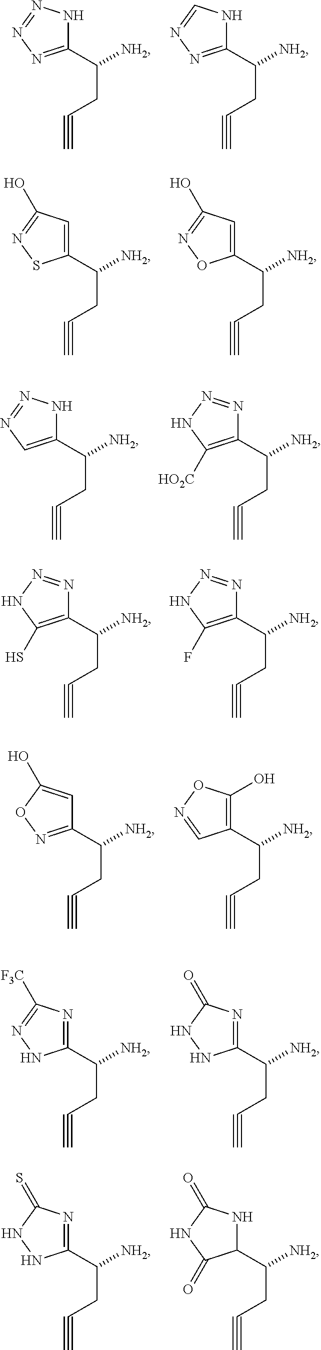

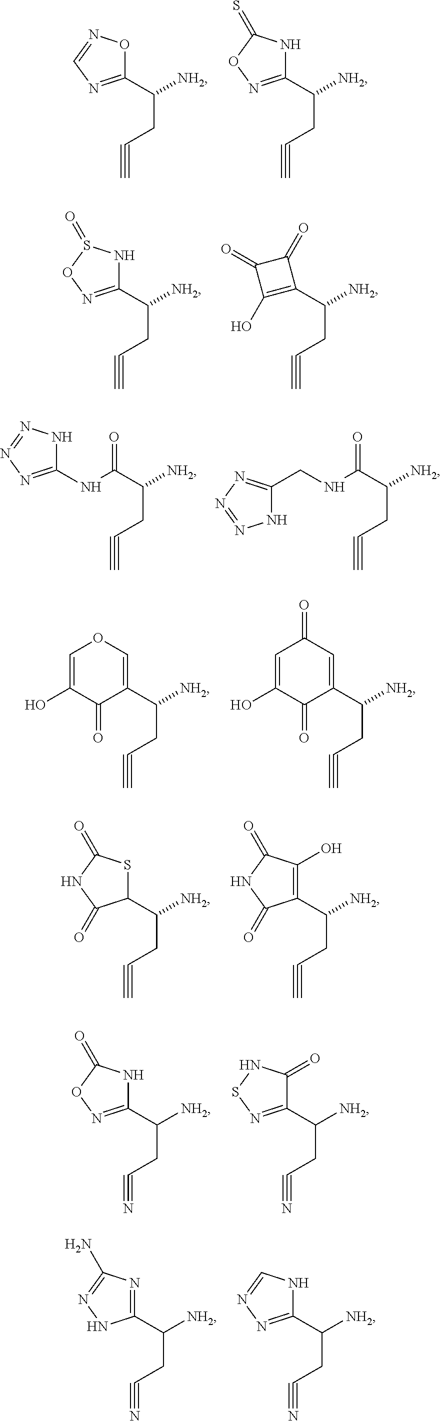

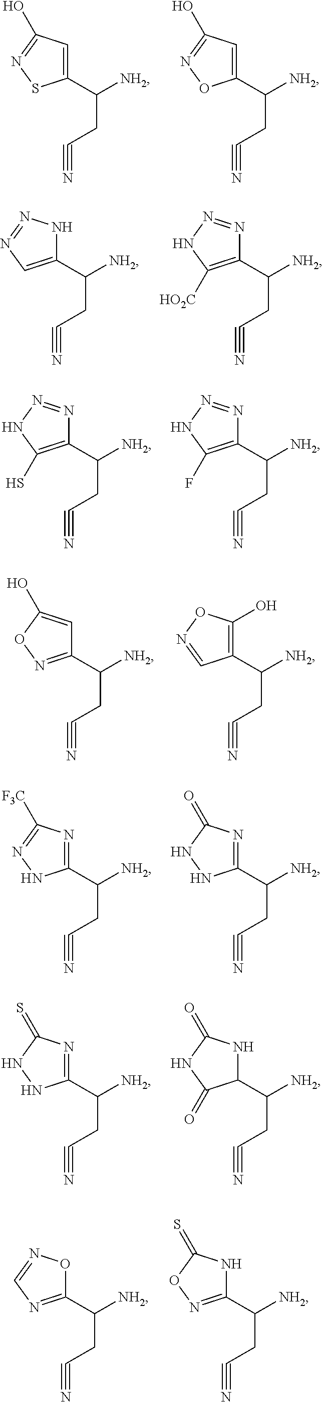

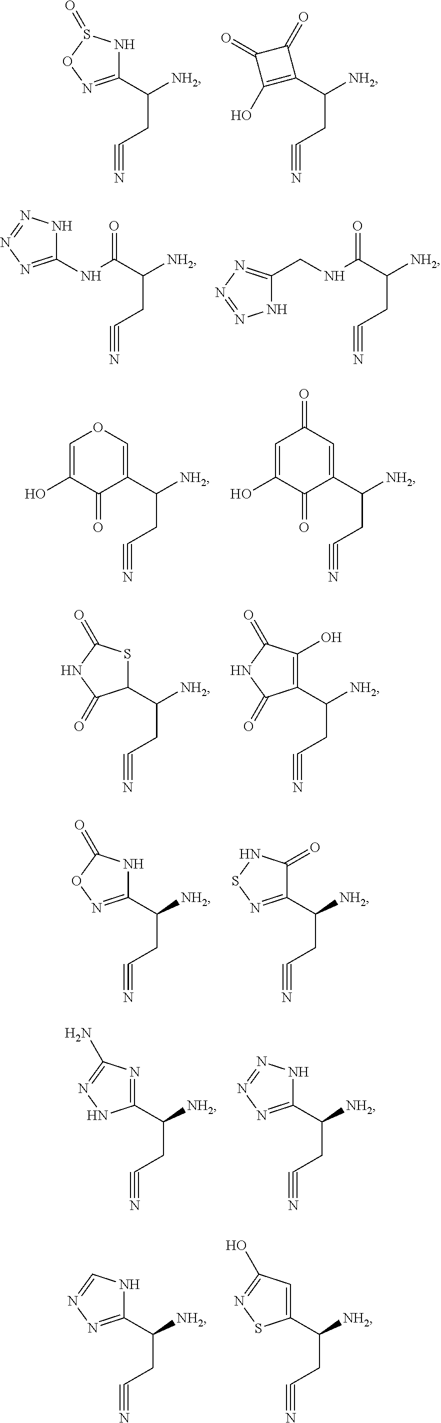

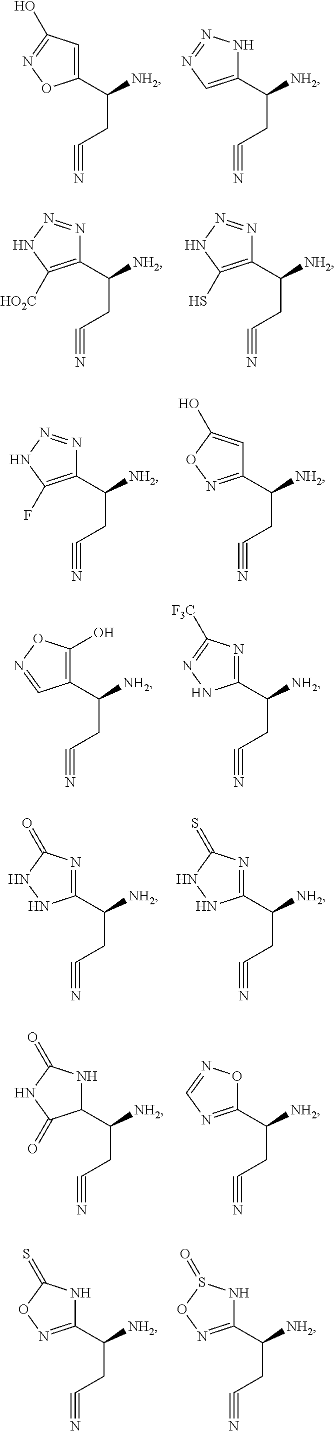

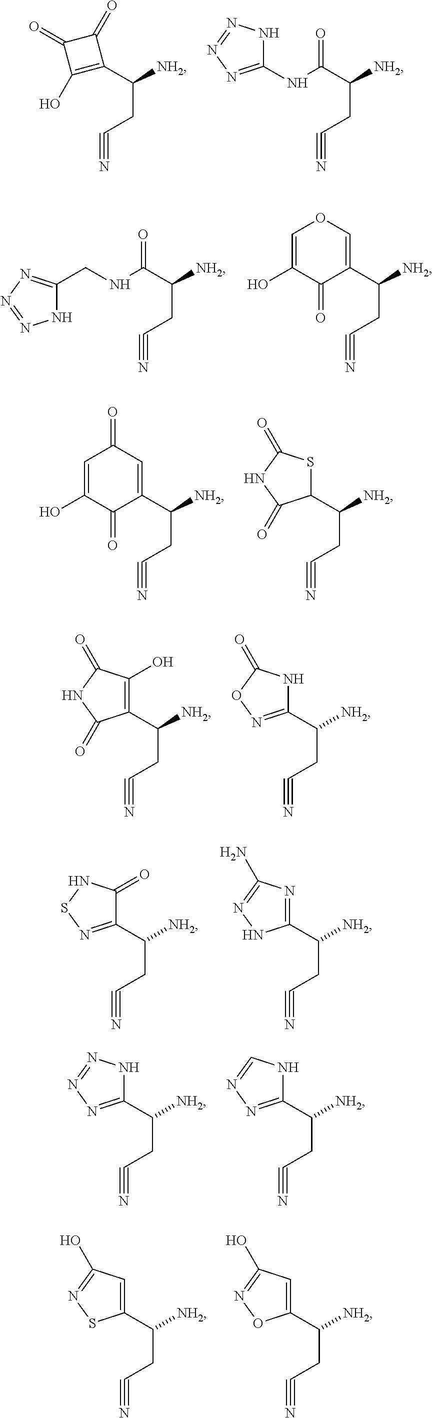

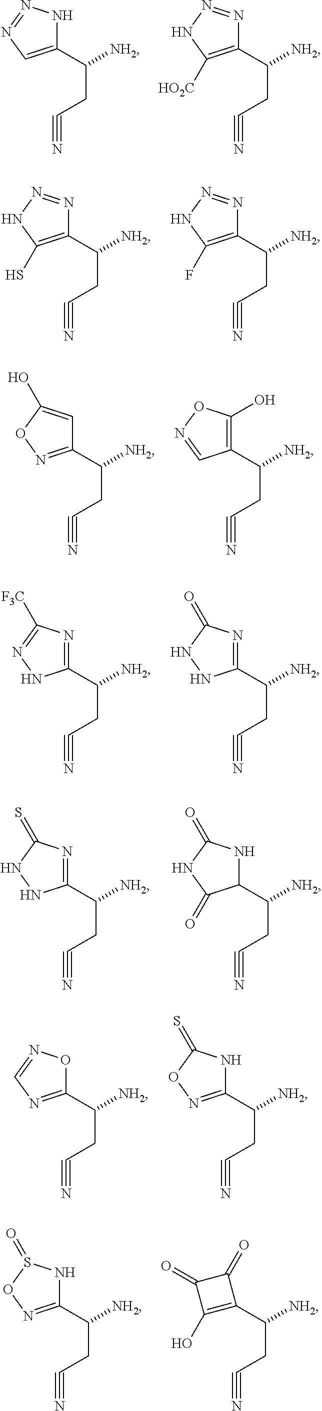

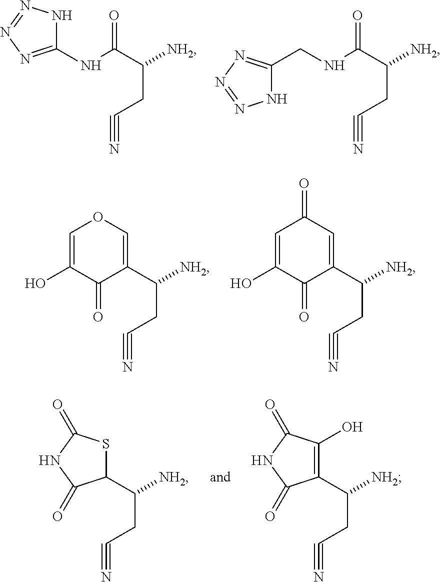 [SMILES: N#CC[C@@H](N)C(=O)Nc1nnn[nH]1 ; N#CC[C@@H](N)C(=O)NCc1nnn[nH]1 ; N#CC[C@@H](N)c1cocc(O)c1=O ; N#CC[C@@H](N)C1=CC(=O)C=C(O)C1=O ; N#CC[C@@H](N)C1SC(=O)NC1=O ; N#CC[C@@H](N)C1=C(O)C(=O)NC1=O]

or a pharmaceutically acceptable salt, solvate, or prodrug thereof.

In some embodiments is a compound selected from:

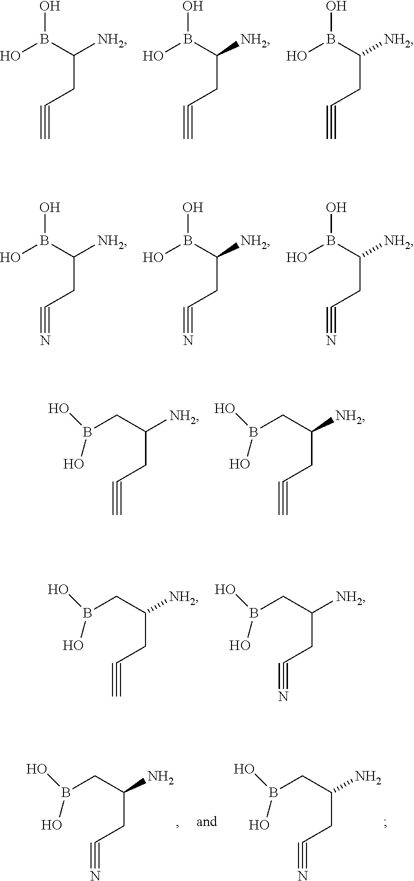

or a pharmaceutically acceptable salt, solvate, or prodrug thereof

Provided herein are pharmaceutical compositions comprising a therapeutically effective amount of a compound of Formula (1-I), (1-II), (1-IIa), (1-III), (1-IV) or (1-IVa), or a pharmaceutically acceptable salt, solvate, or prodrug thereof, and a pharmaceutically acceptable carrier, wherein the compound of Formula (1-I), (1-II), (1-IIa), (1-III), (1-IV) or (1-IVa) is as described herein.

Described herein are compounds of any of Formula (2-I), (2-II), (2-III), (2-IV), (2-V), or (2-VI). Also described herein are pharmaceutically acceptable salts, pharmaceutically acceptable solvates, and pharmaceutically acceptable prodrugs of such compounds. Pharmaceutical compositions that include at least one such compound or a pharmaceutically acceptable salt, pharmaceutically acceptable solvate, or pharmaceutically acceptable prodrug of such compound, are provided. In certain embodiments, isomers and chemically protected forms of compounds having a structure represented by any of Formula (2-I), (2-II), (2-III), (2-IV), (2-V), or (2-VI) are also provided.

In one aspect are compounds having the structure of Formula (2-I):

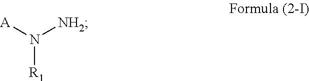

wherein:
A is a carboxylic acid isostere;
R₁ is substituted or unsubstituted C₃-C₆alkyl, substituted or unsubstituted heteroalkyl, substituted or unsubstituted heterocycloalkyl, substituted or unsubstituted aryl, or substituted or unsubstituted heteroaryl; or a pharmaceutically acceptable salt, solvate, or prodrug thereof.

In some embodiments is a compound of Formula (2-I) wherein A is a carboxylic acid isostere selected from:

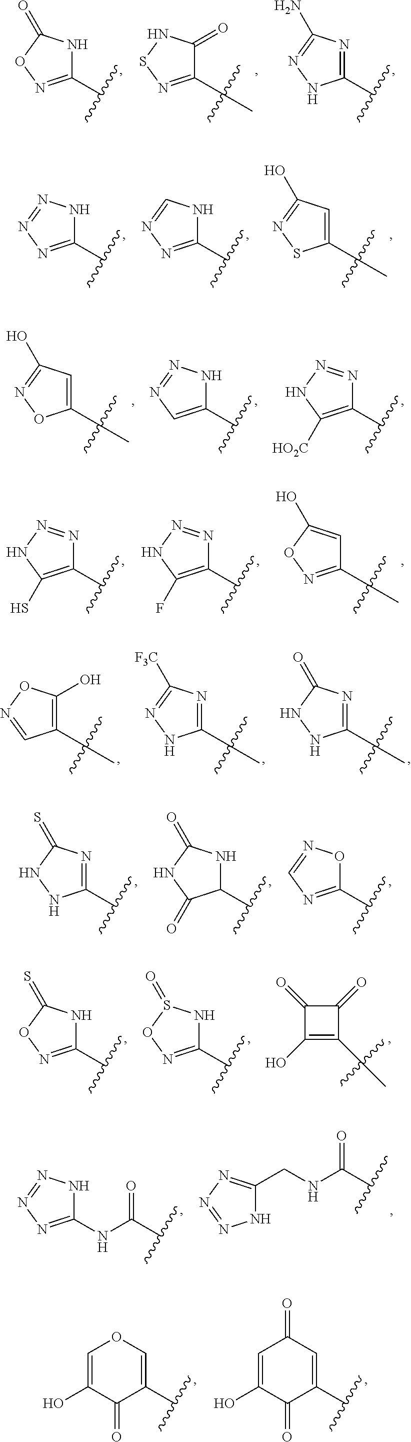

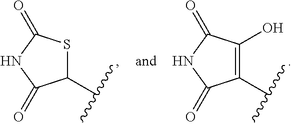 [SMILES: *C1SC(=O)NC1=O ; *C1=C(O)C(=O)NC1=O]

In some embodiments is a compound of Formula (2-I) wherein A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂; wherein each R₄ is independently H, OH, substituted or unsubstituted alkyl, or substituted or unsubstituted aryl; and R₅ is H or C₁-C₆alkyl.

In some embodiments is a compound of Formula (2-I) wherein A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂; wherein each R₄ is independently H, OH, substituted or unsubstituted alkyl, substituted or unsubstituted heteroaryl, or substituted or unsubstituted aryl; and R₅ is H or C₁-C₆alkyl.

In another embodiment is a compound of Formula (2-I) wherein R₁ is H, substituted or unsubstituted C₃-C₆alkyl, substituted or unsubstituted heteroalkyl, substituted or unsubstituted heterocycloalkyl, substituted or unsubstituted aryl, or substituted or unsubstituted heteroaryl. In some embodiments is a compound of Formula (2-I) wherein R₁ is substituted or unsubstituted C₃-C₆alkyl. In further embodiments is a compound of Formula (2-I) wherein R₁ is propyl. In further embodiments is a compound of Formula (2-I) wherein R₁ is butyl. In some embodiments is a compound of Formula (2-I) wherein R₁ is substituted or unsubstituted heteroalkyl. In some embodiments is a compound of Formula (2-I) wherein R₁ is substituted or unsubstituted heterocycloalkyl. In some embodiments is a compound of Formula (2-I) wherein R₁ is substituted or unsubstituted aryl. In some embodiments is a compound of Formula (2-I) wherein R₁ is substituted or unsubstituted heteroaryl.

In another aspect are compounds having the structure of Formula (2-II):

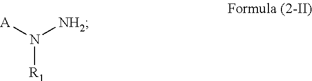

wherein:
R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl;
A is selected from

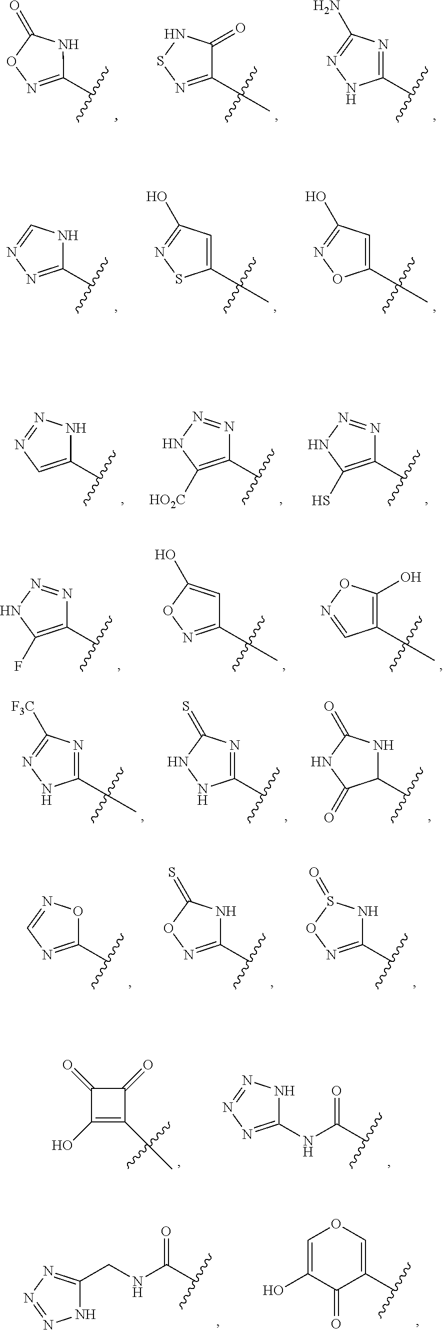

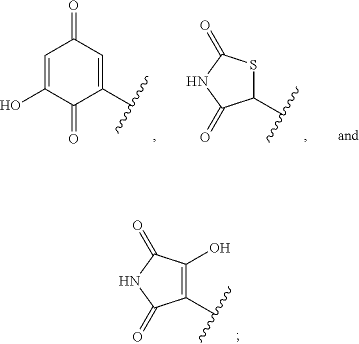 [SMILES: *C1=CC(=O)C=C(O)C1=O ; *C1SC(=O)NC1=O ; *C1=C(O)C(=O)NC1=O]

or a pharmaceutically acceptable salt, solvate, or prodrug thereof

In some embodiments is a compound of Formula (2-II) wherein A is selected from

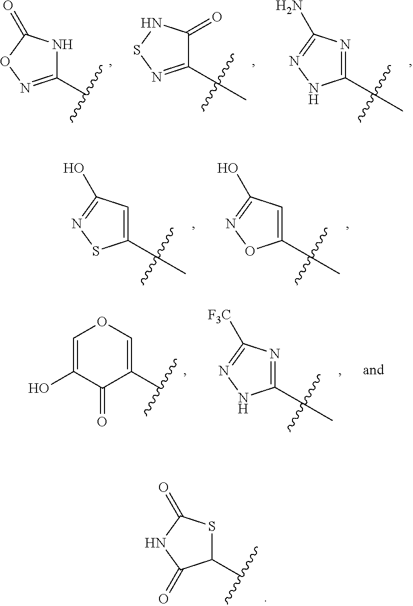

In some embodiments is a compound of Formula (2-II) wherein A is

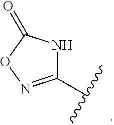 [SMILES: *c1noc(=O)[nH]1]

In some embodiments is a compound of Formula (2-II) wherein A is

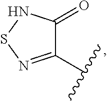 [SMILES: *c1ns[nH]c1=O]

In some embodiments is a compound of Formula (2-II) wherein A is

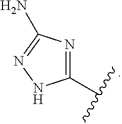 [SMILES: *c1nc(N)n[nH]1]

In some embodiments is a compound of Formula (2-II) wherein A is

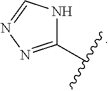 [SMILES: *c1nnc[nH]1]

In some embodiments is a compound of Formula (2-II) wherein A is

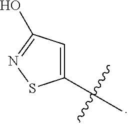 [SMILES: *c1cc(O)ns1]

In some embodiments is a compound of Formula (2-II) wherein A is

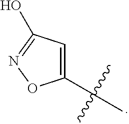 [SMILES: *c1cc(O)no1]

In some embodiments is a compound of Formula (2-II) wherein A is

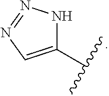 [SMILES: *c1cnn[nH]1]

In some embodiments is a compound of Formula (2-II) wherein A is

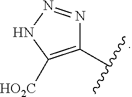 [SMILES: *c1nn[nH]c1C(=O)O]

In some embodiments is a compound of Formula (2-II) wherein A is

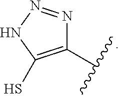 [SMILES: *c1nn[nH]c1S]

In some embodiments is a compound of Formula (2-II) wherein A is

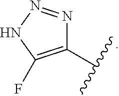 [SMILES: *c1nn[nH]c1F]

In some embodiments is a compound of Formula (2-II) wherein A is

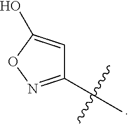 [SMILES: *c1cc(O)on1]

In some embodiments is a compound of Formula (2-II) wherein A is

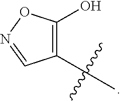 [SMILES: *c1cnoc1O]

In some embodiments is a compound of Formula (2-II) wherein A is

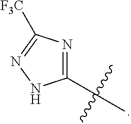 [SMILES: *c1nc(C(F)(F)F)n[nH]1]

In some embodiments is a compound of Formula (2-II) wherein A is

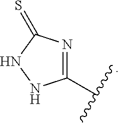 [SMILES: *c1nc(=S)[nH][nH]1]

In some embodiments is a compound of Formula (2-II) wherein A is

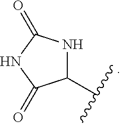 [SMILES: *C1NC(=O)NC1=O]

In some embodiments is a compound of Formula (2-II) wherein A is

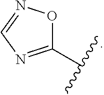 [SMILES: *c1ncno1]

In some embodiments is a compound of Formula (2-II) wherein A is

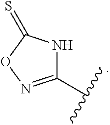 [SMILES: *c1noc(=S)[nH]1]

In some embodiments is a compound of Formula (2-II) wherein A is

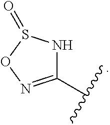 [SMILES: *C1=NOS(=O)N1]

In some embodiments is a compound of Formula (2-II) wherein A is

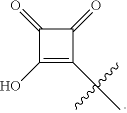 [SMILES: *c1c(O)c(=O)c1=O]

In some embodiments is a compound of Formula (2-II) wherein A is

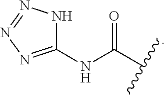 [SMILES: *C(=O)Nc1nnn[nH]1]

In some embodiments is a compound of Formula (2-II) wherein A is

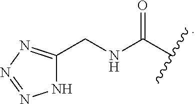 [SMILES: *C(=O)NCc1nnn[nH]1]

In some embodiments is a compound of Formula (2-II) wherein A is

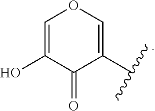 [SMILES: *c1cocc(O)c1=O]

In some embodiments is a compound of Formula (2-II) wherein A is

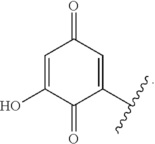 [SMILES: *C1=CC(=O)C=C(O)C1=O]

In some embodiments is a compound of Formula (2-II) wherein A is

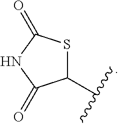 [SMILES: *C1SC(=O)NC1=O]

In some embodiments is a compound of Formula (2-II) wherein A is

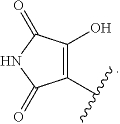 [SMILES: *C1=C(O)C(=O)NC1=O]

In another embodiment is a compound of Formula (2-II) wherein R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl. In some embodiments is a compound of Formula (2-II) wherein R₁ is H. In some embodiments is a compound of Formula (2-II) wherein R₁ is substituted or unsubstituted alkyl. In further embodiments is a compound of Formula (2-II) wherein R₁ is methyl. In further embodiments is a compound of Formula (2-II) wherein R₁ is ethyl. In further embodiments is a compound of Formula (2-II) wherein R₁ is propyl. In further embodiments is a compound of Formula (2-II) wherein R₁ is butyl. In some embodiments is a compound of Formula (2-II) wherein R₁ is substituted or unsubstituted heteroalkyl. In some embodiments is a compound of Formula (2-II) wherein R₁ is substituted or unsubstituted heterocycloalkyl. In some embodiments is a compound of Formula (2-II) wherein R₁ is substituted or unsubstituted aryl. In some embodiments is a compound of Formula (2-II) wherein R₁ is substituted or unsubstituted heteroaryl.

In another aspect are compounds having the structure of Formula (2-III):

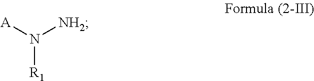

wherein:
R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl;
A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂; wherein each R₄ is independently H, OH, substituted or unsubstituted alkyl, or substituted or unsubstituted aryl; and R₅ is H or C₁-C₆alkyl; or
a pharmaceutically acceptable salt, solvate, or prodrug thereof

In another embodiment is a compound of Formula (2-III) wherein R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl. In some embodiments is a compound of Formula (2-III) wherein R₁ is H. In some embodiments is a compound of Formula (2-III) wherein R₁ is substituted or unsubstituted alkyl. In further embodiments is a compound of Formula (2-III) wherein R₁ is methyl. In further embodiments is a compound of Formula (2-III) wherein R₁ is ethyl. In further embodiments is a compound of Formula (2-III) wherein R₁ is propyl. In further embodiments is a compound of Formula (2-III) wherein R₁ is butyl. In some embodiments is a compound of Formula (2-III) wherein R₁ is substituted or unsubstituted heteroalkyl. In some embodiments is a compound of Formula (2-III) wherein R₁ is substituted or unsubstituted heterocycloalkyl. In some embodiments is a compound of Formula (2-III) wherein R₁ is substituted or unsubstituted aryl. In some embodiments is a compound of Formula (2-III) wherein R₁ is substituted or unsubstituted heteroaryl.

In another embodiment is a compound of Formula (2-III) wherein R₁ is H, and A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂. In another embodiment is a compound of Formula (2-III) wherein R₁ is substituted or unsubstituted alkyl, and A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂. In further embodiments is a compound of Formula (2-III) wherein R₁ is methyl and A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂. In further embodiments is a compound of Formula (2-III) wherein R₁ is ethyl and A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂. In some embodiments is a compound of Formula (2-III) wherein R₁ is substituted or unsubstituted heteroalkyl and A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂. In some embodiments is a compound of Formula (2-III) wherein R₁ is substituted or unsubstituted heterocycloalkyl and A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂. In some embodiments is a compound of Formula (2-III) wherein R₁ is substituted or unsubstituted aryl and A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂. In some embodiments is a compound of Formula (2-III) wherein R₁ is substituted or unsubstituted heteroaryl and A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂. In any of the aforementioned embodiments of Formula (2-III) is a compound of Formula (2-III) wherein A is —SO₃H. In any of the aforementioned embodiments of Formula (2-III) is a compound of Formula (2-III) wherein A is —SO₂NHR₄. In any of the aforementioned embodiments of Formula (2-III) is a compound of Formula (2-III) wherein A is —P(O)(OR₄)₂. In any of the aforementioned embodiments of Formula (2-III) is a compound of Formula (2-III) wherein A is —P(O)(R₄)(OR₄). In any of the aforementioned embodiments of Formula (2-III) is a compound of Formula (2-III) wherein A is —CON(R₄)₂. In any of the aforementioned embodiments of Formula (2-III) is a compound of Formula (2-III) wherein A is —CONHNHSO₂R₄. In any of the aforementioned embodiments of Formula (2-III) is a compound of Formula (2-III) wherein A is —CONHSO₂R₄. In any of the aforementioned embodiments of Formula (2-III) is a compound of Formula (2-III) wherein A is —C(R₄)₂B(OR₅)₂. In any of the aforementioned embodiments of Formula (2-III) is a compound of Formula (2-III) wherein A is —CON(R₄)C(R₄)₂B(OR₅)₂.

In another aspect are compounds having the structure of Formula (2-IV):

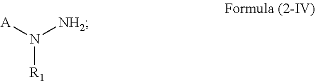

wherein:

A is

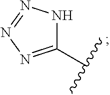 [SMILES: *c1nnn[nH]1]

R₁ is substituted or unsubstituted C₂-C₆alkyl, substituted or unsubstituted heteroalkyl, substituted or unsubstituted heterocycloalkyl, substituted or unsubstituted aryl, or substituted or unsubstituted heteroaryl; or a pharmaceutically acceptable salt, solvate, or prodrug thereof.

In another embodiment is a compound of Formula (2-IV) wherein R₁ is substituted or unsubstituted C₂-C₆alkyl, substituted or unsubstituted heteroalkyl, substituted or unsubstituted heterocycloalkyl, substituted or unsubstituted aryl, or substituted or unsubstituted heteroaryl. In some embodiments is a compound of Formula (2-IV) wherein R₁ is substituted or unsubstituted C₂-C₆alkyl. In further embodiments is a compound of Formula (2-IV) wherein R₁ is ethyl. In further embodiments is a compound of Formula (2-IV) wherein R₁ is propyl. In further embodiments is a compound of Formula (2-IV) wherein R₁ is butyl. In some embodiments is a compound of Formula (2-IV) wherein R₁ is substituted or unsubstituted heteroalkyl. In some embodiments is a compound of Formula (2-IV) wherein R₁ is substituted or unsubstituted heterocycloalkyl. In some embodiments is a compound of Formula (2-IV) wherein R₁ is substituted or unsubstituted aryl. In some embodiments is a compound of Formula (2-IV) wherein R₁ is substituted or unsubstituted heteroaryl.

In another aspect are compounds having the structure of Formula (2-V):

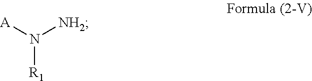

wherein:

A is

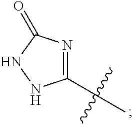 [SMILES: *c1nc(=O)[nH][nH]1]

R₁ is H, substituted or unsubstituted C₃-C₆alkyl, substituted or unsubstituted heteroalkyl, substituted or unsubstituted heterocycloalkyl, substituted or unsubstituted aryl, or substituted or unsubstituted heteroaryl; or a pharmaceutically acceptable salt, solvate, or prodrug thereof.

In another embodiment is a compound of Formula (2-V) wherein R₁ is substituted or unsubstituted C₃-C₆alkyl, substituted or unsubstituted heteroalkyl, substituted or unsubstituted heterocycloalkyl, substituted or unsubstituted aryl, or substituted or unsubstituted heteroaryl. In some embodiments is a compound of Formula (2-V) wherein R₁ is H. In some embodiments is a compound of Formula (2-V) wherein R₁ is substituted or unsubstituted C₃-C₆alkyl. In further embodiments is a compound of Formula (2-V) wherein R₁ is propyl. In further embodiments is a compound of Formula (2-V) wherein R₁ is isopropyl. In further embodiments is a compound of Formula (2-V) wherein R₁ is butyl. In some embodiments is a compound of Formula (2-V) wherein R₁ is substituted or unsubstituted heteroalkyl. In some embodiments is a compound of Formula (2-V) wherein R₁ is substituted or unsubstituted heterocycloalkyl. In some embodiments is a compound of Formula (2-V) wherein R₁ is substituted or unsubstituted aryl. In some embodiments is a compound of Formula (2-V) wherein R₁ is substituted or unsubstituted heteroaryl.

In one aspect are compounds having the structure of Formula (2-VI):

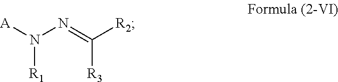

wherein:
A is a carboxylic acid isostere;
R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl;
R₂ is substituted or unsubstituted alkyl, substituted or unsubstituted heteroalkyl, substituted or unsubstituted heterocycloalkyl, substituted or unsubstituted aryl, substituted or unsubstituted heteroaryl, or —CH₂C(O)(substituted or unsubstituted aryl);
R₃ is H, or substituted or unsubstituted alkyl; or
R₂ and R₃ together with the carbon atom to which they are attached form a cycloalkyl or heterocycloalkyl ring; or a pharmaceutically acceptable salt, solvate, or prodrug thereof.

In some embodiments is a compound of Formula (2-VI) wherein A is a carboxylic acid isostere selected from:

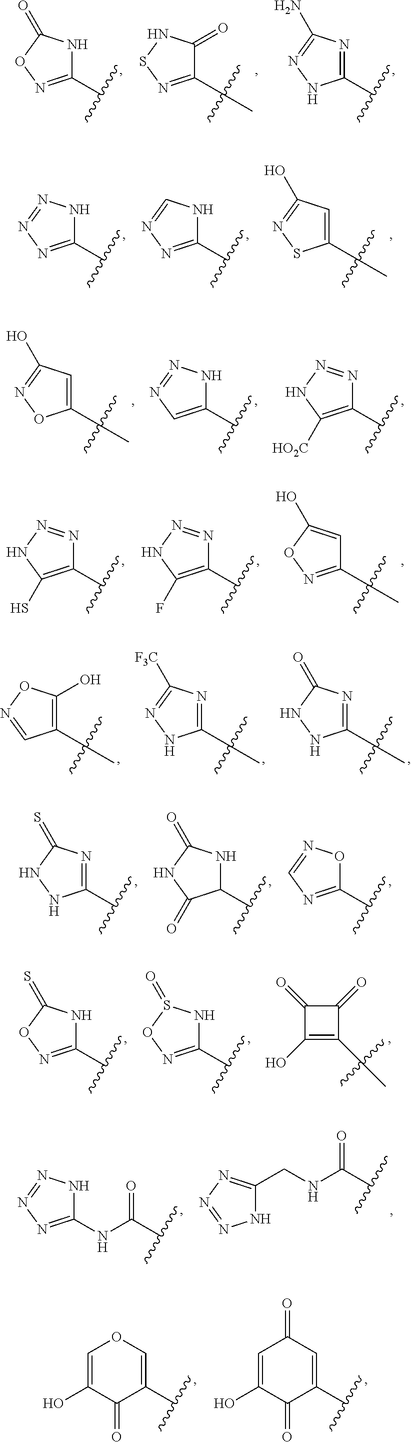

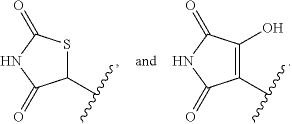 [SMILES: *C1SC(=O)NC1=O ; *C1=C(O)C(=O)NC1=O]

In some embodiments is a compound of Formula (2-VI) wherein A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂; wherein each R₄ is independently H, OH, substituted or unsubstituted alkyl, or substituted or unsubstituted aryl; and R₅ is H or C₁-C₆alkyl.

In another embodiment is a compound of Formula (2-VI) wherein R₁ is substituted or unsubstituted alkyl, substituted or unsubstituted heteroalkyl, substituted or unsubstituted heterocycloalkyl, substituted or unsubstituted aryl, or substituted or unsubstituted heteroaryl. In some embodiments is a compound of Formula (2-VI) wherein R₁ is H. In some embodiments is a compound of Formula (2-VI) wherein R₁ is substituted or unsubstituted alkyl. In further embodiments is a compound of Formula (2-VI) wherein R₁ is methyl. In further embodiments is a compound of Formula (2-VI) wherein R₁ is ethyl. In further embodiments is a compound of Formula (2-VI) wherein R₁ is propyl. In further embodiments is a compound of Formula (2-VI) wherein R₁ is butyl. In some embodiments is a compound of Formula (2-VI) wherein R₁ is substituted or unsubstituted heteroalkyl. In some embodiments is a compound of Formula (2-VI) wherein R₁ is substituted or unsubstituted heterocycloalkyl. In some embodiments is a compound of Formula (2-VI) wherein R₁ is substituted or unsubstituted aryl. In some embodiments is a compound of Formula (2-VI) wherein R₁ is substituted or unsubstituted heteroaryl.

In some embodiments is a compound of Formula (2-VI) wherein R₂ is substituted or unsubstituted alkyl, substituted or unsubstituted heteroalkyl, substituted or unsubstituted heterocycloalkyl, substituted or unsubstituted aryl, substituted or unsubstituted heteroaryl, or —CH₂C(O)(substituted or unsubstituted aryl) and R₃ is H. In some embodiments is a compound of Formula (2-VI) wherein R₂ is substituted or unsubstituted alkyl, substituted or unsubstituted heteroalkyl, substituted or unsubstituted heterocycloalkyl, substituted or unsubstituted aryl, substituted or unsubstituted heteroaryl, or —CH₂C(O)(substituted or unsubstituted aryl) and R₃ is substituted or unsubstituted alkyl. In some embodiments is a compound of Formula (2-VI) wherein R₂ and R₃ together with the carbon atom to which they are attached form a cycloalkyl ring. In some embodiments is a compound of Formula (2-VI) wherein R₂ and R₃ together with the carbon atom to which they are attached form a heterocycloalkyl ring.

In some embodiments is a compound selected from:

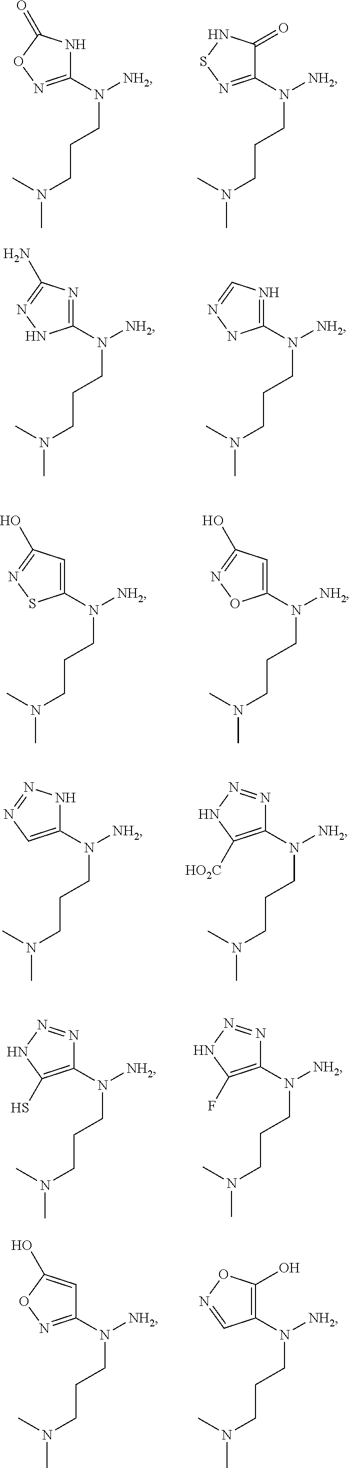

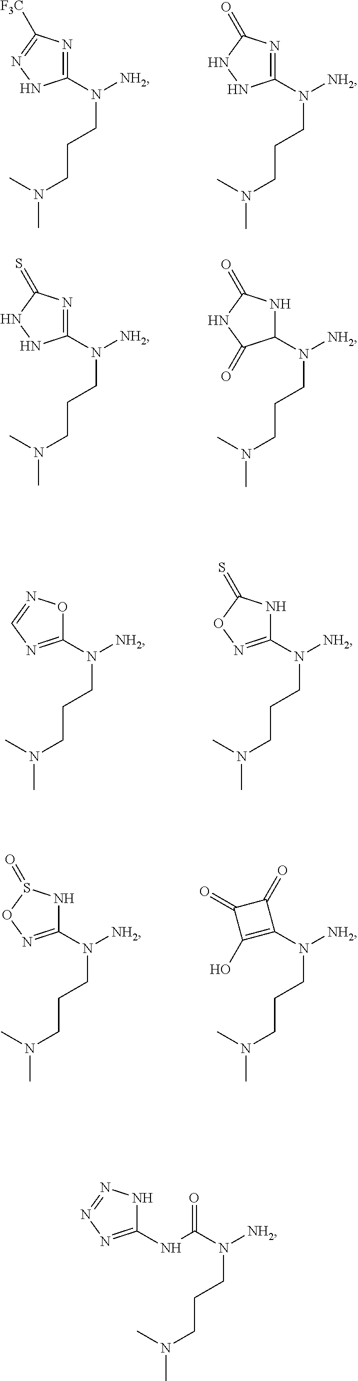

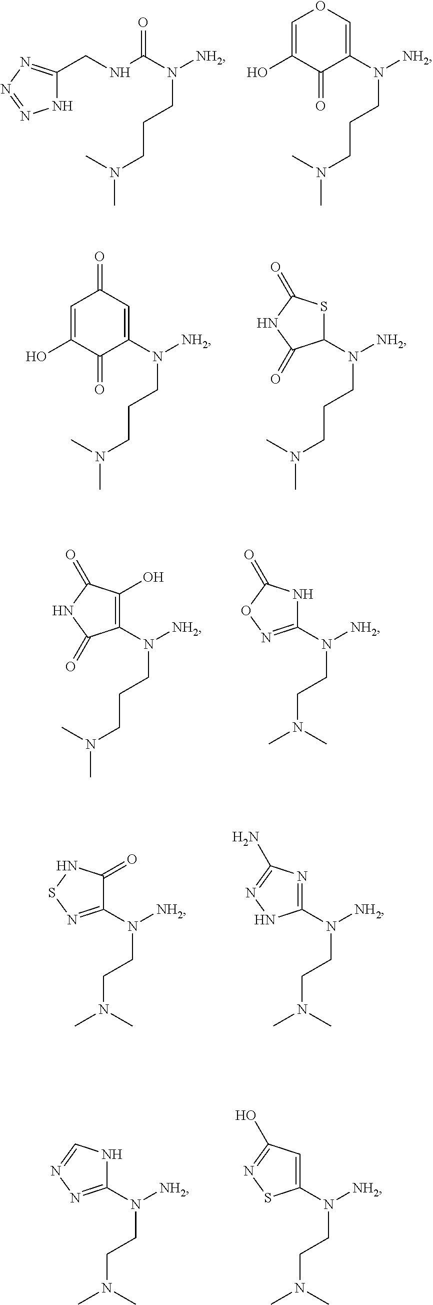

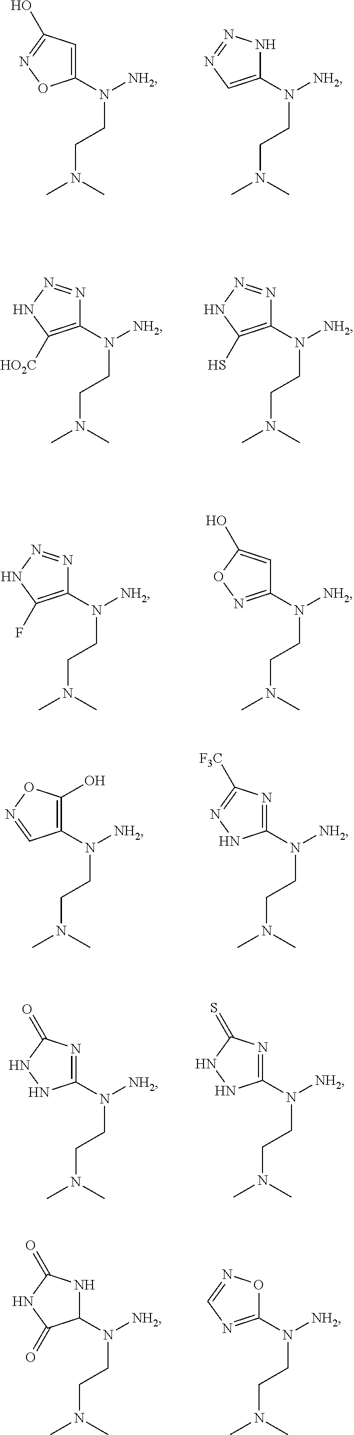

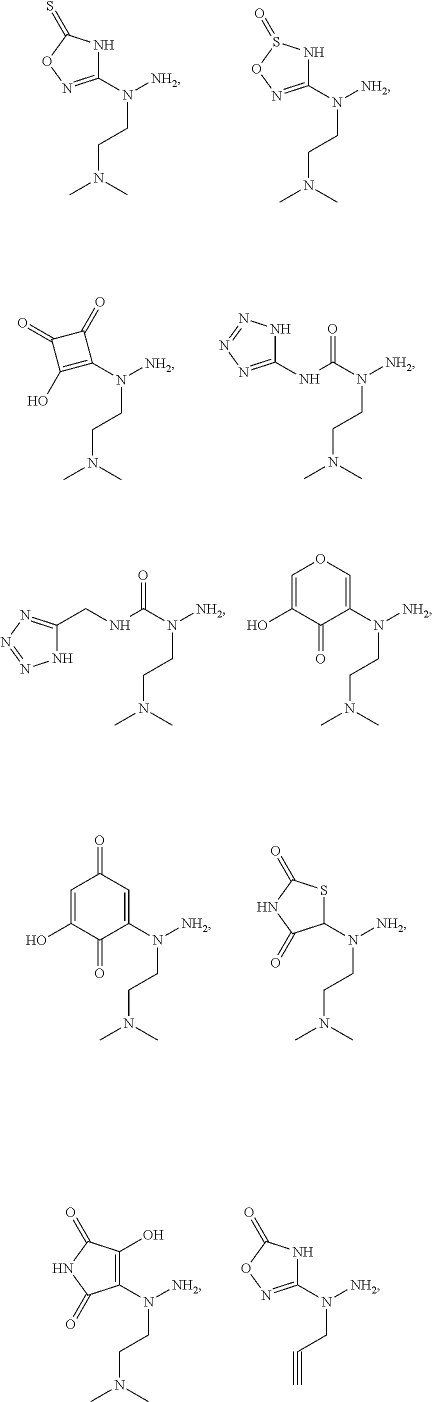

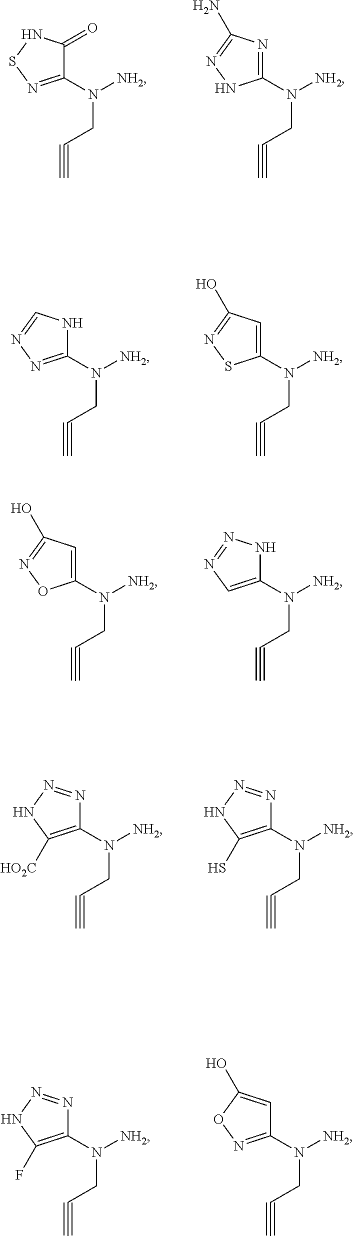

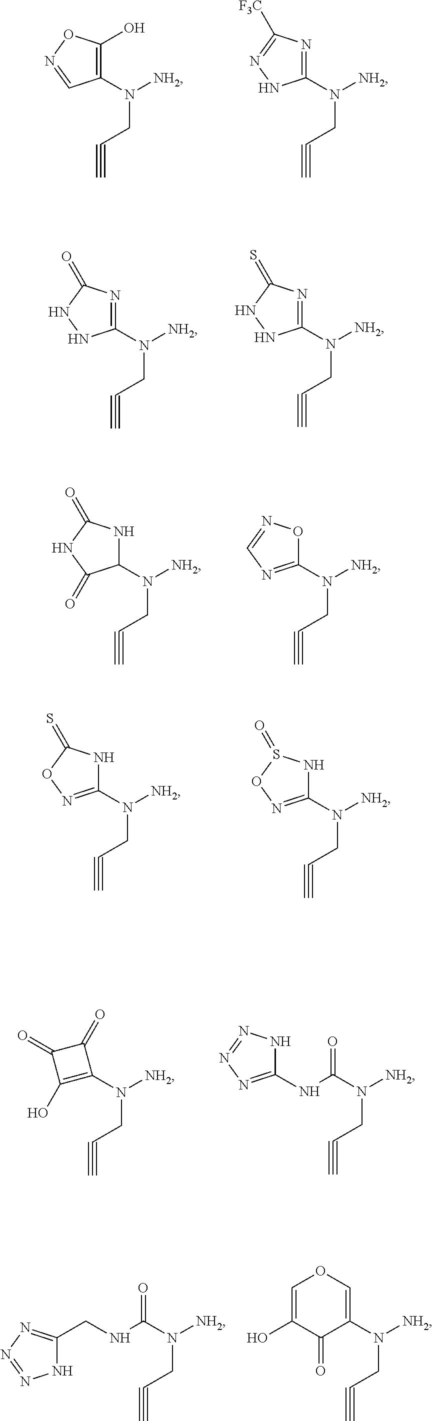

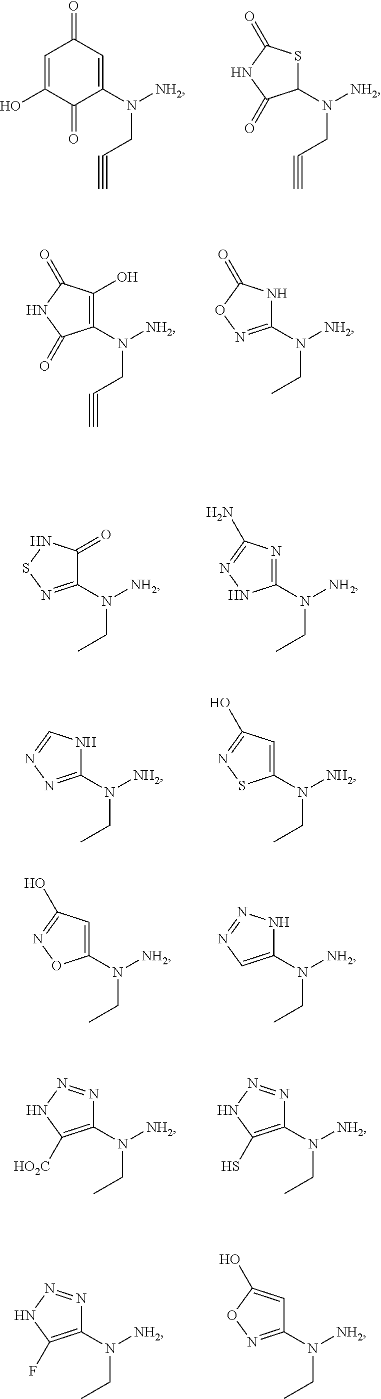

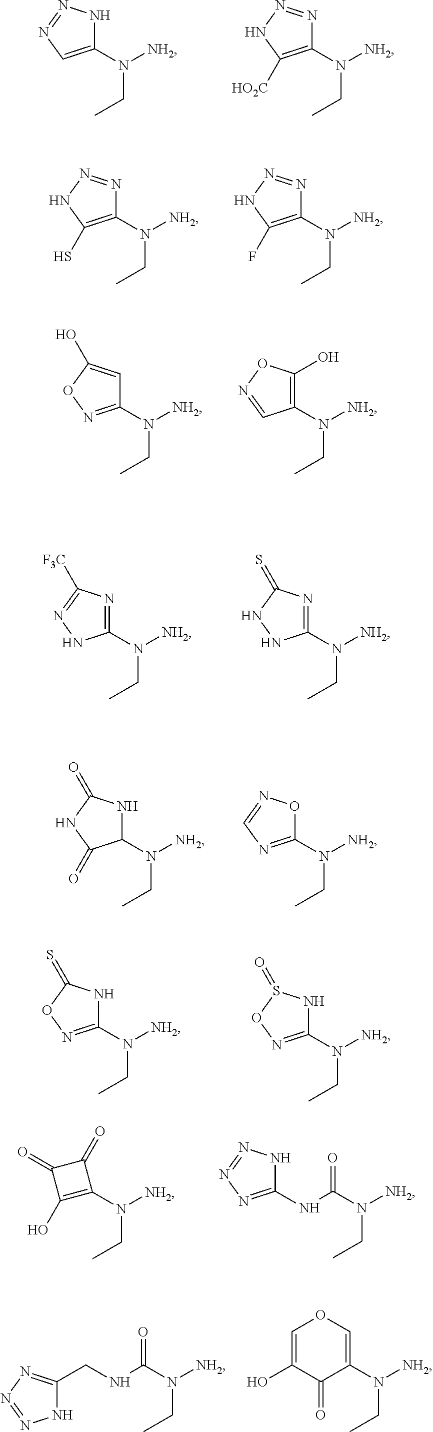

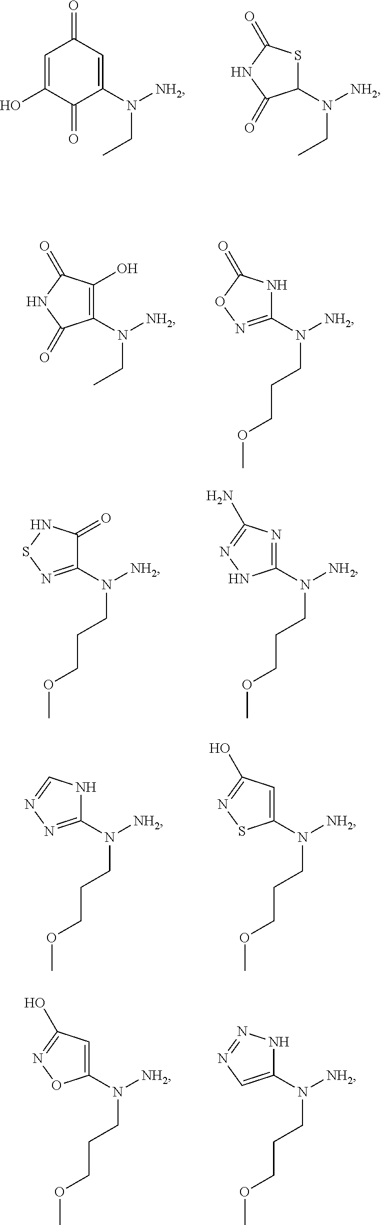

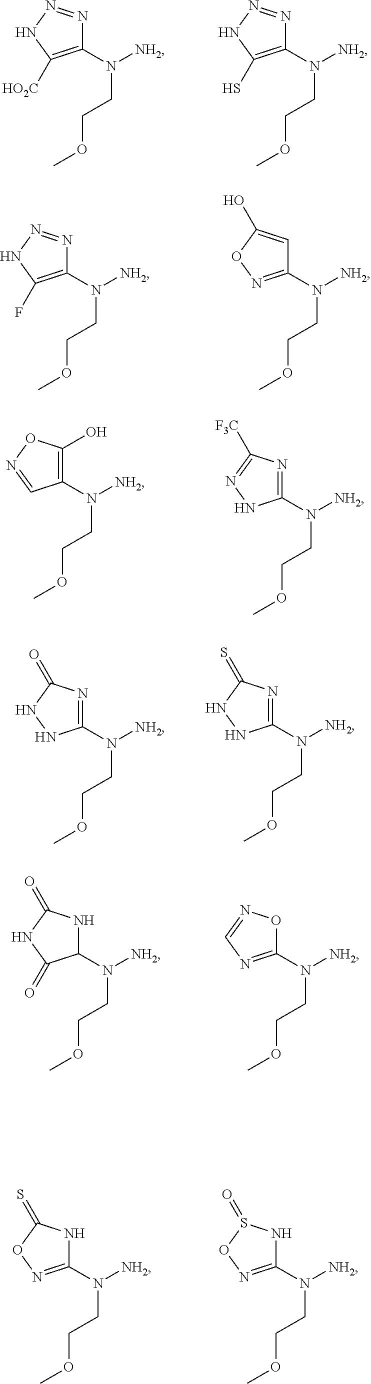

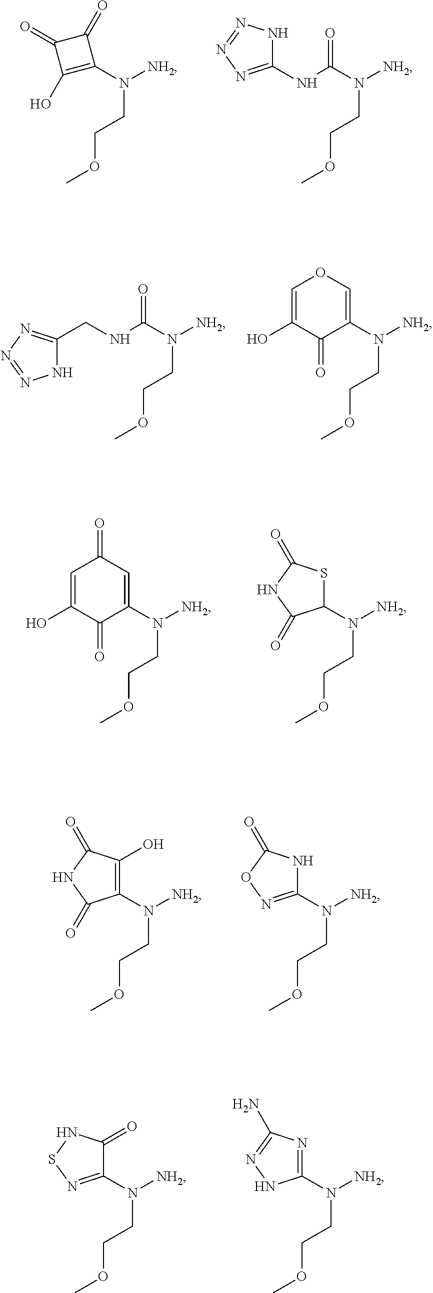

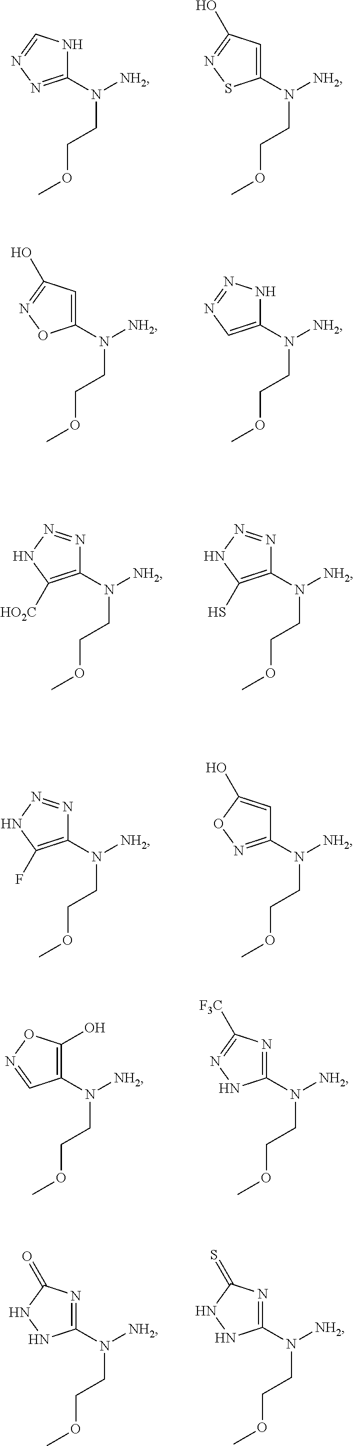

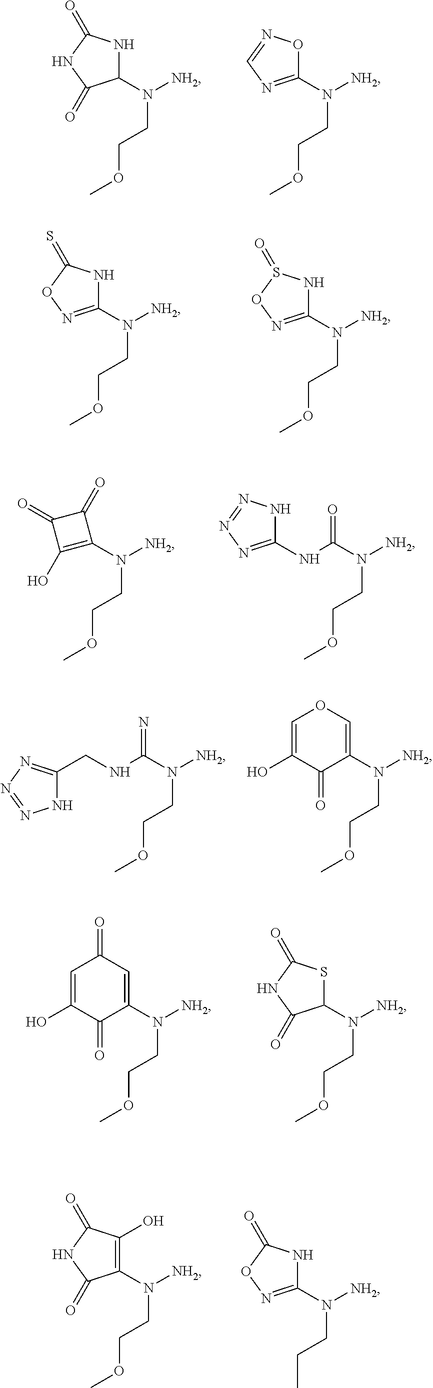

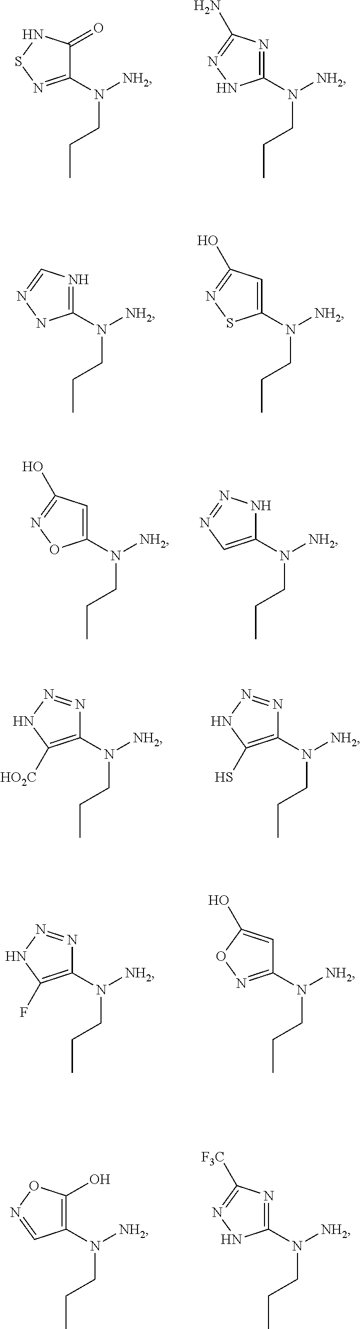

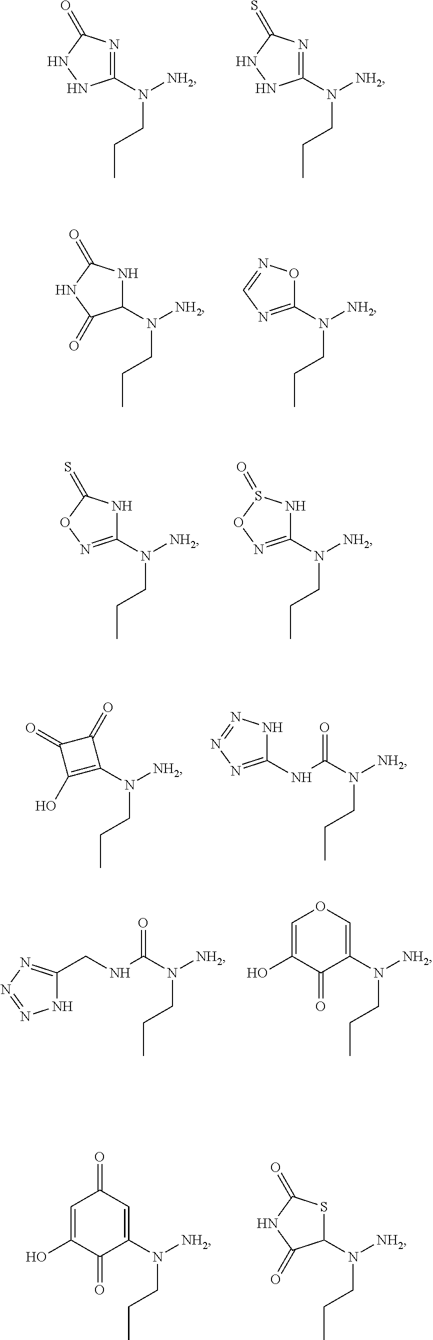

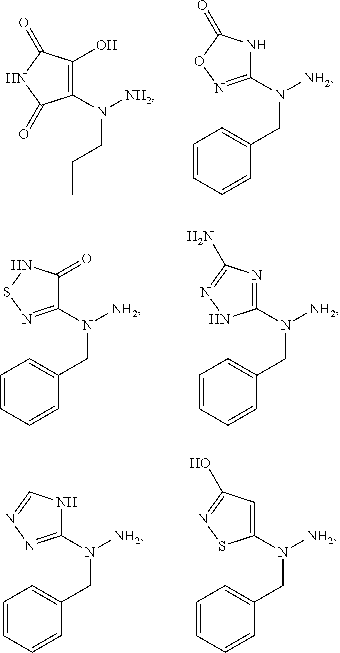

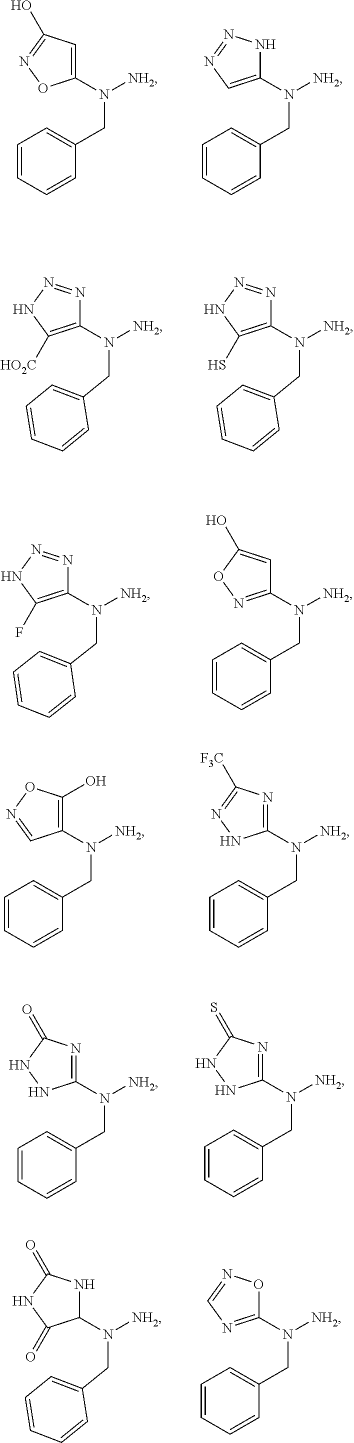

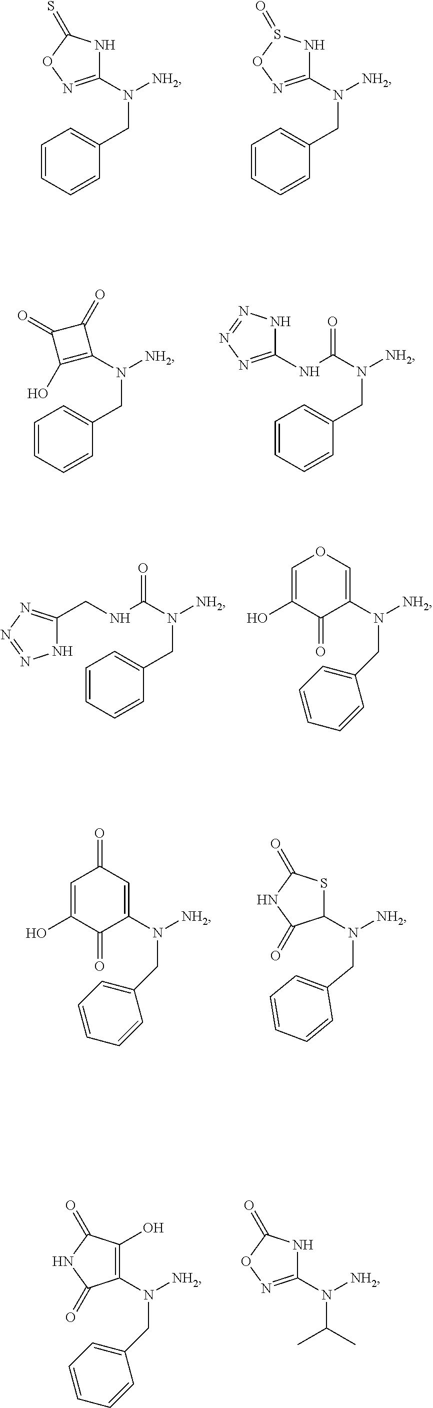

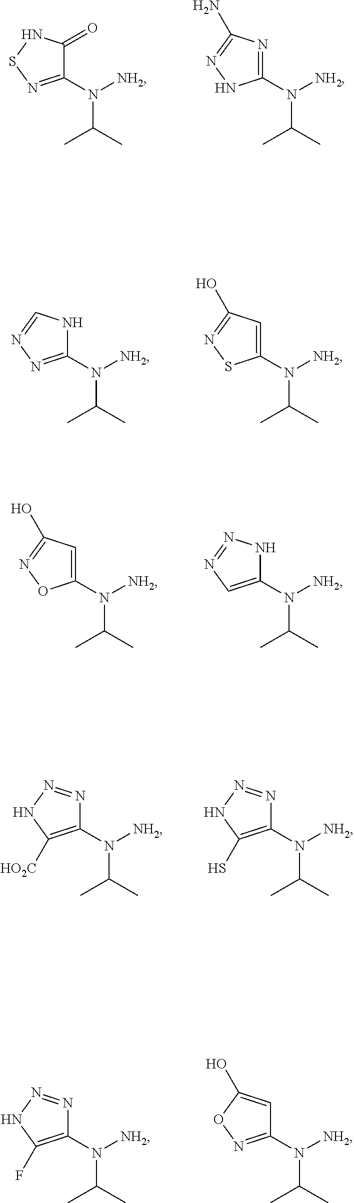

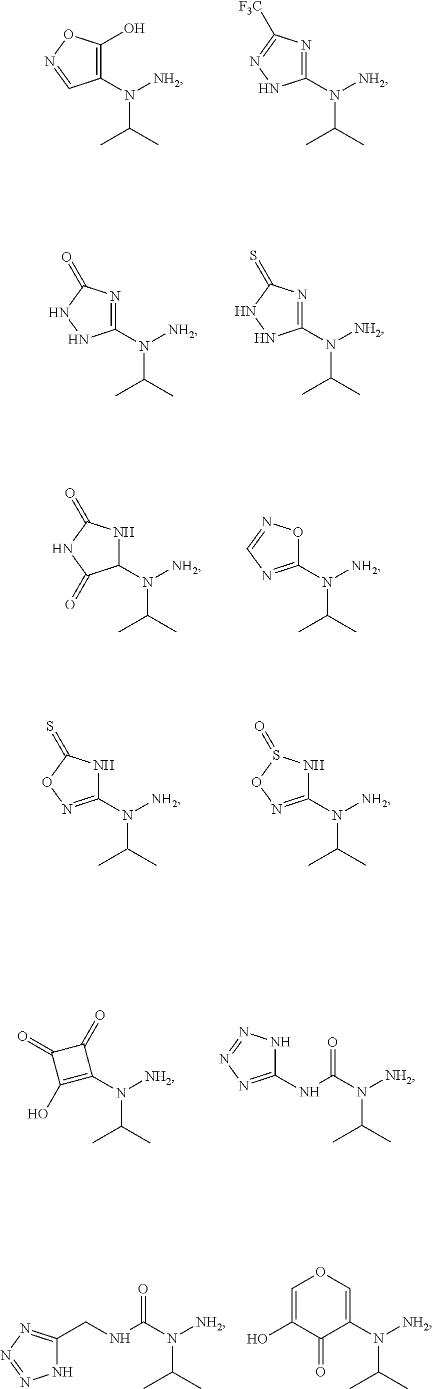

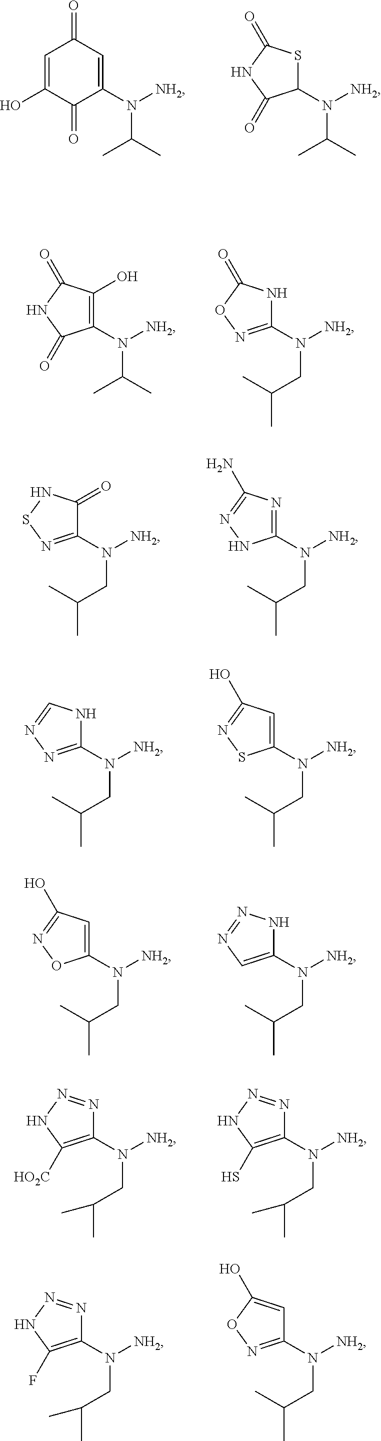

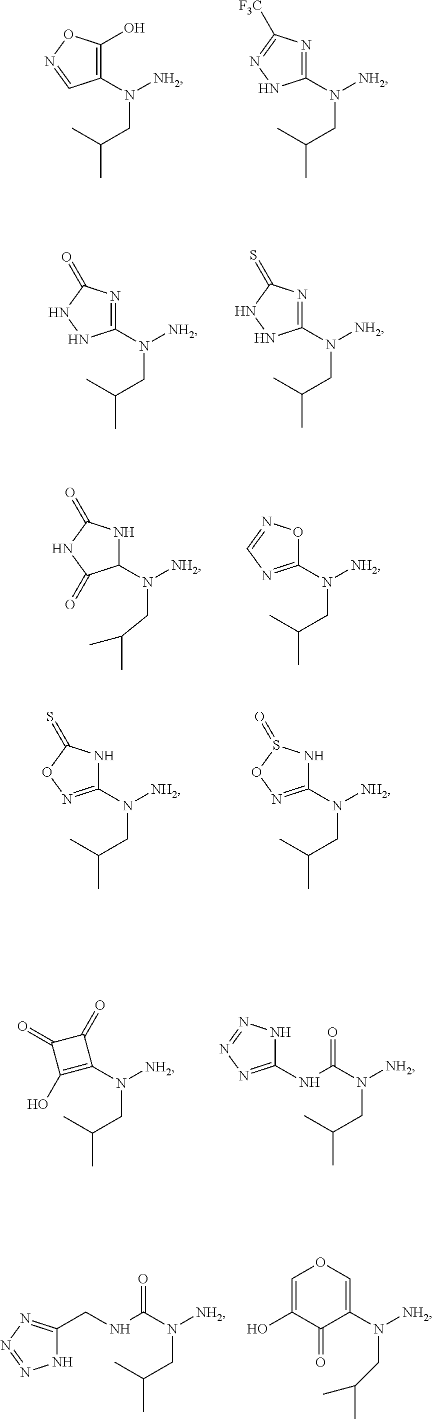

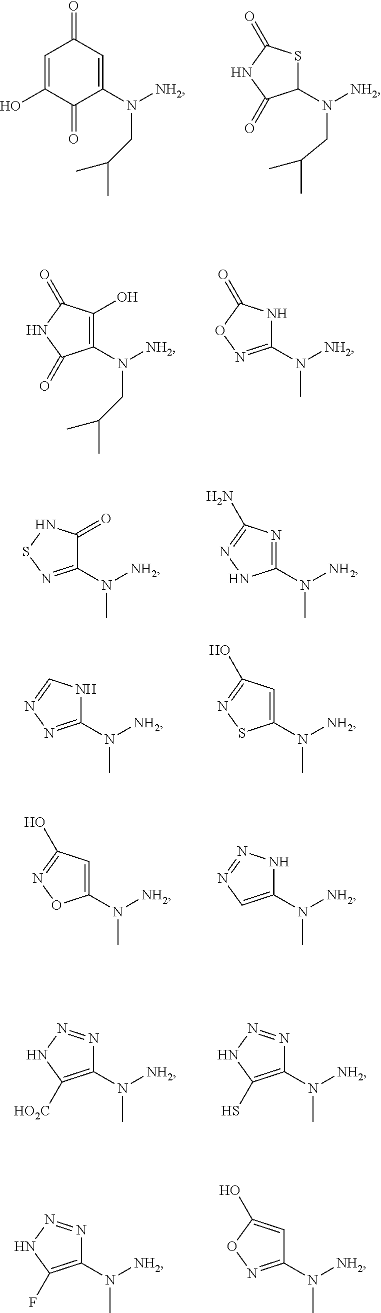

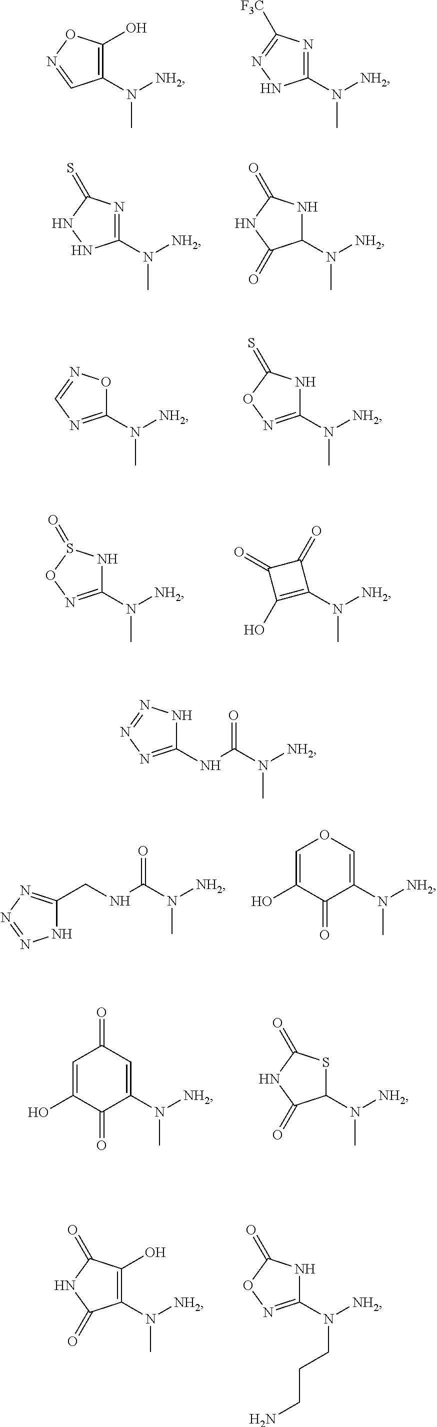

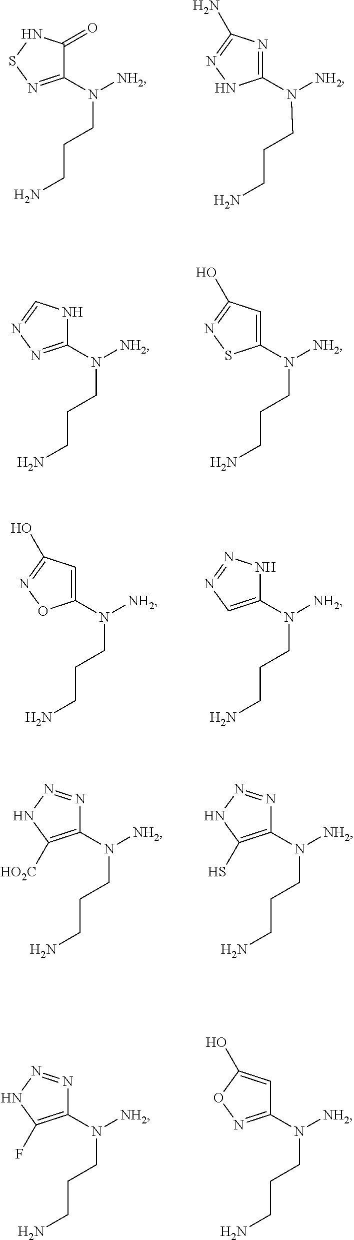

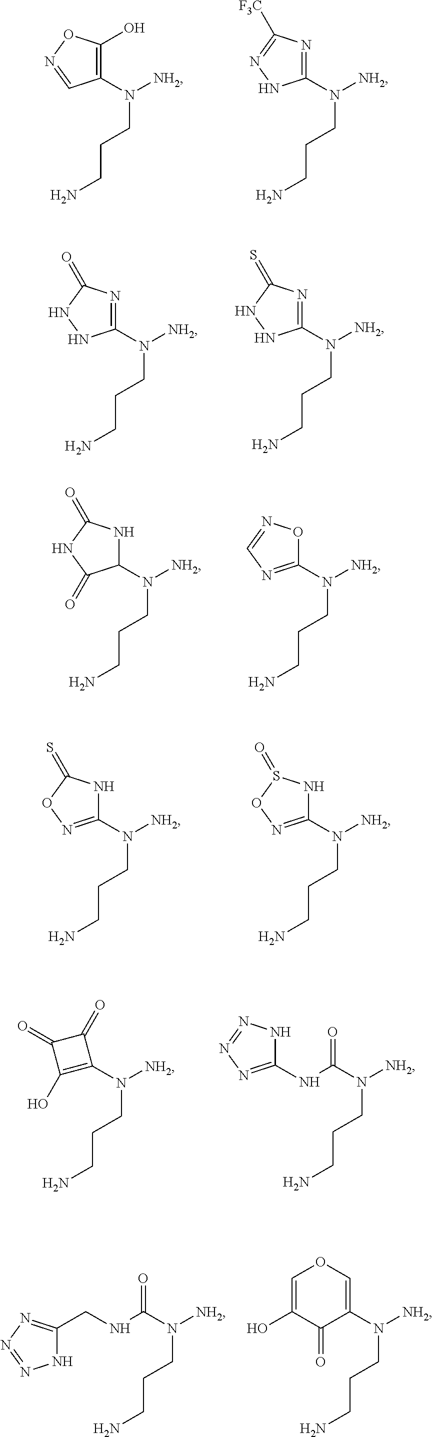

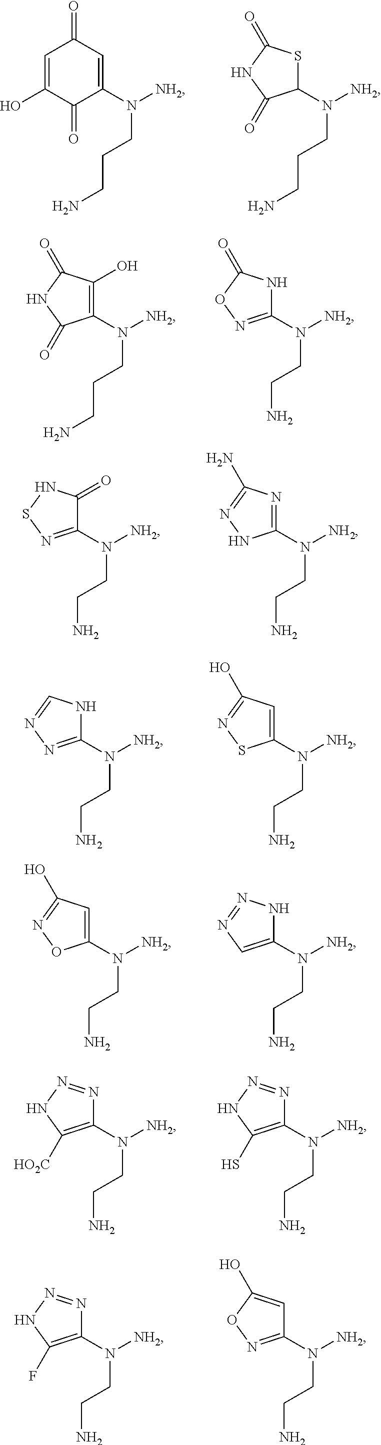

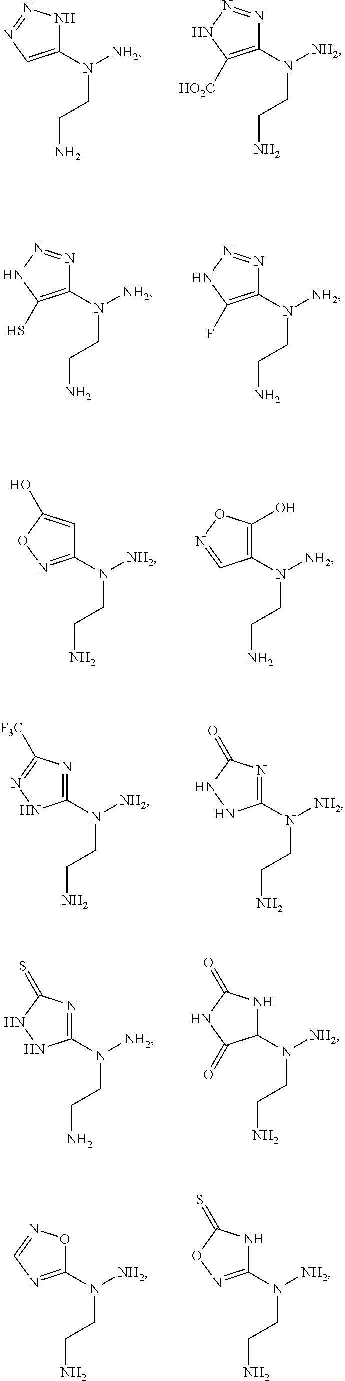

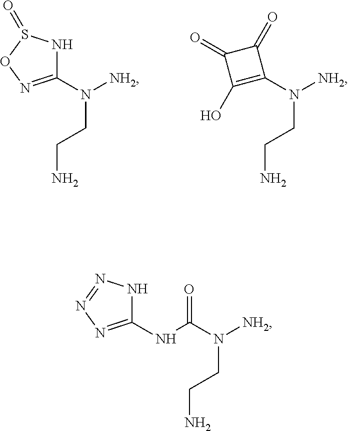 [SMILES: NCCN(N)C1=NOS(=O)N1 ; NCCN(N)c1c(O)c(=O)c1=O ; NCCN(N)C(=O)Nc1nnn[nH]1]

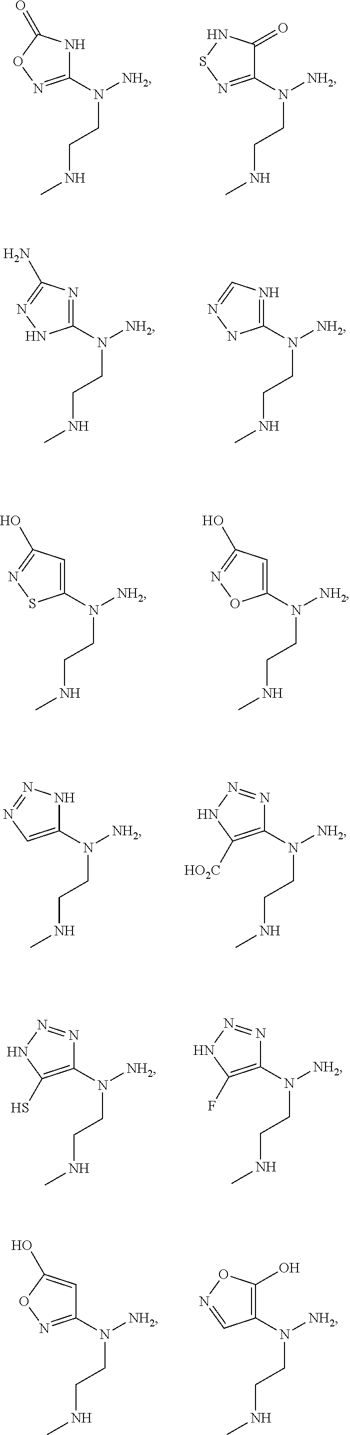

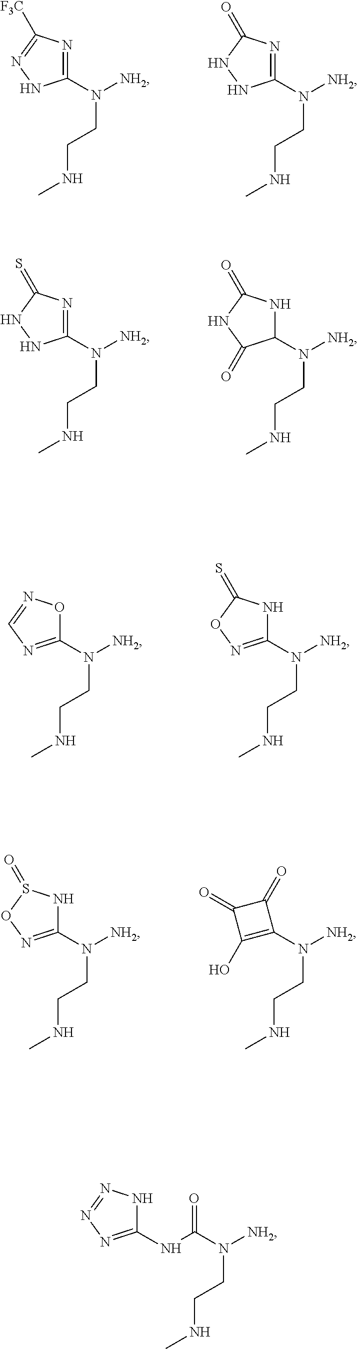

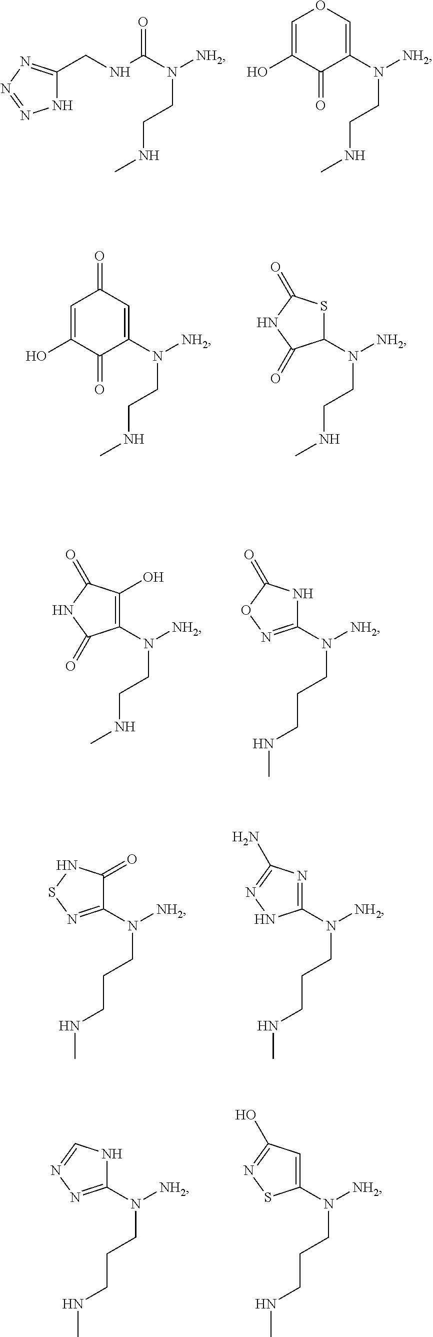

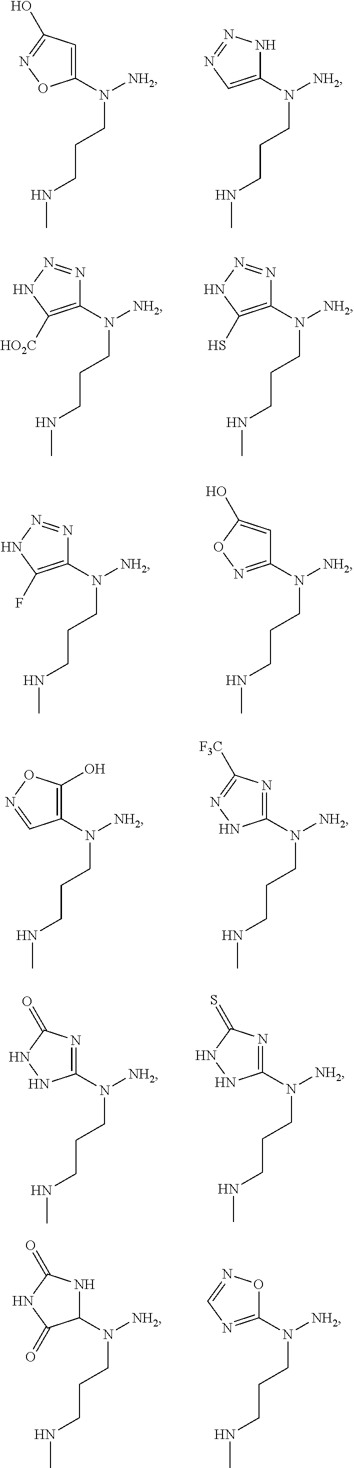

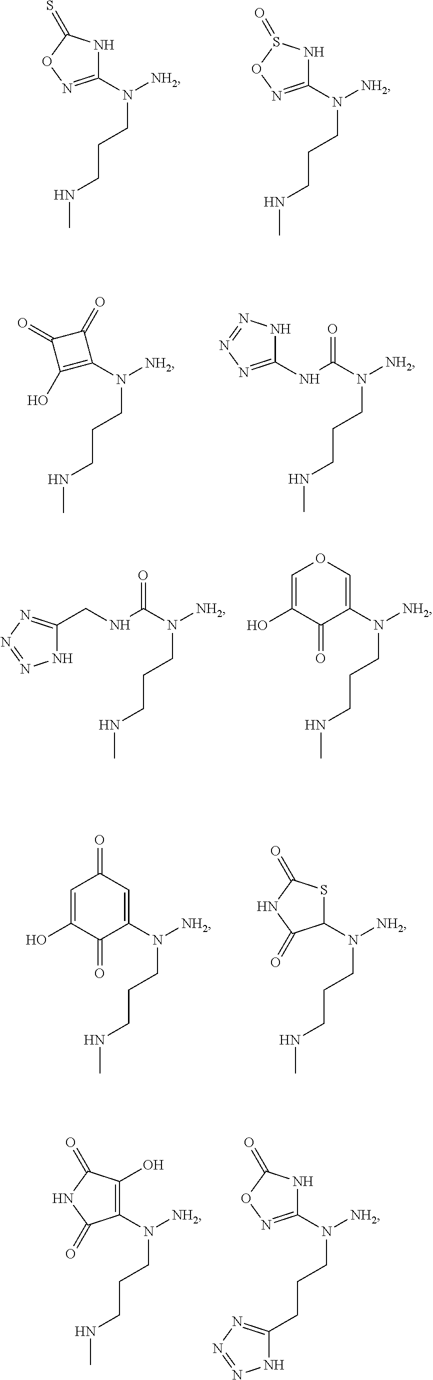

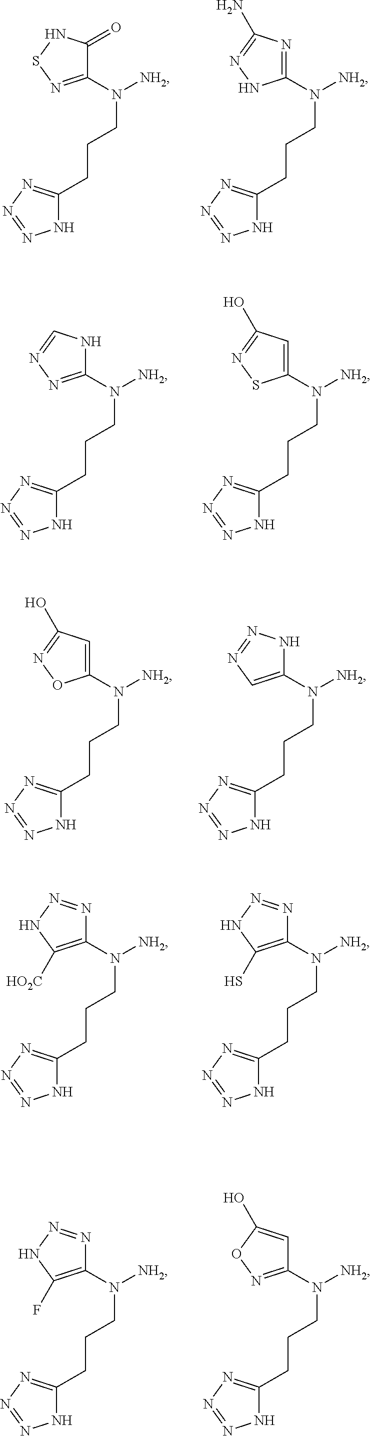

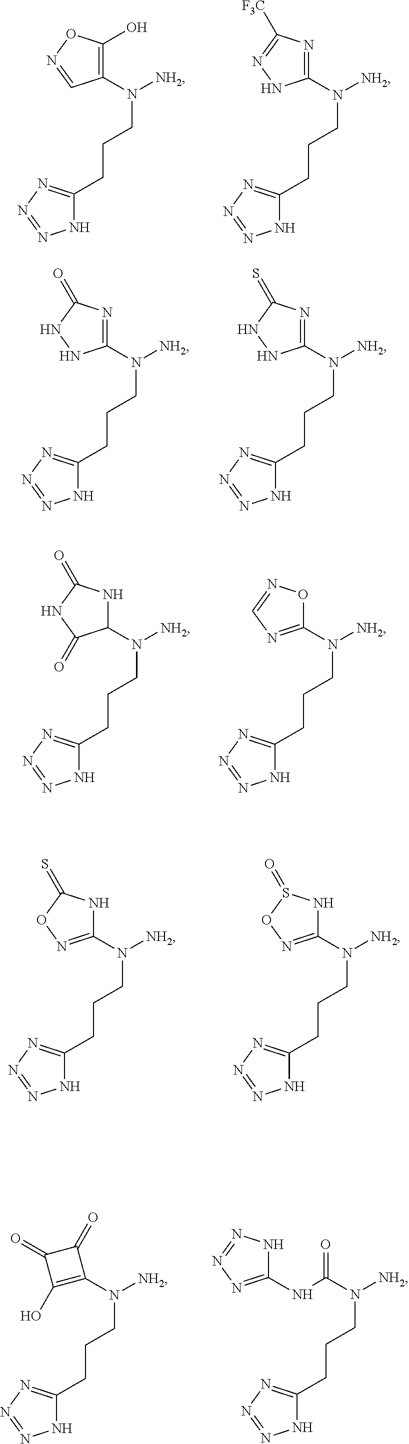

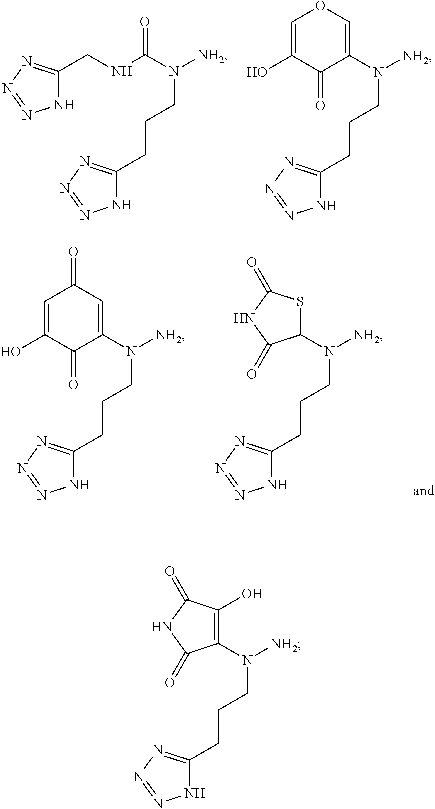

or a pharmaceutically acceptable salt, solvate, or prodrug thereof.

In some embodiments is a compound selected from:

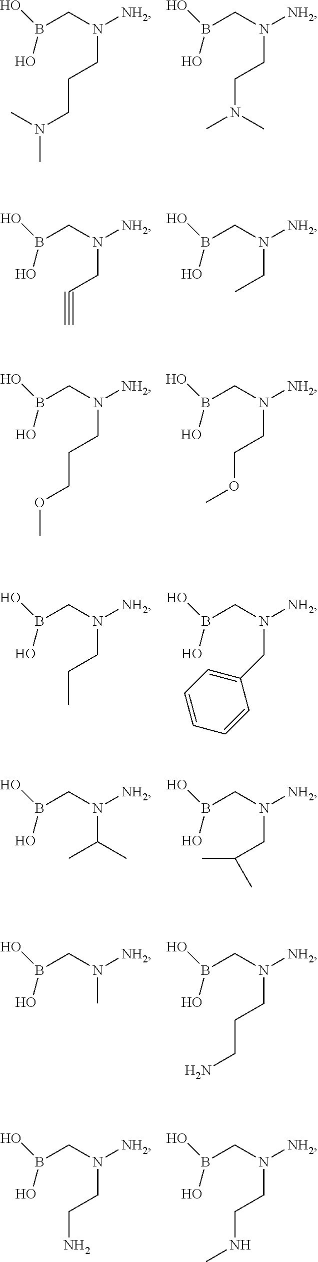

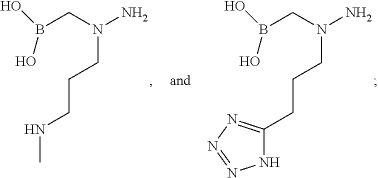

or a pharmaceutically acceptable salt, solvate, or prodrug thereof.

Provided herein are pharmaceutical compositions comprising a therapeutically effective amount of a compound of Formula (2-I), (2-II), (2-III), (2-IV), (2-V), or (2-VI), or a pharmaceutically acceptable salt, solvate, or prodrug thereof, and a pharmaceutically acceptable carrier, wherein the compound of Formula (2-I), (2-II), (2-III), (2-IV), (2-V), or (2-VI) is as described herein.

### Routes of Administration

Suitable routes of administration include, but are not limited to, oral, intravenous, aerosol, parenteral, ophthalmic, pulmonary, transmucosal, transdermal, nasal, and topical administration. In addition, by way of example only, parenteral delivery includes intramuscular, subcutaneous, intravenous, intramedullary injections, as well as intrathecal, direct intraventricular, intraperitoneal, intralymphatic, and/or intranasal injections.

In certain embodiments, a compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-I, Formula 2-II, Formula 2-III, Formula 2-IV, Formula 2-V, or Formula 2-VI) is administered in a local rather than systemic manner, for example, via topical application of the compound directly on to skin, or intravenously, or subcutaneously, often in a depot preparation or sustained release formulation. In specific embodiments, long acting formulations are administered by implantation (for example subcutaneously or intramuscularly) or by intramuscular injection. In yet other embodiments, the compound as described herein is provided in the form of a rapid release formulation, in the form of an extended release formulation, or in the form of an intermediate release formulation. In yet other embodiments, the compound described herein is administered topically (e.g., as a patch, an ointment, or in combination with a wound dressing, or as a wash or a spray). In alternative embodiments, a formulation is administered systemically (e.g., by injection, or as a pill).

### Pharmaceutical Compositions/Formulations

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

Provided herein are pharmaceutical compositions that include a compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) and at least one pharmaceutically acceptable inactive ingredient. In some embodiments, the compounds described herein are administered as pharmaceutical compositions in which compounds as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) are mixed with other active ingredients, as in combination therapy. In other embodiments, the pharmaceutical compositions include other medicinal or pharmaceutical agents, carriers, adjuvants, preserving, stabilizing, wetting or emulsifying agents, solution promoters, salts for regulating the osmotic pressure, and/or buffers. In yet other embodiments, the pharmaceutical compositions include other therapeutically valuable substances.

A pharmaceutical composition, as used herein, refers to a mixture of a compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical formulations described herein are administered to a subject by appropriate administration routes, including but not limited to, oral, parenteral (e.g., intravenous, subcutaneous, intramuscular), intranasal, buccal, topical, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound as described herein (e.g., any CSE inhibitor, including L-propargylglycine, compounds of Formula 1-I, Formula 1-II, Formula 1-Ha, Formula 1-III, Formula 1-IV, Formula 1-IVa, Formula 2-1, Formula 2-II, Formula 2-III, Formula 2-IV, Formula 2-V, or Formula 2-VI) are manufactured in a conventional manner, such as, by way of example only, by means of conventional mixing, dissolving, granulating, dragee-making, levigating, emulsifying, encapsulating, entrapping or compression processes.

The pharmaceutical compositions will include at least one compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-I, Formula 2-II, Formula 2-III, Formula 2-IV, Formula 2-V, or Formula 2-VI) as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, compounds described herein exist in unsolvated form or in solvated forms with pharmaceutically acceptable solvents such as water, ethanol, and the like. The solvated forms of the compounds presented herein are also considered to be disclosed herein.

In some embodiments, the compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI exist as tautomers. All tautomers are included within the scope of the compounds presented herein. As such, it is to be understood that a compound of the Formula (I), (II), (IIa), (III), (IV), or (IVa) or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the present disclosure encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein. For example, tautomerism may be exhibited by a tetrazole group or a triazole group bonded as indicated by the wavy line:

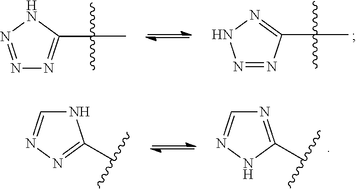

In some embodiments, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI exist as enantiomers, diastereomers, or other steroisomeric forms. The compounds disclosed herein include all enantiomeric, diastereomeric, and epimeric forms as well as mixtures thereof.

In some embodiments, compounds described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrug forms of the herein described compounds, wherein the prodrug is metabolized in vivo to produce a compound of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI as set forth herein are included within the scope of the claims. Prodrug forms of the herein described compounds, wherein the prodrug is metabolized in vivo to produce a compound of Formula 1-I, Formula 1-II, Formula 1-Ia, Formula 1-III, Formula 1-IV, Formula 1-IVa, Formula 2-I, Formula 2-II, Formula 2-III, Formula 2-IV, or Formula 2-V as set forth herein are included within the scope of the claims. In some cases, some of the compounds described herein may be a prodrug for another derivative or active compound. In some embodiments described herein, hydrazones are metabolized in vivo to produce a compound of Formula 1-1, Formula 1-II, Formula 1-IIa, Formula 1-III, Formula 1-IV, Formula 1-IVa, Formula 2-1, Formula 2-II, Formula 2-III, Formula 2-IV, or Formula 2-V. In some embodiments, compounds of Formula 2-VI are metabolized in vivo to produce a compound of Formula Formula 1-I, Formula 1-II, Formula 1-IIa, Formula 1-III, Formula 1-IV, Formula 1-IVa, Formula 2-1, Formula 2-II, Formula 2-III, Formula 2-IV, or Formula 2-V.

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

The pharmaceutical compositions described herein, which include a compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

### Certain Topical Compositions

In some embodiments, compounds as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) are prepared as transdermal dosage forms. In one embodiment, the transdermal formulations described herein include at least three components: (1) a formulation of a compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI); (2) a penetration enhancer; and (3) an optional aqueous adjuvant. In some embodiments the transdermal formulations include additional components such as, but not limited to, gelling agents, creams and ointment bases, and the like. In some embodiments, the transdermal formulation is presented as a patch or a wound dressing. In some embodiments, the transdermal formulation further include a woven or non-woven backing material to enhance absorption and prevent the removal of the transdermal formulation from the skin. In other embodiments, the transdermal formulations described herein can maintain a saturated or supersaturated state to promote diffusion into the skin.

In one aspect, formulations suitable for transdermal administration of compounds described herein employ transdermal delivery devices and transdermal delivery patches and can be lipophilic emulsions or buffered, aqueous solutions, dissolved and/or dispersed in a polymer or an adhesive. In one aspect, such patches are constructed for continuous, pulsatile, or on demand delivery of pharmaceutical agents. Still further, transdermal delivery of the compounds described herein can be accomplished by means of iontophoretic patches and the like. In one aspect, transdermal patches provide controlled delivery of a compound as described herein (e.g., any CSE inhibitor, including L-propargylglycine, compounds of Formula 1-I, Formula 1-II, Formula 1-IIa, Formula 1-III, Formula 14V, Formula 1-IVa, Formula 2-1, Formula 2-II, Formula 2-III, Formula 2-IV, Formula 2-V, or Formula 2-VI). In one aspect, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In further embodiments, topical formulations include gel formulations (e.g., gel patches which adhere to the skin). In some of such embodiments, a gel composition includes any polymer that forms a gel upon contact with the body (e.g., gel formulations comprising hyaluronic acid, pluronic polymers, poly(lactic-co-glycolic acid (PLGA)-based polymers or the like). In some forms of the compositions, the formulation comprises a low-melting wax such as, but not limited to, a mixture of fatty acid glycerides, optionally in combination with cocoa butter which is first melted. Optionally, the formulations further comprise a moisturizing agent.

In certain embodiments, delivery systems for pharmaceutical compounds may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

In alternative embodiments, a compound as described herein (e.g., any CSE inhibitor, including L-propargylglycine, compounds of Formula 14, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) is formulated and presented as a wash or rinse liquid which is used to irrigate the affected area. In further embodiments, a compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) is formulated and presented as a spray which is applied to the affected area.

### Wound Dressings

In one aspect, a compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-I, Formula 2-II, Formula 2-III, Formula 2-IV, Formula 2-V, or Formula 2-VI) is presented as part of a wound dressing. A dressing is an adjunct used for application to a wound to promote healing and/or prevent further harm. A dressing is designed to be in direct contact with a wound. In some embodiments, a wound dressing comprising a CSE inhibitor described herein provides a controlled release of the CSE inhibitor. In other embodiments, a wound dressing comprising a CSE inhibitor described herein provides sustained release of the CSE inhibitor. In other embodiments, a wound dressing comprising a CSE inhibitor described herein provides intermediate release of the CSE inhibitor. In further embodiments, a wound dressing comprising a CSE inhibitor described herein provides intermediate release of the CSE inhibitor. In other embodiments, a wound dressing comprising a CSE inhibitor described herein provides a combination of sustained, intermediate or immediate release of the CSE inhibitor.

Optionally a wound dressing comprising a CSE inhibitor comprises particles of the CSE inhibitor designed for controlled release (e.g., micronized particles, nanosized particles or a mixture thereof, non-sized particles, coated particles for controlled and/or sustained release). In some embodiments, a wound dressing is a gel patch that adheres to the skin at the site of the wound or cutaneous injury or condition. In some embodiments, a gel patch comprises any suitable gelling polymer (e.g., hyaluronan, carbomer polymers, pluronic polymers, PLGA polymers or the like). In some embodiments, a wound dressing comprises a coating on a sticky tape (e.g., medicated bandage or tape). In some embodiments, a wound dressing is a liquid which gels upon contacting the skin and is administered as a spray-on or paint.

In some additional embodiments, a CSE inhibitor is administered topically or systemically in combination with a wound dressing. In some of such embodiments, the wound dressing is non-medicated (i.e., does not comprise the CSE inhibitor). In some other embodiments, the wound dressing comprises a CSE inhibitor as described above.

In further embodiments, a CSE inhibitor is administered topically or systemically in combination with a wound dressing and a bandage.

### Certain Systemically Administered Compositions

In one aspect, a compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some cases it is desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections or drips or infusions, compounds described herein are formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) is formulated for use as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of; such as, by way of example only, gelatin for use in an inhaler or insufflator may be formulated containing a powder mix of the compound described herein and a suitable powder base such as lactose or starch.

Representative intranasal formulations are described in, for example, U.S. Pat. Nos. 4,476,116, 5,116,817 and 6,391,452. Formulations that include a compound of Formula (I) are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

In some embodiments, pharmaceutical formulations of a compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) are in the form of a capsules, including push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added. A capsule may be prepared, for example, by placing the bulk blend of the formulation of the compound described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

All formulations for oral administration are in dosages suitable for such administration.

In one aspect, solid oral dosage forms are prepared by mixing a compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) with one or more of the following: antioxidants, flavoring agents, and carrier materials such as binders, suspending agents, disintegration agents, filling agents, surfactants, solubilizers, stabilizers, lubricants, wetting agents, and diluents.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder

Compressed tablets are solid dosage forms prepared by compacting the bulk blend of the formulations described above. In various embodiments, tablets will include one or more flavoring agents.

In other embodiments, the tablets will include a film surrounding the final compressed tablet. In some embodiments, the film coating can provide a delayed release of the compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) from the formulation. In other embodiments, the film coating aids in patient compliance (e.g., Opadry® coatings or sugar coating). Film coatings including Opadry® typically range from about 1% to about 3% of the tablet weight.

In some embodiments, solid dosage forms, e.g., tablets, effervescent tablets, and capsules, are prepared by mixing particles of a compound with one or more pharmaceutical excipients to form a bulk blend composition. The bulk blend is readily subdivided into equally effective unit dosage forms, such as tablets, pills, and capsules. In some embodiments, the individual unit dosages include film coatings. These formulations are manufactured by conventional formulation techniques.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary useful microencapsulation materials include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IRO, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® 5100, Eudragit® RD100, Eudragit® E100, Eudragit® L12.5, Eudragit® 512.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to a CSE inhibitor, the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Buccal formulations that include a compound as described herein (e.g., any CSE inhibitor, including L-propargylglycine, compounds of Formula 14, Formula 1-II, Formula 1-Ha, Formula 1-III, Formula 1-IV, Formula 1-IVa, Formula 2-1, Formula 2-II, Formula Formula 2-IV, Formula 2-V, or Formula 2-VI) are administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydro lysable) polymeric carrier that also serves to adhere the dosage form to the buccal mucosa. For buccal or sublingual administration, the compositions may take the form of tablets, lozenges, or gels formulated in a conventional manner.

For intravenous injections, a CSE inhibitor is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a pharmaceutical composition described herein is in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch, or sodium starch glycolate, a cellulose such as methylcrystalline cellulose, methylcellulose, microcrystalline cellulose, croscarmellose, or a cross-linked cellulose, such as cross-linked sodium carboxymethylcellulose, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a gum such as agar, guar, locust bean, Karaya, pectin, or tragacanth, sodium starch glycolate, bentonite, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose, hydroxypropylmethylcellulose, hydroxypropylmethylcellulose acetate stearate, hydroxyethylcellulose, hydroxypropylcellulose, ethylcellulose, and microcrystalline cellulose, microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose, glucose, dextrose, molasses, mannitol, sorbitol, xylitol, lactose, a natural or synthetic gum such as acacia, tragacanth, ghatti gum, mucilage of isapol husks, starch, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Binder levels of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the pharmaceutical compositions described herein. The amounts of such additives can be readily determined by one skilled in the art, according to the particular properties desired.

In various embodiments, the particles of a compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In other embodiments, a powder including a compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) is formulated to include one or more pharmaceutical excipients and flavors. Such a powder is prepared, for example, by mixing the compound and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared. Effervescent salts have been used to disperse medicines in water for oral administration.

### Controlled Release Formulations

In some embodiments, the pharmaceutical dosage forms are formulated to provide a controlled release of a compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI). Controlled release refers to the release of the compound from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein are formulated as enteric coated delayed release oral dosage forms, i.e., as an oral dosage form of a pharmaceutical composition as described herein which utilizes an enteric coating to affect release in the small intestine or large intestine. In one aspect, the enteric coated dosage form is a compressed or molded or extruded tablet/mold (coated or uncoated) containing granules, powder, pellets, beads or particles of the active ingredient and/or other composition components, which are themselves coated or uncoated. In one aspect, the enteric coated oral dosage form is in the form of a capsule containing pellets, beads or granules, which include a compound of Formula (I), that are coated or uncoated.

Any coatings should be applied to a sufficient thickness such that the entire coating does not dissolve in the gastrointestinal fluids at pH below about 5, but does dissolve at pH about 5 and above. Coatings are typically selected from any of the following:

Shellac—this coating dissolves in media of pH>7; Acrylic polymers—examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine; Poly Vinyl Acetate Phthalate (PVAP)-PVAP dissolves in pH>5, and it is much less permeable to water vapor and gastric fluids.

Conventional coating techniques such as spray or pan coating are employed to apply coatings. The coating thickness must be sufficient to ensure that the oral dosage form remains intact until the desired site of topical delivery in the intestinal tract is reached.

In other embodiments, the formulations described herein are delivered using a pulsatile dosage form. A pulsatile dosage form is capable of providing one or more immediate release pulses at predetermined time points after a controlled lag time or at specific sites. Exemplary pulsatile dosage forms and methods of their manufacture are disclosed in U.S. Pat. Nos. 5,011,692, 5,017,381, 5,229,135, 5,840,329 and 5,837,284. In one embodiment, the pulsatile dosage form includes at least two groups of particles, (i.e. multiparticulate) each containing the formulation described herein. The first group of particles provides a substantially immediate dose of the compound of Formula (I) upon ingestion by a mammal. The first group ofparticles can be either uncoated or include a coating and/or sealant. In one aspect, the second group of particles comprises coated particles. The coating on the second group of particles provides a delay of from about 2 hours to about 7 hours following ingestion before release of the second dose. Suitable coatings for pharmaceutical compositions are described herein or known in the art.

In some embodiments, pharmaceutical formulations are provided that include particles of a compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) and at least one dispersing agent or suspending agent for oral administration to a subject. The formulations may be a powder and/or granules for suspension, and upon admixture with water, a substantially uniform suspension is obtained.

In some embodiments, particles formulated for controlled release are incorporated in a gel or a patch or a wound dressing.

In one aspect, liquid formulation dosage forms for oral administration and/or for topical administration as a wash are in the form of aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of the compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI), the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

In some embodiments, the liquid formulations also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state, as defined in The USP Pharmacists' Pharmacopeia (2005 edition, chapter 905), for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch, or sodium starch glycolate; a cellulose such as methylcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein include, for example, hydrophilic polymers, electrolytes, Tween® 60 or 80, PEG, polyvinylpyrrolidone, and the carbohydrate-based dispersing agents such as, for example, hydroxypropylcellulose and hydroxypropyl cellulose ethers, hydroxypropyl methylcellulose and hydroxypropyl methylcellulose ethers, carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer, 4-(1,1,3,3-tetramethylbutyl)-phenol polymer with ethylene oxide and formaldehyde (also known as tyloxapol), poloxamers; and poloxamines. In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers; hydroxypropyl methylcellulose and hydroxypropyl methylcellulose ethers; carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenolpolymer with ethylene oxide and formaldehyde; poloxamers; or poloxamines.

Wetting agents suitable for the aqueous suspensions and dispersions described herein include, but are not limited to, cetyl alcohol, glycerol monostearate, polyoxyethylene sorbitan fatty acid esters (e.g., the commercially available Tweens® such as e.g., Tween 20® and Tween 80®, and polyethylene glycols, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, aspartame, chocolate, cinnamon, citrus, cocoa, cyclamate, dextrose, fructose, ginger, glycyrrhetinate, glycyrrhiza (licorice) syrup, monoammonium glyrrhizinate (MagnaSweet®), maltol, mannitol, menthol, neohesperidine DC, neotame, Prosweet® Powder, saccharin, sorbitol, stevia, sucralose, sucrose, sodium saccharin, saccharin, aspartame, acesulfame potassium, mannitol, sucralose, tagatose, thaumatin, vanilla, xylitol, or any combination thereof.

### Methods of Dosing and Treatment Regimens

In one embodiment, the compounds as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) are used in the preparation of medicaments for the treatment of cutaneous injuries or conditions as described herein. In addition, a method for treating any of the diseases or conditions described herein in a subject in need of such treatment, involves administration of pharmaceutical compositions that include at least one compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) or a pharmaceutically acceptable salt, pharmaceutically active metabolite, pharmaceutically acceptable prodrug, or pharmaceutically acceptable solvate thereof, in therapeutically effective amounts to said subject.

In some embodiments, the compounds of Formula Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI are used in the preparation of medicaments for the treatment of a cutaneous injury or condition. In some embodiments, the compounds of Formula Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI are used in the preparation of medicaments for the treatment of an SRBD or conditions as described herein.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

In certain embodiments the dose of drug being administered may be temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but can nevertheless be determined according to the particular circumstances surrounding the case, including, e.g., the specific agent being administered, the route of administration, the condition being treated, and the subject or host being treated. In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously (or over a short period of time) or at appropriate intervals, for example as two, three, four or more sub-doses per day.

In certain embodiments, the administered dose of CSE inhibitor is determined via a step-wise dose escalation wherein a patient's response to the CSE inhibitor is titrated to determine the optimal dose for each individual patient. The titration is optionally carried out under observation (e.g., in a Neonatal Intensive Care Unit (NICU), a Cardiology Unit, or a sleep clinic) and the dose is modified till the desired therapeutic effect is achieved. Measures found in polysomnography reports include the fraction of sleep time spent at each level of oxygen saturation (i.e., the percent time below an oxygen saturation of 90 percent) and/or the mean oxygen saturation. The former quantifies the cumulative exposure to hypoxemia, while the latter may be inversely associated with risk for cardiovascular disease and/or glucose intolerance and/or insulin sensitivity.

In some embodiments, as a patient is started on a regimen of a CSE inhibitor, the patient is also weaned off (e.g., step-wise decrease in dose) a second treatment regimen (e.g., a methylxanthine).

In certain embodiments, the daily administered dose of a CSE inhibitor is a dose such that there are no side-effects that would otherwise occur at a higher dose. Thus, in some embodiments, administration of a CSE inhibitor reduces or prevents occurrence of side-effects such as hemorrhagic shock, edema, myocardial infarction, stroke, inflammatory mononuclear cell infiltration, sepsis and/or metabolic inhibition even after long term and/or chronic usage. In some embodiments, the administered dose of a CSE inhibitor is a dose that regulates breathing during REM and/or NREM sleep.

In one embodiment, the daily dosages appropriate for the compound as described herein (e.g., any CSE inhibitor, including L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) described herein are from about 0.01 to about 10 mg/kg per body weight. In specific embodiments, an indicated daily dosage in a large mammal, including, but not limited to, humans, is in the range from about 0.5 mg to about 1000 mg, conveniently administered in divided doses, including, but not limited to, up to four times a day. In one embodiment, the daily dosage is administered in extended release form. In certain embodiments, suitable unit dosage forms for oral administration comprise from about 1 to 500 mg active ingredient. In other embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

### Combination Therapy

In one embodiment, the CSE inhibitors described herein (e.g., L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) are administered to an individual in need thereof in combination with an anti-inflammatory agent. Examples of such anti-inflammatory agents include and are not limited to analgesics, non-steroidal anti-inflammatory drugs (NSAIDs), COX-2 inhibitors, and the like.

In another embodiment, the CSE inhibitors described herein (e.g., L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) are administered to an individual in need thereof in combination with a pain medication. Examples of such pain medications include and are not limited to paracetamol, the non-steroidal anti-inflammatory drugs (NSAIDs) such as the salicylates, opioid drugs such as morphine and opium, or analogues such as codeine, oxycodone and the like.

In additional embodiments, the CSE inhibitors described herein (e.g., L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) are administered to an individual in need thereof in combination with an antiseptic agent (e.g., hydrogen peroxide, iodine, chlorhexidine, boric acid, benzalkonium chloride (BAC), cetyl trimethylammonium bromide (CTMB), cetylpyridinium chloride (Cetrim, CPC), benzethonium chloride (BZT) and the like.

In further embodiments, the CSE inhibitors described herein (e.g., L-propargylglycine, compounds of Formula 1-1, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) are administered to an individual in need thereof in combination with an anesthetic agent (e.g., benzocaine, lidocaine and the like).

In additional embodiments, the CSE inhibitors described herein (e.g., L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) are administered to an individual in need thereof in combination with one or more agents used to treat allergy, including, but not limited to: antihistamine and decongestant combinations (cetirizine and pseudoephedrine; desloratadine and pseudoephedrine ER; fexofenadine and pseudoephedrine; loratadine and pseudoephedrine); antihistamines (azelastine nasal spray; brompheniramine; brompheniramine oral suspension; carbinoxamine; cetirizine; chlorpheniramine; clemastine; desloratadine; dexchlorpheniramine ER; dexchlorpheniramine oral syrup; diphenhydramine oral; fexofenadine; loratadine; promethazine); decongestants (pseudoephedrine); leukotriene modifiers (montelukast; montelukast granules); nasal anticholinergics (ipratropium); nasal corticosteroids (beclomethasone nasal inhalation; budesonide nasal inhaler; flunisolide nasal inhalation; fluticasone nasal inhalation; mometasone nasal spray; triamcinolone nasal inhalation; triamcinolone nasal spray); nasal decongestants (phenylephrine); nasal mast cell stabilizers (cromolyn nasal spray) and the like.

In further embodiments, the CSE inhibitors described herein (e.g., L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) are administered to an individual in need thereof in combination with antibiotics. In yet other embodiments, the CSE inhibitors described herein (e.g., L-propargylglycine, compounds of Formula 1-I, Formula 1-II, Formula 1-IIa, Formula 1-III, Formula 1-IV, Formula 1-IVa, Formula 2-1, Formula 2-II, Formula 2-III, Formula 2-IV, Formula 2-V, or Formula 2-VI) are administered to an individual in need thereof in combination with a wound dressing.

In some embodiments, a second therapeutic agent is administered in combination with a CSE inhibitor, wherein the second therapeutic agent is an agent that stimulates respiratory drive. In some embodiments, the second therapeutic agent induces metabolic acidosis, thereby increasing respiratory drive. In some embodiments, the second therapeutic agent treats symptoms such as hypertension that are associated with sleep apneas. In some embodiments, the second therapeutic agent is a sleep inducing agent.

Examples of agents suitable for combination therapy with an agent that modulates the activity of the carotid body include carbonic anhydrase inhibitors (e.g., acetazolamide), cholinesterase inhibitors (e.g., donepezil), adenosine inhibitors (e.g., theophylline), progestational agents (e.g., progestone), opiod antagonists (e.g., naloxone), central nervous system stimulants (e.g., nicotine), serotonergic agents (e.g., paroxetine) including selective serotonin reuptake inhibitors (SSRIs), antidepressants (e.g., protriptyline) including conventional and/or tricyclic antidepressants, antihypertensives (e.g., metoprolol, cilazapril, propranolol, atenolol, hydrochlorothiazide), calcium channel antagonists (e.g., isradipine), ACE inhibitors (e.g., spirapril), respiratory stimulants (e.g., doxapram), alpha-2 adrenergic agonists (e.g., clonidine), gama aminobutyric acid agonists (e.g., baclofen), glutamate antagonists (e.g., sabeluzole), or gaseous respiration stimulants such as carbon dioxide.

### Combination Formulations and Kits

Also provided herein are kits for therapies described herein. In some embodiments, the kit comprises a CSE inhibitor and a second treatment regimen. Such kits generally will comprise one or more of the active agent as disclosed herein, and instructions for using the kit.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic.

In certain embodiments, the pharmaceutical compositions are presented in a pack or dispenser device which contains one or more unit dosage forms containing a CSE inhibitor. In another embodiment, the pack for example contains metal or plastic foil, such as a blister pack.

### Assays for Identification of CSE Inhibitors

In some embodiments, CSE inhibitors are identified by use of in vitro assays. By way of example, an in vitro assay for CSE enzyme activity is described in Zhong et al. Chinese Medical Journal, 2009, 122, 326-330. In some embodiments, in vitro enzyme assays are adapted for high-throughput screening (HTS) using any suitable method.

In some embodiments, in vivo assays are used to determine the effect of CSE inhibitor. In some embodiments, an in vivo assay for identifying a CSE inhibitor comprises

(a) preparing organ or tissue homogenates from a test animal that has been administered a test compound; and

(b) calculating H₂S concentration based on absorbance;

wherein a decrease in H₂S concentration indicates that the test compound is a CSE inhibitor. In some embodiments of the aforementioned assay, the test animal is subjected to normoxia, acute hypoxia, chronic intermittent hypoxia, hypercapnia, or a combination thereof. Optional intermediate steps include:

effecting enzymatic reaction on L-cysteine;

quenching the enzymatic reaction with zinc acetate and trichloroacetic acid;

reacting the zinc sulfide with acidic N,N-dimethyl-p-phenylendiamine sulfate and ferric chloride; and

measuring the absorbance of the assay mixture with a micro-plate reader.

In some embodiments, an in vivo assay for identifying a CSE inhibitor comprises

(a) isolating an organ or tissue from a test animal that has been administered a test compound;

(b) challenging the organ or tissue in the recording chamber by perfusing the recording chamber with varying levels of oxygen and/or carbon dioxide; and

(c) recording action potentials;

wherein a decrease in action potential indicates that the test compound is a CSE inhibitor. In some embodiments of the aforementioned assay, the test animal is subjected to normoxia, acute hypoxia, chronic intermittent hypoxia, hypercapnia, or a combination thereof. Optional intermediate steps include:

placing the organ or tissue in a recording chamber superfused with warm physiological saline.

Optional instruments for recording action potentials include a suction electrode on a PowerLab/8P machine.

### EXAMPLES

The following specific examples are to be construed as merely illustrative, and not limitative of the remainder of the disclosure in any way whatsoever.

All synthetic chemistry was performed in standard laboratory glassware unless indicated otherwise in the examples. Commercial reagents were used as received.

### Example 1-1

### Synthesis of 3-amino-3-(1H-tetrazol-5-yl)propionitrile dihydrochloride (7)

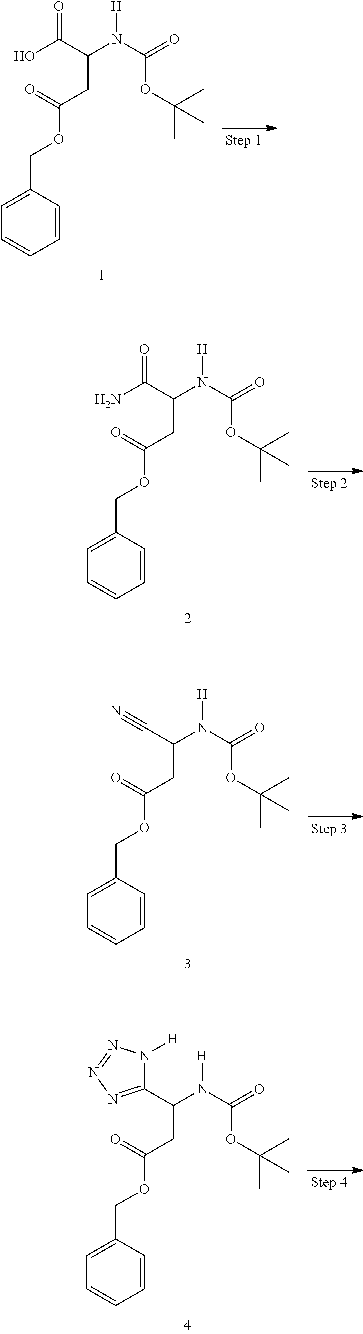 [SMILES: CC(C)(C)OC(=O)NC(CC(=O)OCc1ccccc1)C(=O)O ; CC(C)(C)OC(=O)NC(CC(=O)OCc1ccccc1)C(N)=O ; CC(C)(C)OC(=O)NC(C#N)CC(=O)OCc1ccccc1 ; CC(C)(C)OC(=O)NC(CC(=O)OCc1ccccc1)c1nnn[nH]1]

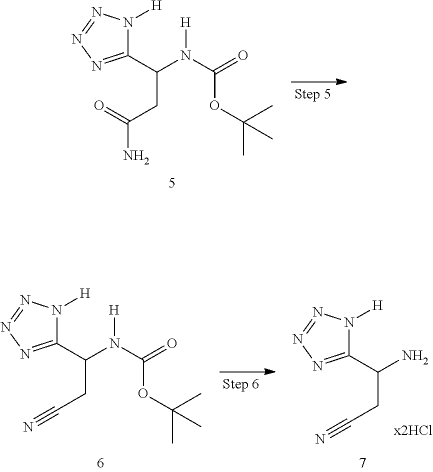 [SMILES: CC(C)(C)OC(=O)NC(CC(N)=O)c1nnn[nH]1 ; CC(C)(C)OC(=O)NC(CC#N)c1nnn[nH]1 ; N#CCC(N)c1nnn[nH]1]

### Step 1: Synthesis of 3-tert-butoxycarbonylamino-succinamic acid benzyl ester (2)

To a solution of 4-(benzyloxy)-2-(tert-butoxycarbonylamino)-4-oxobutanoic acid (1) (4.68 g, 14.46 mmol) and triethylamine (2.42 mL) in anhydrous tetrahydrofuran (73 mL) was added ethyl chloroformate (1.66 mL, 17.36 mmol) at 0° C. After 0.5 h, 25% aqueous ammonia (23.2 mL) was added, and the reaction was stirred for 1 h. The reaction mixture was evaporated and the residue was triturated with water (70 mL) to afford 3-tert-butoxycarbonylamino-succinamic acid benzyl ester (2) (3.30 g, 10.26 mmol, 71%) as a white crystalline solid. ESMS m/z 345 (M+Na)⁺.

### Step 2: Synthesis of 3-tert-butoxycarbonylamino-3-cyanopropionic acid benzyl ester (3)

To a mixture of 3-tert-butoxycarbonylamino-succinamic acid benzyl ester (2) (3.30 g, 10.26 mmol) and pyridine (4.30 mL) in 1,4-dioxane (46 mL) was added trifluoroacetic acid anhydride (2.98 mL, 21.42 mmol) at 0° C. The resulting reaction mixture was stirred for 10 min at 0° C., then warmed to 15° C. and stirred for 30 min. A 10% solution of sodium bicarbonate (50 mL) was added dropwise, the mixture was diluted with water (50 mL) and extracted with ethyl acetate (3×50 mL). The combined organic layers were dried over sodium sulfate, filtered and evaporated. The residue was triturated with n-hexane (30 mL) to give 3-tert-butoxycarbonylamino-3-cyanopropionic acid benzyl ester (3) (2.94 g, 9.66 mmol, 94%) as a pale yellow crystalline solid. ESMS m/z 327 (M+Na)⁺.

### Step 3: Synthesis of 3-tert-butoxycarbonylamino-3-(1H-tetrazol-5-yl)propionic acid benzyl ester (4)

A mixture of 3-tert-butoxycarbonylamino-3-cyanopropionic acid benzyl ester (3) (1.00 g, 3.28 mmol), ammonium chloride (0.25 g, 4.67 mmol) and sodium azide (0.30 g, 4.61 mmol) in N,N-dimethylformamide (20 mL) was stirred at 110° C. for 3 h under nitrogen. The resulting solid was removed by filtration and washed with ethyl acetate (2×5 mL). The filtrate was evaporated and the residue taken up in a mixture of ethyl acetate (20 mL), water (5 mL) and 10% acetic acid (5 mL). The layers were separated and the organic layer dried over sodium sulfate, filtered and evaporated. The crude product was triturated with diisopropyl ether to give 3-tert-butoxycarbonylamino-3-(1H-tetrazol-5-yl)propionic acid benzyl ester (4) (0.55 g, 1.58 mmol, 48%) as an off-white crystalline solid. ESMS m/z 348 (M+H)⁺.

### Step 4: Synthesis of [2-carbamoyl-1-(1H-tetrazol-5-yl)ethyl]carbamic acid tert-butyl ester (5)

A mixture of 3-tert-butoxycarbonylamino-3-(1H-tetrazol-5-yl)propionic acid benzyl ester (4) (0.40 g, 1.15 mmol) and 40% ammonia in methanol (12 mL) was stirred at 70° C. for seven days. The reaction mixture was evaporated and the residue triturated with 2-propanol to give [2-carbamoyl-1-(1H-tetrazol-5-yl)ethyl]carbamic acid tert-butyl ester (5) (0.14 g, 0.54 mmol, 48%) as an off-white crystalline solid. ESMS m/z 257 (M+H)⁺.

### Step 5: Synthesis of [2-cyano-1-(1H-tetrazol-5-yl)ethyl]carbamic acid tert-butyl ester (6)

To a mixture of [2-carbamoyl-1-(1H-tetrazol-5-yl)ethyl]carbamic acid tert-butyl ester (85 mg, 0.33 mmol) (5) and pyridine (134 μL, 1.66 mmol) in anhydrous 1,4-dioxane (18 mL) was added a solution of trifluoroacetic acid anhydride (94 μL, 0.66 mmol) in anhydrous 1,4-dioxane (3 mL) at 10° C. The resulting reaction mixture was stirred for 30 min. A 10% sodium bicarbonate solution was then added dropwise to achieve pH 7. The mixture was diluted with water (10 mL) and washed with dichloromethane (3×20 mL). The aqueous layer was evaporated and the residue suspended in ethanol. The precipitate was removed by filtration and the filtrate was evaporated. The crude product was purified by column chromatography eluting with ethyl acetate:methanol (4:1), and the resulting residue triturated with diethyl ether to give [2-cyano-1-(1H-tetrazol-5-yl)ethyl]carbamic acid tert-butyl ester (6) (77 mg, 0.32 mmol, 97%) as a white solid. ESMS m/z 239 (M+H)⁺.

### Step 6: Synthesis of 3-amino-3-(1H-tetrazol-5-yl)propionitrile dihydrochloride (7)

A mixture of [2-cyano-1-(1H-tetrazol-5-yl)ethyl]carbamic acid tert-butyl ester (6) (48 mg, 0.20 mmol) and 3.8 M hydrogen chloride in 1,4-dioxane (1 mL) was stirred for 1 h. The reaction mixture was evaporated and the residue triturated with diethyl ether to give 3-amino-3-(1H-tetrazol-5-yl)propionitrile dihydrochloride (7) (22 mg, 0.10 mmol, 51%) as a white hygroscopic solid. ESMS m/z 137 (M−H)⁻; ¹H NMR (500 MHz, DMSO-d₆, salt) δ 9.05 (br. s, 3H), 5.19 (dd, J=7.8, 5.4 Hz, 1H), 3.46-3.52 (m, 1H), 3.39-3.45 (m, 1H).

### Example 1-1a

### Synthesis of (S)-3-amino-3-(1H-tetrazol-5-yl)propionitrile dihydrochloride (7a)

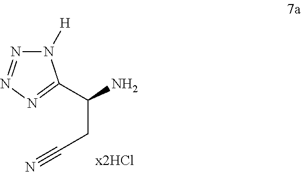

Using the procedure of Example 1-1, but starting with (S)-4-(benzyloxy)-2-(tert-butoxycarbonylamino)-4-oxobutanoic acid, affords (S)-3-amino-3-(1H-tetrazol-5-yl)propionitrile dihydrochloride (7a).

### Example 1-1b

### Synthesis of (R)-3-amino-3-(1H-tetrazol-5-yl)propionitrile dihydrochloride (7b)

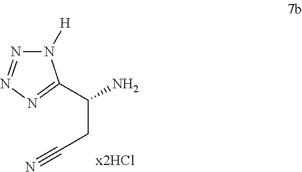

Using the procedure of Example 1-1, but starting with (R)-4-(benzyloxy)-2-(tert-butoxycarbonylamino)-4-oxobutanoic acid, affords (R)-3-amino-3-(1H-tetrazol-5-yl)propionitrile dihydrochloride (7b).

### Example 1-2

### Synthesis of (S)-1-(1H-tetrazol-5-yl)-but-3-ynylamine hydrochloride (12)

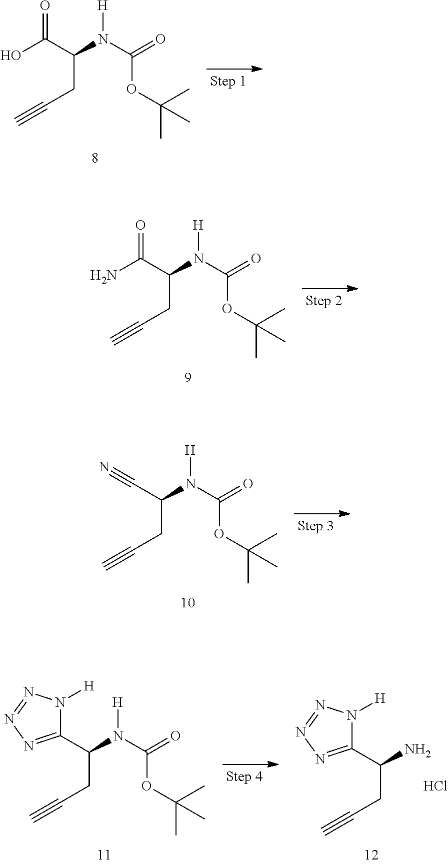

### Step 1: Synthesis of (S)-(1-carbamoyl-but-3-ynyl)-carbamic acid tert-butyl ester (9)

To a pre-cooled (0-5° C.) solution of (S)-2-tert-butoxycarbonylamino-pent-4-ynoic acid (8) (88.86 g, 0.417 mol) in dry tetrahydrofuran (1100 mL) under nitrogen was added N-methylmorpholine (49.0 mL, 44.59 g, 0.441 mol). Ethyl chloroformate (40.5 mL, 46.17 g, 0.425 mol) was added dropwise over 30 min, maintaining the temperature between 0-5° C. The mixture was stirred for 30 min at 0° C., then added dropwise over 30 min to a pre-cooled (0-5° C.) solution of aqueous ammonia (360 mL, 25%) and stirred for 10 min. The aqueous layer was extracted with ethyl acetate (2×250 mL). The combined organic layers were washed with 10% aqueous sodium carbonate (200 mL) and brine (100 mL), and evaporated to yield the crude product (80.50 g). The residue was triturated with water (80 mL) and the collected solid was washed with cold water (2×10 mL) to afford (S)-(1-carbamoyl-but-3-ynyl)-carbamic acid tert-butyl ester (9) (49.10 g, 0.231 mol, 55%) as a white crystalline solid. LCMS (205 nm): 100%, (M+Na)⁺235; TLC in chloroform/acetic acid 20:1, visualized with chlorotoluidine: Rf_{SM}=0.45, Rf_{prod}=0.32.

### Step 2: Synthesis of (S)-(1-cyano-but-3-ynyl)-carbamic acid tert-butyl ester (10)

(S)-(1-carbamoyl-but-3-ynyl)-carbamic acid tert-butyl ester (9) (48.04 g, 0.226 mol) was dissolved in a mixture of pyridine (94 mL, 92.30 g, 1.167 mol) and dry dioxane (940 mL) at 5° C. under nitrogen. Trifluoroacetic anhydride (66 mL, 98.14 g, 0.467 mol) was added dropwise, and the mixture was stirred for 30 min at 5° C., then for 1 h at room temperature. The mixture was concentrated to ca. 250 ml, in vacuo. The residue was added dropwise to saturated aqueous sodium bicarbonate (200 mL), maintaining the pH between 6 and 7 through the addition of solid sodium bicarbonate (99.40 g). Ethyl acetate (200 mL) was added, the inorganic solid was removed by filtration and the solid was extracted with ethyl acetate (100 mL). The combined filtrate was separated and the aqueous layer extracted with ethyl acetate (2×100 mL). The combined organic layers were dried over sodium sulfate and evaporated. The residue was triturated with hexane and the collected solid was washed with hexane (4×20 mL) to give (S)-(1-cyano-but-3-ynyl)-carbamic acid tert-butyl ester (10) (42.63 g, 0.219 mol, 97%) as a tan solid. TLC in chloroform/acetic acid 20:1, visualized with chlorotoluidine: Rf=0.67.

### Step 3: Synthesis of (S)-[1-(1H-tetrazol-5-yl)-but-3-ynyl]-carbamic acid tert-butyl ester (11)

A mixture of (S)-(1-cyano-but-3-ynyl)-carbamic acid tert-butyl ester (10) (40.77 g, 0.212 mol), ammonium chloride (16.81 g, 0.314 mol) and sodium azide (20.42 g, 0.314 mol) in dry DMF (415 mL) was heated at 100° C. under nitrogen for 20 h. The inorganic solid was removed by filtration and the filtrate was evaporated. The residue was partitioned between ethyl acetate (500 mL) and 10% aqueous sodium bicarbonate (250 mL). The aqueous layer was washed with ethyl acetate (2×100 mL), acidified to pH 4 with acetic acid, and extracted with ethyl acetate (2×100 mL). The combined acidic organic layers were washed with brine, dried over sodium sulfate, and evaporated. The crude product was triturated with hexane and the collected solid was washed with hexane (3×50 mL) to give (S)-[1-(1H-tetrazol-5-yl)-but-3-ynyl]-carbamic acid tert-butyl ester (11) (48.05 g, 0.202 mol, 96%) as an off-white crystalline solid. TLC in ethyl acetate/methanol 4:1, visualized with chlorotoluidine: Rfₛₘ=0.95, Rf_{prod}=0.70.

### Step 4: Synthesis of (S)-1-(1H-tetrazol-5-yl)-but-3-ynylamine hydrochloride (12)

(S)-[1-(1H-tetrazol-5-yl)-but-3-ynyl]-carbamic acid tert-butyl ester (11) (43.26 g, 0.182 mol) was dissolved in 3.87 M hydrogen chloride in methanol (405 mL) and stirred at room temperature for 3 h. The mixture was evaporated and the residue was triturated with ethyl acetate (45 mL). The precipitate was washed with ethyl acetate (3×10 mL) to afford (S)-1-(1H-tetrazol-5-yl)-but-3-ynylamine hydrochloride (12) (27.20 g, 0.129 mol, 71%) as a tan crystalline solid. ESMS m/z 138 (M+H)⁺. ¹H NMR (400 MHz, DMSO-d₆) δ 9.17 (s, 3H), 4.92 (m, 1H), 3.02-3.16 (m, 3H); elem. anal.: calc.: C, 34.59; H, 4.64; N, 40.34; Cl, 20.42%, found: C, 33.85, H, 4.64, N, 39.27, Cl, 20.40%; m.p. 166-167° C.; ee: 97%.

### Example 1

### 2a: Synthesis of 1-(1H-tetrazol-5-yl)-but-3-ynylamine hydrochloride (12a)

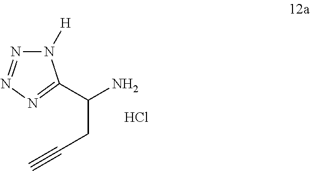

Using the procedure of Example 1-2, but starting with 2-tert-butoxycarbonylamino-pent-4-ynoic acid, afforded 1-(1H-tetrazol-5-yl)-but-3-ynylamine hydrochloride (12a). ESMS m/z 138 (M+H)⁺. ¹H NMR (400 MHz, DMSO-d₆) δ 9.17 (s, 3H), 4.92 (m, 1H), 3.02-3.16 (m, 3H).

### Example 1

### 2b: Synthesis of (R)-1-(1H-tetrazol-5-yl)-but-3-ynylamine hydrochloride (12b)

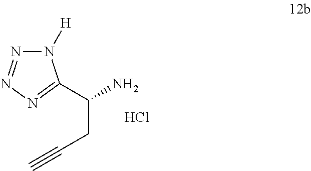

Using the procedure of Example 1-2, but starting with (R)-2-tert-butoxycarbonylamino-pent-4-ynoic acid, affords (R)-1-(1H-tetrazol-5-yl)-but-3-ynylamine hydrochloride (12b).

### Example 1-3

### Synthesis of (S)-3-(1-aminobut-3-ynyl)-1,2,4-oxadiazol-5(4H)-one hydrochloride (15)

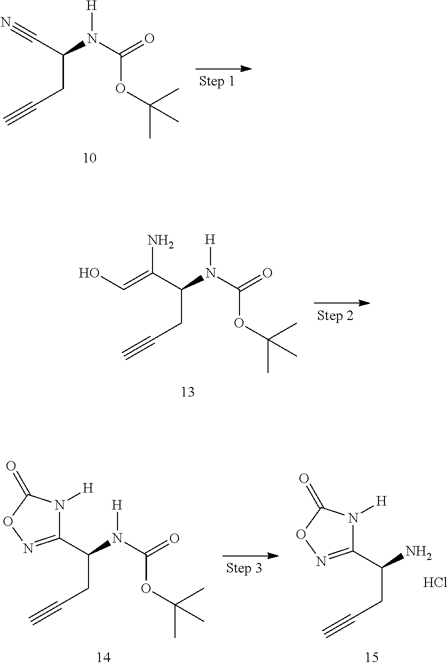 [SMILES: C#CC[C@@H](C#N)NC(=O)OC(C)(C)C ; C#CC[C@H](NC(=O)OC(C)(C)C)/C(N)=C/O ; C#CC[C@H](NC(=O)OC(C)(C)C)c1noc(=O)[nH]1 ; C#CC[C@H](N)c1noc(=O)[nH]1 ; Cl]

### Step 1: Synthesis of (S,Z)-tert-butyl 2-amino-1-hydroxyhex-1-en-5-yn-3-ylcarbamate (13)

To a stirred solution of (S)-(1-cyano-but-3-ynyl)-carbamic acid tert-butyl ester 10 (250 mg, 1.3 mmol) in ethanol (10 ml) was added 50% (w/w) aqueous hydroxylamine (0.36 ml, 5.15 mmol) and the reaction mixture was stirred for 18 h at room temperature. After completion of the reaction, the solvent was concentrated and the resulting residue was suspended in water and extracted with ethyl acetate. The organic layer was washed with brine, dried over sodium sulfate, filtered, and concentrated under reduced pressure to afford (S,Z)-tert-butyl 2-amino-1-hydroxyhex-1-en-5-yn-3-ylcarbamate 13 in 95.7% yield. ¹H NMR (400 MHz, CDCl₃) δ 1.4(s, 9H), 2.5(brs, 2H), 2.8(S, 1H), 4.1 (t, 1H), 5.21 (s, 2H), 6.9(d, 1H), 9.1 (s, 1H).

### Step 2: Synthesis of (S)-(1-cyano-but-3-ynyl)-carbamic acid tert-butyl ester (14)

To a stirred solution of (S,Z)-tert-butyl 2-amino-1-hydroxyhex-1-en-5-yn-3-ylcarbamate 13 (200 mg, 0.9 mmol) in tetrahydrofuran (5 ml) was added 1,1′-carbonyldiimidazole (214 mg, 1.32 mmol) and the mixture was heated at reflux for 5 h under nitrogen atmosphere. After completion of the reaction mixture was cooled and solvent was concentrated under reduced pressure. The crude residue was dissolved in ethyl acetate and extracted with a 1M sodium hydroxide solution. The aqueous layer was diluted with dichloromethane, carefully acidified (pH-3-4) with 1M hydrochloric acid under cooling and extracted with dichloromethane. The organic layers were combined, washed with brine, dried over sodium sulfate, filtered, and evaporated to afford (S)-(1-cyano-but-3-ynyl)-carbamic acid tert-butyl ester 14 in 45% yield. ¹H NMR (400 MHz, DMSO) δ 1.5(s, 9H), 2.7 (d, 2H), 2.9 (s, 1H), 4.6 (d, 1H), 7.5 (d, 1H), 12.4(s, 1H). ESMS (negative mode): 252.15 (M−1).

### Step 3: Synthesis of (S)-3-(1-aminobut-3-ynyl)-1,2,4-oxadiazol-5(4H)-one hydrochloride (15)

To a stirred solution of MeOH—HCl (6 ml) was added (S)-(1-cyano-but-3-ynyl)-carbamic acid tert-butyl ester 14 (90 mg, 0.35 mmol) and the resulting mixture was stirred for 12 h at room temperature. After completion of reaction, solvent was removed under reduced pressure, washed twice with ether and dried under reduced pressure to afford the desired product 6 in 59% yield as off white solid. ¹H NMR (400 MHz, DMSO) δ 2.9 (brs, 2H), 3.22 (s, 1H), 4.61 (t, 1H), 9.0-10.1 (br s, 2H). ELSD Purity: 97.050%; ESMS (negative mode): 152.33 (M−1).

### Example 1-4

### Synthesis of (S)-1-(1H-1,2,4-triazol-5-yl)but-3-yn-1-amine hydrochloride (20)

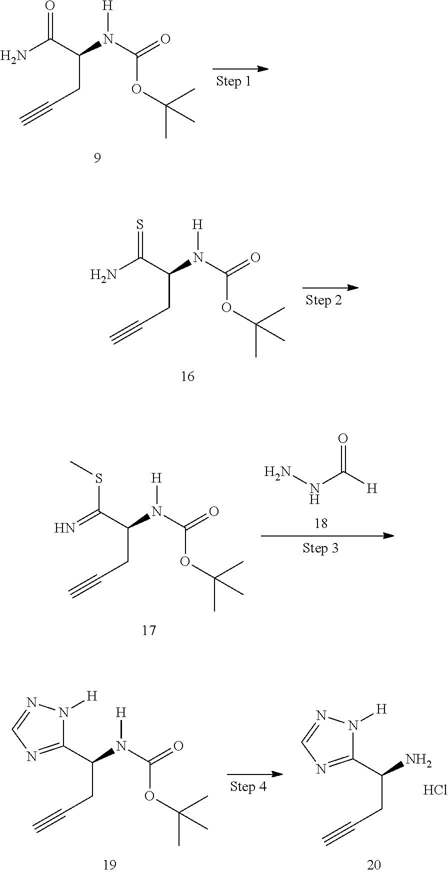

### Step 1: Synthesis of (S)-tert-butyl 1-amino-1-thioxopent-4-yn-2-ylcarbamate (16)

To a stirred solution of (S)-tert-butyl 1-amino-1-oxopent-4-yn-2-ylcarbamate (9) (4.2 g, 19.7 mmol) in THF (40 ml), was added Lawesson's reagent (4 g, 9.8 mmol) and reaction mixture was heated at 50° C. for 1 h. After completion of reaction, solvent was removed under reduced pressure and crude residue was purified by silica gel column chromatography (Ethyl acetate: Hexane=1:2) to obtain (S)-tert-butyl 1-amino-1-thioxopent-4-yn-2-ylcarbamate (16) in 71% yield. ¹H NMR (400 MHz, CD₃OD) δ 1.4 (s, 9H), 2.7 (d, 2H), 2.9 (s, 1H), 4.4 (d, 1H), 6.8 (d, 1H), 9.2 (br s, 1H), 9.8 (br s, 1H).

### Step 2: Synthesis of (S)-methyl 2-(tert-butoxycarbonylamino)pent-4-ynimidothioate (17)

To a stirred solution of (S)-tert-butyl 1-amino-1-thioxopent-4-yn-2-ylcarbamate (16) (3.1 g, 13.5 mmol) in acetonitrile (25 ml) was added methyl iodide (9.6 g, 67.9 mmol) was added and the reaction mixture was heated at 50° C. for 1 h under nitrogen atmosphere. After completion of reaction, solvent was evaporated and the crude residue was washed with diethyl ether and dried to obtain (S)-methyl 2-(tert-butoxycarbonylamino)pent-4-ynimidothioate (17) as a white solid in 88% yield. ESMS (positive mode): 243.15 (M+1).

### Step 3: Synthesis of (S)-tert-butyl 1-(1H-1,2,4-triazol-5-yl)but-3-ynylcarbamate (19)

To a stirred solution of (S)-methyl 2-(tert-butoxycarbonylamino)pent-4-ynimidothioate (17) (1 g, 4.12 mmol) and formyl hydrazide (18) (297 mg, 4.95 mmol) in ethanol (15 ml) was added diisopropylethyl amine (1.6 g, 12.3 mmol) and the reaction mixture was heated to reflux for 4 h. After completion of reaction, solvent was removed under reduced pressure and the crude residue was purified by column chromatography (Ethyl acetate: Hexane=1:1) to obtain (S)-tert-butyl 1-(1H-1,2,4-triazol-5-yl)but-3-ynylcarbamate (19) in 21% yield. ¹H NMR (400 MHz, CDCl3) δ 1.5 (s, 9H), 2.95 (br d, 2H), 5.1 (t, 1H), 5.6 (br s, 1H), 8.1 (br s, 1H), 11.8 (br s, 1H).

### Step 4: Synthesis of (S)-1-(1H-1,2,4-triazol-5-yl)but-3-yn-1-amine hydrochloride (20)

To a stirred solution of MeOH.HCl (10 ml), (S)-tert-butyl 1-(1H-1,2,4-triazol-5-yl)but-3-ynylcarbamate (19) (200 mg, 0.847 mmol) was added and the resulting mixture was stirred for 12 h. After completion of reaction, solvent was removed under reduced pressure, washed twice with ether and dried under reduced pressure to afford (S)-1-(1H-1,2,4-triazol-5-yl)but-3-yn-1-amine hydrochloride (20) in 89% yield as off white solid. ¹H NMR (400 MHz, DMSO) δ 2.9 (d, 2H), 3 (s, 1H), 4.6 (br s, 1H), 8.6(s, 1H), 8.7 (br s, 2H). ELSD Purity: 98.95%; Mass (M+1): 137.1.

### Example 1-5

### Synthesis of (5)-1-(3-(trifluoromethyl)-1H-1,2,4-triazol-5-yl)but-3-yn-1-amine hydrochloride (23)

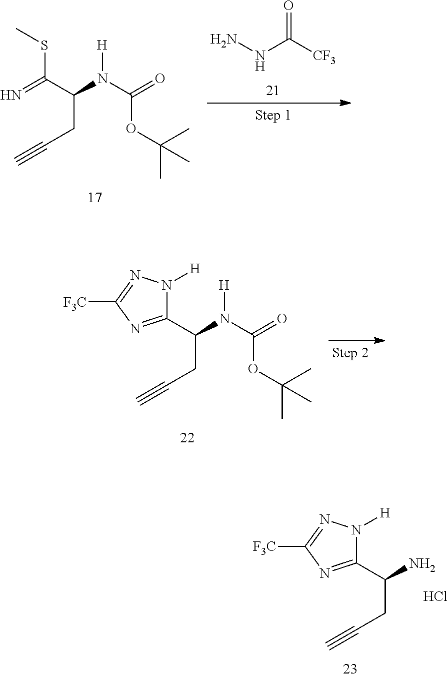 [SMILES: C#CC[C@H](NC(=O)OC(C)(C)C)C(=N)SC ; NNC(=O)C(F)(F)F ; C#CC[C@H](NC(=O)OC(C)(C)C)c1nc(C(F)(F)F)n[nH]1 ; C#CC[C@H](N)c1nc(C(F)(F)F)n[nH]1 ; Cl]

### Step 1: Synthesis of (S)-tert-butyl 1-(3-(trifluoromethyl)-1H-1,2,4-triazol-5-yl)but-3-ynylcarbamate (22)

To a stirred solution of (S)-methyl 2-(tert-butoxycarbonylamino)pent-4-ynimidothioate (17) (1 g, 4.1 mmol) and trifluoromethyl hydrazide (21) (0.634 g, 4.1 mmol) in ethanol (10 ml) was added diisopropylethyl amine (1.6 g, 12.3 mmol) and the reaction mixture was heated to reflux for 5 h. After completion of reaction, solvent was removed under reduced pressure and the crude residue was purified by column chromatography (Ethyl acetate: Hexane=1:1) to obtain (S)-tert-butyl 1-(3-(trifluoromethyl)-1H-1,2,4-triazol-5-yl)but-3-ynylcarbamate (22) 14% yield. ¹H NMR (400 MHz, CD₃OD) δ 1.5 (s, 9H), 2.0 (s, 1H), 2.9 (d, 2H), 5.0 (d, 1H), 5.5 (d, 1H).

### Step 2: Synthesis of (S)-1-(3-(trifluoromethyl)-1H-1,2,4-triazol-5-yl)but-3-yn-1-amine hydrochloride (23)

To a stirred solution of MeOH.HCl (5 ml), (S)-tert-butyl 1-(3-(trifluoromethyl)-1H-1,2,4-triazol-5-yl)but-3-ynylcarbamate (22) (30 mg, 0.1 mmol) was added and the resulting mixture was stirred for 12 h at room temperature. After completion of reaction, solvent was removed under reduced pressure, washed twice with ether and dried under reduced pressure to afford (S)-1-(3-(trifluoromethyl)-1H-1,2,4-triazol-5-yl)but-3-yn-1-amine hydrochloride (23) in 75% yield as off white solid. ¹H NMR (400 MHz, CD₃OD) δ: 2.6 (s, 1H), 3.1 (d, 2H), 4.8 (br s, 1H). ELSD Purity: 89.1%; ESMS: 204.06 (M⁺).

### Example 1-6

### Synthesis of (S)-5-(1-aminobut-3-ynyl)-1H-1,2,4-triazol-3-amine hydrochloride (27)

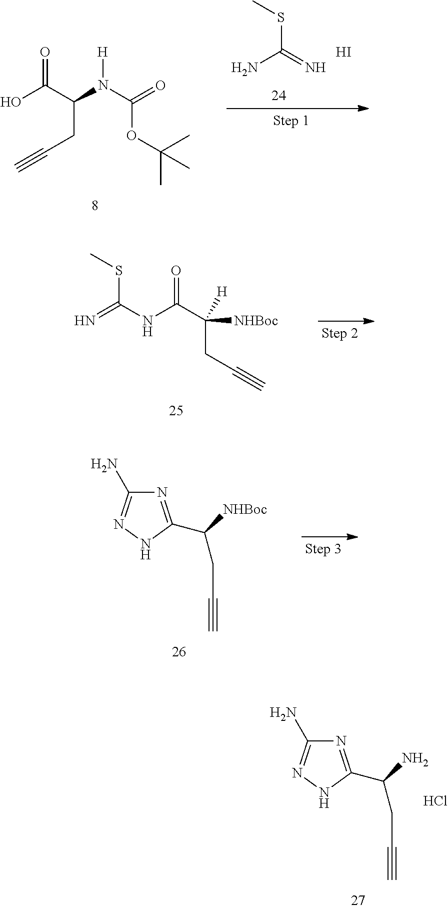

### Step 1: Synthesis of (S)-tert-butyl 1-(imino(methylthio)methylamino)-1-oxopent-4-yn-2-ylcarbamate (25)

To a stirred solution of (S)-2-(tert-butoxycarbonylamino)pent-4-ynoic acid (8) (2.5 g, 11.7 mmol) in dichloromethane (25 ml) was added EDCI (2.72 g, 14.0 mmol), HOBT (1.24 g, 8.21 mmol), methyl carbamimidothioate hydroiodide (24) (2.55 g, 11.7 mmol), and DIPEA (6.13 ml, 35.2 mmole) and the reaction mixture was stirred at room temperature for 12 h under nitrogen atmosphere. After completion of the reaction, the reaction mixture was concentrated under reduced pressure. The crude residue was suspended in water and extracted with ethyl acetate. The organic layers were combined, washed with brine, dried over sodium sulfate, filtered, and concentrated under reduced pressure to obtain crude product which was purified by silica gel column chromatography (EtoAc:Hexane=2:3) to obtain (S)-text-butyl 1-(imino(methylthio)methylamino)-1-oxopent-4-yn-2-ylcarbamate (25) in 60% yield as a pale yellow oil. ¹H NMR (400 MHz, DMSO-d₆) δ 1.4 (s, 9H), 2.4 (s, 3H), 2.6-2.7 (m, 2H), 2.8 (s, 1H), 4.05 (s, 1H), 6.8 (d, 1H), 9.1(br s, 2H). LCMS: 285 (M⁺+1).

### Step 2: Synthesis of (S)-tert-butyl 1-(3-amino-1H-1,2,4-triazol-5-yl)but-3-ynylcarbamate (26)

To a stirred solution of (S)-tert-butyl 1-(imino(methylthio)methylamino)-1-oxopent-4-yn-2-ylcarbamate (25) (1.2 g, 4.21 mmole) in ethanol (25 ml) was added hydrazine monohydrate (0.631 g, 12.6 mmole) and the reaction mixture was heated to reflux for 16 h under nitrogen atmosphere. After completion of the reaction, the reaction mixture was concentrated under reduced pressure and the residue was suspended in water and extracted with ethyl acetate. The organic layers were combined, washed with brine, dried over sodium sulfate, filtered, and concentrated to obtain crude product. The crude residue was purified by silica gel column chromatography (MeOH: dichloromethane=1:9) to afford (S)-tert-butyl 1-(3-amino-1H-1,2,4-triazol-5-yl)but-3-ynylcarbamate (26) in 12% yield as a white solid. ¹H NMR (400 MHz, DMSO) δ 1.4 (s, 9H), 2.6-2.8 (m, 3H), 4.5 (s, 1H), 5.8 (br s, 2H), 6.8 (br s, 1H), 11.8 (br s, 1H). LCMS: 252 (M^{α}+1).

### Step 3: Synthesis of (S)-5-(1-aminobut-3-ynyl)-1H-1,2,4-triazol-3-amine hydrochloride (27)

To a stirred solution of MeOH.HCl (5 ml) was added (S)-text-butyl 1-(3-amino-1H-1,2,4-triazol-5-yl)but-3-ynylcarbamate (26) (30 mg, 0.119 mmol) and the resulting mixture was stirred for 12 h at room temperature. After completion of reaction, solvent was removed under reduced pressure, washed twice with ether and dried under reduced pressure to afford (S)-5-(1-aminobut-3-ynyl)-1H-1,2,4-triazol-3-amine hydrochloride (27) in 72% yield as an off white solid. ¹H NMR (400 MHz, DMSO) δ 2.8 (s, 2H), 3.05 (br s, 1H), 4.35-4.40 (m, 1H), 7.1-7.4 (br s, 2H), 8.7-8.8 (br s, 3H). HPLC Purity: 93.24%; LCMS: 152 (M⁺+1).

### Example 1-7

### Synthesis of (S)-2-amino-N-(2H-tetrazol-5-yl)pent-4-ynamide (29)

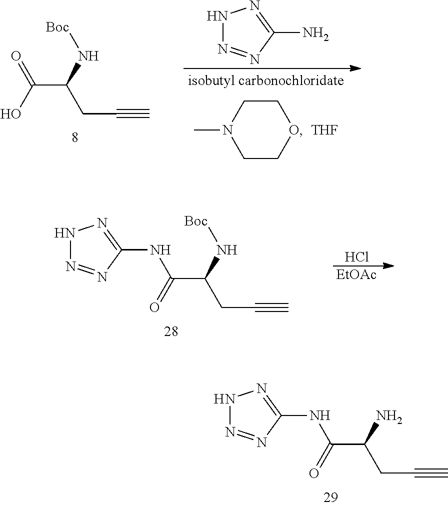

### Step 1: Synthesis of (S)-tert-butyl 1-(2H-tetrazol-5-ylamino)-1-oxopent-4-yn-2-ylcarbamate (28)

1. To a solution of 8 (500 mg, 2.3 mmol) in anhydrous THF (5 mL) was added dropwise 4-methylmorpholine (0.27 mL, 2.8 mmol) followed by isobutyl carbonochloridate (0.23 mL, 2.8 mmol) at 0° C. The suspension was stirred at the same temperature for 30 min prior to addition of 2H-tetrazol-5-amine (200 mg, 2.3 mmol). The mixture was allowed to stir at r.t. for 2 h, then diluted with ethyl acetate (10 mL) and water (15 mL) was added. The organic layer was separated and the aqueous layer was extracted two more times with ethyl acetate (10 mL). The combined organic layers were washed with brine, dried over MgSO₄, filtered and concentrated under reduced pressure to give 28 as a white solid (528 mg). ¹H NMR (400 MHz, DMSO-d₆): δ 1.38 (s, 9H), 2.55-2.60 (m, 2H), 2.91 (s, 1H), 4.34 (d, J=6.8 Hz, 1H), 7.31 (d, J=6.8 Hz, 1H).

### Step 2: Synthesis of (S)-2-amino-N-(2H-tetrazol-5-yl)pent-4-ynamide (29)

2. To a solution of 3 (528 mg, 1.9 mmol) in ethyl acetate (3 mL) was added a solution of HCl gas in ethyl acetate (3 mL, 4 N) at 0° C. The reaction mixture was stirred at room temperature for 16 h. The resulting precipitate was collected by filtration, washed with ethyl acetate (10 mL) and dried to afford (S)-2-amino-N-(2H-tetrazol-5-yl)pent-4-ynamide (29) (100 mg) as a white solid. ¹H NMR (400 MHz, CD₃OD): δ 2.75 (t, J=2.6 Hz, 1H), 3.03 (dd, J=5.6, 2.4 Hz, 2H), 4.39 (t, J=6.2 Hz, 1H). LCMS (ESI): m/z 181.0 [M+1]⁺.

### Example 1-8

### Synthesis of (S)-2-amino-N-(phenylsulfonyl)pent-4-ynamide hydrochloride (31)

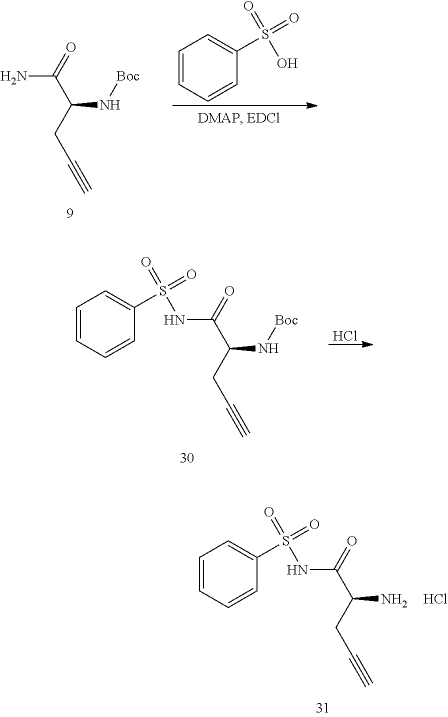 [SMILES: C#CC[C@H](NC(=O)OC(C)(C)C)C(N)=O ; O=S(=O)(O)c1ccccc1 ; C#CC[C@H](NC(=O)OC(C)(C)C)C(=O)NS(=O)(=O)c1ccccc1 ; Cl ; C#CC[C@H](N)C(=O)NS(=O)(=O)c1ccccc1 ; Cl]

### Step 1: Synthesis of (S)-tert-butyl 1-oxo-1-(phenylsulfonamido)pent-4-yn-2-ylcarbamate (30)

3. To a solution of 9 (2.0 g, 9.4 mmol) and benzenesulfonic acid (1.48 g, 9.4 mmol) in dry CH₂Cl₂(20 mL) were added DMAP (1.15 g, 9.4 mmol) and EDCI (1.8 g, 9.4 mmol) at 0° C. After stirring at room temperature for 3 h, the mixture was diluted with ethyl acetate, washed with water and brine, dried over Na₂SO₄, filtered and concentrated under reduced pressure to give a residue which was purified by flash column chromatography (PE/ethyl acetate=10:1) to afford 30 (0.6 g) as a white solid. ¹H NMR (400 MHz, CDCl₃): δ 1.46 (s, 9H), 2.05 (d, J=1.8 Hz, 1H), 2.55-2.62 (m, 1H), 2.68-2.74 (m, 1H), 4.25 (br.s, 1H), 5.21 (br.s, 1H), 7.55 (t, J=7.8 Hz, 2H), 7.66 (t, J=7.9 Hz, 1H), 8.08 (d, J=3.8 Hz, 2H), 9.54 (br.s, 1H).

### Step 2: Synthesis of (S)-2-amino-N-(phenylsulfonyl)pent-4-ynamide hydrochloride (31)

4. 4 M HCl in ethyl acetate (10 mL) was added dropwise to a solution of 30 (600 mg, 1.7 mmol) in ethyl acetate (10 mL) at 0° C. and the reaction mixture was stirred at room temperature for 3 h. The precipitate was filtered, washed with ethyl acetate and dried under reduced pressure to afford (S)-2-amino-N-(phenylsulfonyl)pent-4-ynamide HCl salt (31) (393 mg) as a white solid. ¹H NMR (400 MHz, CD₃OD): δ 2.56 (t, J=2.6 Hz, 1H), 2.81-2.85 (m, 2H), 4.05 (t, J=5.9 Hz, 1H), 7.61 (t, J=7.8 Hz, 2H), 7.72 (t, J=7.4 Hz, 1H), 8.06 (d, J=3.6 Hz, 2H).

### Example 1-9

### Synthesis of (S)-2-amino-N-(methylsulfonyl)pent-4-ynamide hydrochloride (33)

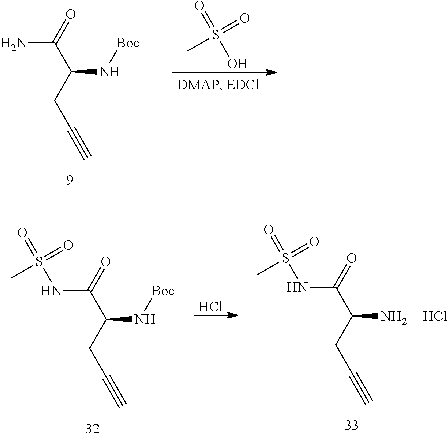

### Step 1: Synthesis of (S)-tert-butyl 1-(methylsulfonamido)-1-oxopent-4-yn-2-ylcarbamate (32)

5. To a solution of 9 (2.0 g, 9.4 mmol) and methanesulfonic acid (0.91 g, 9.4 mmol) in dry CH₂Cl₂(20 mL) were added DMAP (1.15 g, 9.4 mmol) and EDCI (1.8 g, 9.4 mmol). After stirring at room temperature for 3 h, the mixture was diluted with ethyl acetate, washed with water and brine, dried over Na₂SO₄, filtered and concentrated. The residue was purified by flash column chromatography (PE/Ethyl Acetate=10:1) to afford 32 (1.2 g) as a white solid. ¹H NMR (400 MHz, CDCl₃): δ 1.49 (s, 9H), 2.16 (s, 1H), 2.71-2.82 (m, 2H), 3.31 (s, 3H), 4.33 (m, 1H), 5.28 (d, J=7.6 Hz, 1H), 9.35 (br.s, 1H).

### Step 2: Synthesis of (S)-2-amino-N-(methylsulfonyl)pent-4-ynamide hydrochloride (33)

4 M HCl in ethyl acetate (10 mL) was added dropwise to a solution of 32 (765 mg, 2.63 mmol) in ethyl acetate (10 mL) at 0° C. and the reaction mixture was stirred at room temperature for 3 h. The precipitate was filtered and washed with ethyl acetate to afford (S)-2-amino-N-(methylsulfonyl)pent-4-ynamide HCl salt (33) (450 mg) as a white solid. ¹H NMR (400 MHz, CD₃OD): δ 2.73 (t, J=2.6 Hz, 1H), 2.89-2.92 (m, 2H), 3.28 (s, 3H), 4.12 (t, J=5.9 Hz, 1H). LCMS (EST): in/z 191.0 (M+H)⁺.

### Example 2-1

### Synthesis of 3-(1-(1H-tetrazol-5-yl)hydrazinyl)-N,N-dimethylpropan-1-amine (3A)

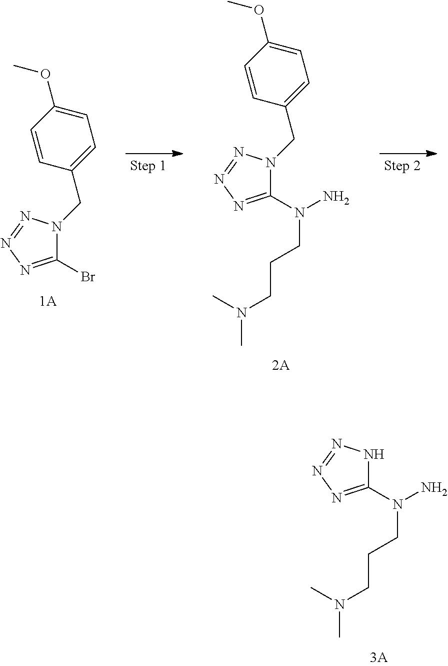 [SMILES: COc1ccc(Cn2nnnc2Br)cc1 ; COc1ccc(Cn2nnnc2N(N)CCCN(C)C)cc1 ; CN(C)CCCN(N)c1nnn[nH]1]

### Step 1: Synthesis of 3-(1-(1-(4-methoxybenzyl)-1H-tetrazol-5-yl)hydrazinyl)-N,N-dimethylpropan-1-amine (2A)

A mixture of 5-bromo-1-(4-methoxybenzyl)-1H-tetrazole (1A) (500 mg, 1.85 mmol) and (3-hydrazinopropyl)dimethylamine (436 mg, 3.72 mmol) in 2-propanol (5 mL) was stirred at 80° C. for 18 h. The reaction mixture was evaporated and the residue was dissolved in a mixture of dichloromethane (20 mL) and brine (10 mL). The layers were separated and the aqueous layer was extracted with dichloromethane (20 mL). The combined organic layers were washed with water (10 mL), dried over sodium sulfate, filtered and evaporated. The crude product was purified by column chromatography eluting with dichloromethane:methanol:triethylamine (100:5:0.5) to give 3-(1-(1-(4-methoxybenzyl)-1H-tetrazol-5-yl)hydrazinyl)-N,N-dimethylpropan-1-amine (2A) (320 mg, 1.05 mmol, 57%) as an orange oil. ESMS m/z 306 (M+H)⁺.

### Step 2: Synthesis of 3-(1-(1H-tetrazol-5-yl)hydrazinyl)-N,N-dimethylpropan-1-amine (3A)

A mixture of 3-(1-(1-(4-methoxybenzyl)-1H-tetrazol-5-yl)hydrazinyl)-N,N-dimethylpropan-1-amine (2A) (120 mg, 0.39 mmol) and 6M hydrochloric acid (1.2 mL) was heated under microwave irradiation at 120° C. for 1.5 h. The reaction mixture was evaporated and the crude product was purified by column chromatography eluting with dichloromethane:methanol:ammonia (4:1:0.2→1:1:0.5). The product was triturated with methanol to give 3-(1-(1H-tetrazol-5-yl)hydrazinyl)-N,N-dimethylpropan-1-amine (3A) (10 mg, 0.05 mmol, 13%) as a white crystalline solid. ESMS m/z 186 (M+H)⁺; ¹H NMR (400 MHz, D₂O) δ 3.55 (t, J=6.5 Hz, 2H), 3.16-3.27 (m, 2H), 2.90 (s, 6H), 2.03-2.13 (m, 2H).

### Example 2-2

### Synthesis of 2-(1-(1H-tetrazol-5-yl)hydrazinyl)-N,N-dimethylethanamine (4A)

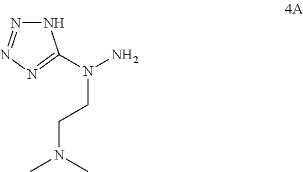

2-(1-(1H-tetrazol-5-yl)hydrazinyl)-N,N-dimethylethanamine (4A) was prepared following a similar procedure as in Example 1. ESMS m/z 172 (M+H)⁺.

### Example 2-3

### Synthesis of 5-(1-(prop-2-ynyl)hydrazinyl)-1H-tetrazole (7A)

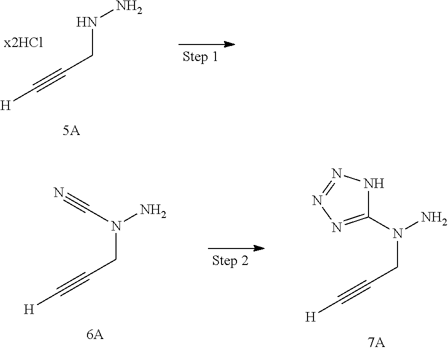 [SMILES: C#CCNN ; C#CCN(N)C#N ; C#CCN(N)c1nnn[nH]1]

### Step 1: Synthesis of 1-(prop-2-ynyl)hydrazinecarbonitrile (6A)

To a solution of cyanogen bromide (0.37 g, 3.49 mmol) in dichloromethane (16.5 mL) was added a mixture of prop-2-ynyl hydrazine dihydrochloride (5A) (0.50 g, 3.49 mmol) and potassium carbonate (0.97 g, 6.99 mmol) in water (10 mL) at 0° C. The reaction mixture was stirred at 0° C. for 1 h. The layers were separated and the aqueous layer was extracted with dichloromethane (2×30 mL). The combined organic layers were dried over magnesium sulfate, filtered and evaporated. The residue was purified by column chromatography eluting with dichloromethane:methanol (100:1) to give 1-(prop-2-ynyl)hydrazinecarbonitrile (6A) (120 mg, 1.26 mmol, 36%) as a pale yellow oil. ESMS m/z 96 (M+H)⁺; ¹H NMR (500 MHz, CDCl₃) δ 4.25 (br. s, 2H), 3.97 (d, J=2.4 Hz, 2H), 2.53 (t, J=2.4 Hz, 1H).

### Step 2: Synthesis of 5-(1-(prop-2-ynyl)hydrazinyl)-1H-tetrazole (7A)

A mixture of 1-(prop-2-ynyl)hydrazinecarbonitrile (6A) (145 mg, 1.52 mmol), sodium azide (119 mg, 1.83 mmol) and ammonium chloride (98 mg, 1.83 mmol) in N,N-dimethylformamide (2 mL) was stirred at 90° C. for 1 h. The resulting mixture was filtered and evaporated. The residue was purified by column chromatography eluting with dichloromethane:methanol:ammonium hydroxide (4:1:0.2) to give the title compound (120 mg, 0.87 mmol, 57%) as a pale yellow gum. ESMS m/z 139 (M+H)⁺; ¹H NMR (500 MHz, DMSO-d₆, salt) δ 4.13 (d, J=2.0 Hz, 2H), 3.05 (br. s, 1H).

### Examples 2-4-2-11

The following compounds were prepared by the method of Example 2-3 using an appropriately functionalized hydrazine in Step 1.

| Ex- | | | | | |
|---|---|---|---|---|---|
| am- | | | ESMS | | |
| ple | Structure | MW | m/z | ¹H NMR | Yield |
| 2-4 | 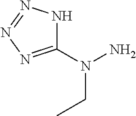 | 128 | 129 | ¹H NMR (500 MHz, DMSO-d₆) salt δ 6.02 (none, 3H), 3.41 (q, J = 7.3 Hz, 2H), 1.10 (t, J = | 32 |
| | | | | 7.3 Hz, 3H). | |
| 2-5 | 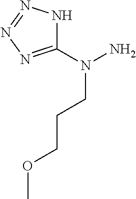 | 172 | 173 | ¹H NMR (500 MHz, DMSO-d₆) δ 4.87 (br. s, 2H), 3.51 (t, J = 7.1 Hz, 2H), 3.36 (t, J = 6.4 Hz, 2H), 3.21 (s, 3H), 1.82-1.90 (m, 2H). | 12 |
| 2-6 | 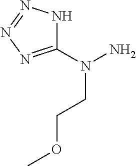 | 158 | 159 | ¹H NMR (500 MHz, CDCl₃) salt δ 5.99 (br. s, 3H), 3.84 (br. s, 2H), 3.74 (t, J = 4.6 Hz, 2H), 3.36 (s, 3H). | 38 |
| 2-7 | 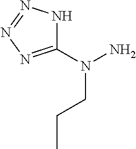 | 142 | 143 | ¹H NMR (500 MHz, D₂O) δ 3.50 (t, J = 7.1 Hz, 2H), 1.62-1.72 (m, 2H), 0.90 (t, J = 7.6 Hz, 3H). | 15 |
| 2-8 | 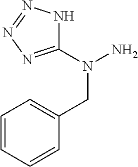 [SMILES: NN(Cc1ccccc1)c1nnn[nH]1] | 190 | 191 | ¹H NMR (500 MHz, DMSO-d₆) salt δ 7.76 (br. s, 3H), 7.27-7.40 (m, 5H), 4.74 (s, 2H). | 9 |
| 2-9 | 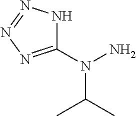 | 142 | 143 | ¹H NMR (500 MHz, DMSO-d₆) δ 4.21-5.12 (m, 2H), 4.09-4.20 (m, 1H), 1.09 (d, J = 6.8 Hz, | 14 |
| | | | | 6H). | |
| 2- 10 | 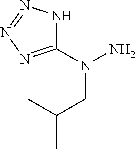 | 156 | 157 | ¹H NMR (400 MHz, D₂O) δ 3.41 (d, J = 7.5 Hz, 2H), 2.06-2.20 (m, 1H), 0.95 (d, J = 6.8 Hz, 6H). | 33 |
| 2- 11 | 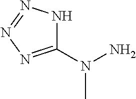 | 114 | 115 | ¹H NMR (500 MHz, DMSO-d₆) δ 14.57 (br. s, 1H), 4.94 (br. s, 2H), 3.14 | 60 |
| | | | | (s, 3H). | |

### Example 2-12

### Synthesis of 3-(1-(1H-tetrazol-5-yl)hydrazinyl)propan-1-amine dihydrochloride (13A)

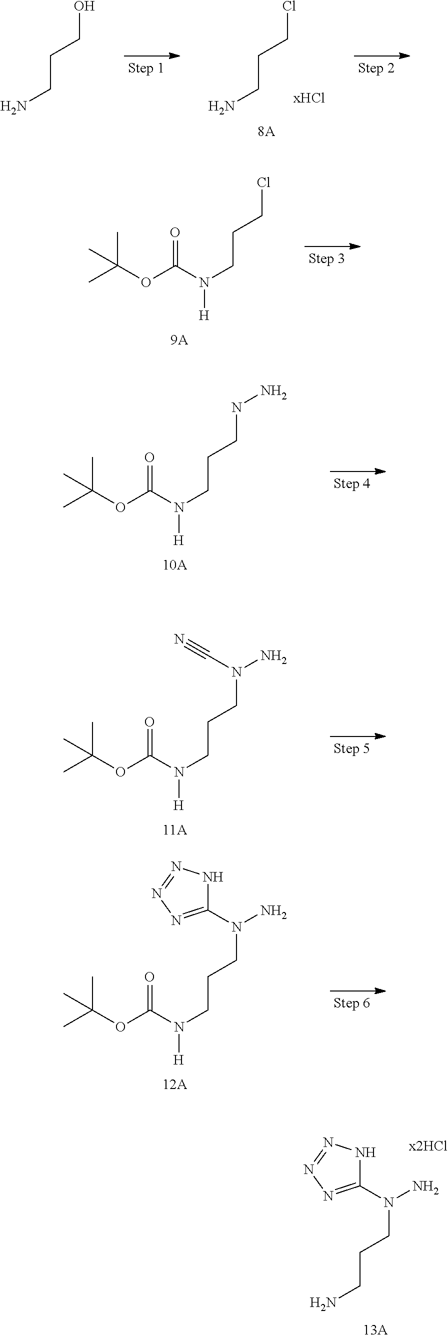

### Step 1: Synthesis of 3-chloropropylamine hydrochloride (8A)

To solution of thionyl chloride (8.68 g, 1.32 mmol) in anhydrous chloroform (30 mL) was added dropwise to 3-aminopropan-1-ol (4.49 g, 59.19 mmol) while maintaining the temperature at 0-10° C. The mixture was allowed to warm to room temperature and then heated at reflux for 3 h. The mixture was cooled to room temperature and the precipitate was collected to give 3-chloropropylamine hydrochloride (8A) (7.07 g, 55.15 mmol, 93%) as a green solid. ESMS m/z 94 (M+H)⁺.

### Step 2: Synthesis of tert-butyl 3-chloropropylcarbamate (9A)

A mixture of 3-chloropropylamine hydrochloride (8A) (5.00 g, 38.46 mmol) and triethylamine (4.11 g, 40.58 mmol) in dichloromethane was stirred at room temperature for 30 min. The mixture was cooled to 0° C. and a solution of di-tert-butyl dicarbonate (8.86 g, 40.58 mmol) in dichloromethane (30 mL) was added. The mixture was stirred at room temperature for 2 h. The reaction mixture was washed with 10% aqueous potassium hydrogen sulfate (40 mL) and water (40 mL). The organic layer was dried over sodium sulfate, filtered and evaporated to give tert-butyl 3-chloropropylcarbamate (9A) (7.89 g, 40.77 mmol, quantitative) as a light brown oil. The crude material was used in the next step without further purification. ESMS m/z 138 (M+H-t-Bu)⁺.

### Step 3: Synthesis of tert-butyl 3-hydrazinylpropylcarbamate (10A)

To a refluxing solution of hydrazine hydrate (6.40 g, 128.00 mmol) in ethanol (14.5 mL) was added a solution of tert-butyl 3-chloropropylcarbamate (9A) (3.80 g, 19.62 mmol) in ethanol (14.5 mL), dropwise over 80 min. The mixture stirred at reflux for 1 h. The reaction mixture was then evaporated and the residue diluted with diethyl ether (60 mL). The two layers were separated. The organic layer was washed with saturated sodium carbonate solution (17 mL) and evaporated to give tert-butyl 3-hydrazinylpropylcarbamate (10A) (1.60 g, 8.45 mmol, 43%) as a yellow oil. The crude material was used in the next step without further purification. ESMS m/z 190 (M+H)⁺.

### Step 4: Synthesis of tert-butyl 3-(1-cyanohydrazinyl)propylcarbamate (11A)

A solution of cyanogen bromide (1.68 g, 15.8 mmol) in dichloromethane (50 mL) was added simultaneously a mixture of tert-butyl 3-hydrazinylpropylcarbamate (10A) (3.00 g, 15.85 mmol) in water (16 mL) and a solution of sodium carbonate (837 mg, 7.90 mmol) in water (16 mL) at 0° C. The reaction mixture was stirred at 0° C. for 1 h. The two layers were then separated. The organic layer was dried over sodium sulfate, filtered and evaporated while maintaining the temperature below 10° C. to give tert-butyl 3-(1-cyanohydrazinyl)propylcarbamate (11A) (2.12 g, 9.89 mmol, 63%) as a yellow oil. ESMS m/z 159 (M+H-t-Bu)⁺.

### Step 5: Synthesis of tert-butyl 3-(1-(1H-tetrazol-5-yl)hydrazinyl)propylcarbamate (12A)

A mixture of tert-butyl 3-(1-cyanohydrazinyl)propylcarbamate (11A) (2.12 g, 9.89 mmol), sodium azide (780 mg, 12.00 mmol) and ammonium chloride (642 mg, 12.00 mmol) in anhydrous N,N-dimethylformamide (20 mL) was stirred at 40° C. for 18 h. The reaction mixture was filtered and evaporated. The residue was purified by column chromatography eluting with chloroform:methanol (95:5) to give tert-butyl 3-(1-(1H-tetrazol-5-yl)hydrazinyl)propylcarbamate (12A) (1.08, 4.20 mmol, 42%) as a yellow oil. ESMS m/z 258 (M+H)⁺.

### Step 6: Synthesis of 3-(1-(1H-tetrazol-5-yl)hydrazinyl)propan-1-amine dihydrochloride (13A)

To tert-butyl 3-(1-(1H-tetrazol-5-yl)hydrazinyl)propylcarbamate (12A) (134 mg, 0.52 mmol) was added a 4.0M solution of hydrogen chloride in methanol (1.5 mL), and the mixture was stirred at room temperature for 2 h. The precipitate was collected and washed with methanol (2×0.5 mL) to give 3-(1-(1H-tetrazol-5-yl)hydrazinyl)propan-1-amine dihydrochloride (13A) (59 mg, 0.26 mmol, 51%) as a white solid. ESMS m/z 158 (M+H)⁺; ¹H NMR (500 MHz, D₂O) δ 3.73 (t, J=5.0 Hz, 2H), 3.13 (m, 2H), 2.14 (m, 2H).

### Examples 2-13-2-15

The following compounds were prepared by the method of Example 2-12 using an appropriately functionalized amine in Step 1.

| Ex- | | | | | |
|---|---|---|---|---|---|
| am- | | | ESMS | | |
| ple | Structure | MW | m/z | ¹H NMR | Yield |
| 2-13 | 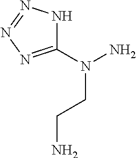 | 143 | 144 | 1H NMR (500 MHz, D2O) δ 3.88 (t, J = 5.6 Hz, 2H), 3.40 (t, J = 5.6 Hz, 2H). | 61 |
| 2-14 | 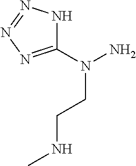 | 157 | 158 | ¹H NMR (500 MHz, D₂O) δ 3.92 (t, J = 5.0 Hz, 2H), 3.45 (t, J = 5.0 Hz, 2H), 2.78 (s, 1H). | 61 |
| 2-15 | 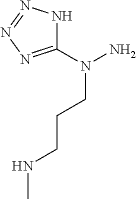 | 171 | 172 | ¹H NMR (500 MHz, D₂O) δ 3.63 (t, J = 5.0 Hz, 2H), 3.07 (m, 2H), 2.67 (s, 3H), 2.06 (m, 2H). | 52 |

### Example 2-16

### Synthesis of 5-(3-(1-(1H-tetrazol-5-yl)hydrazinyl)propyl)-1H-tetrazole (19A)

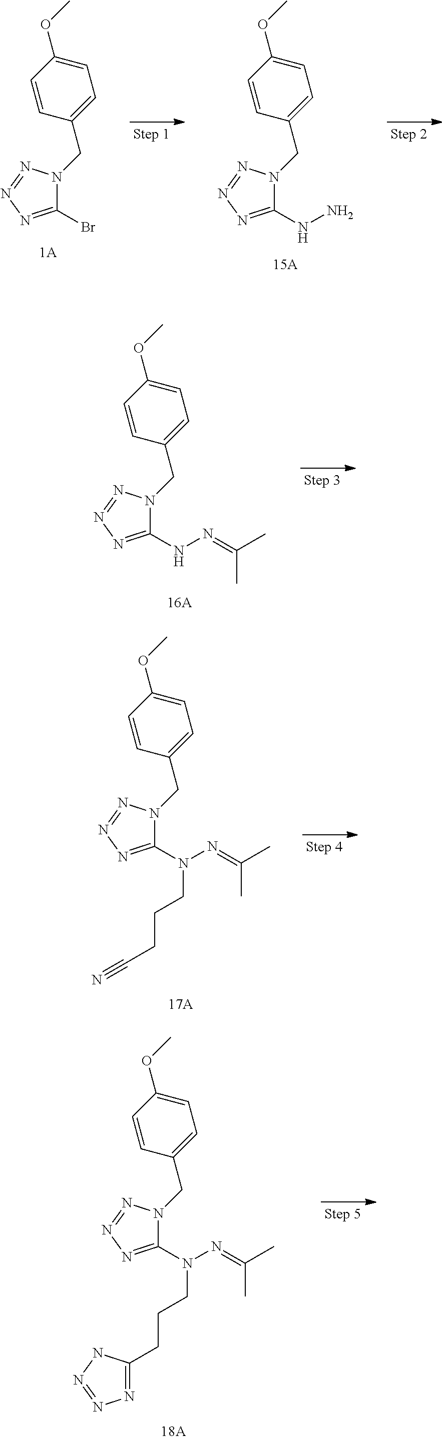 [SMILES: COc1ccc(Cn2nnnc2Br)cc1 ; COc1ccc(Cn2nnnc2NN)cc1 ; COc1ccc(Cn2nnnc2NN=C(C)C)cc1 ; COc1ccc(Cn2nnnc2N(CCCC#N)N=C(C)C)cc1 ; COc1ccc(Cn2nnnc2N(CCCc2nnn[nH]2)N=C(C)C)cc1]

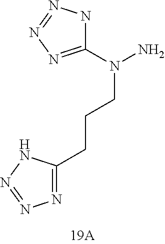 [SMILES: NN(CCCc1nnn[nH]1)c1nnn[nH]1]

### Step 1: Synthesis of 5-hydrazinyl-1-(4-methoxybenzyl)-1H-tetrazole (15A)

A mixture of 5-bromo-1-(4-methoxybenzyl)-1H-tetrazole (1A) (3.10 g, 11.43 mmol) and hydrazine hydrate (2.21 mL, 45.67 mmol) in 2-propanol (25 mL) was stirred at 60° C. for 16 h. 2-Propanol was evaporated, and the residue was triturated with water (20 mL) to give 5-hydrazinyl-1-(4-methoxybenzyl)-1H-tetrazole (15A) (2.04 g, 9.17 mmol, 80%) as an off-white crystalline solid. ESMS m/z 221 (M+H)⁺.

### Step 2: Synthesis of 1-(4-methoxybenzyl)-5-(2-(propan-2-ylidene)hydrazinyl)-1H-tetrazole (16A)

A mixture of [1-(4-methoxybenzyl)-1H-tetrazol-5-yl]hydrazine (15A) (1.80 g, 8.17 mmol), acetone (18 mL) and 3 drops of 4 M hydrochloric acid in diethyl ether was stirred at room temperature for 16 h. The precipitate was collected to give 1-(4-methoxybenzyl)-5-(2-(propan-2-ylidene)hydrazinyl)-1H-tetrazole (16A) (1.91 g, 7.33 mmol, 90%) as an off-white crystalline solid. ESMS m/z 261 (M+H)⁺.

### Step 3: Synthesis of 4-(1-(1-(4-methoxybenzyl)-1H-tetrazol-5-yl)-2-(propan-2-ylidene)hydrazinyl)butanenitrile (17A)

A mixture of 1-(4-methoxybenzyl)-5-(2-(propan-2-ylidene)hydrazinyl)-1H-tetrazole (16A) (0.50 g, 1.92 mmol), sodium hydride (0.12 g, 3.00 mmol, 60% dispersion) and anhydrous tetrahydrofuran (5 mL) was stirred at 0° C. for 0.5 h. 4-Bromobutyronitrile (285.4, 2.87 mmol) was added to the stirred mixture at 0° C., and the reaction mixture was allowed to warm to room temperature and stirred for 18 h. Additional portions of sodium hydride (76 mg, 1.90 mmol, 60%) and 4-bromobutyronitrile (190 μL, 1.90 mmol) were added at room temperature and the reaction mixture was stirred for 24 h and evaporated. The residue was dissolved in water (15 mL) and extracted with dichloromethane (3×15 mL). The combined organic layers were dried over sodium sulfate, filtered and evaporated. The crude product was purified by column chromatography eluting with n-hexane:ethyl acetate (2:3 v/v) to give 4-(1-(1-(4-methoxybenzyl)-1H-tetrazol-5-yl)-2-(propan-2-ylidene)hydrazinyl)butanenitrile (17A) (492 mg, 1.50 mmol, 78%) as a yellow oil. ESMS m/z 328 (M+H)⁺.

### Step 4: Synthesis of 5-(1-(3-(1H-tetrazol-5-ybpropyl)hydrazinyl)-1-(4-methoxybenzyl)-1H-tetrazole (18A)

A mixture of 4-(1-(1-(4-methoxybenzyl)-1H-tetrazol-5-yl)-2-(propan-2-ylidene)hydrazinyl)butanenitrile (17A) (490 mg, 1.50 mmol), sodium azide (117 mg, 1.80 mmol) and ammonium chloride (96 mg, 1.80 mmol) in anhydrous N,N-dimethylformamide (10 mL) was stirred at 90° C. for 18 h. More sodium azide (78 mg, 1.20 mmol) and ammonium chloride (64 mg, 1.20 mmol) was added to the mixture and the stirring was continued further at 90° C. for 18 h. Additional portions of sodium azide (2×78 mg, 1.20 mmol) and ammonium chloride (2×64 mg, 1.20 mmol) were added to the mixture at 24 h intervals and the reaction was stirred at 90° C. After a total of 3 days, the mixture was evaporated, the residue was suspended in 2-propanol (20 mL) and filtered. The filtrate was evaporated to give 5-(1-(3-(1H-tetrazol-5-yl)propyl)hydrazinyl)-1-(4-methoxybenzyl)-1H-tetrazole (18A) (320 mg, 0.97 mmol, 65%) as a yellow oil. (M+H)⁺ 331. The crude product was used in the next step without purification.

### Step 5: Synthesis of 5-(3-(1-(1H-tetrazol-5-yl)hydrazinyl)propyl)-1H-tetrazole (19A)

A mixture of 5-(1-(3-(1H-tetrazol-5-yl)propyl)hydrazinyl)-1-(4-methoxybenzyl)-1H-tetrazole (18A) (320 mg, 0.97 mmol) and 6 M hydrochloric acid in water was stirred at 100° C. for 3 h under microwave heating. The mixture was evaporated and purified by column chromatography eluting with dichloromethane:methanol:ammonia (4:1:0.2→3:2:0.5). The product was recrystallized from ethanol to give 5-(3-(1-(1H-tetrazol-5-yl)hydrazinyl)propyl)-1H-tetrazole (19A) (15 mg, 0.07 mmol, 7%) as an off-white crystalline solid. ESMS m/z 211 (M+H)′; ¹H NMR (400 MHz, MeOH-d4) δ 3.66 (t, J=6.9 Hz, 2H), 3.03 (t, J=7.4 Hz, 2H), 2.23 (quint, J=7.2 Hz, 2H).

### Example 2-17

### Synthesis of (E)-5-(2-benzylidenehydrazinyl)-1H-tetrazole (21A)

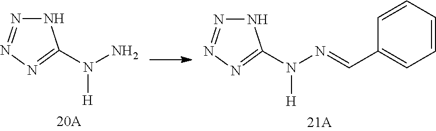 [SMILES: NNc1nnn[nH]1 ; C(=N\Nc1nnn[nH]1)/c1ccccc1]

A mixture of 5-hydrazinyl-1H-tetrazole (20A) (10 mg, 0.10 mmol) and benzaldehyde (11 mg, 0.10 mmol) in 1,4-dioxane (100 μL) was stirred at room temperature for 18 h. The precipitate was collected to afford (E)-5-(2-benzylidenehydrazinyl)-1H-tetrazole (21A) (6.5 mg, 0.03 mmol, 34%) as a white crystalline solid. ESMS m/z 189 (M+H)⁺; ¹H NMR (500 MHz, DMSO-d₆) δ 11.79 (s, 1H), 8.04 (s, 1H), 7.78 (d, J=6.9 Hz, 2H), 7.43 (t, J=7.3 Hz, 2H), 7.36-7.41 (m, 1H).

### Examples 2-18-2-25

The following compounds were prepared by the method of Example 2-17 using an appropriately functionalized aldehyde or ketone.

| | | | ESMS | | |
|---|---|---|---|---|---|
| Example | Structure | MW | m/z | ¹H NMR | Yield |
| 2-18 | 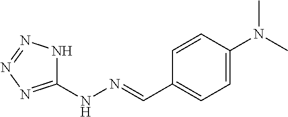 [SMILES: CN(C)c1ccc(/C=N/Nc2nnn[nH]2)cc1] | 231 | 232 | ¹H NMR (500 MHz, DMSO-d₆) δ 11.37 (s, 1H), 7.90 (s, 1H), 7.57 (d, J = 8.8 Hz, 2H), 6.73 (d, J = 8.8 Hz, 2H), 2.96 (s, 6H). | 54 |
| 2-19 | 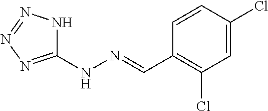 [SMILES: Clc1ccc(/C=N/Nc2nnn[nH]2)c(Cl)c1] | 257 | 257 | ¹H NMR (500 MHz, DMSO-d₆) δ 12.13 (s, 1H), 8.35 (s, 1H), 8.23 (d, J = 8.3 Hz, 1H), 7.68 (d, J = 2.0 Hz, 1H), 7.54 (dd, J = 8.6, 1.7 Hz, 1H). | 88 |
| 2-20 | 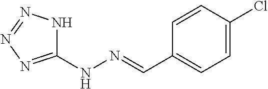 [SMILES: Clc1ccc(/C=N/Nc2nnn[nH]2)cc1] | 223 | 250 | ¹H NMR (400 MHz, DMSO-d₆) δ 11.88 (s, 1H), 8.04 (s, 1H), 7.82 (d, J = 8.5 Hz, 2H), 7.50 (d, J = 8.5 Hz, 2H). | 48 |
| 2-21 | 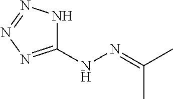 | 140 | 141 | ¹H NMR (500 MHz, DMSO-d₆) δ 15.04 (br. s, 1H), 10.23 (s, 1H), 1.97 (s, 3H), 1.91 (s, 3H). | 89 |
| 2-22 | 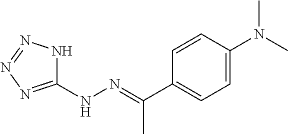 [SMILES: C/C(=N\Nc1nnn[nH]1)c1ccc(N(C)C)cc1] | 245 | 246 | ¹H NMR (400 MHz, DMSO-d₆) δ 10.59 (br. s, 1H), 9.89 (br. s, 1H), 7.83 (d, J = 8.8 Hz, 2H), 6.89 (br. s, 2H), 2.98 (s, 6H), 2.24 (s, 3H). | 72 |
| 2-23 | 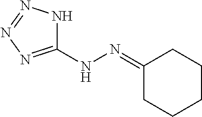 [SMILES: C1CCC(=NNc2nnn[nH]2)CC1] | 180 | 181 | ¹H NMR (500 MHz, DMSO-d₆) δ 10.39 (s, 1H), 2.39-2.43 (m, 2H), 2.23-2.31 (m, 2H), 1.63 (br. s, 2H), 1.51-1.60 (m, 4H). | 35 |
| 2-24 | 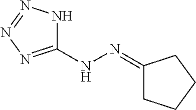 [SMILES: C1CCC(=NNc2nnn[nH]2)C1] | 166 | 167 | ¹H N NMR (500 MHz, DMSO-d₆) δ 10.16 (s, 1H), 2.30-2.41 (m, 4H), 1.73-1.81 (m, 2H), 1.66-1.73 (m, 2H). | 42 |
| 2-25 | 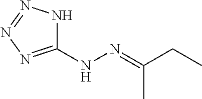 | 154 | 155 | ¹H NMR (500 MHz, DMSO-d₆, 4:1 Z:E isomers) δ 10.34 (br. s, 0.2H), 10.22 (br. s, 0.8H), 2.34 (q, J = 7.3 Hz, 0.4H), 2.26 (q, | 52 |
| | | | | J = 7.3 Hz, 1.6H), 1.95 (s, | |
| | | | | 0.6H), 1.89 (s, 2.4H), | |
| | | | | 1.07 (t, J = 7.3 Hz, | |
| | | | | 2.4H), 1.02 (t, J = 7.3 Hz, | |
| | | | | 0.6H). | |

### Example 2-26

### Synthesis of (E)-3-(2-(1H-tetrazol-5-yl)hydrazono)-1-(4-(dimethylamino)phenyl)propan-1-one (23A)

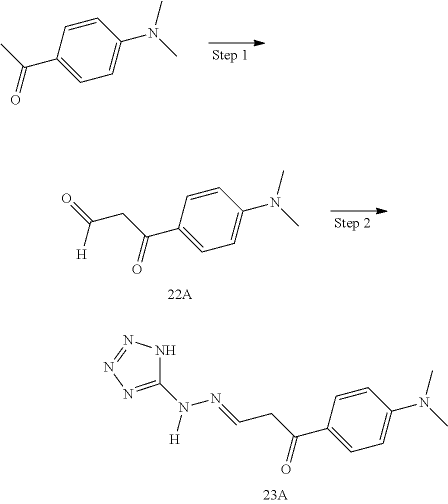 [SMILES: CC(=O)c1ccc(N(C)C)cc1 ; CN(C)c1ccc(C(=O)CC=O)cc1 ; CN(C)c1ccc(C(=O)C/C=N/Nc2nnn[nH]2)cc1]

### Step 1: Synthesis of 3-(4-(dimethylamino)phenyl)-3-oxopropanal (22A)

To a mixture of 4-dimethylaminoacetophenone (1.00 g, 6.13 mmol) and ethyl formate (580 μL, 7.16 mmol) in anhydrous tetrahydrofuran (10 mL) was added 25% sodium methoxide in methanol (1.70 mL, 7.18 mmol) at 0-5° C. The reaction mixture was stirred for 1 h at this temperature, then for 18 h at room temperature. The precipitate was collected and washed with diethyl ether (10 mL) to afford 3-(4-(dimethylamino)phenyl)-3-oxopropanal (22A) (0.53 g, 2.48 mmol, 40%) as a pale yellow crystalline solid. ESMS m/z 192 (M+H)⁺.

### Step 2: Synthesis of (E)-3-(2-(1H-tetrazol-5-yl)hydrazono)-1-(4-(dimethylamino)phenyl)propan-1-one (23A)

To a mixture of 3-(4-(dimethylamino)phenyl)-3-oxopropanal (22A) (50 mg, 0.23 mmol) and ethanol (3 mL) was added (1H-tetrazol-5-yl)hydrazine hydrochloride (37 mg, 0.27 mmol) at 0° C. The reaction mixture was stirred at this temperature for 10 min. The precipitate was collected and washed with ethanol (1 ml) to afford (E)-3-(2-(1H-tetrazol-5-yl)hydrazono)-1-(4-(dimethylamino)phenyl)propan-1-one (23A) (31 mg, 0.11 mmol, 48%, 4:1 E:Z) as a pale yellow crystalline solid. ESMS m/z 274 (M+H)⁺; ¹H NMR (500 MHz, DMSO-d₆) δ 11.41 (s, 0.8H), 10.93 (s, 0.2H), 7.80-7.89 (m, 2H), 7.56 (t, J=5.6 Hz, 0.8H), 7.09 (t, J=5.1 Hz, 0.2H), 6.71-6.78 (m, 2H), 4.10 (d, J=5.4 Hz, 0.4H), 3.93 (d, J=5.9 Hz, 1.6H), 3.04 (s, 1.2H), 3.03 (s, 4.8H).

### Example 2-27

### Synthesis of (E)-N,N-dimethyl-4-((2-methyl-2-(1H-tetrazol-5-yl)hydrazono)methyl)aniline (26A)

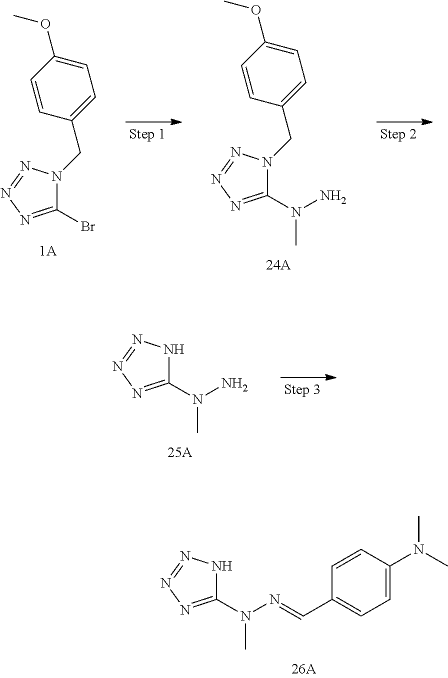

### Step 1: Synthesis of 1-(4-methoxybenzyl)-5-(1-methylhydrazinyl)-1H-tetrazole (24A)

A mixture of 5-bromo-1-(4-methoxybenzyl)-1H-tetrazole (1A) (600 mg, 2.23 mmol) and methyl hydrazine (235 μL, 4.46 mmol) in 2-propanol (5.3 mL) was stirred at 60° C. for 20 h. The reaction mixture was evaporated, and the residue was recrystallized from 2-propanol to give 1-(4-methoxybenzyl)-5-(1-methylhydrazinyl)-1H-tetrazole (24A) (54 mg, 0.23 mmol, 10%) as an off-white crystalline solid. ESMS m/z 235 (M+H)⁺.

### Step 2: Synthesis of 5-(1-methylhydrazinyl)-1H-tetrazole (25A)

A mixture of 1-(4-methoxybenzyl)-5-(1-methylhydrazinyl)-1H-tetrazole (24A) (391 mg, 1.67 mmol) and 10% palladium/carbon (195 mg) in methanol (4 mL) was stirred under a hydrogen atmosphere for 20 h. The reaction mixture was filtered through Celite and the filtrate was evaporated. The crude product was recrystallized from 2-propanol (3 mL) to give 5-(1-methylhydrazinyl)-1H-tetrazole (25A) (72 mg, 0.63 mmol, 38%) as an off-white crystalline solid. ESMS m/z 114 (M+H)⁺; ¹H NMR (500 MHz, DMSO-d₆) δ 14.57 (br. s, 1H), 4.94 (br. s, 2H), 3.14 (s, 3H).

### Step 3: Synthesis of (E)-N,N-dimethyl-4-((2-methyl-2-(1H-tetrazol-5-yl)hydrazono)methyl)aniline (26A)

A mixture of 5-(1-methylhydrazinyl)-1H-tetrazole (25A) (20 mg, 0.18 mmol), 4-dimethylaminobenzaldehyde (26 mg, 0.17 mmol) and a drop of 3.8M hydrogen chloride solution in 1,4-dioxane was stirred in 1,4-dioxane (400 μL) at room temperature for 18 h. The precipitate was collected to afford (E)-N,N-dimethyl-4-((2-methyl-2-(1H-tetrazol-5-yl)hydrazono)methyl)aniline (26A) (17 mg, 0.07 mmol, 37%) as a white crystalline solid. ESMS m/z 246 (M+H)⁺; ¹H NMR (500 MHz, DMSO-d₆) δ 7.84 (s, 1H), 7.78 (d, J=8.8 Hz, 2H), 6.92 (br. s, 2H), 3.54 (s, 3H), 3.00 (s, 6H).

### Example 2-28

### Synthesis of 5-(1-methyl-2-(propan-2-ylidene)hydrazinyl)-1H-tetrazole (27A)

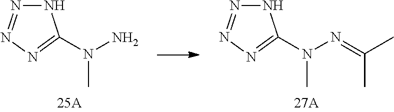

A mixture of 5-(1-methylhydrazinyl)-1H-tetrazole (25A) (20 mg, 0.18 mmol) and acetone (500 μL) was stirred at room temperature for 18 h. The reaction mixture was evaporated to give 5-(1-methyl-2-(propan-2-ylidene)hydrazinyl)-1H-tetrazole (27A) (27 mg, 0.18 mmol, 100%) as a pale yellow oil. ESMS m/z 155 (M+H)⁺; ¹H NMR (500 MHz, CDCl₃) δ 3.31 (s, 3H), 2.13 (s, 3H), 2.12 (br. s, 3H).

### Example 2-29

### Synthesis of 2-methyl-5-(1-methylhydrazinyl)-2H-tetrazole (28A)

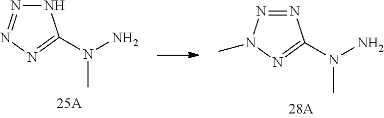

To a mixture of 5-(1-methylhydrazinyl)-1H-tetrazole (25A) (100 mg, 0.87 mmol) and methanol (25 mL) was added a solution of diazomethane (8.76 mmol) in diethyl ether (60 mL) at 0° C. The reaction mixture was stirred for 1 h at 0° C., then at room temperature for 18 h. The reaction mixture was evaporated and the crude product was purified by column chromatography eluting with n-hexane: ethyl acetate (3:2) to give 2-methyl-5-(1-methylhydrazinyl)-2H-tetrazole (28A) (6 mg, 0.04 mmol, 5%) as a pale yellow crystalline solid. ESMS m/z 129 (M+H)⁺. ¹H NMR (500 MHz, MeOH-d₄) δ 4.18 (s, 3H), 3.15 (s, 3H), structure determined by ¹H,¹⁵N HMBC.

### Example 2-30

### Synthesis of 5-(2-(1-ethoxycyclopropyl)hydrazinyl)-1H-tetrazole (29A)

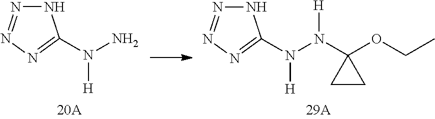

A mixture of 5-hydrazinyl-1H-tetrazole dihydrochloride (20A) (150 mg, 0.87 mmol), sodium acetate (141 mg, 1.72 mmol) and (1-ethoxycyclopropoxy)trimethylsilane (174 μL, 0.87 mmol) in ethanol (7.5 mL) was stirred at 80° C. for 16 h. The reaction mixture was evaporated and the residue was triturated with anhydrous tetrahydrofuran (10 mL). The filtrate was evaporated and the crude product was recrystallized from 2-propanol (3 mL). The product was collected and washed with diisopropyl ether (1 mL) to afford 5-(2-(1-ethoxycyclopropyl)hydrazinyl)-1H-tetrazole (29A) (30 mg, 0.16 mmol, 18%) as an off-white crystalline solid. ESMS m/z 185 (M+H)⁺; ¹H NMR (500 MHz, DMSO-d₆) δ 14.47 (br. s, 1H), 8.44 (s, 1H), 6.23 (s, 1H), 3.56 (q, J=6.9 Hz, 2H), 1.03 (t, J=6.9 Hz, 3H), 0.77-0.83 (m, 2H), 0.71-0.77 (m, 2H).

### Example 2-31

### Synthesis of 5-(1-ethylhydrazinyl)-1H-1,2,4-triazole (37A)

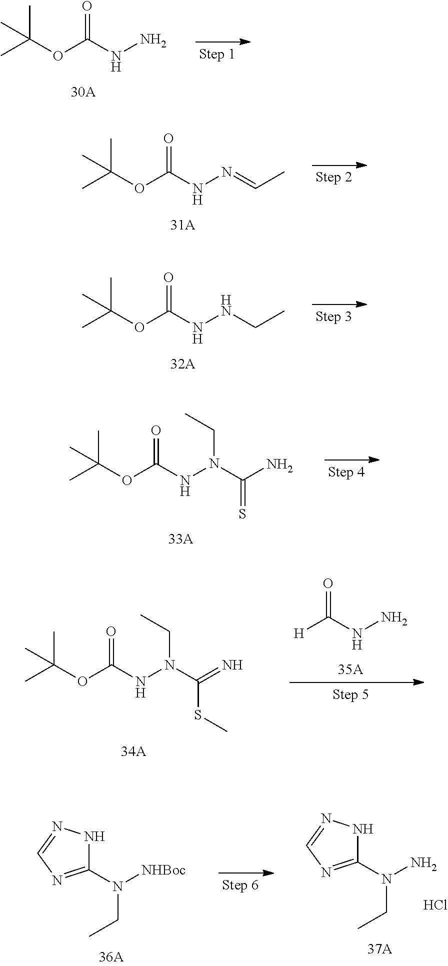

### Step 1: Synthesis of (E)-tert-butyl 2-ethylidenehydrazinecarboxylate (31A)

To a stirred solution of tert-butyl hydrazinecarboxylate (30A) (2.0 g, 12.6 mmol) in toluene (15 ml) was added acetaldehyde (0.7 ml, 13.9 mmol). The solution was heated to 50° C. for 1 h and then stirred at RT for 24 h. The mixture was concentrated to give (E)-tert-butyl 2-ethylidenehydrazinecarboxylate (31A) as colorless oil (97.5%). ESMS: 159 (M⁺+1).

### Step 2: Synthesis of tert-butyl 2-ethylhydrazinecarboxylate (32A)

To a stirred solution of (E)-tert-butyl 2-ethylidenehydrazinecarboxylate (31A) (6.6 g, 37.0 mmol) in THF (50 mL) at −78° C. was added DIBAL (31 ml, 92.6 mmol) as a 1.5 M solution in toluene. The reaction was maintained at −78° C. for 2 h and then −40° C. for 2 h. The mixture was then warmed to room temperature before Rochelle's salt (aqueous potassium sodium tartrate) solution was added and the reaction mixture stirred at room temperature overnight. The organic phase was separated and the aqueous phase extracted with Et₂O (2×75 ml). The combined organic extracts were washed with brine, dried (Na₂SO₄), filtered, and concentrated in vacuo. Purification by flash chromatography on silica gel gave tert-butyl 2-ethylhydrazinecarboxylate (32A) as colorless oil (3.0 g, 49%). ESMS: 183 (M⁺+23).

### Step 3: Synthesis of tert-butyl 2-carbamothioyl-2-ethylhydrazinecarboxylate (33A)

To a stirred solution of tert-butyl 2-ethylhydrazinecarboxylate (32A) (5.8 g, 36.6 mmol) in ethyl acetate (30 mL) was added TMSSCN (4.8 g, 36.6 mmol) and the reaction mixture was heated at reflux for 5 h. After completion of reaction, solvent was evaporated under reduced pressure to obtain tert-butyl 2-carbamothioyl-2-ethylhydrazinecarboxylate (33A) in 48% yield. ¹H NMR (400 MHz, CD₃OD) δ 1.2 (t, 3H), 1.5 (s, 9H), 4.1(br s, 2H), 6.2 (br s, 2H), 6.4 (br s, 1H).

### Step 4: Synthesis of tert-butyl 2-ethyl-2-(imino(methylthio)methyl)hydrazinecarboxylate (34A)

To a stirred solution of tert-butyl 2-carbamothioyl-2-ethylhydrazinecarboxylate (33A) (3 g, 13.2 mmol) in acetonitrile (30 ml) was added methyl iodide (9.38 g, 66 mmol) and the reaction mixture was heated at 60° C. for 1 h. After completion of reaction solvent was evaporated under reduced pressure and the crude residue washed with diethyl ether and was dried to obtain tert-butyl 2-ethyl-2-(imino(methylthio)methyl)hydrazinecarboxylate (34A) in 94% yield. ESMS: 234.1 (M⁺+1).

### Step 5: Synthesis of tert-butyl 2-ethyl-2-(1H-1,2,4-triazol-5-yl)hydrazinecarboxylate (36A)

To a stirred solution of tert-butyl 2-ethyl-2-(imino(methylthio)methyl)hydrazinecarboxylate (34A) (500 mg, 2.14 mmol) and formyl hydrazide (35A) (155 mg, 2.57 mmol) in dimethyl formamide (10 ml) was added diisopropylethyl amine (830 mg, 6.43 mmol) and the reaction mixture was heated to reflux for 15 h. After completion of reaction, water was added to reaction mixture, and extracted with ethyl acetate (2×50 ml). The organic layer was separated and dried over sodium sulfate, filtered and concentrated under reduced pressure to afford the crude product. The crude product was purified by silica gel column chromatography to obtain tert-butyl 2-ethyl-2-(1H-1,2,4-triazol-5-yl)hydrazinecarboxylate (36A) in 12% yield. ¹H NMR (400 MHz, CD₃OD) δ 1.2 (t, 3H), 1.5 (s, 9H), 3.6 (br s, 2H), 7.7 (br s, 1H).

### Step 6: Synthesis of 5-(1-ethylhydrazinyl)-1H-1,2,4-triazole hydrochloride (37A)

To a stirred solution of MeOH.HCl (5 ml) was added tert-butyl 2-ethyl-2-(1H-1,2,4-triazol-5-yl)hydrazinecarboxylate (36A) (40 mg, 0.17 mmol) and the resulting mixture was stirred for 12 h at room temperature. After completion of reaction, solvent was removed under reduced pressure, washed twice with ether and dried under reduced pressure to afford 5-(1-ethylhydrazinyl)-1H-1,2,4-triazole (37A) in 75% yield. ¹H NMR (400 MHz, CD3OD) δ 1.2 (t, 3H), 3.6 (q, 2H), 8.5 (s, 1H), 10.2-10.4 (brs, 2H). HPLC Purity: 90.89%; ESMS: 127.85 (M⁺).

### Example 2-32

### Synthesis of 3-(1-methylhydrazinyl)-1,2,4-oxadiazol-5(4H)-one hydrochloride (42A)

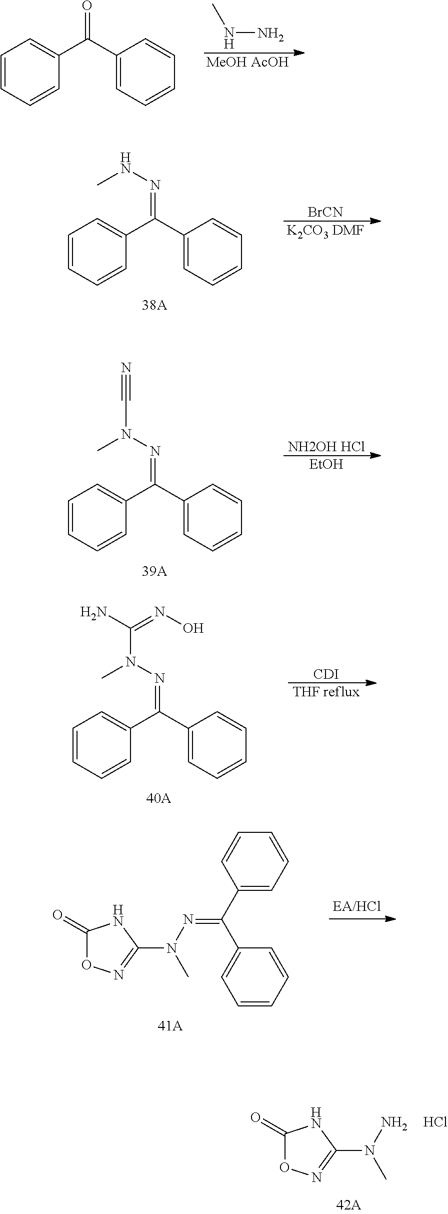

### Step 1: Synthesis of 1-(diphenylmethylene)-2-methylhydrazine (38A)

To a solution of benzophenone (18 g, 100 mmol) in MeOH (200 mL) and AcOH (200 mL) was added methylhydrazine (12 mL, 100 mmol) at 20° C. After stirring at 70° C. for 3 h, the mixture was concentrated, and diluted with ethyl acetate (10 mL) and water (15 mL). The organic layer was separated. The aqueous layer was washed with ethyl acetate (10 mL×2). The combined organic layers were washed with brine, dried over Na₂SO₄, filtered and concentrated under reduced pressure to afford 38A as white oil (10.5 g). LCMS (ESI): m/z 211.1 (M+1)⁺.

### Step 2: Synthesis of 2-(diphenylmethylene)-1-methylhydrazinecarbonitrile (39A)

A mixture of 38A (10.5 g, 50 mmol) and BrCN (5.3 g, 50 mmol) in DMF (100 mL) was heated to 50° C., then K₂CO₃ was added and stirred at 50° C. overnight. EtOAc (250 mL) was added and the solution was washed with brine (250 mL×3), dried over Na₂SO₄, filtered and concentrated under reduced pressure to afford a residue which was purified by flash column chromatography (PE/ethyl acetate=20:1) to afford 39A (5 g) as a white solid. ¹H NMR (400 MHz, CDCl₃): δ 3.39 (s, 3H), 7.35-7.40 (m, 4H), 7.45-7.49 (m, 1H), 7.52-7.55 (m, 5H). LCMS (EST): m/z 236 (M+1)⁺.

### Step 3: Synthesis of (Z)-2-(diphenylmethylene)-N′-hydroxy-1-methylhydrazinecarboximidamide (40A)

A mixture of 39A (5.5 g, 20 mmol), hydroxylamine hydrochloride (2.2 g, 30 mmol) and AcONa (3.2 g, 40 mmol) in EtOH (80 mL) was stirred at 25° C. overnight. The solvent was removed under reduced pressure and EtOAc (100 mL) was added, washed with water and brine (100 mL), dried over Na₂SO₄ and concentrated under reduced pressure to afford 40A (5.5 g) as a yellow solid. LCMS (ESI): m/z 269.1 (M+1)′.

### Step 4: Synthesis of 3-(2-(diphenylmethylene)-1-methylhydrazinyl)-1,2,4-oxadiazol-5(4H)-one (41A)

A mixture of 40A (5.5 g, 20 mmol) and CDI (5 g, 30 mmol) in THF (60 mL) was stirred at reflux for 5 h. The mixture was cooled, concentrated under reduced pressure and purified with flash column chromatography (PE/ethyl acetate=3:1) to afford 41A (4 g) as a yellow solid. LCMS (ESI): m/z 295.1 (M+1)⁺.

### Step 5: Synthesis of 3-(1-methylhydrazinyl)-1,2,4-oxadiazol-5(4H)-one hydrochloride (42A)

To a solution of 41A (0.9 g, 9 mmol) in ethyl acetate (10 mL) was added 4 M HCl in ethyl acetate (15 mL) and the mixture was stirred at room temperature overnight. The mixture was then filtered and the solid was washed with ethyl acetate and MeOH to afford 3-(1-methylhydrazinyl)-1,2,4-oxadiazol-5(4H)-one HCl salt (42A) (0.25 g, 1.5 mmol) as a white solid. ¹H NMR (400 MHz, DMSO-d₆): δ 3.07 (s, 3H). LCMS (ESI): m/z 131.1 (M+1)⁺.

### Example 2-33

### Synthesis of N-ethyl-1H-tetrazole-5-carbohydrazide (46A)

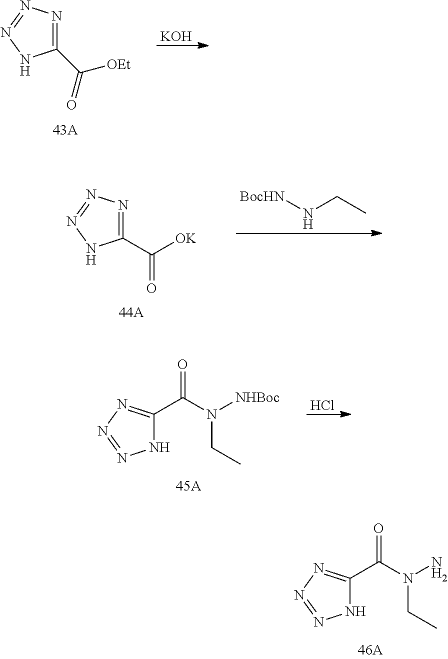

### Step 1: Synthesis of potassium 1H-tetrazole-5-carboxylate (44A)

To a solution of 43A (500 mg, 3.5 mmol) in EtOH (10 mL) was added KOH (591 mg, 0.1 M). The mixture was stirred at RT for 3 min and then filtered. The solid was washed with cold EtOH and then dried under vacuum to afford 44A (500 mg) as a white solid. The compound was used in the next step without further purification.

### Step 2: Synthesis of tert-butyl 2-ethyl-2-(1H-tetrazole-5-carbonyl)hydrazinecarboxylate (45A)

To a mixture of 44A (500 mg, 3.3 mmol) and tert-butyl 2-ethylhydrazinecarboxylate (631 mg, 3.9 mmol) in dichloromethane (20 mL) was added HOBT (533 mg, 3.9 mmol) and EDCI (863 mg, 4.9 mmol). The mixture was stirred at RT for 14 h, washed with water and then the organic layer was concentrated. The residue was purified by HPLC to afford 45A (220 mg) as an oil. ¹H NMR (400 MHz, CDCl₃): 1.32 (t, J=7.2 Hz, 3H), 1.42 (s, 9H), 3.84 (q, J=7.2 Hz, 2H), 7.56 (br.s, 1H).

### Step 3: Synthesis of N-ethyl-1H-tetrazole-5-carbohydrazide (46A)

A mixture of 45A (220 mg) in 4 M HO/ethyl acetate (15 mL) was stirred at RT for 0.5 h. The solution was concentrated, and the solid was washed with ethyl acetate and filtered to afford N-ethyl-1H-tetrazole-5-carbohydrazide (46A) (150 mg) as a white solid. ¹H NMR (400 MHz, DMSO-d₆): 1.15-1.33 (m, 3H), 3.63 (m, 1H), 4.04 (br. s., 1H). LCMS (ESI): m/z 157.1 [M+1]¹.

### Example 2-34

### Synthesis of 1-ethyl-N-(1H-tetrazol-5-yl)hydrazinecarboxamide (49A)

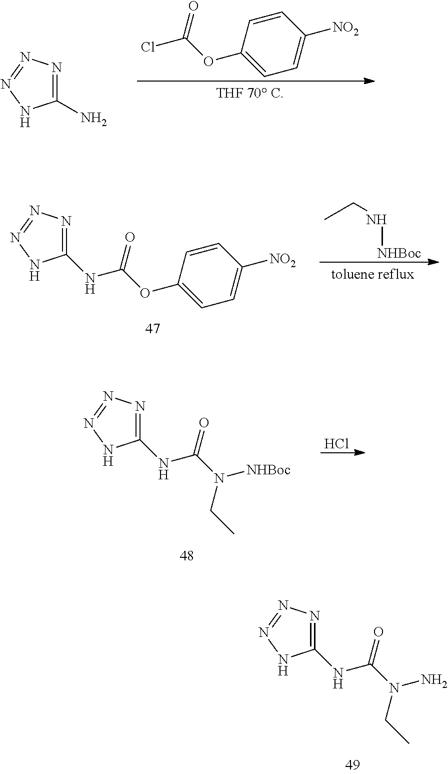

### Step 1: Synthesis of 4-nitrophenyl 1H-tetrazol-5-ylcarbamate (47A)

A mixture of 2H-tetrazol-5-amine (100 mg, 1.18 mmol) and 4-nitrophenyl carbonochloridate (718.6 mg, 3.54 mmol) in THF (20 mL) was heated to reflux and stirred for 3 h. The solvent was evaporated and the residue was purified on silica gel column (ethyl acetate/PE=1:10) to afford 47A (330 mg) as a white solid.

### Step 2: Synthesis of tert-butyl 2-(1H-tetrazol-5-ylcarbamoyl)-2-ethylhydrazinecarboxylate (48A)

A mixture of 47A (310 mg, 1.24 mmol) and tert-butyl 2-ethylhydrazinecarboxylate (294.1 mg, 1.86 mmol) in toluene (20 mL) was heated and refluxed for 3 h. The reaction solution was cooled to room temperature, washed with water and concentrated. The residue was purified by preparative HPLC to afford 48A (193 mg) as an oil.

### Step 3: Synthesis of 1-ethyl-N-(1H-tetrazol-5-yl)hydrazinecarboxamide (49A)

48A (193 mg) in 4 N HCl-ethyl acetate (15 mL) was stirred at RT for 30 min. The solvent was removed. The residue was washed with ethyl acetate and filtered to afford the compound 1-ethyl-N-(1H-tetrazol-5-yl)hydrazinecarboxamide (49A) (100 mg) as a white solid. ¹H NMR (400 MHz, D₂O): δ 1.07 (t, J=7.2 Hz, 3H), 3.47 (q, J=7.2 Hz, 2H). LCMS (ESI): m/z 172.1 [M+1]⁺.

### Example 2-35

### Synthesis of 1-ethyl-N-(phenylsulfonyl)hydrazinecarboxamide (54A)

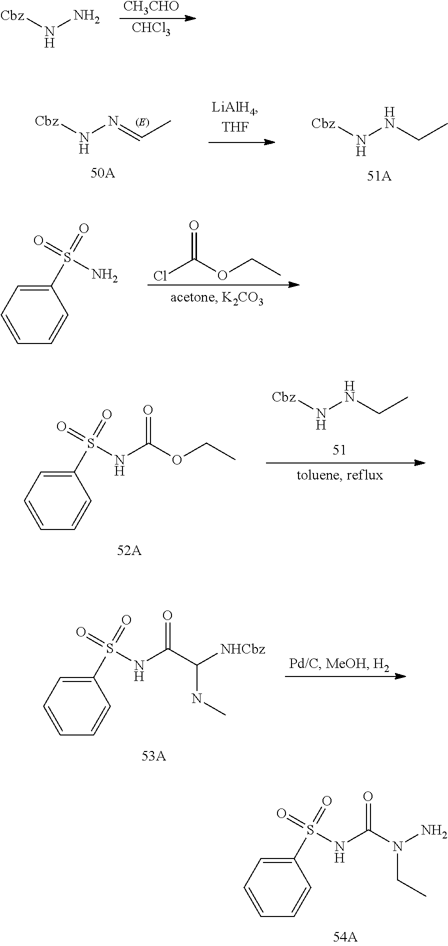

### Step 1: Synthesis of (E)-benzyl 2-ethylidenehydrazinecarboxylate (50A)

A mixture of benzyl hydrazinecarboxylate (5 g, 30.08 mmol), MgSO₄ (5 g) in 30 mL of CHCl₃ was added anhydrous CH₃CHO (2 g, 45.13 mmol) at 0° C. The mixture was stirred at room temperature for 2 h, then filtered and concentrated to afford 50A (5.9 g) as a yellow solid, which was used in the next step without further purification.

### Step 2: Synthesis of benzyl 2-ethylhydrazinecarboxylate (51A)

A mixture of LiAlH₄ (1.37 g, 36 mmol) in 30 mL of THF was added 50A (5.9 g, crude product from previous step) in 40 mL of THF at 0° C. under nitrogen atmosphere, stirred for 1 h at 0° C. and then 1 h at room temperature. Water (1.37 mL) was then added dropwise followed by 10% aq. NaOH (1.37 mL). The mixture was filtered, concentrated and then purified by flash chromatography on a silica gel (eluting with 5%-50% PE in ethyl acetate) to afford 51A (3.5 g) as a white solid. ¹H NMR (400 MHz, CDCl₃): δ 1.08 (t, J=7.2 Hz, 3H), 2.84-2.98 (m, 2H), 3.63 (s, 1H), 5.15 (s, 2H), 6.60 (br. s, 1H), 7.30-7.44 (m, 5H).

### Step 3: Synthesis of ethyl phenylsulfonylcarbamate (52A)

A mixture of 51A (1.0 g, 6.36 mmol), K₂CO₃ (2.2 g, 15.92 mmol) in 50 mL of acetone and ethyl chloroformate (4.04 g, 37.2 mmol) in 5 mL of acetone was stirred at reflux for 1 h. The solvent was evaporated and the residue was dissolved in water, acidified with cone. HCl (pH=1) and extracted with ethyl acetate twice. The combined organic layer was washed with water twice and concentrated to afford 52A (1.1 g) as colorless oil. ¹H NMR (400 MHz, CDCl₃): δ 1.23 (t, J=7.2 Hz, 3H), 4.13-4.20 (m, 2H), 7.56-7.62 (m, 2H), 7.64-7.71 (m, 1H), 7.86 (s, 1H), 8.06-8.09 (m, 2H).

### Step 4: Synthesis of benzyl 2-ethyl-2-(phenylsulfonylcarbamoyl)hydrazinecarboxylate (53A)

A mixture of 52A (704 mg, 3.07 mmol) and 51A (1.2 g, 6.14 mmol) in 20 mL of toluene was stirred at reflux overnight. The mixture was cooled, concentrated and purified by flash column chromatography (eluting with 5%-20% PE in ethyl acetate) to afford 53A (569 mg) as white solid. ¹H NMR (400 MHz, CDCl₃): δ 1.07 (t, J=7.2 Hz, 3H), 3.60 (br. s, 2H), 5.18 (s, 2H), 6.92 (s, 1H), 7.28-7.48 (m, 5H), 7.49-7.53 (m, 2H), 7.61-7.65 (m, 1H), 8.04 (d, J=7.6 Hz, 2H), 8.64 (br.s, 1H).

### Step 5: Synthesis of 3-(1-methylhydrazinyl)-1,2,4-oxadiazol-5(4H)-one hydrochloride (54A)

A mixture of 53A (400 mg, 1.06 mmol) and Pd/C (0.2 g) in 40 mL of MeOH was stirred at room temperature for 2 h under an atmosphere of hydrogen (balloon). The mixture was filtered, concentrated and the solid was washed with ethyl acetate/PE (1:1) to afford 1-ethyl-N-(phenylsulfonyl)hydrazinecarboxamide (54A) (100 mg) as white solid. ¹H NMR (400 MHz, DMSO-d₆): δ 0.99 (t, J=7.2 Hz, 3H), 3.25-3.35 (m, 3H), 6.70 (br.s, 2H), 7.53-7.70 (m, 3H), 7.90-7.95 (d, J=7.2 Hz, 2H). LCMS (ESI): m/z 244.0 [M+1]⁺.

### Biological Examples

### Measurements of H₂S Levels

H₂S levels in the liver were assayed as follows. Briefly, liver tissue homogenates were prepared in 100 mM potassium phosphate buffer, pH 7.4+0.5% Triton-X100. The enzyme reaction was carried out in 96 well, deep square well plates with 700 μl Glass Insert (Waters Corporation Cat. #186000349) with TFE/Silicone MicroMat sealing covers (Sun-SRI Cat. #400 026). In the outer well in a total volume of 200 μl the assay mixture contained (in final concentration): L-cysteine, (5 mM); pyridoxal 5′-phosphate, (50 μM); potassium phosphate buffer, pH 7.4, (100 mM); and tissue homogenate (500 μg protein). The glass insert contained 100 μl alkaline zinc acetate solution (1% in 0.1N NaOH) to trap the generated H₂S. The reaction mixture was incubated at 37° C. for 3 h and at the end of the reaction, 100 μl NN-dimethyl-p-phenylenediamine sulfate (20 μM in 7N HCl) and 100 μl ferric chloride (30 μM in 1.2N HCl) was added to the glass insert. Absorbance was measured at 671 nm using a micro-plate reader. A standard curve relating the concentration of Na₂S and absorbance was used to calculate H₂S concentration and expressed as nanomoles of H₂S formed per hour per milligram protein.

### Example 3-1

### CSE In Vitro Assay

Test compounds (from DMSO stock solutions) were added to (final concentrations) 20 ug/ml enzyme solution (human, mouse or rat recombinant CSE) plus 50 uM PLP in assay buffer (100 mM potassium phosphate pH 7.6) in 96 well plates in total volume of 190 ul. Plates were incubated for 30 minutes at room temperature before the addition of 10 ul of 200 mM (20× final in assay buffer) DL-Homocysteine substrate to each well. Plates were incubated at 37° C. for 3 hours. 50 ul 20 mM DMPDA in 7.2N HCl was added to each well followed by 50 ul 30 mM FeCl₃ in 1.2N HCl. Plates were incubated for 10 minutes with shaking at room temperature and then absorbance at 671 nm read in Promega GloMax microplate reader.

| Example | IC₅₀ (μM) |
|---|---|
| 1-1 | C |
| 1-2 | A |
| 1-3 | A |
| 1-4 | A |
| 1-5 | C |
| 1-6 | C |
| 1-7 | B |
| 1-8 | B |
| 1-9 | B |
| 2-1 | B |
| 2-2 | C |
| 2-3 | A |
| 2-4 | A |
| 2-5 | C |
| 2-6 | B |
| 2-7 | B |
| 2-8 | C |
| 2-9 | C |
| 2-10 | C |
| 2-11 | A |
| 2-12 | A |
| 2-13 | A |
| 2-14 | B |
| 2-15 | A |
| 2-16 | B |
| 2-17 | B |
| 2-18 | B |
| 2-19 | C |
| 2-20 | A |
| 2-21 | A |
| 2-22 | A |
| 2-23 | A |
| 2-24 | A |
| 2-25 | A |
| 2-26 | A |
| 2-27 | B |
| 2-28 | A |
| 2-29 | C |
| 2-31 | C |
| 2-32 | B |
| 2-33 | C |
| 2-34 | C |
| 2-35 | C |

IC₅₀ (μM) A < 10 μM; 10 μM ≦ B ≦ 100 μM; C > 100 μM

### Example 3-2

### Comb Burn Model for Cutaneous Burn

A comb burn wound model was used to assess recovery of zone of stasis and healing of intermediate areas between systematically created deep partial thickness cutaneous burn injury.

### Methods

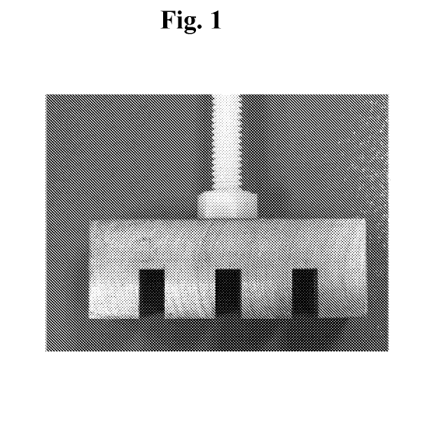

Reproducible deep partial thickness cutaneous burns were created as follows. On Day 0, adult male Sprague-Dawley rats were anesthetized, monitored, weighed and shaved (dorsal aspect), then depilated with commercial agent as per laboratory standard protocol. The shaved areas were then prepared with chlorhexidine. Brass combs, with four prongs (10×25 mm) separated by three 5 mm notches (FIG. 1) were heated to 100° C. in a dry bath and applied to prepped rat skin (1 set on each flank, left and right) in order to create 4 burn injuries, separated by 3 interspaces of unburned skin on either side of the animal, offset from the spine. Combs were applied for 30 seconds and only the weight of the comb was used to apply the burns. After injury creation, animals were assigned to treatment groups as follows:

3 animals were assigned to the L-propargylglycine group; 3 animals were assigned to the Compound 1 group; and 3 animals were assigned to the Control/Sham group (Vehicle alone).

Laser Doppler imaging (LDI), and digital photography of the left wound area were performed immediately before injury and immediately after wound creation. Baseline biopsies were taken from uninjured skin.

Approximately 60 minutes after wound creation, animals were given the first dose of treatment compound or vehicle. Animals receiving L-propargylglycine received 100 mg/kg IP on Day 0, and no further treatment on Days 1-6, as this compound was demonstrated to have a very long half-life. Animals in the 5-(1-methylhydrazinyl)-1H-tetrazole (Compound 1) group received 20 mg/kg PO (via oral gavage) on Day 0 and then once daily for the remainder of the time course. Control animals received similar volumes of vehicle (sterile saline) IP on Day 0. Animals were given a one-time dose of Buprenex for pain after injury and dosing, and then recovered from anesthesia and returned to sterile cages.

The following day (Day 1), approximately 24 hours after burn wound creation, animals were weighed and anesthetized. Digital photos and LDI were taken of the left wound area, and 2 mm punch biopsies were taken from a burn wound (1 biopsy) and the zone of stasis (1 biopsy, interspace area) from the right wound area and formalin fixed. Biopsy sites were closed with prolene sutures. Treatment was administered as described above for the animals receiving Compound 1. Animals in other groups received no vehicle or compound. This daily procedure including daily imaging, sampling, and treatment continued for an additional 5 days, for the remainder of the 6-day duration of the experiment.

On Day 6, animals were again weighed and anesthetized with the described daily procedure performed. After sample acquisition, animals were euthanized while under anesthesia with a necropsy performed. The entire wound areas were excised and preserved for future histological and molecular study.

### Digital Photo Assessment

All photos of injuries from treated animals were placed into a slide show and paired against a randomly selected control animal wound photo from the same time point. The slides were randomized and then evaluated by 3 blinded, independent graders who evaluated each set of images. The graders were asked to rate each of the three pairs of interspaces/zones of stasis to see if one appeared better (improved, less injured, more viable), equal to/unchanged, or worse (more injured, less viable) compared to the second set of interspaces. The grader was unaware of the order of the photos and which photos were control or experimental. Data was grouped by compound number. The final rating of “better”, “equivalent”, or “worse” was determined if at least 2 out of 3 graders agreed. If 0/3 agreed, then the wound was recorded as unchanged relative to control. Three animals with 3 interspaces each allowed for 9 interspaces total to be evaluated per treatment group. Data for each treatment group was entered into analysis in two categories, as number of interspaces deemed less injured versus number of interspaces deemed worse or more injured, for each experimental time point (day). A Chi-squared test was performed to look for significant associations.

### LDI Analysis

Regions of interest encompassing the total interspace areas on the left side injuries were identified on flux files of LDI images. Perfusion units (PUs) were then calculated for the defined regions of interest, averaged, and compared to values obtained for post-injury creation, which will serve as baseline.

### Results

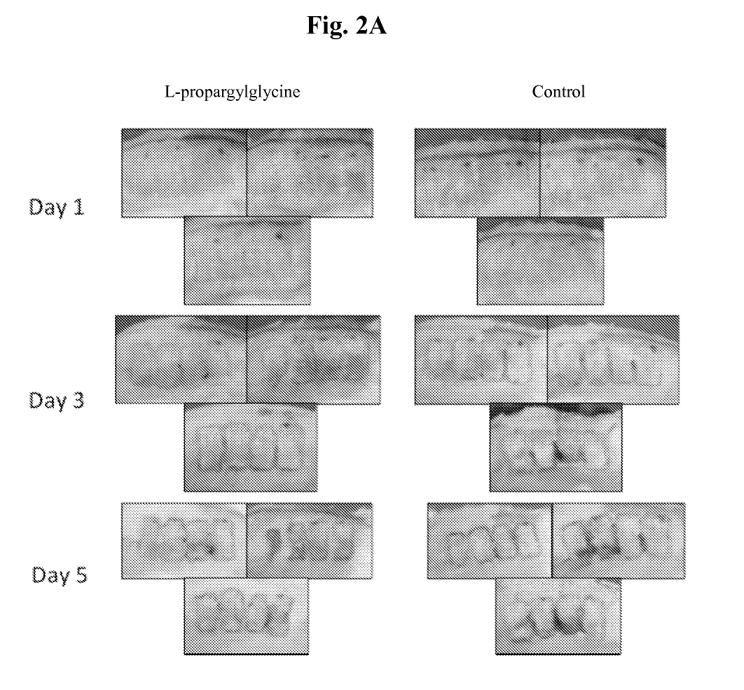

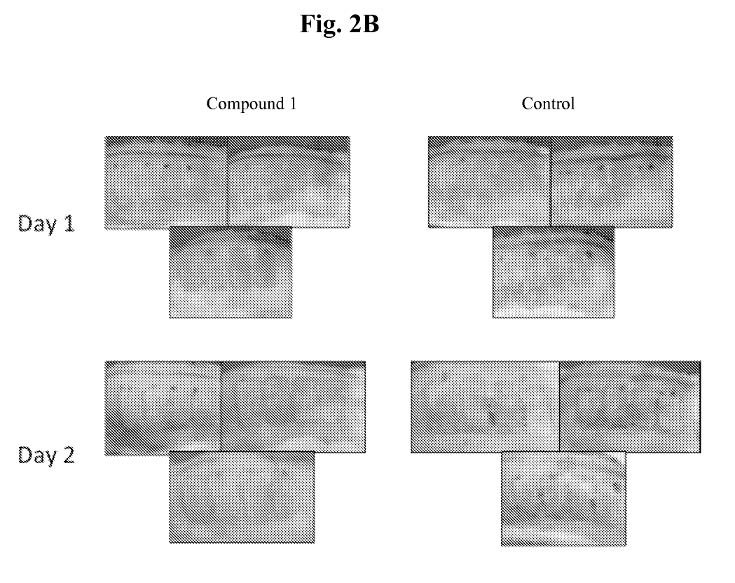

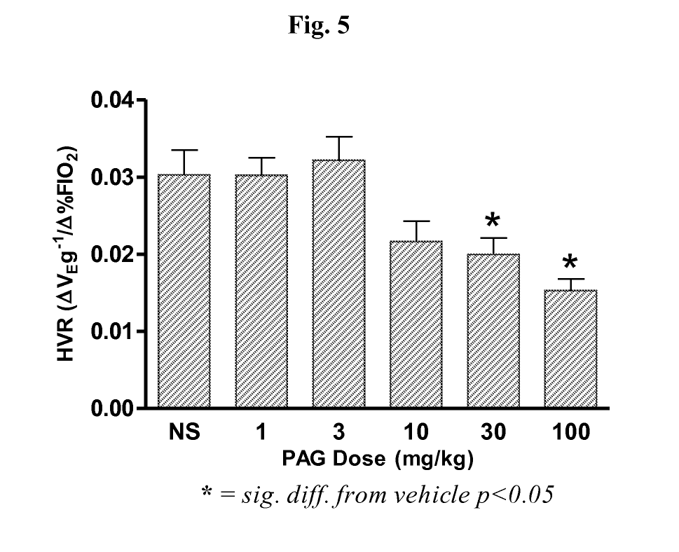

Upon gross examination of injuries over the time course, a differential burn progression can be seen in treated animals (both compounds) over controls. In animals receiving treatment with either L-propargylglycine (FIG. 2A) or 5-(1-methylhydrazinyl)-1H-tetrazole (Compound 1) (FIG. 2B), the zones of stasis/interspaces appeared to maintain viability over the time course, while the same areas in control animals began to convert and become more necrotic around Day 2.

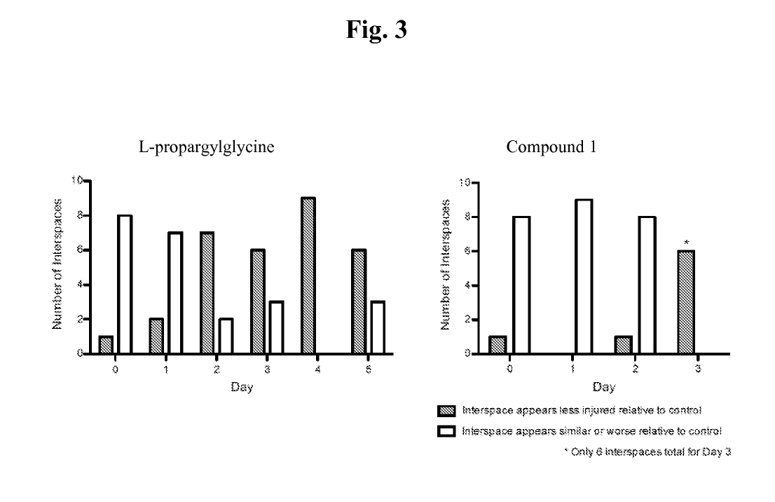

Using a grading system to compare treated interspaces versus interspaces in control animals by assigning a “less injured” versus “worse/the same” scale demonstrated that within 48-72 hours, most interspaces in treated animals appeared less injured than controls (FIG. 3) and the data were significantly distributed (L-propargylglycine p=0.0007 and Compound 1 p<0.001).

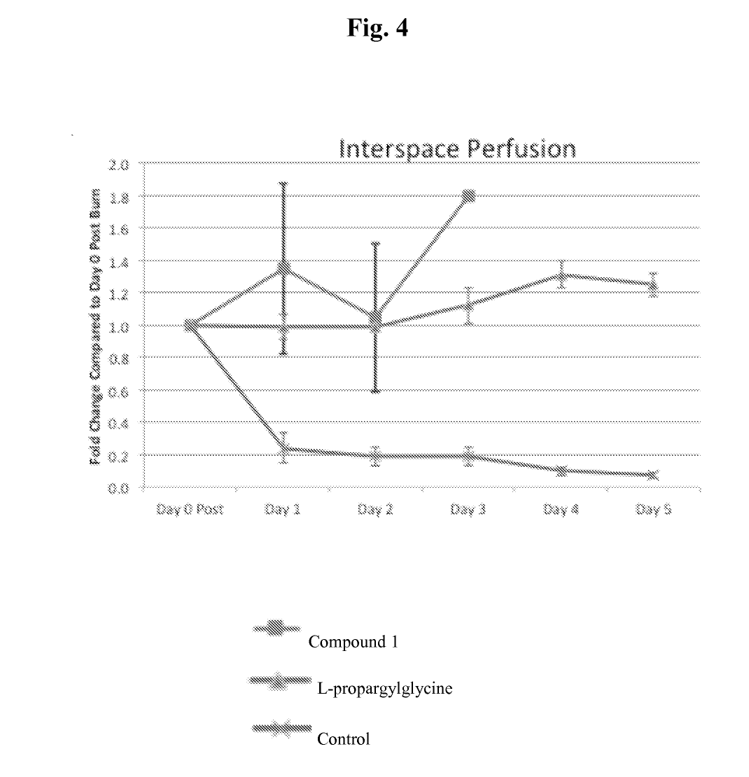

LDI analysis revealed a decrease in perfusion over time in the interspace areas in control animals (FIG. 4). This corresponds with the conversion of these areas to more damaged, less viable tissue. Conversely, perfusion is maintained in the zones of stasis in animals treated with L-propargylglycine (FIG. 4). A similar trend of maintenance of perfusion is indicated in animals treated with Compound 1 (FIG. 4).

### Example 3-3

### Dose Response to dl-Propargylglycine (PAG) in the Rat Hypoxic Ventilatory Response (HVR) Assay

### Animals

Male Sprague Dawley rats weighing 343±17 g (Mean±SD); weight range 316-381 g, were obtained from Harlan Laboratories and maintained on a 12 hour light:dark cycle (6 am lights on) with food and water ad libitum.

### Apparatus

DSI: Rat unrestrained whole body plethysmography chamber (˜8″ diameter plexiglass), ACQ 7700 Acquisition Interface, Validyne DP45 low range differential pressure transducers, Ponemah software.

Sable Systems FC-10, CA-10, FB-8, MFC-4, RH-300

Alicat MC-series mass flow control valves

Nitrogen, Oxygen, Carbon Dioxide, Air

### Drugs and Administration

Male Saline (0.9% NaCl) was obtained from Baxter Scientific (Lot#C802850).

dl-Propargyl Glycine (PAG) was obtained from Sigma (Cat #P7888; Lot#BCBD1765V) and solubilized in 0.9% NaCl. Drugs were prepared fresh on the day of use.

Test compounds were administered via the intraperitoneal route at a dose volume of 3 mUkg.

### Methods

Animals were pre-habituated to the test environment on 3 separate 1-hour periods prior to the test day. Following administration of test compounds animals were placed inside the whole body plethysmography chamber. The animals were allowed to acclimate to the environment for 60 minutes while breathing a 21% O₂ balance N₂ gas mix. After acclimation, the following gas mix protocol was followed:

| Cycle | Dura- | Oxy- | Nitro- | |
|---|---|---|---|---|
| # | tion | gen % | gen % | Samples made |
| 1 a | 30 min | 21 | 79 | Baseline/rat1/rat2/rat3/rat4 |
| 1 b | 30 min | 10 | 90 | Baseline/rat1/rat2/rat3/rat4 |
| 2 a | 30 min | 21 | 79 | Baseline/rat2/rat3/rat4/rat1 |
| 2 b | 30 min | 10 | 90 | Baseline/rat2/rat3/rat4/rat1 |
| 3 a | 30 min | 21 | 79 | Baseline/rat3/rat4/rat1/rat2 |
| 3 b | 30 min | 10 | 90 | Baseline/rat3/rat4/rat1/rat2 |

Cycles were designated to be the periods of both normoxia and hypoxia. Each experiment consisted of 3 cycles (or repeated sets) of the HVR to a normoxic-hypoxic shift. The cycles were 30 minutes in duration to allow for serial sampling from baseline and all 4 WBP test chambers. Each sampling period was of 4 minutes duration and was in a varying order but was always preceded by a 4 minute baseline. The sampling periods were calculated to allow for equilibration of the gas mixture in the WBP chamber. Switching of the multiplexer (MUX) line sampler system was achieved through use of a user defined program. The gas mixtures were likewise switched automatically using a user defined custom mix program and the Sable Systems/Alicat Multi Flow Controller utility.

Analysis of data was performed in GraphPad Prism (v5) using embedded 1-way Analysis of Variance (ANOVA) followed by Dunnett's or Tukey's MCT post hoc testing where appropriate. Dual data sets were analyzed using the Student's t-test. Bartlett's Equal Variance tests were performed on all data sets, however failure of equal variance was not considered to impinge the validity of the test.

### Results

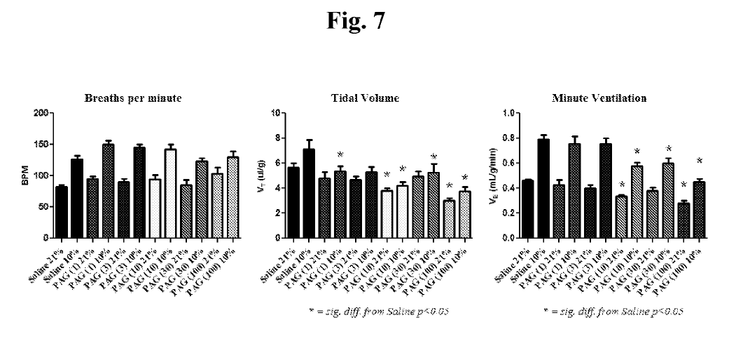

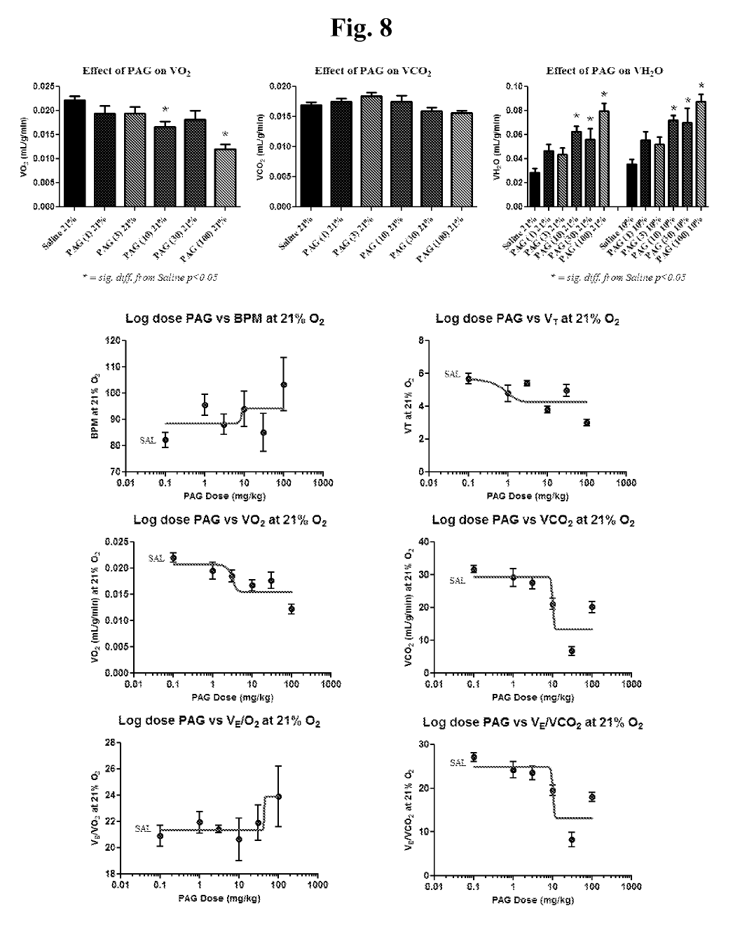

Male SD rats subjected to a change in FiO₂ from 21% to 10% show typical respiratory and metabolic function accommodation to the hypoxic change. An increase in tidal volume and respiratory rate produces an increase in the minute ventilation (V_{E}) of approximately 75% in saline treated rats when readings are averaged across 3 cycles of normoxia to hypoxia (FIGS. 7, 8 & 9). PAG treatment blunted the increase in V_{E} when administered at 10, 30 or 100 mg/kg but not at 1 or 3 mg/kg. V_{E} at an FiO2 of 21% was reduced significantly by PAG at 10 and 100 mg/kg when compared to saline (p<0.05, ANOVA+Dunnett's) and at 10% FiO₂, PAG at 10, 30 and 100 mg/kg significantly reduced V_{E} when compared to saline (p<0.05, ANOVA+Dunnett's) (FIG. 5).

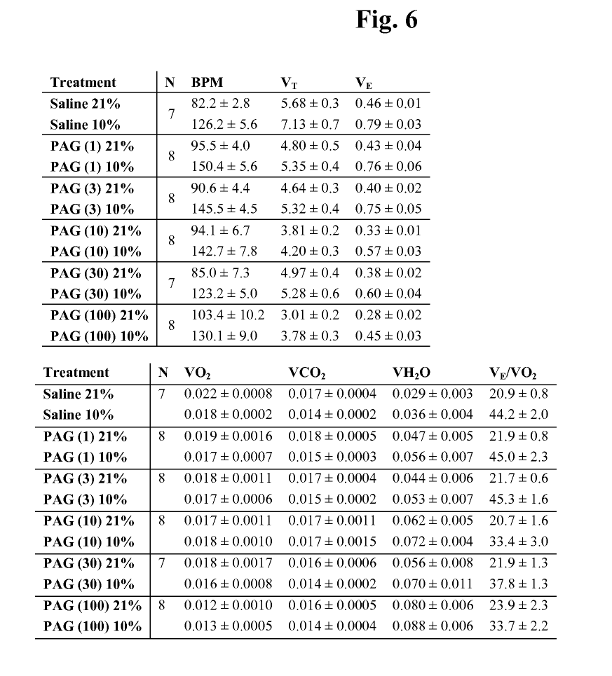

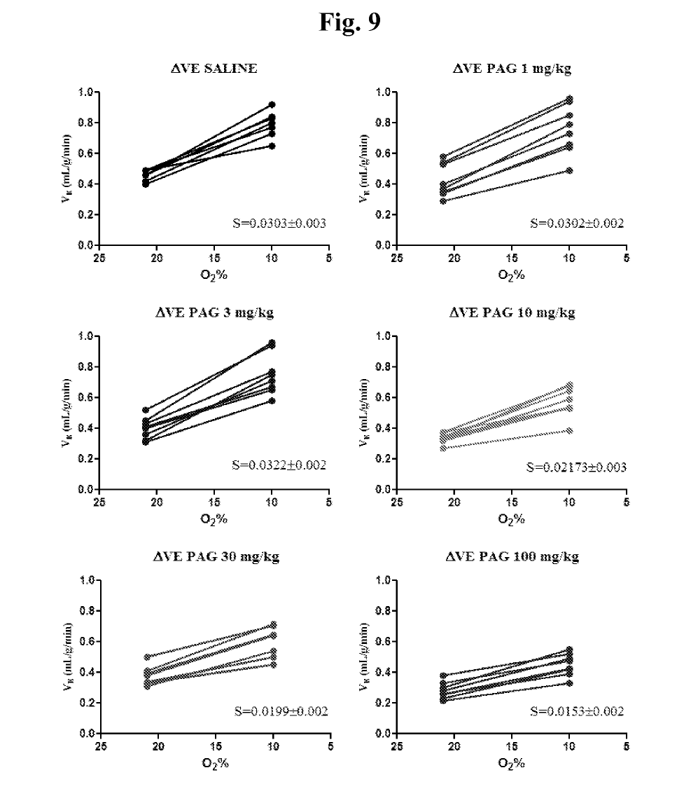

Under normoxic conditions PAG significantly decreased VO₂ at 10 and 100 mg/kg (p<0.05; ANOVA+Dunnett's MCT; FIGS. 6 and 8). There was no effect of PAG on VCO₂. VH2O was significantly impacted by PAG at 10, 30 and 100 mg/kg under both normoxic and hypoxic conditions (p<0.05, ANOVA+Dunnett's MCT; FIGS. 6 and 8). The hypoxic ventilatory response or the change in slope of minute ventilation to a shift in FiO₂ from 21% to 10% was significantly impacted by PAG. We measured the slope of the response (ΔV_{E})). PAG had no effect below a dose of 10 mg/kg but at 30 and 100 mg/kg significantly reduced ΔV_{E} (FIGS. 6, 9 & 10; p<0.05 ANOVA+Dunnett's).

### Example 3-4

### Effect of Carotid Sinus Nerve Transaction on the Rat Hypoxic Ventilator Response (HVR)

### Animals

Male Sprague Dawley rats from Harlan Laboratories weighing 350±31 g (Mean±SD); were maintained on a 12 hour light:dark cycle (6 am lights on) with food and water ad libitum.

### Apparatus

DSI: Rat unrestrained whole body plethysmography chamber (˜8″ diameter plexiglass), ACQ 7700 Acquisition Interface, Validyne DP45 low range differential pressure transducers, Ponemah software.

Sable Systems FC-10, CA-10, FB-8, MFC-4, RH-300

Alicat MC-series mass flow control valves

Nitrogen, Oxygen, Air

### Drugs and Administration

Male Saline (0.9% NaCl) was obtained from Baxter Scientific (Lot#C802850).

L-Propargyl Glycine (L-PAG) was solubilized in saline at 33.3 mg/ml.

Test KetaVed (ketamine HCl), AnaSed (xylazine) and Metacam (meloxicam) were obtained from MWI Veterinary Supply. A 90 mg/kg ketamine/10 mg/kg xylazine mixture was administered at 3 ml/kg with co vehicle saline. Meloxicam was administered at 1 mg/kg.

### Methods

Carotid sinus nerve (CSN) transaction—Under appropriate anesthesia the rat's neck is shaved and prepped for surgery with betadine solution. An incision is made in the throat area extending about 2 cm. The underlying musculature is incised to expose the carotid bifurcation on both sides. A silk suture is looped around the proximal external carotid and retracted laterally for control. The superior cervical ganglion is exposed where it lies dorsal to the carotid bifurcation and connective tissue are removed to expose the hypoglossal nerve bundle. The glossopharyngeal nerve is exposed as it lies under (superior to) the hypoglossal nerve. The carotid sinus nerve is transected where it originates on the exposed part of the glossopharyngeal and ascends to the carotid bifurcation.

Animals were pre-habituated to the test environment on 2 separate 45 minute periods prior to the test day. Following administration of test compounds animals were placed inside the whole body plethysmography chamber. The animals were allowed to acclimate to the environment for 60 minutes while breathing a 21% O₂ balance N₂ gas mix. After acclimation, the following gas mix protocol was followed:

| Cycle | Dura- | Oxy- | Nitro- | |
|---|---|---|---|---|
| # | tion | gen % | gen % | Samples made |
| 1 a | 30 min | 21 | 79 | Baseline/rat1/rat2/rat3/rat4 |
| 1 b | 30 min | 10 | 90 | Baseline/rat1/rat2/rat3/rat4 |
| 2 a | 30 min | 21 | 79 | Baseline/rat2/rat3/rat4/rat1 |
| 2 b | 30 min | 10 | 90 | Baseline/rat2/rat3/rat4/rat1 |

Cycles were designated to be the periods of both normoxia and hypoxia. Each experiment consisted of 2 cycles (or repeated sets) of the HVR to a normoxic-hypoxic shift. The cycles were 30 minutes in duration to allow for serial sampling from baseline and all 4 WBP test chambers. Each sampling period was of 4 minutes duration and was in a varying order but was always preceded by a 4 minute baseline. The sampling periods were calculated to allow for equilibration of the gas mixture in the WBP chamber. Switching of the multiplexer (MUX) line sampler system was achieved through use of a user defined program. The gas mixtures were likewise switched automatically using a user defined custom mix program and the Sable Systems/Alicat Multi Flow Controller utility.

Analysis of data was performed in GraphPad Prism (v5) using embedded 1-way Analysis of Variance (ANOVA) followed by Dunnett's or Tukey's MCT post hoc testing where appropriate. Bartlett's Equal Variance tests were performed on all data sets, however failure of equal variance was not considered to impinge the validity of the test.

### Results

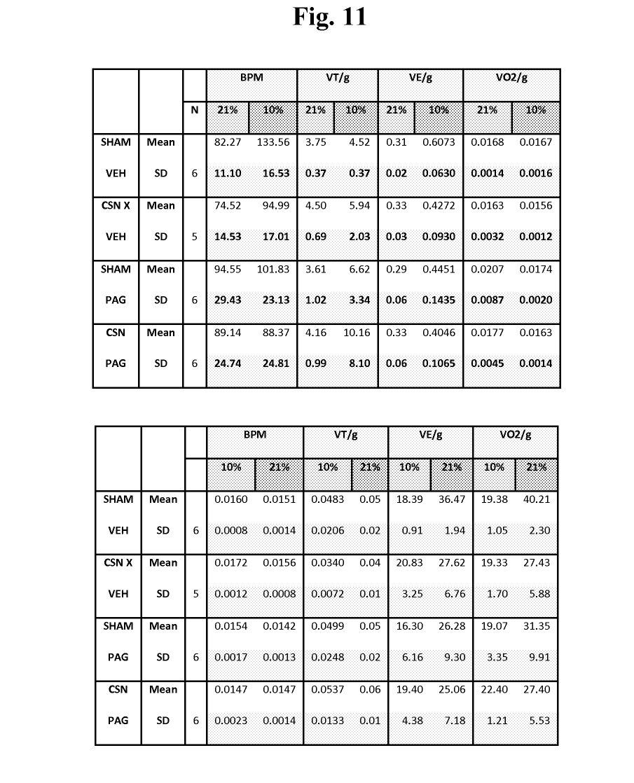

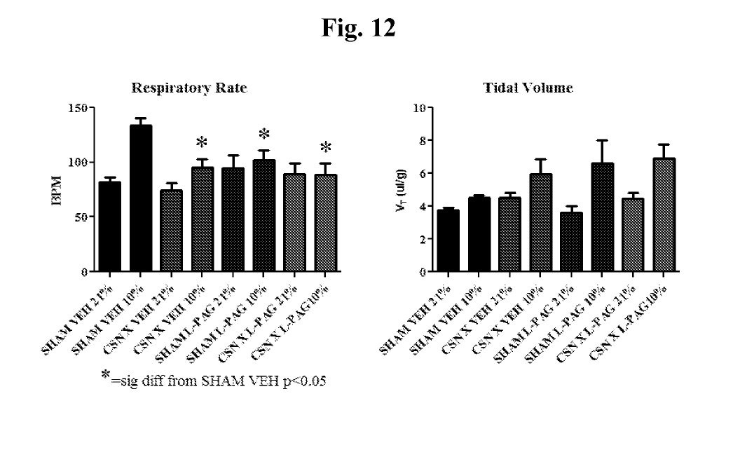

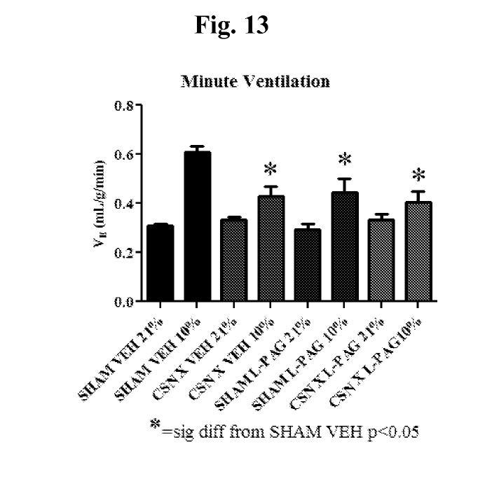

Sham rats subjected to a change in FiO₂ from 21% to 10% show typical respiratory and metabolic function accommodation to the hypoxic change. Minute ventilation (V_{E}) was increased by 95% when FiO₂ was changed from 21% to 10% with saline. Following CSN transection minute ventilation increased by only 29%. When 100 mg/kg L-PAG was administered prior to testing the V_{E} increased by 51% and 22%, respectively, in sham and CSN transection animals (FIGS. 11, 12 and 13).

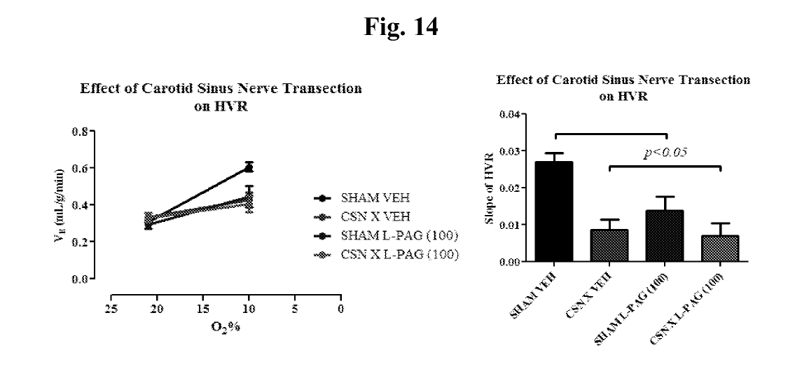

The hypoxic ventilatory response or the change in slope of minute ventilation to a shift in FiO₂ from 21% to 10% was significantly impacted by CSN transection with and without L-PAG at 100 mg/kg (p<0.05 ANOVA+Tukey's). 100 mg/kg L-PAG significantly reduced HVR in sham animals (p<0.05 ANOVA+Tukey's) but did not change the HVR in CSN transection animals suggesting that the effect of L-PAG in sham animals was directed through the intact CB/CSN (FIG. 14).

### Example 3-5

### Administration of CSE inhibitor to Treat AOP

A premature infant born at 30 weeks is diagnosed with Apnea of Prematurity (AOP) after he experiences several episodes of cessation of respiration for 20 seconds. The infant is administered a CSE inhibitor intravenously. The infant receives a loading dose of the CSE inhibitor at a concentration of 6 mg/kg. The dose is then lowered to 3 mg/kg every 10 hours. The number of apneas declines. There are no apneas after 35 weeks of age. The treatment is stopped at 37 weeks of age.

### Example 3-6

### Combination of CSE inhibitor and CPAP Therapy to Treat AOP

A premature infant born at 32 weeks is diagnosed with Apnea of Prematurity (AOP) after he experiences cessation of breathing for 10 seconds accompanied by bradycardia. The infant is administered CPAP therapy with nasal prongs. The CPAP is set at 5 cm H₂O. The infant is further administered a CSE inhibitor intravenously. The infant receives a loading dose of the CSE inhibitor at a concentration of 4 mg/kg. The dose is then lowered to 1 mg/kg every 8 hours. The number of apneas declines. There are no apneas after 34 weeks of age. The treatment is stopped at 36 weeks of age.

### Example 3-7

### Combination of CSE inhibitor and Aminophylline to Treat AOP

A premature infant born at 28 weeks is diagnosed with Apnea of Prematurity (AOP) after she experiences several episodes of cessation of breathing for 15 seconds. The infant is administered aminophylline. The loading dose of aminophylline is 2.5 mg/kg. The concentration is then reduced to 0.5 mg/kg and administered every 12 hours. The infant is further administered a CSE inhibitor intravenously. The infant receives a loading dose of the CSE inhibitor at a concentration of 2 mg/kg. The dose is then lowered to 0.5 mg/kg and administered every 12 hours. There are no apneas after 31 weeks of age. The treatment is stopped at 32 weeks of age.

### Example 3-8

### Clinical Trial

The purpose of this study is to assess if Compound 1 is as safe and efficacious as a patch to achieve wound healing in subjects with burn injuries.

Ages Eligible for Study: 6 Years to 65 Years. Genders Eligible for Study: Both.

Study Type: Interventional.

Study Design:

Allocation: Randomized.

Intervention Model: Single Group Assignment

Masking: Open Label

Primary Purpose: Treatment

Inclusion Criteria: Written informed consent obtained from either the subject or the subject's legally acceptable representative prior to screening activities. Total burn injuries measuring <=20% TBSA to include a deep partial thickness/full thickness area. The selected test area consisting of a contiguous, deep partial thickness/full thickness burn wound between 2% and 8% TBSA, which can be divided into two approximate halves or two bilateral injuries (each measuring between 1% and 4% TBSA).

Exclusion Criteria: 4th or 5th degree burns. Test area with infection as determined clinically. Venous or arterial vascular disorder directly affecting a designated test area. Known immune deficiency disorder, either congenital or acquired. Chronically malnourished as determined clinically by the investigator prior to surgery (Investigators are responsible for determining subjects are chronically malnourished during the screening process. Severe respiratory problems or concurrent head trauma at hospital admission. Any chronic condition requiring the use of systemic corticosteroids 30 days prior to study entry and anytime during the course of the study. Known or newly diagnosed diabetics requiring insulin. Concurrent participation in another clinical trial in which an investigational agent is used. (Subjects must not have been enrolled in another clinical trial within 30 days of enrolling in this trial).

40 patients are enrolled. Patients are treated with Compound 1 (oral dose, twice daily) for 1 week. The effects of Compound 1 therapy is determined by clinical observation and assessment of patient health via interviews with patients.

### Example 3-9

### Assay for Testing Tissue CSE Inhibitory Activity

H₂S biosynthesis in tissue homogenates was measured as follows: skin tissue was homogenized in ice-cold 100 mM potassium phosphate buffer (pH 7.4). An optimal w/v ratio of 1:10, as previously determined from experiments, was used. The assay mixture (500 μl) contained tissue homogenate (430 μl), L-cysteine (10 mM; 20 μl), pyridoxal 5′-phosphate (2 mM; 20 μl), and saline (30 μl) or in some cases DL-propargylglycine (30 μl, 25-2500 μM). Incubation was carried out in tightly sealed eppendorf vials. After incubation (37° C., 30 min), zinc acetate (1% w/v, 250 μl) was injected to trap generated H₂S followed by trichloroacetic acid (10% w/v, 250 μl) to precipitate protein and thus stop the reaction. Subsequently, N,N-dimethyl-p-phenylenediamine sulfate (20 μM; 133 μl) in 7.2 M HCl was added followed by FeCl₃ (30 μM; 133 μl) in 1.2 M HCl, and absorbance (670 nm) of aliquots of the resulting solution (300 μl) was determined 15 minutes thereafter using a 96-well microplate reader (Tecan Systems Inc.).

All standards and samples were assayed in duplicate. The H₂S concentration of each sample was calculated according to a standard curve of NaHS (3.125-250 μM) and the enzyme activity was expressed as nanomoles H₂S formed per milligram of tissue samples. Soluble protein mass in the skin tissue was determined using the Bradford assay, (Bio-Rad, Hercules, Calif.).

### Example 3-10

### Clinical Trial to Determine Efficacy of CSE Inhibitor as Adjuvant to CPAP in Individuals with Cheyne-Stokes Breathing

Study Type: Interventional

Allocation: Randomized

Endpoint Classification: Efficacy Study

Intervention Model: Parallel Assignment

Masking: Double Blind (Subject, Caregiver, Investigator, Outcomes Assessor)

Primary Purpose: Treatment

Primary Outcome Measures: Change in the Apnea Hypopnea Index (AHI)

Secondary Outcome Measures: Safety and tolerability

### Intervention

CSE inhibitor compound of Formula (1-I), (1-II), (1-IIa), (1-III), (1-IV), (1-IVa), (2-I), (2-II), (2-III), (2-IV), (2-V), or (2-VI) is taken orally 1 hour before bedtime at a dose of 15 mg/kg. One hour after CSE inhibitor is taken, patient utilizes CPAP device. Study duration is 4 weeks.

### Inclusion Criteria:

Presence of Congestive heart failure (CHF) defined as ejection fraction ≦40% by history of echocardiography data and New York heart Association (NYHA) class II-III and of typical cyclic crescendo and decrescendo change in breathing amplitude AHI≧10 and <60 and majority of the apneas to be ≧60% central in origin.

Loop-Gain of greater than 1

No response to treatment with CPAP

### Exclusion Criteria:

Loop-Gain less than 1

Positive response to CPAP

Unstable angina pectoris

Acute coronary syndrome less then 3 months ago

Stroke less then 6 weeks ago

Thoracal myopathy

Advanced COPD

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for treating a cutaneous injury or condition selected from a cutaneous burn, a cutaneous contracture, cutaneous scarring, cutaneous skin ulcers, pustules, blisters, staphylococcal scalded skin syndrome, toxic epidermal necrolysis, Stevens-Johnson Syndrome, epidermolysis bullosa and toxic shock syndrome in an individual in need thereof comprising administering a therapeutically effective amount of a cystathionine gamma lyase (CSE) inhibitor to the individual in need thereof.

2. The method of claim 1, wherein the cutaneous injury or condition is a cutaneous burn.

3.-10. (canceled)

11. The method of claim 1, wherein the CSE inhibitor is administered orally, intravenously, topically on the skin, or as a wash for the affected area.

12.-14. (canceled)

15. The method of claim 1, wherein the CSE inhibitor is administered in combination with an anti-inflammatory agent, a pain medication, an antiseptic agent, a local anesthetic, or a wound dressing.

16.-22. (canceled)

23. The method of claim 1, wherein the CSE inhibitor is L-propargylglycine or beta-cyanoalanine.

24. (canceled)

25. The method of claim 1, wherein the CSE inhibitor is a compound of Formula (1-I) having the structure: 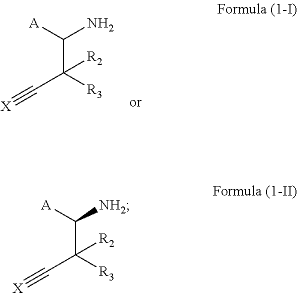
wherein:
A is a carboxylic acid isostere;
X is CR₁, or N;
R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl; and
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;
or a pharmaceutically acceptable salt, solvate, or prodrug thereof.

26. (canceled)

27. The method of claim 25 wherein A is a carboxylic acid isostere selected from

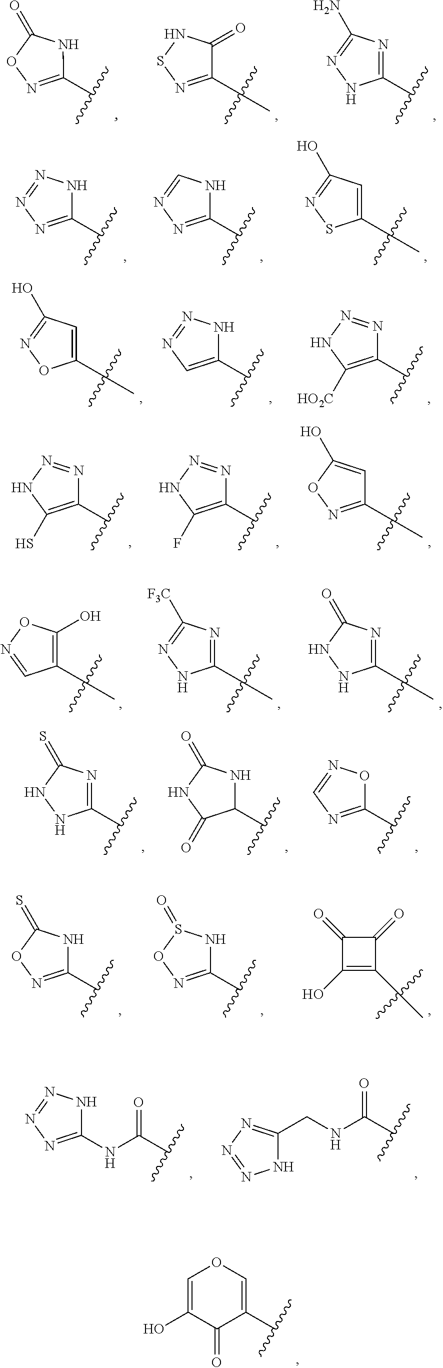

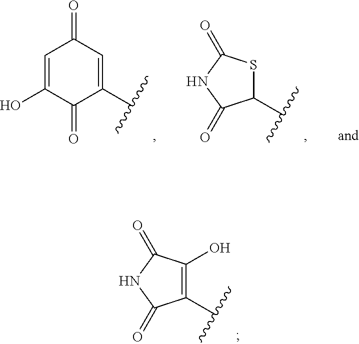 [SMILES: *C1=CC(=O)C=C(O)C1=O ; *C1SC(=O)NC1=O ; *C1=C(O)C(=O)NC1=O]

28. (canceled)

29. The method of claim 25 wherein X is N.

30. The method of claim 25 wherein X is CR₁.

31. The method of claim 30 wherein R₁ is H, substituted or unsubstituted alkyl, or substituted or unsubstituted heteroalkyl.

32. The method of claim 31 wherein R₁ is H.

33. The method of claim 31 wherein R₁ is CH₃.

34. The method of claim 32 wherein R₂ and R₃ are each H, and A is

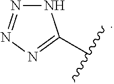 [SMILES: *c1nnn[nH]1]

35. The method of claim 1, wherein the CSE inhibitor is a compound of Formula (2-I) having the structure: 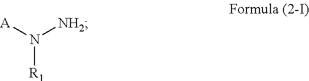
wherein:
A is a carboxylic acid isostere; and
R₁ is substituted or unsubstituted C₃-C₆alkyl, substituted or unsubstituted heteroalkyl, substituted or unsubstituted heterocycloalkyl, substituted or unsubstituted aryl, or substituted or unsubstituted heteroaryl; or a pharmaceutically acceptable salt, solvate, or prodrug thereof.

36. The method of claim 1, wherein the CSE inhibitor is a compound of Formula (2-II) having the structure: 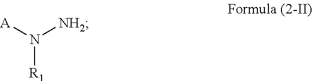
wherein:
R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl; and
A is selected from 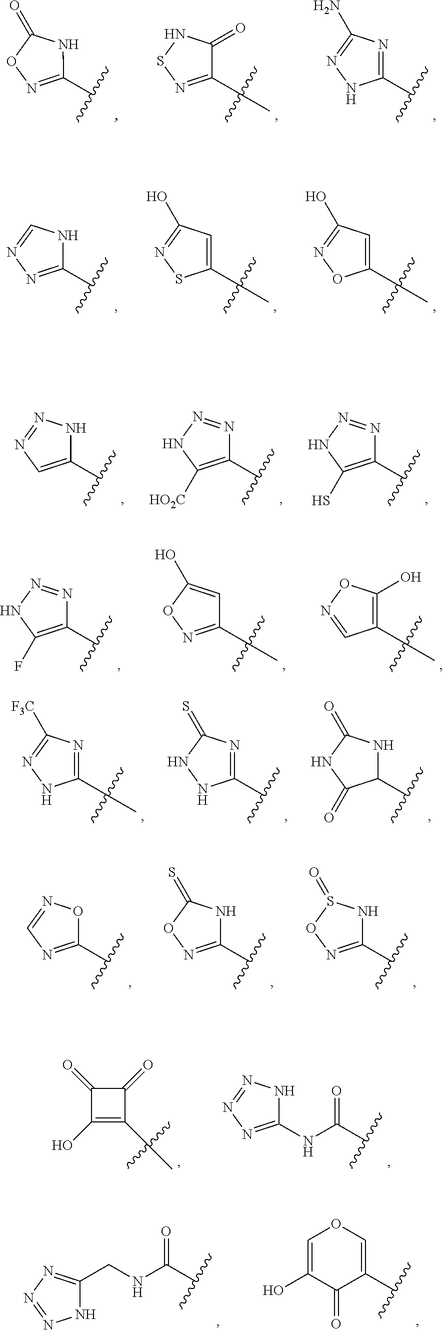 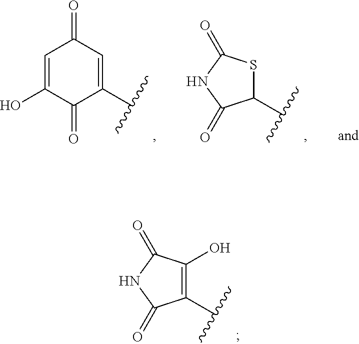 [SMILES: *C1=CC(=O)C=C(O)C1=O ; *C1SC(=O)NC1=O ; *C1=C(O)C(=O)NC1=O]
a pharmaceutically acceptable salt, solvate, or prodrug thereof.

37.-39. (canceled)

40. The method of claim 36 wherein R₁ is H.

41. The method of claim 36 wherein R₁ substituted or unsubstituted C₁-C₄alkyl.

42. (canceled)

43. (canceled)

44. The method of claim 1, wherein the CSE inhibitor is a compound of Formula (2-IV) having the structure: 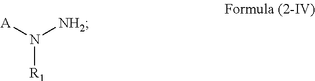
wherein:
A is 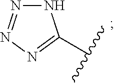 [SMILES: *c1nnn[nH]1]
and
R₁ is substituted or unsubstituted C₂-C₆alkyl, substituted or unsubstituted heteroalkyl, substituted or unsubstituted heterocycloalkyl, substituted or unsubstituted aryl, or substituted or unsubstituted heteroaryl; or a pharmaceutically acceptable salt, solvate, or prodrug thereof.

45. (canceled)

46. The method of claim 44 wherein R₁ is H.

47. (canceled)

48. The method of claim 44 wherein R₁ is —CH₂CH₃.

49.-52. (canceled)

53. The method of claim 1, wherein the CSE inhibitor is 2-aminopent-4-ynoic acid, (S)-2-aminopent-4-ynoic acid, 2-amino-3-cyanopropanoic acid, (S)-2-amino-3-cyanopropanoic acid, 2-hydrazinylacetic acid hydrochloride, 2-(2-(propan-2-ylidene)hydrazinyl)acetic acid, 4-((2-(1H-tetrazol-5-yl)hydrazinyl)methyl)-N,N-dimethylaniline, (E)-4-((2-(1H-tetrazol-5-yl)hydrazono)methyl)-N,N-diethylaniline, (E)-1-((2-(1H-tetrazol-5-yl)hydrazono)methyl)naphthalen-2-ol, (E)-5-(2-(benzo[d][1,3]dioxol-5-ylmethylene)hydrazinyl)-1H-tetrazole, (E)-4-((2-(1H-tetrazol-5-yl)hydrazono)methyl)phenol, (E)-5-(2-(4-nitrobenzylidene)hydrazinyl)-1H-tetrazole, (E)-5-(2-(furan-2-ylmethylene)hydrazinyl)-1H-tetrazole, 5-hydrazinyl-1H-tetrazole, 5-(1-methylhydrazinyl)-1H-tetrazole, 5-(1-methylhydrazinyl)-1H-1,2,4-triazol-3(2H)-one, 5-(1-ethylhydrazinyl)-1H-1,2,4-triazol-3(2H)-one, or 5-(hydrazinylmethyl)-1H-tetrazole.
